SEQUENCE LISTING (58 sequences)

SEQ ID NO: 1 - DNA, 1260 bp, Severe acute respiratory syndrome coronavirus 2
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 2 - DNA, 3822 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 3 - DNA, 21290 bp, Severe acute respiratory syndrome coronavirus 2
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 4 - DNA, 197 bp, Artificial Sequence
  synthetic polynucleotide
cggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaag

SEQ ID NO: 5 - DNA, 36 bp, Artificial Sequence
  synthetic oligonucleotide
  misc_feature (1)..(2): phosphorothioate bond
  misc_feature (2)..(3): phosphorothioate bond
  misc_feature (3)..(4): phosphorothioate bond
  misc_feature (4)..(5): phosphorothioate bond
  misc_feature (5)..(6): phosphorothioate bond
  misc_feature (6)..(7): phosphorothioate bond
tttttttgggttatcttcaacctaggacttttctat

SEQ ID NO: 6 - DNA, 30 bp, Artificial Sequence
  synthetic oligonucleotide
ccaacctgaagaagaatcaccaggagtcaa

SEQ ID NO: 7 - DNA, 32 bp, Artificial Sequence
  synthetic oligonucleotide
  misc_feature (1)..(1): 5' 6-FAM (fluorescein)
tttttttttttttttaggagtcaaataacttc

SEQ ID NO: 8 - DNA, 39 bp, Artificial Sequence
  synthetic oligonucleotide
  misc_feature (1)..(2): phosphorothioate bond
  misc_feature (2)..(3): phosphorothioate bond
  misc_feature (3)..(4): phosphorothioate bond
  misc_feature (4)..(5): phosphorothioate bond
  misc_feature (5)..(6): phosphorothioate bond
  misc_feature (6)..(7): phosphorothioate bond
  misc_feature (39)..(39): 3' biotin
tttttttatgtaaagcaagtaaagttttttttttttttt

SEQ ID NO: 9 - DNA, 22 bp, Artificial Sequence
  synthetic oligonucleotide
gaccratcctgtcacctctgac

SEQ ID NO: 10 - DNA, 24 bp, Artificial Sequence
  synthetic oligonucleotide
agggcattytggacaaakcgtcta

SEQ ID NO: 11 - DNA, 22 bp, Artificial Sequence
  synthetic oligonucleotide
tcctcaactcactcttcgagcg

SEQ ID NO: 12 - DNA, 21 bp, Artificial Sequence
  synthetic oligonucleotide
cggtgctcttgaccaaattgg

SEQ ID NO: 13 - DNA, 20 bp, Artificial Sequence
  synthetic oligonucleotide
ttccgacgtgctcgaacttt

SEQ ID NO: 14 - DNA, 21 bp, Artificial Sequence
  synthetic oligonucleotide
ccaacacggttgtgacagtga

SEQ ID NO: 15 - DNA, 17 bp, Artificial Sequence
  synthetic oligonucleotide
tcctccggcccctgaat

SEQ ID NO: 16 - DNA, 23 bp, Artificial Sequence
  synthetic oligonucleotide
gaaacacggacacccaaagtagt

SEQ ID NO: 17 - DNA, 25 bp, Artificial Sequence
  synthetic oligonucleotide
tcttcatcaccatacttttctgtta

SEQ ID NO: 18 - DNA, 23 bp, Artificial Sequence
  synthetic oligonucleotide
gccaaaaaattgtttccacaata

SEQ ID NO: 19 - DNA, 28 bp, Artificial Sequence
  synthetic oligonucleotide
  misc_feature (1)..(1): 5' 6-FAM (fluorescein)
  misc_feature (9)..(10): ZEN quencher
  misc_feature (28)..(28): 3IABkFQ (3' Iowa Black FQ)
ccaccaataatgatagagtcagcacaca

SEQ ID NO: 20 - DNA, 197 bp, Artificial Sequence
  synthetic polynucleotide
cttctctggatttaacacactttctgtacaatccctttgagtgcgtgacaaatgtttcacctaaattcaaggctttaagtttagctccaccaataatgatagagtcagcacacaaagccaaaaatttatttacaagcttaaagaatgtctgaacactctccttaatttcctttgcacaggtgacaatttgtccaccg

SEQ ID NO: 21 - DNA, 18 bp, Artificial Sequence
  synthetic oligonucleotide
cggtggacaaattgtcac

SEQ ID NO: 22 - DNA, 22 bp, Artificial Sequence
  synthetic oligonucleotide
cttctctggatttaacacactt

SEQ ID NO: 23 - DNA, 51 bp, Artificial Sequence
  synthetic oligonucleotide
tcagcacacaaagccaaaaatttatttttctgtgcaaaggaaattaaggag

SEQ ID NO: 24 - DNA, 49 bp, Artificial Sequence
  synthetic oligonucleotide
tattggtggagctaaacttaaagccttttctgtacaatccctttgagtg

SEQ ID NO: 25 - DNA, 28 bp, Artificial Sequence
  synthetic oligonucleotide
ttacaagcttaaagaatgtctgaacact

SEQ ID NO: 26 - DNA, 27 bp, Artificial Sequence
  synthetic oligonucleotide
ttgaatttaggtgaaacatttgtcacg

SEQ ID NO: 27 - DNA, 23 bp, Artificial Sequence
  synthetic oligonucleotide
tgagtacgaacttatgtactcat

SEQ ID NO: 28 - DNA, 22 bp, Artificial Sequence
  synthetic oligonucleotide
ttcagatttttaacacgagagt

SEQ ID NO: 29 - DNA, 42 bp, Artificial Sequence
  synthetic oligonucleotide
accacgaaagcaagaaaaagaagttcgtttcggaagagacag

SEQ ID NO: 30 - DNA, 44 bp, Artificial Sequence
  synthetic oligonucleotide
ttgctagttacactagccatccttaggttttacaagactcacgt

SEQ ID NO: 31 - DNA, 18 bp, Artificial Sequence
  synthetic oligonucleotide
cgctattaactattaacg

SEQ ID NO: 32 - DNA, 19 bp, Artificial Sequence
  synthetic oligonucleotide
gcgcttcgattgtgtgcgt

SEQ ID NO: 33 - DNA, 19 bp, Artificial Sequence
  synthetic oligonucleotide
tctattgccatacccacaa

SEQ ID NO: 34 - DNA, 23 bp, Artificial Sequence
  synthetic oligonucleotide
ggtgttttgtaaatttgtttgac

SEQ ID NO: 35 - DNA, 47 bp, Artificial Sequence
  synthetic oligonucleotide
cattcagttgaatcaccacaaatgtgtgttaccacagaaattctacc

SEQ ID NO: 36 - DNA, 45 bp, Artificial Sequence
  synthetic oligonucleotide
gttgcaatatggcagtttttgtacattgggtgtttttgtcttgtt

SEQ ID NO: 37 - DNA, 24 bp, Artificial Sequence
  synthetic oligonucleotide
actgatgtcttggtcatagacact

SEQ ID NO: 38 - DNA, 25 bp, Artificial Sequence
  synthetic oligonucleotide
taaaccgtgctttaactggaatagc

SEQ ID NO: 39 - DNA, 19 bp, Artificial Sequence
  synthetic oligonucleotide
aagatgagattggcatggc

SEQ ID NO: 40 - DNA, 19 bp, Artificial Sequence
  synthetic oligonucleotide
gcaagggacttcctgtaac

SEQ ID NO: 41 - DNA, 38 bp, Artificial Sequence
  synthetic oligonucleotide
ctccaaccgactgctgtctttggcttgactcaggattt

SEQ ID NO: 42 - DNA, 42 bp, Artificial Sequence
  synthetic oligonucleotide
cccaaagttcacaatgtggccgcatctcatatttggaatgac

SEQ ID NO: 43 - DNA, 18 bp, Artificial Sequence
  synthetic oligonucleotide
accttcaccgttccagtt

SEQ ID NO: 44 - DNA, 22 bp, Artificial Sequence
  synthetic oligonucleotide
ggactttgattgcacattgttg

SEQ ID NO: 45 - DNA, 17 bp, Artificial Sequence
  synthetic oligonucleotide
ttgatgagctggagcca

SEQ ID NO: 46 - DNA, 18 bp, Artificial Sequence
  synthetic oligonucleotide
caccctcaatgcagagtc

SEQ ID NO: 47 - DNA, 41 bp, Artificial Sequence
  synthetic oligonucleotide
gtgtgaccctgaagactcggttttagccactgactcggatc

SEQ ID NO: 48 - DNA, 45 bp, Artificial Sequence
  synthetic oligonucleotide
cctccgtgatatggctcttcgtttttttcttacatggctctggtc

SEQ ID NO: 49 - DNA, 20 bp, Artificial Sequence
  synthetic oligonucleotide
atgtggatggctgagttgtt

SEQ ID NO: 50 - DNA, 20 bp, Artificial Sequence
  synthetic oligonucleotide
catgctgagtactggacctc

SEQ ID NO: 51 - DNA, 28 bp, Artificial Sequence
  synthetic oligonucleotide
  misc_feature (1)..(1): 5' Cy5
  misc_feature (9)..(10): TAO quencher
  misc_feature (28)..(28): 3IAbRQSp (3' Iowa Black RQ)
tgtgtgctgactctatcattattggtgg

SEQ ID NO: 52 - DNA, 29 bp, Artificial Sequence
  synthetic oligonucleotide
  misc_feature (1)..(1): 5' Cy5
  misc_feature (9)..(10): TAO quencher
  misc_feature (29)..(29): 3IAbRQSp (3' Iowa Black RQ)
ttgctttcgtggtattcttgctagttaca

SEQ ID NO: 53 - DNA, 28 bp, Artificial Sequence
  synthetic oligonucleotide
  misc_feature (1)..(1): 5' Cy5
  misc_feature (9)..(10): TAO quencher
  misc_feature (28)..(28): 3IAbRQSp (3' Iowa Black RQ)
actgaatgcagcaatcttttgttgcaat

SEQ ID NO: 54 - DNA, 28 bp, Artificial Sequence
  synthetic oligonucleotide
  misc_feature (1)..(1): 5' 6-FAM (fluorescein)
  misc_feature (9)..(10): ZEN quencher
  misc_feature (28)..(28): 3IABkFQ (3' Iowa Black FQ)
cggttggagcgagcatcccccaaagttc

SEQ ID NO: 55 - DNA, 30 bp, Artificial Sequence
  synthetic oligonucleotide
  misc_feature (1)..(1): 5' 6-FAM (fluorescein)
  misc_feature (9)..(10): ZEN quencher
  misc_feature (30)..(30): 3IABkFQ (3' Iowa Black FQ)
aagtaattgaaaagacactcctccacttat

SEQ ID NO: 56 - DNA, 52 bp, Artificial Sequence
  synthetic oligonucleotide
ttgactcctggtgattcttcttcaggttggccctccctccctccctcccttt

SEQ ID NO: 57 - DNA, 45 bp, Artificial Sequence
  synthetic oligonucleotide
ttgactcctggtgattcttcttcaggttggttttccaaccacttc

SEQ ID NO: 58 - DNA, 228 bp, Severe acute respiratory syndrome coronavirus 2
atgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaa

(19) United States
(12) Patent Application Publication
(10) Pub. No.: US 2023/0203567 A1
(43) Pub. Date: Jun. 29, 2023

(54) ISOTHERMAL METHODS, COMPOSITIONS, KITS, AND SYSTEMS FOR DETECTING NUCLEIC ACIDS

(71) Applicant: PRESIDENT AND FELLOWS OF HARVARD COLLEGE, Cambridge, MA (US)

(72) Inventors: Youngeun KIM, Cambridge, MA (US); Jocelyn Yoshiko KISHI, Cambridge, MA (US); Thomas E. SCHAUS, Cambridge, MA (US); Adam YASEEN, Cambridge, MA (US); Peng YIN, Cambridge, MA (US); Sinem K. SAKA, Cambridge, MA (US); Kuanwei SHENG, Cambridge, MA (US); Nikhil GOPALKRISHNAN, Cambridge, MA (US); Jiyoun JEONG, Cambridge, MA (US)

(73) Assignee: PRESIDENT AND FELLOWS OF HARVARD COLLEGE, Cambridge, MA (US)

(21) Appl. No.: 17/996,846

(22) PCT Filed: Apr. 21, 2021
(86) PCT No.: PCT/US2021/028426

§ 371 Date: Oct. 21, 2022

Related U.S. Application Data
(60) Provisional application No. 63/134,010, filed on Jan. 5, 2021.
(60) Provisional application No. 63/091,528, filed on Oct. 14, 2020.
(60) Provisional application No. 63/082,019, filed on Sep. 23, 2020.
(60) Provisional application No. 63/046,400, filed on Jun. 30, 2020.
(60) Provisional application No. 63/044,513, filed on Jun. 26, 2020.
(60) Provisional application No. 63/024,084, filed on May 13, 2020.
(60) Provisional application No. 63/019,018, filed on May 1, 2020.
(60) Provisional application No. 63/013,818, filed on Apr. 22, 2020.

(51) Int. Cl. C12Q 1/6816; C12Q 1/6853; C12N 15/113

(52) U.S. Cl. CPC C12Q 1/6816; C12N 15/1137; C12Q 1/6853; C12Q 2600/16

(57) ABSTRACT

The technology described herein is directed to methods, kits, compositions, devices, and systems for detecting a target nucleic acid, such as a viral RNA. In one aspect, described herein are methods of detecting the target nucleic acid. In other aspects, described herein are compositions, kits, devices, and systems suitable to practice the methods described herein to detect the target nucleic acid.

### CROSS-REFERENCE TO RELATED APPLICATIONS

This application claims benefit under 35 U.S.C. § 119(e) of U.S. Provisional Application No. 63/013,818 filed Apr. 22, 2020; U.S. Provisional Application No. 63/019,018 filed May 1, 2020; U.S. Provisional Application No. 63/024,084 filed May 13, 2020; U.S. Provisional Application No. 63/044,513 filed Jun. 26, 2020; U.S. Provisional Application No. 63/046,400 filed Jun. 30, 2020; U.S. Provisional Application No. 63/082,019 filed Sep. 23, 2020; U.S. Provisional Application No. 63/091,528 filed Oct. 14, 2020; U.S. Provisional Application No. 63/134,010 filed Jan. 5, 2021; the contents of each of which are incorporated herein by reference in their entireties.

### GOVERNMENT SUPPORT

This invention was made with government support under Grant No GM133052 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Apr. 20, 2021, is named 002806-097400WOPT_SL.txt and is 49,222 bytes in size.

### TECHNICAL FIELD

The technology described herein relates to isothermal methods, compositions, kits, and systems for amplifying, detecting, and identifying nucleic acids.

### BACKGROUND

Recent innovations in isothermal amplification of specific target analyte sequences, paired with visual readout of the result have brought the prospect of highly sensitive point-of-care (POC) diagnostics that are fast, cheap, and use readily accessible equipment. For example, Loop-Mediated Isothermal Amplification (LAMP), recombinase polymerase amplification (RPA) and Helicase-dependent isothermal DNA amplification (HDA) are isothermal amplification methods that can be used to detect a target nucleic acid. However, detection of the amplicon in many of these assays are not specific or sensitive. For example, LAMP is frequently used to test for the presence or absence of specific nucleic acid targets in a sample by coupling the amplification with a reporting scheme. A reporting scheme is an observable output, like a color change or fluorescence emission, that is only produced when the target is present, or that shows a distinguishable difference from the output produced when no target is present. The two most common reporting schemes for LAMP are colorimetric output and fluorescent output. In colorimetric output, the LAMP reaction is supplemented with a dye (e.g. phenol red) that changes color in response to a change in pH. Amplification of DNA results in a change in the pH of the solution, which is visualized by the naked eye or a machine as a color change. In fluorescent output, the LAMP reaction is supplemented with a conditionally fluorescent DNA binding dye. The fluorescence increases significantly in the presence of DNA amplicons, which is detected by a fluorescent reader. The drawbacks of these reporting techniques are two-fold. First, they are not sequence specific and hence any spurious amplification (to which all amplification schemes are prone) will result in a false positive. Second, they cannot produce distinct reporting based on the target sequence and hence cannot distinguish between multiple targets.

RPA-amplified DNA detection schemes with lateral flow device (LFD) readout rely on non-DNA signals such as fluorophores or biotin, initially on separate primers but brought together during amplification. These have intrinsically limited specificity, since RPA is error prone, and primer ‘dimers’ or other non-specific connections result in positive signals on LFD. There have been several demonstrations the application of RPA products to LFDs for rapid visual detection of target amplicons, but they lack the capability of checking the target amplicon in a sequence specific way which would eliminate the problem of false positives from RPA background amplicons.

Thus, there is a great need for methods, compositions and kits for detecting target nucleic acids with minimal background during detection and that address one or more of the above noted issues.

### SUMMARY

In several aspects, the compositions and methods provided herein are based, in part, on the discovery of a scheme for sequence-specific reporting of nucleic acid targets using catalytic probe digestion.

In one aspect, provided herein is a method for detecting a target nucleic acid in a sample. Generally, the method comprises hybridizing a nucleic acid probe to an amplicon from amplification of a target nucleic acid. The probe comprises a reporter molecule capable of producing a detectable signal. The hybridized nucleic acid probe is cleaved with a double-strand specific exonuclease, e.g., an exonuclease having 5′ to 3′ exonuclease activity. After cleavage, the reporter molecule from the cleaved probe is detected. Alternatively, or in addition, detecting, e.g., with a sequence specific method, any remaining uncleaved nucleic acid probes.

The step of hybridizing the nucleic acid probe and/or cleaving the hybridized nucleic acid probe can be simultaneous with the amplification of the target nucleic acid. In some embodiments, the step of hybridizing the nucleic acid probe and/or cleaving the hybridized nucleic acid probe is after the amplification of the target nucleic acid.

In some embodiments, a detectable signal from the reporter molecule is quenched when the nucleic acid probe is not hybridized to the amplicon. For example, the detectable signal from the reporter molecule can be quenched by a quencher molecule. Accordingly, in some embodiments of any one of the aspects, the nucleic acid probe further comprises a quencher molecule capable of quenching a detectable signal produced by the reporter molecule. The quencher molecule can quench the detectable signal from the reporter molecule when the nucleic acid probe is not hybridized to the amplicon.

The nucleic acid probe can be designed to hybridize anywhere in the amplicon. Accordingly, in some embodiments of any one of the aspects, the nucleic acid probe can comprise a nucleotide sequence substantially complementary or identical to a nucleotide sequence of the target nucleic acid and/or a primer in used in the amplification of the target nucleic acid. For example, the nucleic acid probe can comprise a nucleotide sequence substantially identical to a primer used in the amplification of the target nucleic acid. In some embodiments, the nucleic acid probe comprises a nucleotide sequence substantially complementary to a nucleotide sequence at an internal position of the amplicon.

Generally, the nucleic acid probe hybridizes to a single-stranded region of the amplicon. Accordingly, in some embodiments, the method further comprises a step of preparing a single-stranded amplicon. For example, the target nucleic acid can be asymmetrically amplified to produce a single-stranded amplicon. In another non-limiting example, the target nucleic acid can be amplified to produce a double-stranded amplicon and a single-stranded amplicon prepared from the double-stranded amplicon. Exemplary methods for producing single-stranded amplicons are described herein. In some embodiments, the target nucleic acid can be amplified such that the amplicon comprises a single-stranded region, e.g., LAMP amplification.

In some embodiments of any one of the aspects, the step of hybridizing a probe with amplicon is in the presence of a surfactant e.g., SDS, and/or a reagent capable of hybridizing/localizing a single-strand nucleic acid strand to a double-stranded nucleic acid. Some exemplary reagents capable of localizing a single-strand nucleic acid strand to a double-stranded nucleic acid include, but are not limited to, recombinases, single-stranded binding proteins, Cas proteins, zinc finger nucleases, transcription activator-like effector nucleases (TALENs), and the like.

The method described herein can be performed in a device. For example, the methods described herein can be performed in a device comprising two or more chambers. In some embodiments, the device comprises means for moving a fluid irreversibly from a first chamber to a second chamber.

In yet another aspect, provided herein is a composition comprising: an exonuclease having 5′->3′ cleaving activity; a primer set for amplifying a target nucleic acid; and a nucleic acid probe comprising a reporter molecule.

In another aspect, provided herein is a kit for detecting a target nucleic acid in a sample, the kit comprising: an exonuclease having 5′->3′ cleaving activity; a primer set for amplifying a target nucleic acid; and a nucleic acid probe comprising a reporter molecule.

In some embodiments of any of the aspects provided herein, amplification is by LAMP and the primer set comprises a forward outer primer (F3), a reverse outer primer (R3), a forward inner primer (FIP), and a reverse inner primer (RIP). In some further embodiments of this aspects, the primer set further comprises a forward loop primer (LF) and a reverse loop primer (LR).

In some embodiments of any of the aspects provided herein, one or more components of a kit or a composition described herein can be disposed in a device. For example, a device comprising two or more chambers and means for moving a fluid irreversibly from a first chamber to a second chamber. One exemplary means for moving a fluid from a first chamber to a second chamber comprises actuation by a built-in spring. For example, a built-in spring whose potential energy is released by a solenoid trigger.

In some embodiments of any of the aspects provided herein, the device further comprises means for detecting a signal. For example, the device comprises means for detecting a fluorescent signal from the reporter molecule.

The methods, kits and compositions described herein can be used for multiplex detection of two or more target nucleic acid simultaneously.

### BRIEF DESCRIPTION OF THE DRAWINGS

FIGS. 1A-1B is a series of schematics showing strategies for creating ssDNA product from RPA amplification. FIG. 1A is a schematic showing that standard RPA can be used to produce double-stranded amplicons, followed by exonuclease-based digestion of one of the strands. The protected strand can have phosphorothioate (PT) bonds on its 5′ end or other modifications. The digestion of the other strand can be facilitated by a phosphorylated 5′ end (phos). FIG. 1B is a schematic showing that asymmetric RPA, whereby one primer (e.g. blue) is included in excess of the other one, can be used to generate double- and single-stranded products.

FIGS. 2A-2B is a series of schematics showing strategies for reducing potential spurious extension of ssRPA product. FIG. 2A is a schematic showing that stochastic termination of symmetrical RPA with stochastic dideoxynucleotide triphosphate (ddNTP) termination with intrinsic polymerase, additional polymerase, additional terminal transferase, or another enzyme can be used to prevent further extension of the sequence. As shown, SEQ ID NO: 56 is TTGACTCCTGGTGATTCTTCTTCAGGTTGGCCCTCCCTCCCTCCCTCCCTTT. FIG. 2B is a schematic showing that the 5′ end of the primer can also be modified or re-designed to either reduce the chance of the tail folding on the amplicon sequence, or as shown to promote the formation of a self-folding tail structure that should not extend. Hybridization modeled with NUPACK software. As shown, SEQ ID NO: 57 is TTGACTCCTGGTGATTCTTCTTCAGGTTGGTTTTCCAACCACTTC.

FIG. 3 shows RPA amplification of different copy numbers of RNA (starting material). Gel electrophoresis data indicates that RPA can successfully amplify product down to approximately 3 copies. As control, negative control (with no RNA template or starting material) was run on the same gel. “dsDNA” indicates post-RPA samples, when amplicons remain in double-stranded product. “ssDNA” indicates post-exo treatment, in which double-stranded product is digested to only leave single-stranded target band.

FIGS. 4A-4C is a series of schematics and images showing detection of ssDNA with Lateral Flow Devices (LFDs). FIG. 4A is a lateral flow device schematic with a single test line where latex bead-conjugates assemble only in the presence of a target. FIG. 4B is a series of images showing that a streptavidin test line can detect 10 pM of biotinylated target DNA, which tethers a nanoparticle-conjugated complementary strand in place. FIG. 4C is a series of images demonstrating multiplexed and patterned detection of two distinct nucleic acid sequences (e.g., DNA₁ and DNA₂) performed on a single LFD strip.

FIGS. 5A-5C is a series of schematics showing strategies for toehold-based detection of amplicons. FIG. 5A is a schematic showing that single-stranded amplicons can be detected through a toehold-mediated strand displacement reaction. The best specificity checks occur in the middle region of the sequence (e.g., Inner sequence, red rectangle), which would not be detectable on primer dimers that might be produced during the RPA step. FIG. 5B is a schematic showing that stoppers (e.g. spacers or other modifications that prevent polymerase extension) and extra sequences can be incorporated into one or both primers to create ssDNA tails in RPA products. These tails can then serve as toeholds for toehold-mediated strand displacement-based detection of the target amplicon sequence (e.g., rectangles indicate potential sequences to detect). FIG. 5C is a schematic showing that primers can contain a modification that can be cleaved after the RPA step to expose single-stranded tails (e.g. a Uracil base that is cleaved by the USER enzyme). These tails can then serve as toeholds for sequence-specific detection via toehold-mediated strand displacement. Displacement of complementary strands from desired target can be accomplished with two (or more) strands, one from a toehold at each end.

FIGS. 6A-6B is a series of schematics showing a full demonstration of the methods and assays described herein. FIG. 6A is a schematic showing that RPA amplification can occur in as little as 5 minutes, optionally followed by a short (e.g., 1 min) heat inactivation of RPA enzymes and exonuclease digestion of one strand (e.g., 1 min). FIG. 6B is a schematic showing that single-stranded target amplicons are detected using an LFD via sequence-specific hybridization. The correct target amplicon sequence successfully tethers a latex bead-conjugated complementary strand to the test line via another complementary biotinylated strand.

FIG. 7 is an image showing that LFD can detect amplified product of ~ 3 copies of RNA. LFD strips show a red test line that indicate presence of target (at red arrow that says “Detection”). RPA product without exonuclease treatment (still remaining in double-stranded product) cannot be detected on LFD. Therefore, only when ssRPA is applied (RPA+exo) can single-stranded target be detected.

FIG. 8 is a schematic comparing the present disclosure (e.g., ssRPA) to other SARS-CoV-2 methods, e.g., DNA endonuclease-targeted CRISPR trans reporter (DETECTR), specific high-sensitivity enzymatic reporter unlocking (SHERLOCK), and the quantitative reverse transcription polymerase chain reaction (qRT-PCR) workflow used by the Centers for Disease Control and Prevention (CDC) and the World Health Organization (WHO).

FIG. 9 shows an exemplary schematic of a system as described herein.

FIG. 10 shows a schematic for fluorescence readout of single-stranded amplicon sequence via toehold-mediated strand displacement. Fluorescence signal is produced upon displacement of a fluorophore-labeled strand from a proximal quencher-labeled strand.

FIGS. 11A-11C is a series of images and graphs showing experimental validation of a fluorescent readout. FIG. 11A is a schematic showing the workflow steps, HI = Heat Inactivation, exo + F/Q = combined lambda exonuclease digestion and incubation with fluorophore/quencher-labeled strands. FIG. 11B shows visible detection (left, image and text) and real-time PCR fluorescence detection (right, graph) of negative (no template RPA) and positive (approx. 10^5 copies of cultured and heat-inactivated SARS-CoV-2 genome RNA) samples. FIG. 11C shows validation (visible image to left, and fluorescence measurements in graph to right) of sequence-specific detection using a distinct viral input (Rhinovirus, heat-inactivated) as negative control and RPA amplicon from approximately 3 copies of SARS-CoV-2 genome RNA.

FIGS. 12A-12G is a series of schematics and images showing the ssRPA assay design, workflow, and characterization. FIG. 12A is a schematic showing that the key to ssRPA design is the rapid generation of millions of ssDNA copies from a single RNA target. ssDNA output offers straightforward specific readout by fluorescence or colorimetric/visual methods such as lateral flow devices. FIG. 12B is a schematic showing an exemplary ssRPA method. Step 1: Target viral RNA region (of domains a-b-c-d) is reverse transcribed into cDNA via extension of the reverse primer (d*) by the Reverse Transcriptase in the reaction mixture. Subsequently, the cDNA gets amplified via isothermal RPA at 42° C. by templated extension of the forward (a) and reverse primers (d*). The forward primer has a 6-nucleotide long polyT segment with phosphorothioate bonds. Step 2a: Products of RPA are transferred to the exo/LFD buffer that contains T7 exonuclease (a dsDNA-specific 5′ to 3′ exonuclease) and detection probes. The resulting mixture is incubated for 1 min at ambient temperature and reverse strand of the dsDNA amplicon products get preferentially digested yielding ssDNA amplicon (a-b-c-d) homologous to the target RNA sequence. Step 2b: The 3′ biotin (b*) and 5′ FAM (c*) modified detection probes make the assay directly compatible with commercially available test strips that feature a streptavidin test line and gold nanoparticles conjugated to rabbit anti-FAM IgG at the conjugate pad. Step 3: The test strip is vertically inserted into the resulting 50 µl mixture. The right ssDNA amplicon acts as a bridge that binds both the biotin-probe and the FAM-probe independently resulting in immobilization of the complex at the test line, where formation of a colored line indicates a positive result. The control line formed of rabbit secondary antibodies captures the remaining gold nanoparticle conjugates by binding to rabbit anti-FAM IgG. FIG. 12C is a schematic of the timeline of the assay, showing the incubation conditions and duration of the 3 main steps in ssRPA: (1) RT-RPA, (2) exonuclease digestion and (3) lateral flow. The test line and control line can be visualized as early as 1-3 min or as late as 10+ min without false positives. FIG. 12D is a schematic showing rhe basic equipment needed for the ssRPA. FIG. 12E is a series of lateral flow strip images, showing the sensitivity of ssRPA-LFD, as demonstrated by serial dilution from 100,000 copies down to 3 copies per reaction. 5 µl genomic viral RNA in DNase/RNase-free water was used as input for the 50 µl reaction volume. After RT-RPA for 5 minutes at 42° C., 2.5 µl product was transferred into 50 µl of the exo/LFD buffer. Following 1 min T7 exonuclease digestion at room temperature, samples were applied to commercial HybriDetect™ strips for ≥1 min. A time series for the same strip is shown in each column. FIG. 12F is a series of lateral flow strip images; specificity was shown in the background of 7 other respiratory virus genomic samples, including one of the common cold coronaviruses, spiked into DNase/RNase-free water and subject to ssRPA by using SARS-CoV-2 spike gene specific primers and detection probes. SARS- CoV-2 was used as a positive control. Catalog numbers for BEI (SARS-CoV-2) or other samples (ATCC) are listed in Methods. Strips show the readout at 10 min of lateral flow. FIG. 12G is a series of lateral flow strip images; 3 copies SARS-CoV-2 viral isolate was spiked in presumed negative human saliva. The same strip is shown after 1 or 2.5 min of lateral flow.

FIG. 13 is a dot plot showing the quantification of genomic RNA. Routine RTqPCR was performed simultaneously on quantitative full length RNA and genomic RNA samples used in detection (see e.g., FIGS. 12A-12G). A linear fit to quantitative RNA was used to estimate genomic RNA dilutions, with results and confidence intervals shown (see e.g., Methods). For example, at a genomic dilution yielding a Ct of 31.4, the concentration (count per ul) is estimated to be 1170, with a 95% confidence interval of 866-1580.

FIGS. 14A-14B shows gels and full-length images of strips in FIGS. 12E and 12F. FIG. 14A is an image of a denaturing PAGE gel showing the results of a 5 minute RT-RPA and subsequent 1 minute T7 exonuclease digestions, as well as the addition of LFD biotin and FAM probes, for the series dilution of genomic SARS-CoV-2 sample shown in FIG. 12E. Results show strong product bands over a large dynamic range of copy number. Full length LFD strips from FIG. 12E are also shown, indicating the copy count. FIG. 14B shows a corresponding gel, performed as in FIG. 14A, showing specificity data of FIG. 12F. Although products of RPA appear with non-SARS-CoV-2 templates, they are not specific to SARS-CoV-2 and do not activate the LFD in FIG. 12F.

FIG. 15 is a line graph showing the verification of background virus presence by qPCR. To confirm the presence of viruses demonstrating SARS-CoV-2 specificity in FIGS. 14A-14B, qPCR with virus-specific primers was performed using the virus samples of FIGS. 14A-14B and the respective primer pairs shown (see e.g., SEQ ID NO: 9-18). Positive and Negative sample controls resulted in the qPCR EvaGreen™ signals shown, with all 5 negative samples maintaining base signal levels.

FIGS. 16A-16B shows Gel and LFDs of full virus-spiked human saliva samples. FIG. 16A is an image of a denaturing PAGE gel showing the results of a 5 minute, 5′ spike RT-RPA and subsequent 1 minute T7 exonuclease digestions, as well as the addition of LFD biotin and FAM probes, for a series dilution of genomic SARS-CoV-2 sample (BEI) into raw, pooled, human saliva. Saliva was used at a volume of 5 ul in a 50 ul RPA reaction, and spiked with the copy number indicated. Results show strong product bands over 5 orders of magnitude in concentration, including a sample with an expected quantity of 3 copies. No band is seen without spiking. FIG. 16B shows corresponding HybriDetect™ LFDs at a time series of LFD incubations, showing the expected positive test lines for the same samples as in FIG. 16A, within 1-2 min.

FIGS. 17A-17B shows gel and LFDs of saliva samples inactivated by different treatments. Contrived saliva samples pre-mixed with 0 or 3 copies of viral RNA (BEI) were either heated at 95C for 10 min (left) or mixed 1:1 with Lucigen QuickExtract™ DNA extraction solution and heated to 95C for 5 min (right), and both cooled and added to the standard, 5 min, 5′ spike ssRPA reaction. They were then treated with 1 min of T7 exonuclease and run on a denaturing PAGE gel or HybriDetect™ LFDs. FIG. 17A shows an image of the PAGE gel. FIG. 17B is an LFD time series, showing the expected true positive and true negative results, indicating the process is compatible with this pre-treatment. Note that the QuickExtract™ treatment reduced the quantity of RNA copies into the ssRPA mix by half, to an average of 1.5 copies per reaction.

FIGS. 18A-18B shows gel and full-length LFDs for SARS-CoV-2 fragment synthetic RNA. FIG. 18A is an image of a denaturing PAGE gel showing the results of a 5 minute, 5′ spike RT-RPA and subsequent 1 minute T7 exonuclease digestion with addition of LFD biotin and FAM probes present, for the series dilution of SARS-CoV-2 synthetic fragment RNA (IDT). Results show strong product bands for average quantities of 3 and 3 copies/sample, and no amplification products were visible for 0.3 copies and 0.03 copies/sample. FIG. 18B shows HybriDetect LFD strips of the same samples as FIG. 18A shown at 1 and 2 minutes, indicating proper product in only 3 copies/lane samples.

FIGS. 19A-19B shows gel and full-length LFDs for SARS-CoV-2 full-length synthetic RNA. FIG. 19A is an image of a denaturing PAGE gel, showing the results of a 5 minute, 5′ spike RT-RPA and subsequent 1 minute T7 exonuclease digestion with addition of LFD biotin and FAM probes present, for the series dilution of SARS-CoV-2 full-length synthetic RNA (TwistBio™). Results show strong product bands for average quantities of 3 and 3 copies/sample, and no amplification products were visible for 0.3 copies and 0.03 copies/sample. FIG. 19B shows HybriDetect™ LFD strips of the same samples as FIG. 19A shown at 1 and 2 minutes, indicating proper product in only 3 copies/lane samples.

FIGS. 20A-20B shows gel and full-length LFDs for SARS-CoV-2 viral sample RNA. FIG. 20A is an image of a denaturing PAGE gel, showing the results of a 5 minute, 5′ spike RT-RPA and subsequent 1 minute T7 exonuclease digestion with addition of LFD biotin and FAM probes present, for the series dilution of SARS-CoV-2 inactivated virus (BEI), quantified by qPCR (see e.g., FIG. 13). Results show strong product bands for average quantities of 3 and 3 copies/sample, and no amplification products were visible for 0.3 copies and 0.03 copies/sample. FIG. 20B shows HybriDetect™ LFD strips of the same samples as FIG. 20A, shown at 1, 2, and 5 minutes, indicating proper product in only 3 copies/lane samples.

FIG. 21 demonstrates the requirement of exonuclease treatment for positive LFD results. HybriDetect™ LFDs are shown at 1, 2, and 5 minutes for 10,000 or 3 copies/sample amplification using 3′ spike as a target. For each pair of strips at a given sample concentration, strips were run with sample before or after 1 minute of T7 exonuclease treatment in the exo/LFD buffer, as with other experiments. Only exonuclease-treated samples bound the biotin and FAM probes required to localize nanoparticles to the test line.

FIGS. 22A-22B shows gel and LFDs demonstrating negative results with missing RPA reaction components. FIG. 22A is an image of a denaturing PAGE gel, showing results of nearly complete ssRPA reaction targeting the 5′ spike domain of 100 copies of the full virus (BEI). When template, magnesium, or either primer is missing, no product band is formed. A positive control is shown in the last lane, run with all components. FIG. 22B shows corresponding HybriDetect™ LFDs of the sample samples from FIG. 22A, demonstrating a positive in the full reaction only.

FIG. 23 shows images of LFDs, demonstrating the components required. An ssRPA reaction was completed, targeting 100 copies of the 3′ spike domain, and supplying 100 copies of full virus (BEI) as a target. Exonuclease treatment was performed for 1 min and the LFD was run for the time series shown. When the biotin or FAM probes were missing, no band was seen. Only when the same product was mixed with both probes did a positive test line appear.

FIGS. 24A-24B is a series of schematics showing various implementations of the present disclosure. FIG. 24A shows ssRPA detection using two probes (e.g., b* and c*). FIG. 24B shows ssRPA detection using biotin on the ‘a’ primer and a single probe b*c*FAM. Note that the biotin and FAM can be switched such that FAM is linked to the ‘a’ primer and the single probe is b*c*biotin.

FIGS. 25A-25C are a series of schematics showing toehold switching based LFD detection.

FIG. 26 is a schematic showing a time comparison for different assays and steps.

FIGS. 27A-27G are a series of schematics, images, and graphs showing ssRPA assay design, workflow, and characterization. FIG. 27A is a schematic showing that the key to ssRPA design is the rapid generation of millions of ssDNA copies from a single RNA target. ssDNA output offers straightforward specific readout by fluorescence or colorimetric/visual methods such as lateral flow devices. FIG. 27B is a schematic showing an exemplary ssRPA method. Step 1: Target viral RNA region (of domains a-b-c-d) is reverse transcribed into cDNA via extension of the reverse primer (d*) by the Reverse Transcriptase in the reaction mixture¹⁸. Subsequently, the cDNA is amplified via isothermal RPA at 42° C. by templated extension of the forward (a) and reverse primers (d*). The forward primer has a 6-nucleotide long poly-T segment with phosphorothioate bonds. Step 2a: Products of RPA are amended with SDS and transferred to the exo/LFD buffer that contains T7 exonuclease (a dsDNA-specific 5′ to 3′ exonuclease) and detection probes. The resulting mixture is incubated for 1 min at ambient temperature and reverse strand of the dsDNA amplicon products get preferentially digested yielding ssDNA amplicon (a-b-c-d) homologous to the target RNA sequence. Step 2b: The 3′ biotin (b*) and 5′ FAM (c*) modified detection probes make the assay directly compatible with commercially available test strips that feature a streptavidin test line and gold nanoparticles conjugated to rabbit anti-FAM IgG at the conjugate pad. Step 3: The test strip is vertically inserted into the resulting 50 µl mixture. The right ssDNA amplicon acts as a bridge that binds both the biotin-probe and the FAM-probe independently resulting in immobilization of the complex at the test line, where formation of a colored line indicates a positive result. The control line formed of rabbit secondary antibodies captures the remaining gold nanoparticle conjugates by binding to rabbit anti-FAM IgG. FIG. 27C is a schematic of the timeline of the assay, showing the incubation conditions and duration of the 3 main steps in ssRPA: (1) RT-RPA, (2) exonuclease digestion and (3) lateral flow. The test line and control line can be visualized as early as 1-2 min or as late as 60⁺ min without false positives. FIG. 27D is a series of lateral flow strip images, showing the sensitivity of ssRPA-LFD, as demonstrated by serial dilution from 1,000,000 copies down to 3 copies per reaction. A 5 µl volume of genomic viral RNA in DNase/RNase-free water was used as input for a 50 µl reaction volume. After RT-RPA for 5 minutes at 42° C., 2.5 µl product was transferred into 50 µl the exo/LFD buffer. Following 1 min T7 exonuclease digestion at room temperature, samples were applied to commercial HybriDetect™ strips for ≥1 min. A time series for the same strip is shown in each column. FIG. 27E is a series of lateral flow strip images; using the same procedure as in FIG. 27D, but spiking 10 copies into SARS-CoV-2-negative human saliva in 20 repeats, the LoD was estimated as below 10 copies. A no-template negative control is shown. FIG. 27F is a series of lateral flow strip images; specificity was shown by testing 8 other respiratory virus genomic samples, including 4 other coronaviruses, spiked in DNase/RNase-free water and subject to ssRPA by using SARS-CoV-2 spike gene specific primers and detection probes, with SARS-CoV-2 as a positive control. Catalog numbers for virus materials are listed in Methods. Strips show the readout at 10 min of lateral flow. FIG. 27G is a schematic showing an exemplary method; clinical samples were taken as NP swabs in VTM, NP swabs in water, or saliva, from SARS-CoV-2 positive and negative patients alike, underwent a 5 min extraction protocol with 50% dilution (see e.g., Methods), and tested at 10% v/v into ssRPA.

FIG. 28 is an image of a gel showing that a higher magnesium concentration makes the kinetics faster in RPA, leading to more amplified product. For higher magnesium (e.g., 28 mM Mg final concentration), the target output is stronger as shown in gel. Target band that appears near 75 nt is much stronger for 28 mM Mg than 14 mM Mg.

FIGS. 29A-29D are a series of schematics showing alternative strategies of sequence-specific probe binding after isothermal amplification. FIG. 29A is a schematic that shows the overall process. FIG. 29B is a schematic showing that standard RPA can be used to produce double-stranded amplicons, followed by binding of the detection probes to the amplicon that is rendered accessible by the action of RPA proteins and optionally buffer additives like SDS. FIG. 29C is a schematic showing an example timeline for the assay. FIG. 29D shows LFD strips where viral RNA input was detected with this workflow at 10 or 100 copies of input amount, but not detected in absence of the input (0 copies).

FIG. 30 is a series of schematics showing the colorimetric detection of the RNA target after isothermal amplification and sequence specific probe binding following up exonuclease-mediated ssDNA generation or direct probe access to the dsDNA amplicon that is rendered accessible. In this case the probes carry nanoparticles whose optical properties change based on the particle density. In absence of amplicon binding, the diffuse nanoparticle probes make the solution red. Binding to the target creates aggregation of the nanoparticles, making the solution turn purple. The color change hence indicates the presence of the target amplicon in solution. This method is also applicable to concatemeric amplicons such as those generated by LAMP and RCA via fast aggregation of the nanoparticles on the sequence repeats of the amplicon.

FIGS. 31A-31B is a series of schematics and images showing an exemplary workflow and results of a method as described herein. FIG. 31A is a schematic showing the workflow to produce double-stranded amplicons with isothermal amplification, followed by ssDNA digestion by exonuclease activity and reduction of the background interactions, which may cause false positives on the lateral flow membrane, by subsequent addition of SDS to the sample before the LFD run. FIG. 31B shows LFD strips LFD strips where viral RNA input (at 2000 copies) was detected with 2 different cognate target-probe pairs (+, # 1 and 2), but no false positive was obtained with non-cognate pairs (NC, #3and 4) or in the negative sample (-, no amplicon).

FIG. 32 is a series of schematics and images showing optional LFD pre-treatment for improved detection accuracy. The pretreatment of LFD strip by drying it with SDS as depicted in the schematic provides reduction of the unspecific background interaction in cases where there is significant unspecific interaction between the LFD test line and the assay components. The pretreatment inhibits false positive band formation in absence of the target amplicon (indicated by “-”). SDS or other additives (as described herein) can be applied in advance of the assay and stored for short or extended periods. The photo shows LFD output where a false positive test line is observed for untreated or water-pretreated strips, but it is eliminated in SDS-pretreated strips without interfering with the positive line formation in presence of the target (right-most strip, indicated by “+”).

FIG. 33 is a schematic showing exemplary RPA workflows.

FIG. 34 is a schematic showing exemplary LAMP workflows.

FIG. 35 is a schematic showing exemplary HDA workflows.

FIG. 36 is a schematic showing exemplary HDA workflows.

FIG. 37 is a line graph showing the effect of crowding agents on the reaction efficiency for HDA.

FIGS. 38A-38C is a series of schematics, images, and graphs showing exemplary fluorescence cleavage data. FIG. 38A is a schematic showing use of fluorescence cleavage probe to generate amplicon sequence-specific fluorescence signal. Upon binding of the fluorescence probe (which contains a fluorophore and a quencher, so that baseline signal is low) to the specific amplicon, the fluorophore gets cleaved from the quencher while the strand is digested by exonuclease. This separates the fluorophore from the quencher and results in increased signal. An alternative form of the cleavage probe contains a biotin and a fluorescein modification, and cleavage can be read out on an LFD based on separation of the biotin and fluorescein modifications. FIG. 38B is fluorescence cleavage probe data showing comparisons between phosphorylated (P primer) and non-phosphorylated primers used in a 5 minute RPA reaction and followed by either heat inactivation (HI) or no heat inactivation (no HI) at 95 C for 1.5 minutes. Time course of fluorescence is shown on the right for presence (+) or absence (-) of 20 fM target strand input to RPA reaction. Mobile device picture of fluorescence after time course with tubes on a blue light transilluminator with amber filter cover (left). FIG. 38C is fluorescence cleavage probe data showing time course of fluorescence (right) comparisons following 5 minute RPA reaction with different spiked concentrations of target in saliva and followed by heat inactivation (HI) at 95 C. Mobile device picture of fluorescence after time course with (white-walled) tubes on a blue light transilluminator with amber filter cover (top left). After transfer of samples to clear tubes, a second picture was taken from an alternate angle (bottom left).

FIGS. 39A-39B is a series of schematics showing a double-labeled nucleic acid probe. FIG. 39A demonstrates a double-labeled nucleic acid probe is originally in a quenched state since the labels are held in close proximity to each other. The probe hybridizes to a target in a sequence specific manner, creating a double stranded region. A double strand specific exonuclease recognizes this region and digests the probe, separating the labels and activating them. FIG. 39B shows the enzyme can act in a catalytic manner, since once the probe is digested the target is free to bind further probes, which are subsequently digested. This creates an amplified reporting mechanism.

FIG. 40 shows the mechanism of Digest-LAMP illustrated with a fluorescent probe.

FIG. 41 shows Digest-LAMP reporting methods. i. LFD readout, ii. Colorimetric readout, iii. Fluorescent readout and iv. Multiplexed readout.

FIG. 42 shows Digest-LAMP detection of SARS-CoV-2. 100 copies and 50 copies of SARS-CoV-2 RNA (in water) from a commercial source were added to a Digest-LAMP reaction. Each reaction was performed in duplicate. We successfully amplified and detected SARS-CoV-2 RNA inside 30 minutes using a commercial real time PCR instrument. In addition, we tested saliva samples from anonymized patients, one of whom was putative COVID positive while the other was putative COVID negative. The saliva samples were treated by heating to 95° C. for 5 minutes and added to a Digest-LAMP reaction at 5% of total reaction volume. We successfully amplified and detected SARS-CoV-2 RNA inside 30 minutes in the putative positive COVID sample while no target was detected in the putative negative sample. We successfully detected a human control gene (RNaseP) in the negative sample with Digest-LAMP, thus ruling out amplification inhibitory effects.

FIG. 43 is a schematic showing an exemplary two-part nucleic acid probe, wherein a portion of the first part of the probe hybridizes to a portion of the second part of the probe. The first (or second) part of the probe comprises a quencher molecule (indicated by “Q”), and the second (or first) part of the probe comprises a reporter molecule (indicated by the star).

FIGS. 44A-44B is a series of schematics and graphs showing specific detection of target and amplified signal production by catalytic turnover of digest probes. FIG. 44A is a schematic showing the assay using Bst full length as the exonuclease. FIG. 44B is a series of line graphs showing fluorescence for different concentrations of the nucleic acid probe (e.g., 10 nM to 100 nM; see e.g., top graph) or for negative controls (e.g., no target nucleic acid, bottom left graph; e.g., no Bst enzyme, bottom right graph).

FIG. 45 is a dot plot showing the temperature robustness of digest probes. Almost complete probe cleavage is obtained over a wide temperature range from 50° C. to 65° C. and partial cleavage is obtained for temperatures down to 30° C.

FIG. 46 is a series of line graphs showing the superior specificity of Digest-LAMP versus LAMP detection by double-strand DNA (dsDNA) specific fluorescent stain SYTO-9. Digest-LAMP using a nucleic acid probe as described herein (left graph) only produces signal above detection threshold in the presence of target (positive control) while SYTO-9 LAMP (right graph) detection leads to false positive signals in the absence of a target due to spurious amplification.

FIG. 47 is a line graph showing the specificity of Digest-LAMP for infectious disease detection. Digest-LAMP only produces signal above detection threshold in the presence of target (as indicated here by dotted arrow, SARS-CoV-2 RNA) while no signal is produced when it is challenged with other infectious viral pathogens like Influenza, Rhino virus, RSV, etc., or even other coronaviruses.

FIG. 48 is a line graph showing the superior signal of Digest-LAMP versus Molecular Beacon technology. The lowest signal is produced when no Bst Full Length enzyme is used, in which case the probe binds to the LAMP amplicon but is not digested, similar to molecular beacon technology. As increasing amounts of Bst Full Length enzyme is included in reactions, the corresponding signal also increases as more probe is digested.

FIG. 49 is a line graph showing the robust detection of SARS-CoV-2 RNA with Digest-LAMP, with all twenty repeats (solid lines) of the experiment successfully amplifying in the presence of 100 copies of SARS-CoV-2 RNA and no amplification in the absence of SARS-CoV-2 RNA (dotted lines).

FIGS. 50A-50B is a series of line graphs showing multiplexed detection of SARS-CoV-2 RNA and a human specimen control in the same tube with Digest-LAMP. FIG. 50A shows the COVID channel. All twenty repeats (solid lines) of the experiment successfully amplified in the presence of 100 copies of SARS-CoV-2 RNA, and there was no amplification in the absence of SARS-CoV-2 RNA (dotted lines). FIG. 50B shows the ACTB1 channel. All twenty-two repeats (twenty solid lines and two dotted lines) of the experiment successfully amplified, indicating the presence of clinical nasal elute (human specimen control) in the samples.

FIG. 51 is a line graph and chart showing COVID tests for the presence of SARS-CoV-2 performed on nasal samples from COVID positive and COVID negative patients. Samples were tested with both Digest-LAMP and RT-qPCR for the presence of SARS-CoV-2 RNA, and a high degree of concordance (16/17 agreement for COVID positive and 10/10 agreement for COVID negative) was found between Digest-LAMP and RT-qPCR results, indicating the usefulness of Digest-LAMP as a diagnostic for infectious diseases.

FIG. 52 is a line graph and chart showing COVID tests for the presence of SARS-CoV-2 performed on saliva samples from COVID positive and COVID negative patients. Samples were tested with both Digest-LAMP and RT-qPCR for the presence of SARS-CoV-2 RNA and 100% concordance (5/5 agreement for COVID positive and 5/5 agreement for COVID negative) was found between Digest-LAMP and RT-qPCR results, indicating the usefulness of Digest-LAMP as a diagnostic for infectious diseases.

FIGS. 53A-53E is a series of schematics showing probe and assay set-ups. FIG. 53A shows probe binding to the single-stranded region flanked by hairpin stems. FIG. 53B shows probe binding to one of the hairpin loops. FIG. 53C shows probe binding to partially exposed overlap to neighboring regions flanked by hairpin stems. FIGS. 53D-53E shows different possible probe configurations. Black dots on probes represent quenchers while stars represent fluorophores.

FIGS. 54A-54C is a series of schematics and graphs showing the specific detection of double-stranded target by digest probes. Double strand specific exonucleases can partially digest double-stranded target molecules (i.e., double-stranded DNA) and facilitate the sequence-specific target recognition by digest probes. FIG. 54A is a schematic showing that double-strand specific exonucleases act on the 5′-ends of the double-stranded target molecules (i.e., double-stranded DNA) and convert these molecules into partial duplexes whose strands are separable at typical digestion reaction temperatures ranging from 50° C. to 65° C. Once the (partially-digested) strands are separated, the probe binding site becomes available for the catalytic binding-and-digestion cycle of digest probes. Thus, double-stranded target molecules can be detected in simple one-step incubation. FIG. 54B is a schematic showing that the double-stranded target detection can additionally benefit from 5′-end protection techniques, which can prevent the target strand (i.e., the strand containing the digest probe binding site) from being fully digested by double strand specific exonucleases. The 5′-end of the non-target strand is left unprotected for removal by double strand specific digestion. FIG. 54C is a line graph showing the digest probe fluorescence signal recorded in the presence (solid and dashed lines) and absence (dotted line) of dsDNA targets. Detectable fluorescence signal above the background is generated only when the dsDNA target molecules are present. The solid line represents the detection of half-protected dsDNA target molecules (i.e., protection only at the 5′-end of the target strand) and the dashed line represents the detection of unprotected dsDNA target molecules.

FIG. 55 is a series of graphs showing amplified signal production by catalytic turnover of digest probes. The top graph shows the fluorescence signals of digest probes at various probe concentrations (20 nM to 100 nM). A fixed amount (20 nM) of unprotected dsDNA target molecules are presented in these experiments. Both the plateau time and the end-point fluorescence increase with probe concentration. The bottom graphs show the fluorescence signals without dsDNA target (bottom left graph) and without the Bst Full Length polymerase (bottom right graph). The probe concentration was fixed at 100 nM in these reactions.

FIG. 56 is dot plot showing the temperature robustness of digest probes. The cleavage efficiencies of digest probes at various probe-to-target (dsDNA) ratios are plotted as a function of incubation temperature. Each point represents the probe cleavage percentage after 30 min of incubation in the presence of unprotected dsDNA target molecules. The cleavage efficiency remains high (>90%) for the probe-to-target ratio of 1:1 at all temperatures tested. For the other ratios, the temperature range between 60° C. to 65° C. results in the highest cleavage efficiency.

FIG. 57 is a schematic showing an additional detection scheme of a double-stranded nucleic acid target by combining digest probes with single-strand binding (SSB) proteins. In case when both of the two 5′-ends of double-stranded target are prevented from digestion by double strand specific exonucleases (i.e., having protection at both 5′-ends), and hence; the digest probe binding site is not accessible for probe binding, single strand binding (SSB) proteins can be added to the reaction to assist probe binding.

### DETAILED DESCRIPTION

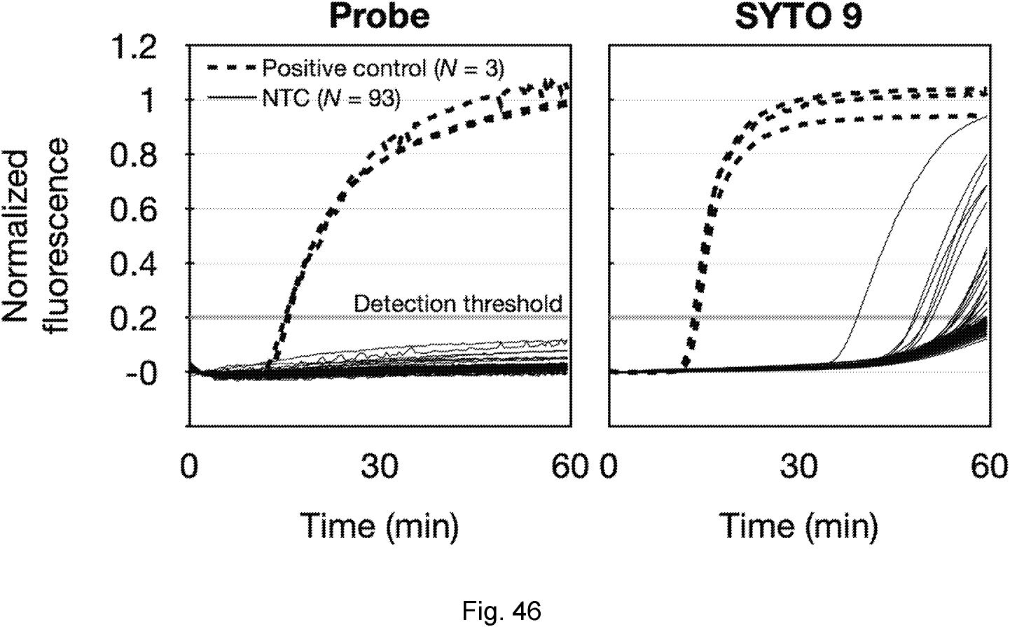

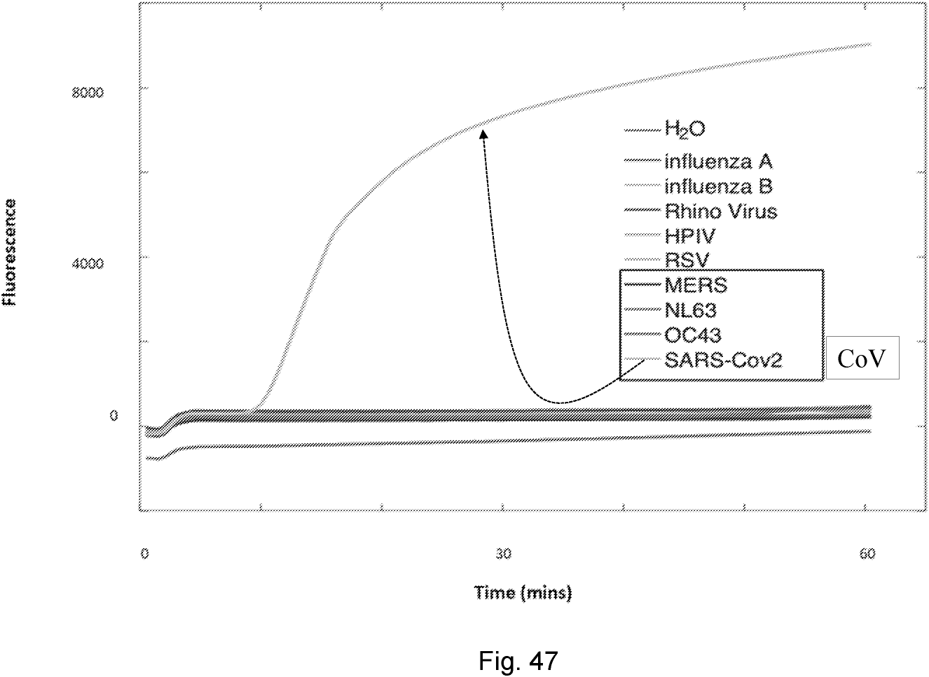

Embodiments of the technology described herein are directed at isothermal methods, compositions, kits, and systems for detecting nucleic acids. In several aspects, the compositions and methods provided herein are based, in part, on the discovery of a scheme for sequence-specific reporting of nucleic acid targets using catalytic probe digestion. Such catalytic probes can decrease the fall-positive rates of detection assays (see e.g., FIG. 46). Such methods also allow for highly specific detection of viruses, such as SARS-CoV-2 (see e.g., FIGS. 47, 49-52).

### Methods

The compositions and methods provided herein are based, in part, on the discovery of a scheme for sequence-specific reporting of nucleic acid targets using catalytic probe digestion.

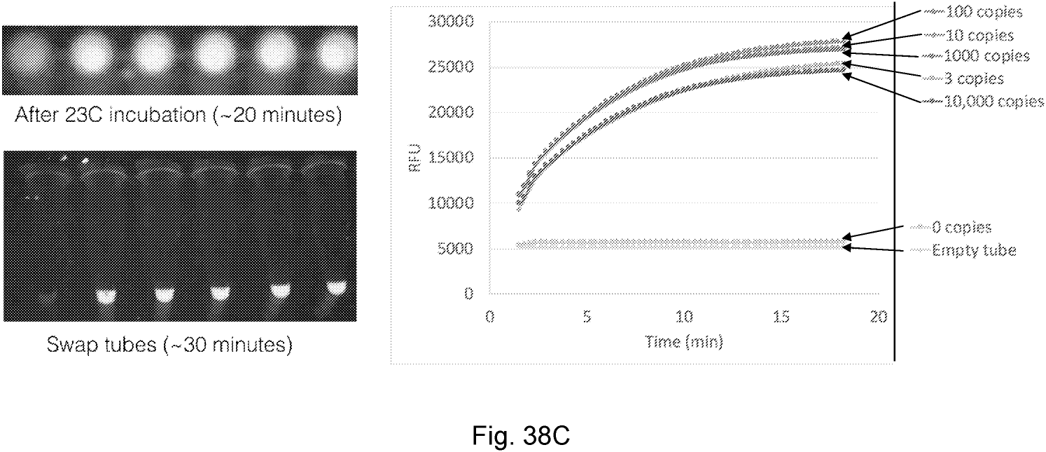

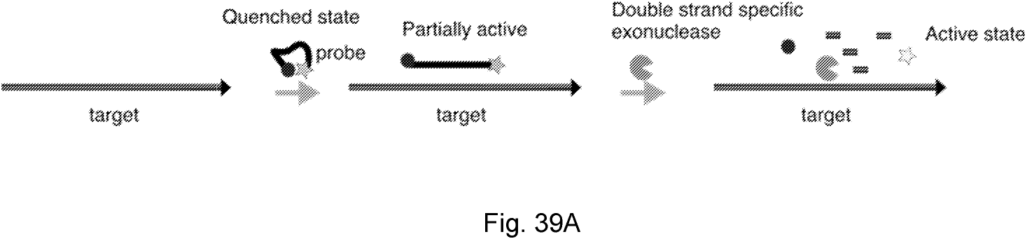

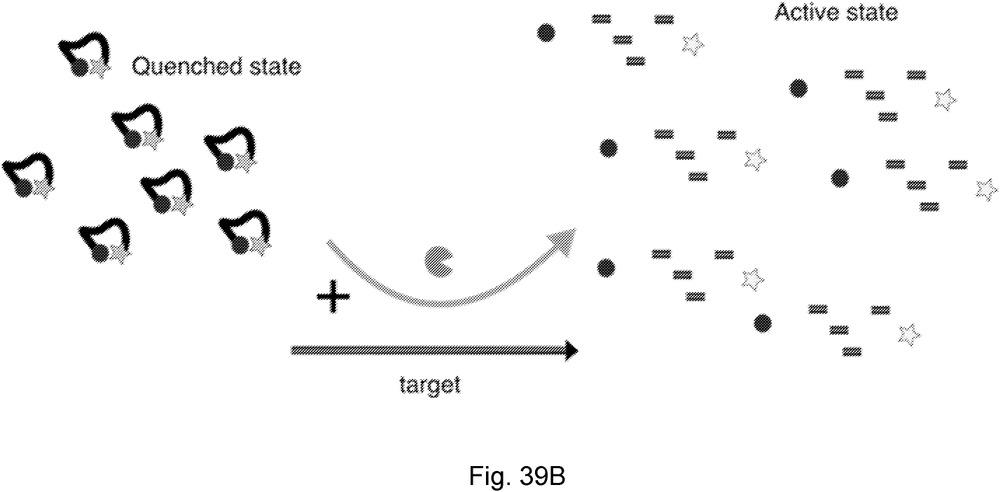

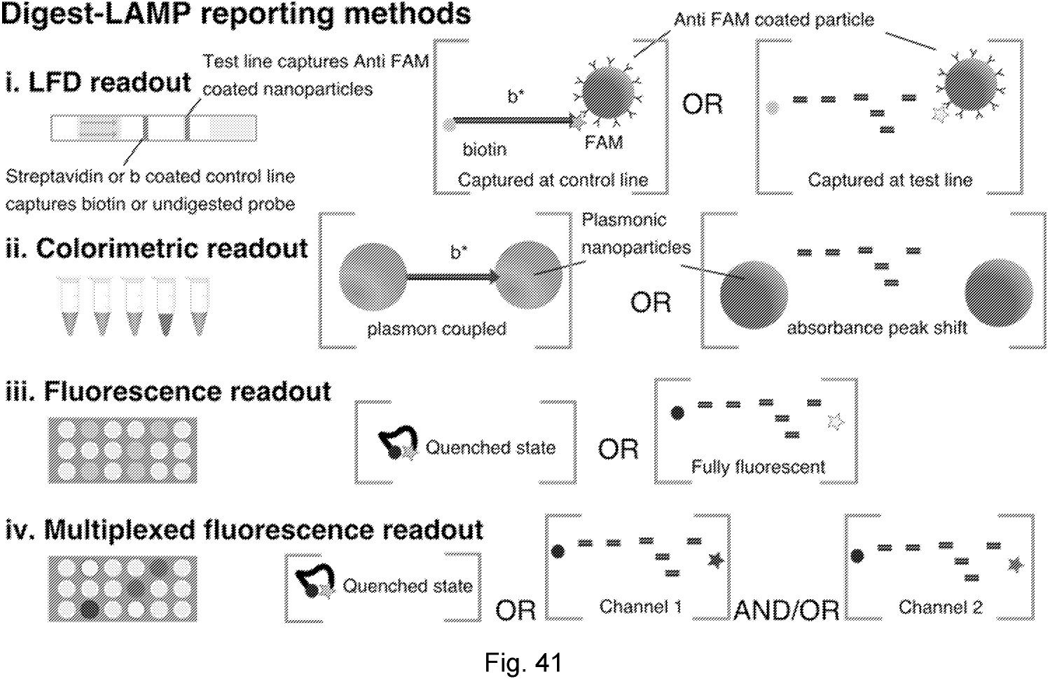

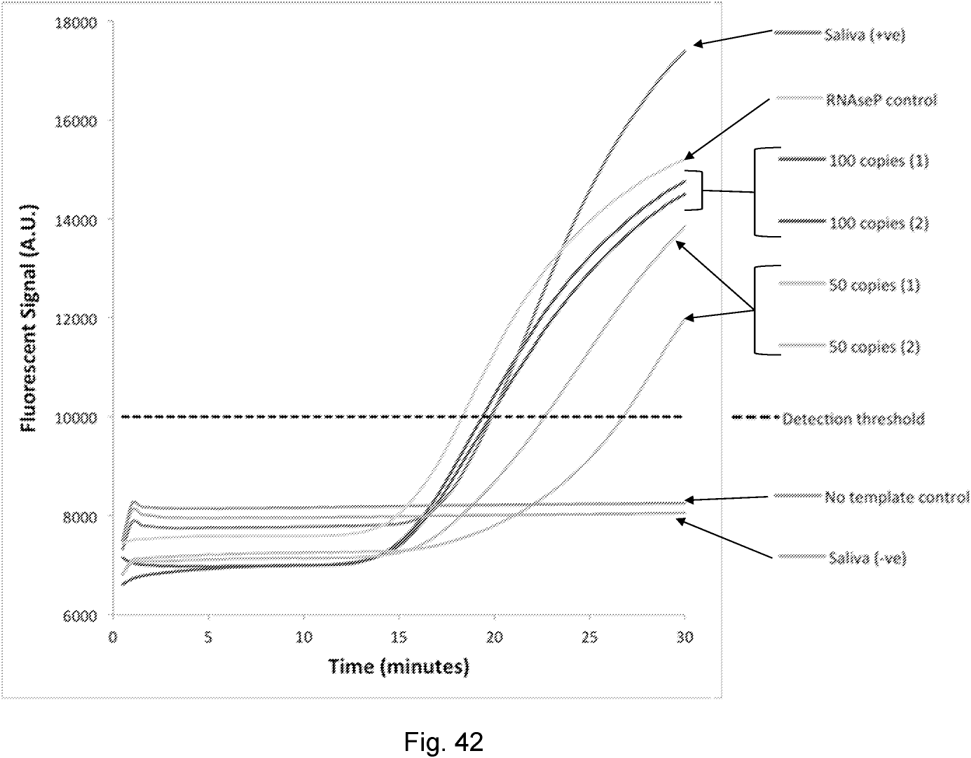

The fundamental strategy for detecting a target nucleic acid is depicted, e.g., in FIGS. 39-42.

In one aspect, provided herein is a method for detecting a target nucleic acid in a sample, the method comprising: (a) hybridizing a nucleic acid probe to an amplicon from an amplification of a target nucleic acid; (b) cleaving the hybridized nucleic acid probe with a double-strand specific exonuclease having 5′ to 3′ exonuclease activity; and (c) detecting the reporter molecule from the cleaved nucleic acid probe and/or detecting any remaining uncleaved nucleic acid probe.

In another aspect, provided herein is a method for detecting a target nucleic acid in a sample, the method comprising: (a) hybridizing a nucleic acid probe to an amplicon from amplification of a target nucleic acid, wherein the nucleic acid probe comprises a nucleotide sequence substantially complementary or identical to a nucleotide sequence of the target nucleic acid or a primer in used in the amplification of the target nucleic acid, wherein the nucleic acid probe comprises a reporter molecule capable of producing a detectable signal, and wherein said amplification is Loop-mediated Isothermal Amplification (LAMP); (b) cleaving the hybridized nucleic acid probe with a double-strand specific exonuclease having 5′ to 3′ exonuclease activity; and (c) detecting the reporter molecule from the cleaved nucleic acid probe and/or detecting any remaining uncleaved nucleic acid probe.

In another aspect, provided herein is a composition comprising: (a) an exonuclease having 5′->3′ cleaving activity; (b) a primer set for amplifying a target nucleic acid via LAMP; and (c) a nucleic acid probe comprising a reporter molecule,

In yet another aspect, provided herein is a composition comprising: (a) an exonuclease having 5′->3′ cleaving activity; (b) a primer set for amplifying a target nucleic acid via LAMP and wherein and the primer set comprises a forward outer primer (F3), a reverse outer primer (R3), a forward inner primer (FIP), and a reverse inner primer (RIP); and (c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set.

As used herein, “a nucleic acid probe” is used to refers to the nucleic acid strand that hybridizes to the target nucleic acid sequence. Multiple nucleic acid probes can be used in the same reaction.

In some embodiments of any of the aspects, the nucleic acid probe comprises a nucleotide sequence substantially complementary to a primer used in the amplification of the target nucleic acid. In some embodiments, the nucleic acid probe comprises a nucleotide sequence substantially identical to a primer used in the amplification of the target nucleic acid. In some embodiments of any of the aspects, the nucleic acid probe comprises a nucleotide sequence identical to a nucleotide sequence of the target nucleic acid. In some embodiments, the nucleic acid probe comprises a nucleotide sequence substantially complementary to a nucleotide sequence at an internal position of the amplicon.

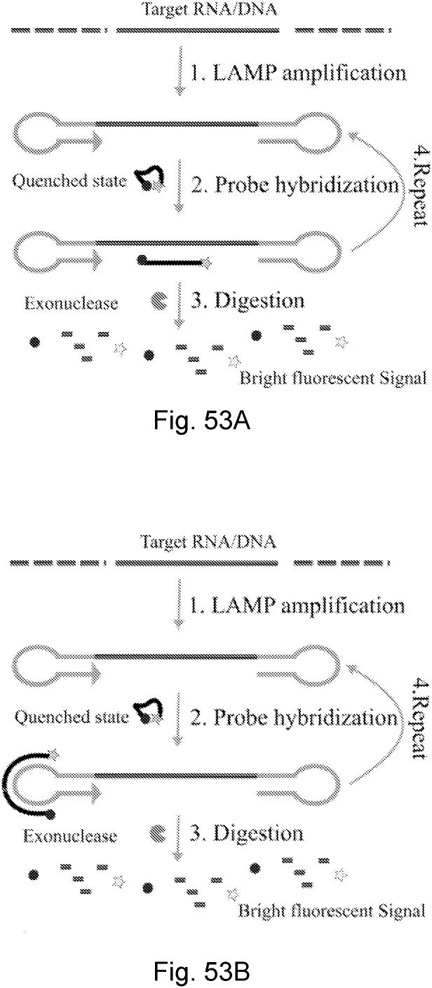

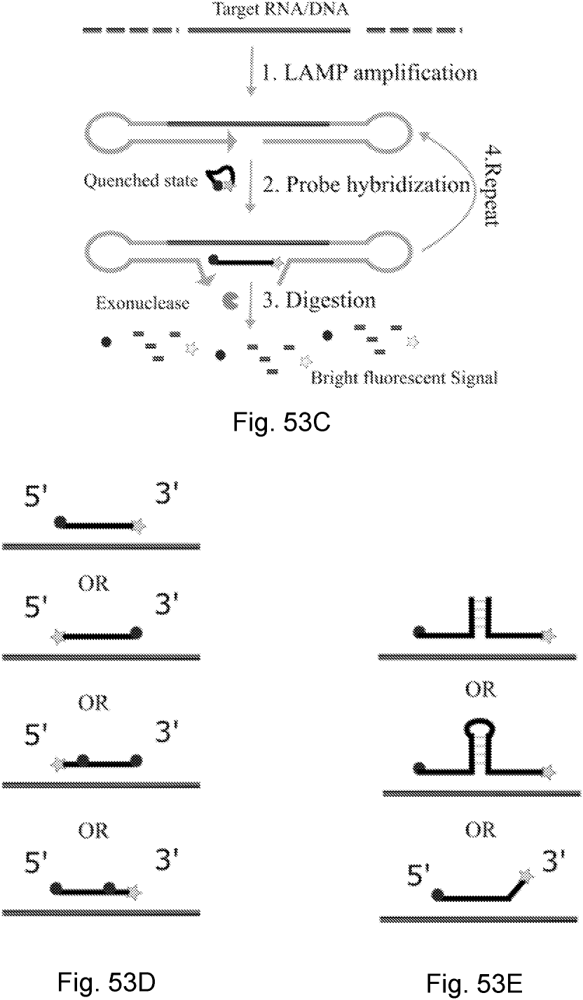

In some embodiments, the amplification method is LAMP, and the nucleic acid probe binds to the single-stranded region of the LAMP amplicon flanked by hairpin stems (see e.g., FIG. 53A). In some embodiments, the amplification method is LAMP, and the nucleic acid probe binds to one of the hairpin loops of the LAMP amplicon (see e.g., FIG. 53B). In some embodiments, the amplification method is LAMP, and the nucleic acid probe binds to a region of the LAMP amplicon that is partially covered by hairpin stems, including part of the single-stranded region of the LAMP amplicon (see e.g., FIG. 53C).

In some embodiments, the nucleic acid probe comprises at least one reporter molecule and at least one quencher molecule, each of which can each be at the 5′ end, 3′ end, or internal to the probe. In some embodiments, the nucleic acid probe comprises a 5′ quencher molecule and a 3′ reporter molecule. In some embodiments, the nucleic acid probe comprises a 3′ quencher molecule and a 5′ reporter molecule. In some embodiments, the nucleic acid probe comprises at least two quencher molecules. In some embodiments, the nucleic acid probe comprises a 5′ quencher molecule, an internal quencher, and a 3′ reporter molecule. In some embodiments, the nucleic acid probe comprises a 3′ quencher molecule, an internal quencher, and a 5′ reporter molecule. (see e.g., FIG. 53D).

In some embodiments, the nucleic acid probe comprises a first nucleic acid strand and a second nucleic acid strand. The first and second strands can hybridize to the amplicon at positions next, e.g., within 1, 2, 3, 4 or 5 nucleotides to each other. In some embodiments, the first and second strand form a double-stranded region with each other when hybridized to the amplicon. In some embodiments, the first and second strands are linked to each other. In some embodiments, the nucleic acid probe comprises at least two strands (e.g., 2, 3, 4, 5 or more), wherein the first strand comprises a region that is substantially complementary to a region in the second strand, wherein the second strand comprises a region that is substantially complementary to a region in the third strand, wherein the third strand comprises a region that is substantially complementary to a region in the fourth strand, wherein the fourth strand comprises a region that is substantially complementary to a region in the fifth strand, etc. In some embodiments, the first, second, third, fourth, fifth, etc., strands are linked to each other. In some embodiments, the nucleic acid probe forms a hairpin structure when hybridized to the amplicon. In some embodiments, the nucleic acid probe comprises a single-stranded region when hybridized to the amplicon (see e.g., FIG. 53E).

In some embodiments, the nucleic acid probe comprises one of SEQ ID NOs: 7-8, 19, 51-55 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 7-8, 19, 51-55 that maintains the same function (e.g., hybridization and detection).

In some embodiments, the nucleic acid probe comprises one of SEQ ID NOs: 19, 51-55 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 19, 51-55 that maintains the same function (e.g., hybridization and detection).

In some embodiments, the nucleic acid probe comprises one of SEQ ID NOs: 51-55 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 51-55 that maintains the same function (e.g., hybridization and detection).

In some embodiments, the nucleic acid probe comprises one of SEQ ID NOs: 51-53 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 51-53 that maintains the same function (e.g., hybridization and detection).

In some embodiments of any of the aspects, the nucleic acid probe comprises a primer. As used herein, the term “primer” is used to describe a sequence of DNA (or RNA) that is paired with one strand of DNA and provides a free 3′-OH at which a DNA polymerase starts synthesis of a deoxyribonucleotide chain. Preferably, the primer is composed of an oligonucleotide. The exact lengths of the primers will depend on many factors, including temperature and source of primer. For example, depending on the complexity of the target nucleic acid sequence, the oligonucleotide primer typically contains 15-40 or more nucleotides, although it may contain fewer nucleotides. Short primer molecules generally require cooler temperatures to form sufficiently stable hybrid complexes with a template.

In some embodiments, the nucleic acid primer comprises one of SEQ ID NOs: 5-6, 9-18, 21-50, 56-57 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 5-6, 9-18, 21-50, 56-57 that maintains the same function (e.g., amplification).

In some embodiments of any of the aspects, the nucleic acid probe or the primer provided herein is used in the amplification of the target nucleic acid. As used herein, the term “amplifying” refers to a step of submitting a nucleic acid sequence to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing. Methods of amplifying and synthesizing nucleic acid sequences are known in the art. For example, see U.S. Pat. Nos. 7,906.282, 8,367,328, 5,518,900, 7,378,262, 5,476,774, and 6,638,722, contents of all of which are incorporated by reference herein in their entirety.

In some embodiments, the amplification is Loop-mediated Isothermal Amplification (LAMP). LAMP allows for the amplification of target DNA using strand displacement DNA synthesis using primer sets without the need for a thermocycler. In contrast to PCR techniques, LAMP provides high specificity, efficiency, and rapidity under isothermal conditions to amplify a target sequence. LAMP is described in detail, e.g. in Notomi T, et al. “Loop-mediated isothermal amplification of DNA.” Nucleic Acids Res. 2000;28(12):E63, which is incorporated herein by reference in its entirety.

Thus, the methods and compositions provided herein can comprise a primer or a primer set that amplify the detection region of the target nucleic acid, creating many copies.

In some embodiments of any of the aspects, the primer set provided herein comprises a forward outer primer (F3), a reverse outer primer (R3), a forward inner primer (FIP), and a reverse inner primer (RIP). In some embodiments of any of the aspects, the primer set further comprises a forward loop primer (LF), and a reverse loop primer (LR).

The advantage of the methods provided herein is that the hybridization step or cleaving of the hybrid nucleic acid probe can be carried out simultaneously with amplification of the target nucleic acid. In other words, the amplification, hybridization and cleaving steps can be performed in a single reaction vessel. Furthermore, each digestion event acts to ‘check’ the sequence of the target or amplicon it binds to, thus ensuring a very sequence specific output signal.

In some embodiments, said hybridizing the nucleic acid probe or cleaving the hybridized nucleic acid probe is after the amplification of the target nucleic acid. As a non-limiting example, said hybridizing or cleaving the hybridized nucleic acid probe is performed at least 5 seconds, at least 10 seconds, at least 30 seconds, at least 45 seconds, at least 1 minute (min), at least 2 min, at least 3 min, at least 4 min, at least 5 min, at least 6 min, at least 7 min, at least 8 min, at least 9 min, at least 10 min, at least 20 min, at least 30 min, at least 40 min, at least 50 min, at least 60 min or more after the amplification.

In some embodiments, said hybridizing the nucleic acid probe or cleaving the hybridized nucleic acid probe is performed after isolation or purification of the amplicons from the amplification of the target nucleic acid. In other words, the method comprises a step of isolating or purifying the amplicon from the amplification reaction prior to hybridizing the nucleic acid probe or cleaving the hybridized nucleic acid probe.

The methods provided herein can be accomplished using a variety of reporting mechanisms for the detection of the target nucleic acid sequence. In some embodiments of any of the aspects provided herein, the reporter molecule provided herein produces a detectable signal for facile identification of the presence of the target nucleic acid. The target nucleic acids and compositions provided herein are discussed further below.

The methods described herein allow fast detection of target nucleic acids. The total time from starting the assay and detecting a signal can be few minutes to less than 2 hours. For clarity, starting the assay means adding reagents to the sample for amplifying the target nucleic acids. The total time from starting the assay and detecting a signal can be from about 15 minutes to about 90 minutes. Thus, the total time, from starting the assay to detecting a signal can be about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 70 minutes, about 75 minutes, about 80 minutes, or about 90 minutes.

In some embodiments of any of the aspects, the total time for the methods described herein can be at most 15 minutes, at most 16 minutes, at most 17 minutes, at most 18 minutes, at most 19 minutes, at most 20 minutes, at most 21 minutes, at most 22 minutes, at most 23 minutes, at most 24 minutes, at most 25 minutes, at most 26 minutes, at most 27 minutes, at most 28 minutes, at most 29 minutes, at most 30 minutes, at most 31 minutes, at most 32 minutes, at most 33 minutes, at most 34 minutes, at most 35 minutes, at most 36 minutes, at most 37 minutes, at most 38 minutes, at most 39 minutes, at most 40 minutes, at most 41 minutes, at most 42 minutes, at most 43 minutes, at most 44 minutes, at most 45 minutes, at most 46 minutes, at most 47 minutes, at most 48 minutes, at most 49 minutes, at most 50 minutes, at most 51 minutes, at most 52 minutes, at most 53 minutes, at most 54 minutes, at most 55 minutes, at most 56 minutes, at most 57 minutes, at most 58 minutes, at most 59 minutes, at most 60 minutes, at most 70 minutes, at most 75 minutes, at most 80 minutes, or at most 90 minutes.

In some embodiments, the total time for the methods described herein can be for about 15 minutes to about 45 minutes. For example, the total time for the methods described herein can be about 20 minutes to about 40 minutes, or from about 25 minutes to about 35 minutes.

The step of hybridizing the probe to the amplicon and/or cleaving the hybridized probe with the exonuclease can be performed at a temperature between from about 20° C. to about 75° C. For example, step of hybridizing the probe to the amplicon and/or cleaving the hybridized probe with the exonuclease can be performed at about 25° C. to about 70° C., from about 30° C. to about 65° C. or from about 35° C. to about 60° C. In some embodiments, the step of hybridizing the probe to the amplicon and/or cleaving the hybridized probe with the exonuclease can be performed at a temperature at 65° C. In some embodiments, the amplification, hybridization and cleaving steps are performed at a constant temperature.

### Nucleic Acid Modifications to the Nucleic Acid Probes and Primers

At least one nucleic acid probe or primer strand provided herein can independently comprise one or more nucleic acid modifications known in the art. For example, the nucleic acid probe can independently comprise non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs can be modified at the ribose, phosphate, and/or base moiety.

Exemplary nucleic acid modifications include, but are not limited to, nucleobase modifications, sugar modifications, inter-sugar linkage modifications, conjugates (e.g., ligands), and combinations thereof. In one embodiment, a modification does not include replacement of a ribose sugar with a deoxyribose sugar as occurs in deoxyribonucleic acid. Nucleic acid modifications are known in the art, see, e.g., US20160367702; US20190060458; U.S. Pat. No. 8,710,200; and U.S. Pat. No. 7,423,142, which are incorporated herein by reference in their entireties.

Exemplary modified nucleobases include, but are not limited to, thymine (T), inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

Exemplary sugar modifications include, but are not limited to, 2′-Fluoro, 3′-Fluoro, 2′-OMe, 3′-OMe, and acyclic nucleotides, e.g., peptide nucleic acids (PNA), unlocked nucleic acids (UNA) or glycol nucleic acid (GNA).

In some embodiments, a nucleic acid modification can include replacement or modification of an inter-sugar linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2—N(CH3)—N(CH3)—), amide-3 (3′—CH2—C(═O)—N(H)—5′) and amide-4 (3′—CH2—N(H)—C(═O)—5′), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′—S—CH2—O—5′), formacetal (3′—O—CH2—O—5′), oxime, methyleneimino, methykenecarbonylamino, methylenemethylimino (MMI, 3′—CH2—N(CH3)—O—5′), methylenehydrazo, methylenedimethylhydrazo, methyleneoxymethylimino, ethers (C3′—O—C5′), thioethers (C3′—S—C5′), thioacetamido (C3′—N(H)—C(═O)—CH2—S—C5′, C3′—O—P(O)—O—SS—C5′, C3′—CH2—NH—NH—C5′, 3′—NHP(O)(OCH3)—O—5′ and 3′—NHP(O)(OCH3)—O—5′.

In some embodiments of any of the aspects, 2′-modified nucleoside comprises a modification selected from the group consisting of2′-halo (e.g., 2′-fluoro), 2′-alkoxy (e.g., 2′-Omethyl, 2′-Omethylmethoxy and 2′-Omethylethoxy), 2′-aryloxy, 2′-O-amine or 2′-O-alkylamine (amine NH2; alkylamino, dialkylamino, heterocyclyl, arylamino, diaryl amino, heteroaryl amino, dihet.eroaryl amino, ethylene diamine or polyamino), O-CH₂CH₂(NCH₂CH₂NMe₂)₂, methyleneoxy (4′-CH₂-O-2′) LNA, ethyleneoxy (4′-(CH₂)₂-O-2′) ENA, 2′-amino (e.g. 2′-NH₂, 2′-alkylamino, 2′-dialkylamino, 2′-heterocyclylamino, 2′-arylamino, 2′-diaryl amino, 2′-heteroaryl amino, 2′-diheteroaryl amino, and 2′-amino acid); NH(CH₂CH₂NH)ₙCH₂CH₂-AMINE (AMINE = NH2, alkylamino, dialkylamino, heterocyclyl, arylamino, diaryl amino, heteroaryl amino, or diheteroaryl amino), -NHC(O)R (R = alkyl, cycloalkyl, aryl, aralkyl, heteroaryl or sugar), 2′-cyano, 2′-mercapto, 2′-alkyl-thio-alkyl, 2′-thioalkoxy, 2′-thioalkyl, 2′-alkyl, 2′-cycloalkyl, 2′-aryl, 2′-alkenyl and 2′-alkynyl.

In some embodiments of any of the aspects, the inverted nucleoside is dT.

In some embodiments of any of the aspects, the 5′-modified nucleotide comprises a 5′-modification selected from the group consisting of 5′-monothiophosphate (phosphorothioate), 5′-monodithiophosphate (phosphorodithioate), 5′-phosphorothiolate, 5′-alpha-thiotriphosphate, 5′-beta-thiotriphosphate, 5′-gamma-thiotriphosphate, 5′-phosphoramidates, 5′-alkylphosphonate, 5′-alkyletherphosphonate, a detectable label, and a ligand; or the 3′-modified nucleotide comprises a 3′-modification selected from the group consisting of 3′-monothiophosphate (phosphorothioate), 3′-monodithiophosphate (phosphorodithioate), 3′-phosphorothiolate, 3′-alpha-thiotriphosphate, 3′-beta-thiotriphosphate, 3′-gamma-thiotriphosphate, 3′-phosphoramidates, 3′-alkylphosphonate, 3′-alkyletherphosphonate, a detectable label, and a ligand.

In some embodiments of any of the aspects, the 5′-modified nucleotide comprises a detectable label or reporter molecule at the 5′-end. Non-limiting examples of detectable labels or reporter molecules are described further herein. In embodiments wherein the detectable label or reporter molecule is not a nucleic acid, such a detectable label (e.g., a fluorophore) can inhibit 5′->3′ cleaving activity of a 5′->3′ exonuclease.

In some embodiments, nucleic acid modifications can include peptide nucleic acids (PNA), bridged nucleic acids (BNA), morpholinos, locked nucleic acids (LNA), glycol nucleic acids (GNA), threose nucleic acids (TNA), or other xeno nucleic acids (XNA) described in the art.

In some embodiments of any of the aspects, the nucleic acid probe comprises at least one nucleic acid modification capable of increasing a melting temperature (Tm) of the nucleic acid probe for hybridizing with a complementary strand relative to a nucleic acid probe lacking said modification. Non-limiting examples of modifications that increase melting temperature include, locked nucleic acid (LNA) bases, minor groove binders (MGBs), 5-hydroxybutynyl-2′-deoxyuridine (SuperT), 5-Me-pyridines, 2-amino-deoxyadenosine, Trimethoxystilbene, RNA bases, methylated RNA bases, 2′ Fluoro bases, and pyrene.

In some embodiments of any of the aspects, the nucleic acid probe comprises at least one nucleic acid modification capable of inhibiting extension by a polymerase. For example, the nucleic acid probe lacks a 3′-OH group. In some embodiments, the 3′-OH group of the nucleic acid probe can be blocked, e.g., the hydrogen is replaced with some other group.

In some embodiments of any of the aspects, at least one of the primers used in the amplification step provided herein comprises a nucleic acid modification. In some embodiments, the primer is capable of inhibiting the 5′->3′ cleaving activity of the exonuclease. For example, one or more of the primers used for the amplification of the target nucleic acid comprises a nucleic acid modification capable of inhibiting the 5′->3′ cleaving activity of a 5′->3′ exonuclease. Nucleic acid modifications that can inhibit 5′->3′ cleaving activity of a 5′->3′ exonuclease are known in the art, such as modified internucleotide linkages, modified nucleobase, modified sugar, and any combinations thereof. Exemplary modifications include, but are not limited to 1, 2, 3, 4, 5, 6 or more modified internucleotide linkages, such as phosphorothioates; an inverted nucleoside or 5′->5′ internucleotide linkage; a 3′->3′ internucleotide linkage; a 2′-OH or a 2′-modified nucleoside; a 5′-modified nucleotide; 3′-modified nucleotide; a 2′->5′ linkage; an abasic nucleoside; an acyclic nucleoside; a spacer; left-handed DNA; nucleotides with non-canonical nucleobases; replacement of 5′-OH group; or any combinations thereof.

The modification capable of inhibiting 5′->3′ cleaving activity can be present anywhere in the primer. For example, it can be at the 5′-end or terminus, at an internal position, or at a position within the 5′-terminal, e.g., within positions within 1, 2, 3, 4, 5, 6, 7, 8, 9, 10, 11, 12, 13, 14 or 15 from the 5′-end. In some embodiments of any of the aspects, the nucleic acid modification is located at the 5′-end of the primer. In some embodiments of any of the aspects, the modification is a phosphorothioate base, a spacer modification, 2′-O-Methyl RNA, 5′ inverted dideoxy-dT base, and/ or 2′ Fluoro bases.

In some embodiments of any of the aspects, the spacer is located at the 3′ end of one or both primers. In some embodiments of any of the aspects, the spacer is located at an internal location of one or both primers. In some embodiments of any of the aspects, the spacer is located at the 5′ end of one or both primers. Non-limiting examples of spacers include the C3 spacer (phosphoramidite); hexanediol; 1′,2′-Dideoxyribose (dSpacer); PC (Photo-Cleavable) Spacer; Spacer 9 (a triethylene glycol spacer); and Spacer 18 (an 18-atom hexa-ethyleneglycol spacer).

In some embodiments of any of the aspects, the left-handed DNA is located at the 3′ end of one or both primers. In some embodiments of any of the aspects, the left-handed DNA is located at the 5′ end of one or both primers. In some embodiments of any of the aspects, the left-handed DNA is located at the 5′ end and 3′ end of one or both primers. In some embodiments of any of the aspects, the left-handed DNA is Z-DNA. Z-DNA is one of the possible double helical structures of DNA. It is a left-handed double helical structure in which the helix winds to the left in a zigzag pattern, instead of to the right, like the more common B-DNA form. Z-DNA is one of three biologically active double-helical structures along with A- and B-DNA. Many enzymes (e.g., exonucleases) that use right-handed DNA as a substrate cannot use left-handed DNA as substrate.

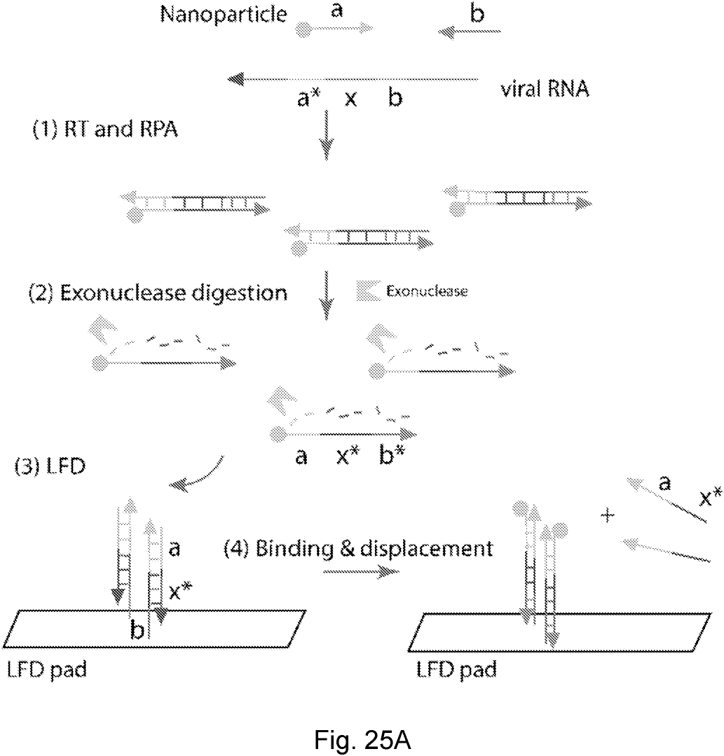

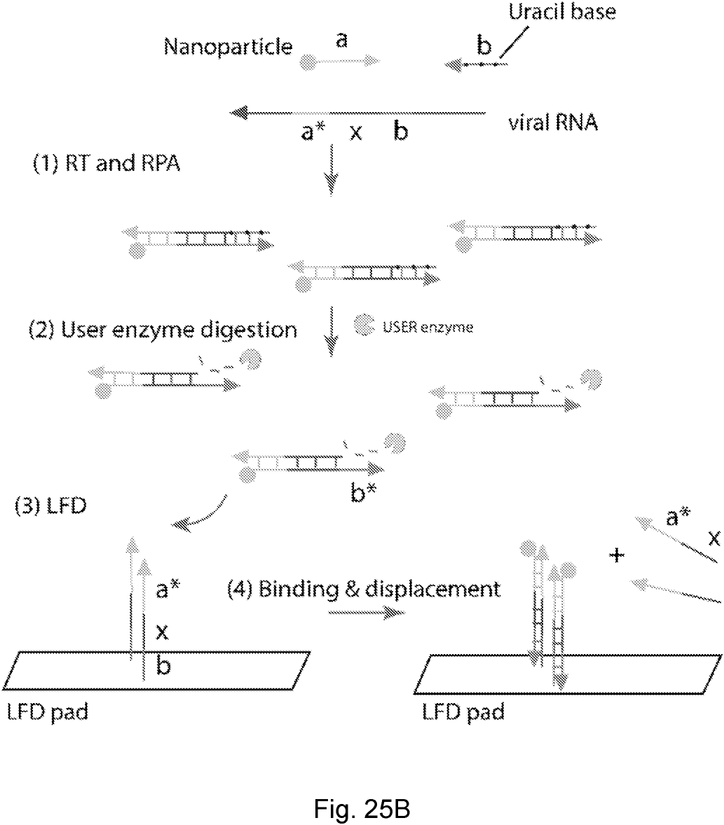

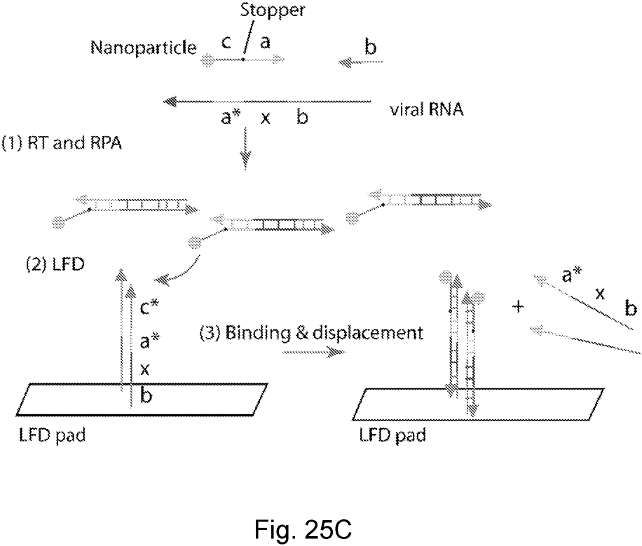

In some embodiments of any of the aspects, the nucleic acid modification capable of inhibiting 5′->3′ cleaving activity of a 5′->3′ exonuclease comprises a linkage to a bulk end group, such as a protein, an antibody, a spacer, a nonconventional nucleotide linking chemistry (as described further herein), other crosslinkers, or a nanoparticle (see e.g., FIG. 25A). Nanoparticles can include crystalline or amorphous particles with a particle size from about 2 to about 750 nanometers. Boehmite alumina can have an average particle size distribution from 2 to 750 nm. In some embodiments of any of the aspects, the nanoparticle is a metal nanoparticle. Non-limiting examples of metal nanoparticles include gold, silver, palladium or titanium nanoparticles or combinations thereof. In some embodiments of any of the aspects, the nanoparticle is of a sufficient size to reduce or prevent the 5′->3′ exonuclease from acting on the linked nucleic acid. In some embodiments of any of the aspects, the nanoparticle is linked to the 5′ end of the nucleic acid.

In some embodiments of any of the aspects, one or both of the first or second primers (e.g., the second primer or the first and second primers) comprises a nucleic acid modification that enhances 5′->3′ cleaving activity of the 5′->3′ exonuclease. In some embodiments of any of the aspects, the nucleic acid modification capable of enhancing 5′->3′ cleaving activity of a 5′->3′ exonuclease is a 5′ modification selected from the group consisting of: 5′-OH, phosphate group, 5′-monophosphate; 5′-diphosphate or a 5′-triphosphate.

It will be apparent to those skilled in the art that various modifications and variations can be made to improve the stability of the nucleic acid probe or primer strand.

### Exonuclease Digestion

The methods, kits and compositions provided herein rely on digestion of a nucleic acid strand, e.g., the probe strands via an exonuclease enzyme. Exonucleases are enzymes that work by cleaving nucleotides one at a time from the end (exo) of a polynucleotide chain. A hydrolyzing reaction that breaks phosphodiester bonds at either the 3′ or the 5′ end occurs. Without wishing to be bound by a theory, the exonuclease recognizes and digests the hybridized probe separating the reporter molecules and activating them for detection of the target nucleic acid.

In some embodiments, the exonuclease having the 5′ to 3′ exonuclease activity is a thermostable exonuclease. In some embodiments, the exonuclease having the 5′ to 3′ exonuclease activity is active at a higher temperature, e.g., 60° C. to 65° C. In some embodiments, the exonuclease is a Bst full length exonuclease. In some embodiments, multiple exonuclease enzymes are used. Non-limiting examples of exonuclease enzymes that can be used include, Bst Full Length, Taq DNA polymerase, T7 Exonuclease, Exonuclease VIII, Exonuclease VIII truncated, Lambda exonuclease, T5 Exonuclease, RecJf, and any combination thereof.

In some embodiments of any of the aspects, the exonuclease has polymerase activity. In some embodiments of any of the aspects, the exonuclease lacks polymerase activity.

In some embodiments of any of the aspects, the exonuclease is Bst DNA Polymerase. In some embodiments of any of the aspects, the exonuclease is Bst DNA Polymerase Full Length (“Bst Full Length” or “Bst FL”), which is the full length polymerase from Bacillus stearothermophilus. Bst Full Length has 5′ → 3′ polymerase and double-strand specific 5′ → 3′ exonuclease activity, but lacks 3′ → 5′ exonuclease activity. In some embodiments of any of the aspects, the exonuclease is selected from the group consisting of Bst Full Length (e.g., NEB M0328S), Bst Large Fragment (e.g., NEB M0275S), Bst 2.0 (e.g., NEB M0537S), Bst 2.0 WarmStart (e.g., NEB M0538S), and Bst 3.0 (e.g., NEB M0374S).

In some embodiments of any of the aspects, the exonuclease is provided (i.e., added to the reaction mixture) at a concentration of 0.1 U/µL to 5 U/µL. As used herein, one unit of Bst full length defined as the amount of enzyme that will incorporate 10 nmol of dNTP into acid insoluble material in 30 minutes at 65° C.

As a non-limiting example, the exonuclease (e.g., Bst FL) is provided at a concentration of at least 0.1 U/µL, at least 0.2 U/µL, at least 0.3 U/µL, at least 0.4 U/µL, at least 0.5 U/µL, at least 0.6 U/µL, at least 0.7 U/µL, at least 0.8 U/µL, at least 0.9 U/µL, at least 1.0 U/µL, at least 1.1 U/µL, at least 1.2 U/µL, at least 1.3 U/µL, at least 1.4 U/µL, at least 1.5 U/µL, at least 1.6 U/µL, at least 1.7 U/µL, at least 1.8 U/µL, at least 1.9 U/µL, at least 2.0 U/µL, at least 2.1 U/µL, at least 2.2 U/µL, at least 2.3 U/µL, at least 2.4 U/µL, at least 2.5 U/µL, at least 2.6 U/µL, at least 2.7 U/µL, at least 2.8 U/µL, at least 2.9 U/µL, at least 3.0 U/µL, at least 3.1 U/µL, at least 3.2 U/µL, at least 3.3 U/µL, at least 3.4 U/µL, at least 3.5 U/µL, at least 3.6 U/µL, at least 3.7 U/µL, at least 3.8 U/µL, at least 3.9 U/µL, at least 4.0 U/µL, at least 4.1 U/µL, at least 4.2 U/µL, at least 4.3 U/µL, at least 4.4 U/µL, at least 4.5 U/µL, at least 4.6 U/µL, at least 4.7 U/µL, at least 4.8 U/µL, at least 4.9 U/µL, at least 5.0 U/µL, at least 5.1 U/µL, at least 5.2 U/µL, at least 5.3 U/µL, at least 5.4 U/µL, at least 5.5 U/µL, at least 5.6 U/µL, at least 5.7 U/µL, at least 5.8 U/µL, at least 5.9 U/µL, at least 6.0 U/µL, at least 6.1 U/µL, at least 6.2 U/µL, at least 6.3 U/µL, at least 6.4 U/µL, at least 6.5 U/µL, at least 6.6 U/µL, at least 6.7 U/µL, at least 6.8 U/µL, at least 6.9 U/µL, at least 7.0 U/µL, at least 7.1 U/µL, at least 7.2 U/µL, at least 7.3 U/µL, at least 7.4 U/µL, at least 7.5 U/µL, at least 7.6 U/µL, at least 7.7 U/µL, at least 7.8 U/µL, at least 7.9 U/µL, at least 8.0 U/µL, at least 8.1 U/µL, at least 8.2 U/µL, at least 8.3 U/µL, at least 8.4 U/µL, at least 8.5 U/µL, at least 8.6 U/µL, at least 8.7 U/µL, at least 8.8 U/µL, at least 8.9 U/µL, at least 9.0 U/µL, at least 9.1 U/µL, at least 9.2 U/µL, at least 9.3 U/µL, at least 9.4 U/µL, at least 9.5 U/µL, at least 9.6 U/µL, at least 9.7 U/µL, at least 9.8 U/µL, at least 9.9 U/µL, at least 10 U/µL, at least 20 U/µL, at least 30 U/µL, at least 40 U/µL, or at least 50 U/µL.

The treatment with the exonuclease can be for any desired time. For example, the hybridized probes can be contacted with the exonuclease for a period of from about 15 seconds to about 2 hours. In some embodiments, the treatment with the exonuclease is for about 1 minutes. As a non-limiting example, the treatment with the exonuclease is for about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 70 minutes, about 75 minutes, about 80 minutes, or about 90 minutes.

In some embodiments of any of the aspects, the treatment with the exonuclease is for at most 5 minutes, at most 6 minutes, at most 7 minutes, at most 8 minutes, at most 9 minutes, at most 10 minutes, at most 11 minutes, at most 12 minutes, at most 13 minutes, at most 14 minutes, at most 15 minutes, at most 16 minutes, at most 17 minutes, at most 18 minutes, at most 19 minutes, at most 20 minutes, at most 21 minutes, at most 22 minutes, at most 23 minutes, at most 24 minutes, at most 25 minutes, at most 26 minutes, at most 27 minutes, at most 28 minutes, at most 29 minutes, at most 30 minutes, at most 31 minutes, at most 32 minutes, at most 33 minutes, at most 34 minutes, at most 35 minutes, at most 36 minutes, at most 37 minutes, at most 38 minutes, at most 39 minutes, at most 40 minutes, at most 41 minutes, at most 42 minutes, at most 43 minutes, at most 44 minutes, at most 45 minutes, at most 46 minutes, at most 47 minutes, at most 48 minutes, at most 49 minutes, at most 50 minutes, at most 51 minutes, at most 52 minutes, at most 53 minutes, at most 54 minutes, at most 55 minutes, at most 56 minutes, at most 57 minutes, at most 58 minutes, at most 59 minutes, at most 60 minutes, at most 70 minutes, at most 75 minutes, at most 80 minutes, or at most 90 minutes.

In some embodiments, treatment with exonuclease is for about 15 minutes to about 45 minutes. For example, treatment with exonuclease is for about 20 minutes to about 40 minutes for from about 25 minutes to about 35 minutes.

In some embodiments, the methods, kits and compositions provided herein further comprises a DNA polymerase. A “polymerase” refers to an enzyme that performs template-directed synthesis of polynucleotides, e.g., DNA and/or RNA. The term encompasses both the full length polypeptide and a domain that has polymerase activity. DNA polymerases are well-known to those skilled in the art, including but not limited to DNA polymerases isolated or derived from Pyrococcusfuriosus, Thermococcuslitoralis, and Thermotogamaritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.). Polymerases include both DNA-dependent polymerases and RNA-dependent polymerases such as reverse transcriptase. At least five families of DNA-dependent DNA polymerases are known, although most fall into families A, B and C. There is little or no sequence similarity among the various families. Most family A polymerases are single chain proteins that can contain multiple enzymatic functions including polymerase, 3′ to 5′ exonuclease activity and 5′ to 3′ exonuclease activity. Family B polymerases typically have a single catalytic domain with polymerase and 3′ to 5′ exonuclease activity, as well as accessory factors. Family C polymerases are typically multi-subunit proteins with polymerizing and 3′ to 5′ exonuclease activity. In E.coli, three types of DNA polymerases have been found, DNA polymerases I (family A), II (family B), and III (family C). In eukaryotic cells, three different family B polymerases, DNA polymerases α, δ, and ε, are implicated in nuclear replication, and a family A polymerase, polymerase γ, is used for mitochondrial DNA replication. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

In some embodiments of any of the aspects, the DNA polymerase used in the amplification step is a strand-displacing polymerase. The term strand displacement describes the ability to displace downstream DNA encountered during synthesis. In some embodiments of any of the aspects, at least one (e.g. 1, 2, 3, or 4) strand-displacing DNA polymerase is selected from the group consisting of: Polymerase I Klenow fragment, Bst polymerase, Phi-29 polymerase, and Bacillus subtilis Pol I (Bsu) polymerase.

The exonuclease digests the nucleic acid probe provided herein and release the reporter molecule from the nucleic acid composition to produce a detectable signal. (e.g., fluorescence or chemiluminescence).

### Reporter Molecules

The methods and compositions provided herein rely on a reporter molecule capable of producing a detectable signal.

In some embodiments of any of the aspects, the nucleic acid probe provided herein comprises a plurality of reporter molecules. In some embodiments, at least two reporter molecules in the plurality of reporter molecules are different. This allows for the detection of multiple target nucleic acids in one reaction, i.e., multiplexed detection. In some embodiments, at least two target nucleic acids are detected, e.g., at least 2, at least 3, at least 4, at least 5, at least 6, at least 7, at least 8, at least 9, at least 10 or more different target nucleic acids are detected. In some embodiments, each target nucleic acid is detected with a nucleic acid probe comprising a distinguishable reporter molecule.

In some embodiments of any of the aspects, the reporter molecule provided herein is selected from the group consisting of: fluorescent molecules/fluorophores, radioisotopes, chromophores, enzymes, enzyme substrates, chemiluminescent moieties, bioluminescent moieties, echogenic substances, non-metallic isotopes, optical reporters, paramagnetic metal ions, and ferromagnetic metals.

In other embodiments, a detection reagent (e.g., a primer, a probe, etc.) is labeled with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS ; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Alexa Fluor 546™; Alexa Fluor 568™; Alexa Fluor 594™; Alexa Fluor 633™; Alexa Fluor 647™; Alexa Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™ -1; BOBO™ -3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™ -1; BO-PRO™ -3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP - Cyan Fluorescent Protein; Chlorophyll; Chromomycin A; Chromomycin A; CMFDA; Coelenterazine ; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200 ; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine ; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

Many suitable forms of these fluorescent compounds are available and can be used. Additional fluorophore examples include, but are not limited to fluorescein, phycoerythrin, phycocyanin, o-phthalaldehyde, fluorescamine, Cy3™, Cy5™, allophycocyanin, Texas Red, peridinin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetramethylrhodamine isothiocyanate (TRITC)), biotin, phycoerythrin, AMCA, CyDyes™, 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g., cyanine dyes such as Cy3, Cy5, etc.; BODIPY dyes and quinoline dyes.

Other exemplary detectable labels include luminescent and bioluminescent markers (e.g., biotin, luciferase (e.g., bacterial, firefly, click beetle and the like), luciferin, and aequorin), radiolabels (e.g., 3H, 125I, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

In some embodiments of any of the aspects, a detectable label can be a radiolabel including, but not limited to ³H, ¹²⁵I, ³⁵S, ¹⁴C, ³²P, and ³³P. Suitable non-metallic isotopes include, but are not limited to, ¹¹C, ¹⁴C, ¹³N, ¹⁸F, ¹²³I, ¹²⁴I, and ¹²⁵I. Suitable radioisotopes include, but are not limited to, ⁹⁹mTc, ⁹⁵Tc, ¹¹¹In, ⁶²Cu, ⁶⁴Cu, Ga, ⁶⁸Ga, and ¹⁵³Gd. Suitable paramagnetic metal ions include, but are not limited to, Gd(III), Dy(III), Fe(III), and Mn(II). Suitable X-ray absorbers include, but are not limited to, Re, Sm, Ho, Lu, Pm, Y, Bi, Pd, Gd, La, Au, Au, Yb, Dy, Cu, Rh, Ag, and Ir.

In some embodiments, the detectable label is a fluorophore or a quantum dot. Without wishing to be bound by a theory, using a fluorescent reagent can reduce signal-to-noise in the imaging/readout, thus maintaining sensitivity.

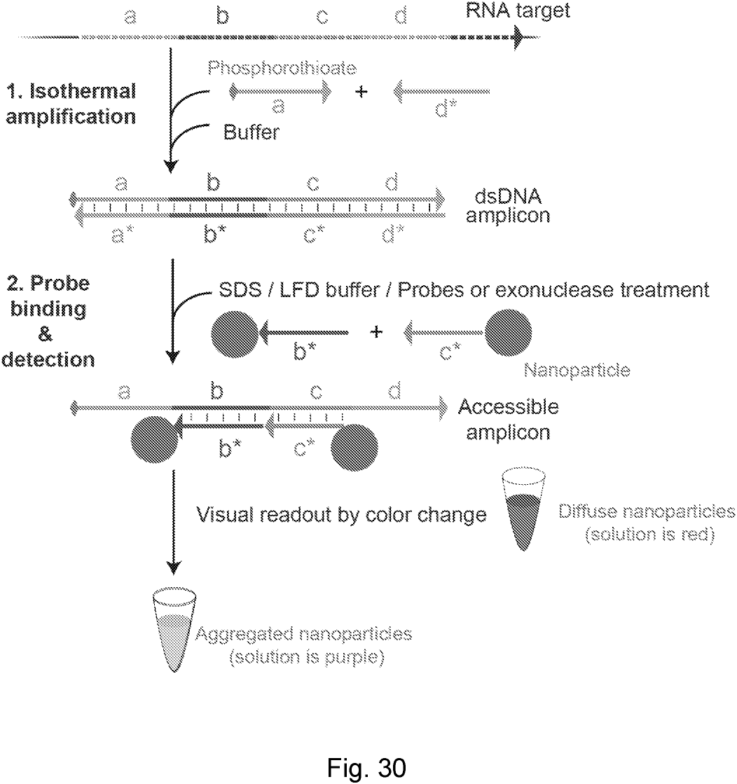

In some embodiments of any of the aspects, the reporter molecule comprises a nanoparticle whose optical properties change based on the particle density (see e.g., FIG. 30). For example, at least two nucleic acid probes specific to the single-stranded amplicon can each be linked to such a nanoparticle (e.g., at the 5′ end and/or 3′ end of each). In absence of amplicon binding, the diffuse nanoparticle probes cause the solution to be a first color (e.g., red). Binding to the target amplicon creates aggregation of the nanoparticles, causing the solution turn a second color (e.g., purple). The color change hence indicates the presence of the target amplicon in solution. As a non-limiting example, gold nanoparticles can exhibit color changes in solution depending on the gold nanoparticle density. In some embodiments of any of the aspects, the nanoparticles are aggregated by conjugating them or binding them to functional groups on the detection probes, e.g., during the detection step.

### Quenchers

In some embodiments of any of the aspects, the nucleic acid probe further comprises a quencher molecule. The quencher can act to decrease a detectable property, e.g., the intensity, color, etc. of the detectable signal from a reporter molecule provided herein. In some embodiments, the quencher molecule is at the 5′ end of the nucleic acid probe. In some embodiments, the quencher molecule is at the 3′ end of the nucleic acid probe. In some embodiments, the quencher molecule is at an internal position of the nucleic acid probe. In some embodiments, the quencher molecule is at an internal position of the nucleic acid probe, such as in a stem or loop structure of the probe. In some embodiments, a first quencher molecule is at the 5′ end of the nucleic acid probe, and a second quencher molecule is at an internal position of the nucleic acid probe. In some embodiments, a first quencher molecule is at the 3′ end of the nucleic acid probe and a second quencher molecule is at an internal position of the nucleic acid probe.

Multiple quencher molecules can be used. In some embodiments, the nucleic acid probe comprises 2, 3, 4, 5 or more quencher molecules, which can be the same or different from each other. In some embodiments of any of the aspects, the nucleic acid probe further comprises at least one additional quencher molecule. It is noted that when two or more quencher molecule are present, they can be independently located anywhere in the nucleic acid probe. For example, one quencher can be at one end of the probe and the second quencher can be at an internal position of the probe. For example, the first quencher molecule can be at an internal position of the probe and the second quencher molecule can be at the 3′-end of the probe. In some other embodiments, the first quencher molecule can be at an internal position of the probe and the second quencher molecule can be at the 5′-end of the probe.

In some embodiments of any one of the aspects, the probe comprises at least one reporter molecule and at least two quencher molecules. For example, the probe comprises at least one reporter molecules and at least two quencher molecules, where one reporter molecule is at a first end of the probe, a first quencher molecules is at an internal position of the probe and a second quencher molecule is at an internal position or a second end of the probe. For example, the reporter molecule is at the 5′-end of the probe, first quencher molecule is at an internal position of the probe, and the second quencher molecule is at 3′-end of the probe.

In some embodiments, the probe comprises a reporter molecule at an internal position of the probe and a quencher molecule at an end position, e.g., 5′- or 3′-end of the probe.

In some embodiments of any of the aspects, the nucleic acid probe comprises a 5′ fluorophore (e.g., Cy5 or FAM), an internal Zen or Tao quencher molecule, and a 3′ Iowa Black quencher molecule. Further non-limiting examples of nucleic acid probes are provided below in Table 5 (see also SEQ ID NOs: 19, 51-55 and Table 4).

Table 5: Exemplary Quencher Molecule Configurations; the reporter molecule can be any known in the art or described herein (e.g., a fluorophore; e.g., Cy5, FAM).

| | | |
|---|---|---|
| 5′ position of nucleic acid probe | internal position of nucleic acid probe | 3′ position of nucleic acid probe |
| Reporter Molecule (e.g., Cy5) | | Iowa Black (e.g., Iowa Black RQ) |
| Reporter Molecule (e.g., Cy5) | TAO | Iowa Black (e.g., Iowa Black RQ) |
| Iowa Black (e.g., Iowa Black RQ) | | Reporter Molecule (e.g., Cy5) |
| Iowa Black (e.g., Iowa Black RQ) | TAO | Reporter Molecule (e.g., Cy5) |
| Reporter Molecule (e.g., FAM) | | Iowa Black (e.g., Iowa Black FQ) |
| Reporter Molecule (e.g., FAM) | ZEN | Iowa Black (e.g., Iowa Black FQ) |
| Iowa Black (e.g., Iowa Black FQ) | | Reporter Molecule (e.g., FAM) |
| Iowa Black (e.g., Iowa Black FQ) | ZEN | Reporter Molecule (e.g., FAM) |

The reporter molecule and the quencher molecule can be positioned such that the quencher molecule quenches a detectable signal produced by the reporter molecule when the probe is not hybridized to the amplicon. In some embodiments, the reporter molecule and the quencher molecule can be positioned such that the quencher molecule also quenches the detectable signal from the reporter molecule when the nucleic acid probe is hybridized to the amplicon. Generally, the reporter molecule and the quencher molecule (e.g., first or second quencher molecule) are separated by at least 4 nucleotides. In some embodiments, the reporter molecule and the quencher molecule (e.g., first or second quencher molecule) are separated by at least 9 nucleotides. For example, the reporter molecule and the quencher molecule (e.g., first or second quencher molecule) are separated by at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides. In some embodiments, the reporter molecule and the quencher molecule (e.g., first or second quencher molecule) are separated by no more than 50 nucleotides. For example, the reporter molecule and the quencher molecule (e.g., first or second quencher molecule) are separated by no more than 30 nucleotides, no more than 25 nucleotides, no more than 20 nucleotides, no more than 19 nucleotide, no more than 18 nucleotides, no more than 17 nucleotides, no more than 16 nucleotides, no more than 15 nucleotides, no more than 14 nucleotides, no more than 13 nucleotides, no more than 12 nucleotides, no more than 11 nucleotides, no more than 10 nucleotides, no more than 9 nucleotides, no more than 8 nucleotides, no more than 7 nucleotides, no more than 6 nucleotides, no more than 5 nucleotides, or no more than 4 nucleotides.

The reporter molecule and the first and second quencher molecules can be positioned such that the quencher molecules quench a detectable signal produced by the reporter molecule when the probe is not hybridized to the amplicon. In some embodiments, the reporter molecule and the first and second quencher molecule can be positioned such that the quencher molecules also quench the detectable signal from the reporter molecule when the nucleic acid probe is hybridized to the amplicon. Generally, the first and second quencher molecules are separated by at least 4 nucleotides. In some embodiments, the first and second quencher molecules are separated by at least 19 nucleotides. For example, the first and second quencher molecules are separated by at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides. In some embodiments, the first and second quencher molecules are separated by no more than 50 nucleotides. For example, the first and second quencher molecules are separated by no more than 30 nucleotides, no more than 25 nucleotides, no more than 20 nucleotides, no more than 19 nucleotide, no more than 18 nucleotides, no more than 17 nucleotides, no more than 16 nucleotides, no more than 15 nucleotides, no more than 14 nucleotides, no more than 13 nucleotides, no more than 12 nucleotides, no more than 11 nucleotides, no more than 10 nucleotides, no more than 9 nucleotides, no more than 8 nucleotides, no more than 7 nucleotides, no more than 6 nucleotides, no more than 5 nucleotides, or no more than 4 nucleotides.

In some embodiments, the reporter molecule and the first quencher molecule are separated by at least 4 nucleotides, and, independently the first and second quencher molecules are separated by at least 4 nucleotides. For example, the reporter molecule and the first quencher molecule are separated by at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides, and independently the first and second quencher molecules are separated by at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides.

In some embodiments, the reporter molecule and the first quencher molecule are closer to each other relative to the distance between the first and second quencher molecule. In some other embodiments, the first and second quencher molecule are closer to each other relative to the distance between the reporter molecule and the first/second quencher molecule.

When the probe is hybridized with the target nucleic acid sequence, the exonuclease having the 5′ to 3′ exonuclease activity digests its 5′-end portion or its 5′-end and releases either the reporter molecule or the quencher molecule located on its 5′- end portion or its 5′-end, thereby unquenching the detectable signal of the reporter molecule to generate a detectable signal indicative of the target nucleic acid sequence.

In some embodiments of any of the aspects, the quencher molecule quenches the detectable signal from the reporter molecule when the nucleic acid probe is not hybridized to the amplicon. In some embodiments of any of the aspects, quencher molecule quenches the detectable signal from the reporter molecule when the nucleic acid probe is not hybridized to a complementary nucleic acid strand. In some embodiments, the quencher molecule quenches the detectable signal from the reporter molecule when the nucleic acid probe is hybridized to a complementary nucleic acid strand.

In some embodiments of any of the aspects, the quenching is partial quenching or complete quenching. As used herein the term “completely quenched” refers to the inability to detect any signal from the reporter molecule, i.e., 100% quenched or 0% detectable signal (e.g., fluorescence). As used herein the term “partially quenched” refers detectable signal from the reporter molecule that is reduced compared to the full detectable signal from the reporter molecule. In some embodiments of any of the aspects, “partially quenched” refers to signal from the reporter molecule that is reduced by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.5%, at least 99.9% or more.

In some embodiments of any of the aspects, the at least one quencher molecule quenches the specific wavelength of the fluorescence emitted by the reporter molecule in the nucleic acid probe. As a non-limiting example, some fluorophores, such as TET, HEX, and FAM, with an emission range between 500 nm to 550 nm are quenched by quenchers, such as Black hole quencher 1 (BHQ1) and Dabcyl, with an absorption range of 450 nm to 550 nm. Similarly, TMR, Texas red, ROX, Cy3, and Cy5 are quenched by BHQ2. See e.g., Marras, Selection of fluorophore and quencher pairs for fluorescent nucleic acid hybridization probes, Methods Mol Biol. 2006;335:3-16; the content of which is incorporated herein by reference in its entirety.

In some embodiments of any of the aspects, the quencher molecule is a dark quencher. A dark quencher (also known as a dark sucker) is a substance that absorbs excitation energy from a reporter molecule, e.g., a fluorophore, and dissipates the energy as heat; while a typical (fluorescent) quencher re-emits much of this energy as light. Non-limiting examples of quencher molecules (e.g., non-fluorescent or dark quenchers that dissipate energy absorbed from a fluorescent dye) include the Black Hole Quenchers™ (Biosearch Technologies™); Iowa Black quenchers (e.g., Iowa Black FQ™ (“3IABkFQ”) and Iowa Black RQ™ (e.g., “3IAbRQSp”)); Eclipsed® Dark Quenchers (Epoch Biosciences™), Zen™ quenchers (Integrated DNA Technologies™; “e.g., “ZEN”); TAO™ quenchers (Integrated DNA Technologies™; “e.g., “TAO”); Dabcyl (4-(4′-dimethylaminophenylazo)benzoic acid); Qxl™ quenchers; QSY® quenchers; and IRDye® QC-1. Additional non-limiting examples of quenchers are also provided in U.S. Pat. No. 6,465,175, 7,439,341, 12/252,721, 7,803,536, 12/853,755, 7,476,735, 7,605,243, 7,645,872, 8,030,460, 13/224,571, 8,916,345, the contents of each of which are incorporated herein by reference in their entireties.

In some embodiments of any of the aspects, the quencher molecule is an Iowa Black® quencher. In some embodiments of any of the aspects, the Iowa Black® quencher is preferably at the 5′ or 3′ position of the nucleic acid probe. In some embodiments of any of the aspects, the quencher molecule is Iowa Black® FQ, which has a broad absorbance spectra ranging from 420 to 620 nm with peak absorbance at 531 nm (i.e., the green-yellow region of the visible light spectrum). In some embodiments, Iowa Black® FQ (e.g., “3IABkFQ”) is used to quench fluorescein or other fluorescent dyes that emit in the green to pink spectral range. In some embodiments of any of the aspects, the quencher molecule is Iowa Black® RQ, which has a broad absorbance spectra ranging from 500 to 700 nm with peak absorbance at 656 nm (i.e., the orange-red region of the visible light spectrum). In some embodiments, Iowa Black® RQ (e.g., “3IAbRQSp”) is used to quench Texas Red®, Cy5, or other fluorescent dyes that emit in the red spectral range.

In some embodiments of any of the aspects, the quencher molecule is a ZEN quencher. In some embodiments of any of the aspects, the ZEN quencher is preferably at an internal position of the nucleic acid probe. See e.g., Lennox et al., Mol Ther Nucleic Acids. 2013 Aug; 2(8): e117; U.S. Pat. 8916345, 9506059; the contents of each of which are incorporated herein by reference in their entireties. ZEN can quench a similar range of fluorophores as Iowa Black® FQ, e.g., FAM, SUN, JOE, HEX, or MAX. In some embodiments, the nucleic acid probe comprises ZEN, Iowa Black® FQ, and a reporter molecule such as FAM.

In some embodiments of any of the aspects, the quencher molecule is a TAO quencher. In some embodiments of any of the aspects, the TAO quencher is preferably at an internal position of the nucleic acid probe. TAO can quench a similar range of fluorophores as Iowa Black® RQ, e.g., Cy3, ATTO550, ROX, Texas red, ATTO647N, or Cy5. In some embodiments, the nucleic acid probe comprises TAO, Iowa Black® RQ, and a reporter molecule, such as Cy5.

In some embodiments of any of the aspects, the quencher molecule is a black hole quencher. The Black Hole Quenchers™ are structures comprising at least three radicals selected from substituted or unsubstituted aryl or heteroaryl compounds, or combinations thereof, wherein at least two of the residues are linked via an exocyclic diazo bond (see, e.g., International Publication No. WO2001086001). Black Hole Quenchers (BHQ) are capable of quenching across the entire visible spectrum. Non-limiting examples of Black Hole Quenchers include BHQ-0 (430-520 nm); BHQ-1 (480-580 nm, 534 nm absorbance (abs) max); BHQ-2 (520-650 nm, 544 nm abs max); BHQ-3 (620-730 nm, 672 nm abs max); and BHQ-10 (480-550 nm, 516 nm abs max; Water Soluble).

In some embodiments of any of the aspects, the quencher molecule is Dabcyl (4-(4′-dimethylaminophenylazo)benzoic acid) or a derivative thereof. Dabcyl absorbs in the green region of the visible light spectrum (e.g., 346-489 nm, with a peak absorbance at 474 nm) and can be used with fluorescein or other fluorophores that emit in the green region.

In some embodiments of any of the aspects, the quencher molecule is an Eclipse® Dark Quencher. The absorbance maximum for the Eclipse Quencher is at 522 nm, compared to 479 nm for Dabcyl. In addition, the structure of the Eclipse Quencher is substantially more electron deficient than that of Dabcyl and this leads to better quenching over a wider range of dyes, especially those with emission maxima at longer wavelengths (red shifted) such as Redmond Red and Cyanine 5. In addition, with an absorption range from 390 nm to 625 nm, the Eclipse Quencher is capable of effective quenching of a wide range of fluorophores.

In some embodiments of any of the aspects, the quencher molecule is a QSY® quencher. Non-limiting examples of QSY quenchers include QSY35 (410-500 nm, 475 nm max abs), QSY7 (500-600 nm, 560 nm max abs), QSY21 (590-720 nm, 661 nm abs max), and QSY9 (500-600 nm, 562 nm abs max).

In some embodiments of any of the aspects, the quencher molecule is a Qxl™ quencher. Qxl™ quenchers span the full visible spectrum. Non-limiting examples of QXL quenchers include QXL490 (495 nm abs max, can be used as a quencher for EDANS, AMCA, and most coumarin fluorophores), QXL520 (~ 520 nm abs max, can be used as a quencher for FAM), QXL570 (578 nm abs max, can be used as a quencher for rhodamines (such as TAMRA, sulforhodamine B, ROX) and Cy3 fluorophores), QXL610 (~610 nm abs max, can be used as a quencher for ROX), and QXL670 (668 nm abs max, can be used as a quencher for Cy5 and Cy5-like fluorophores such as HiLyte™ Fluor 647).

In some embodiments of any of the aspects, the quencher molecule is IRDye QC-1. IRDye QC-1 quenches dyes from the visible to the near-infrared range (500-900 nm, max abs 737 nm).

TABLE 6

Exemplary Quencher Molecules

| Quencher | Structure | Abs (nm) | Abs Max (nm) |
|---|---|---|---|
| ZEN | 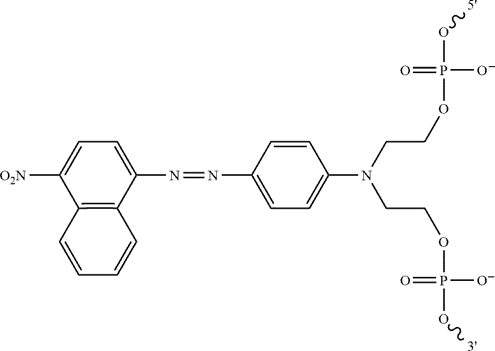 | ~420-620 | ~530 |
| BHQ-0 | 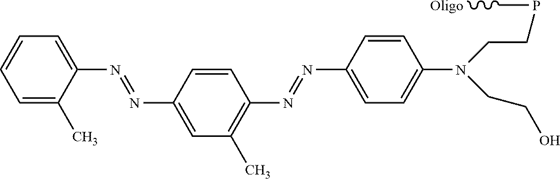 | 430-520 | 495 |
| BHQ-1 | 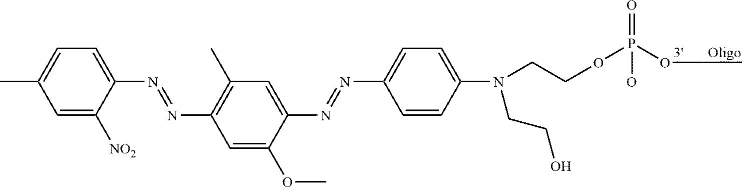 | 480-580 | 534 |
| BHQ-2 | 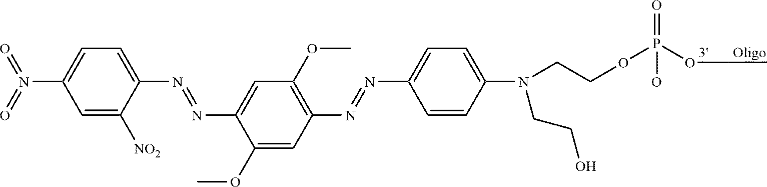 | 520-650 | 544 |
| BHQ-3 | 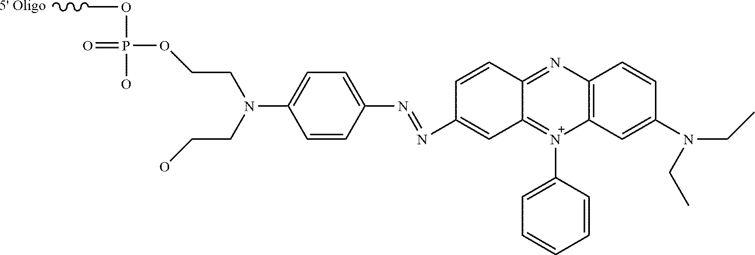 | 620-730 | 672 |
| BHQ-10 | 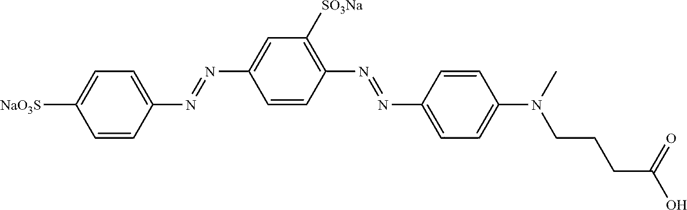 | 480-550 | 516 |
| Dabcyl | 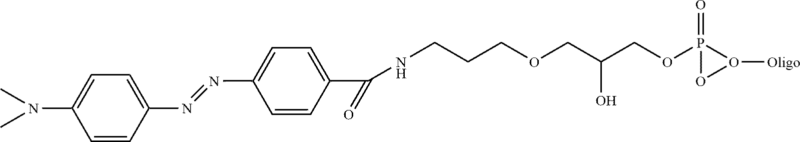 | 346-489 | 474 |
| Eclipse | 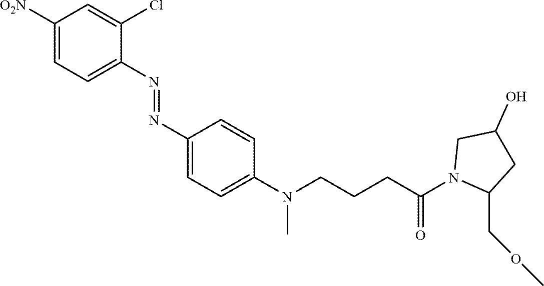 [SMILES: COCC1CC(O)CN1C(=O)CCCN(C)c1ccc(/N=N/c2ccc([N+](=O)[O-])cc2Cl)cc1] | 390-625 | 522 |
| QSY 21 | 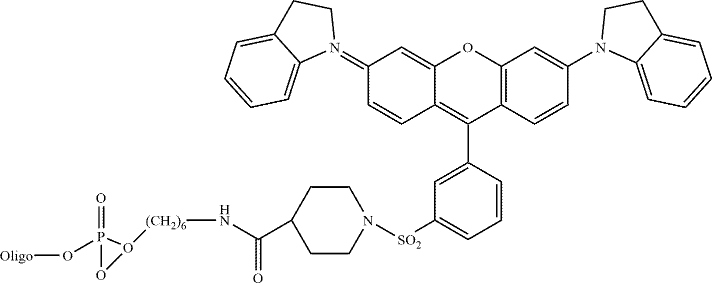 | 590-720 | 661 |
| QSY 35 | 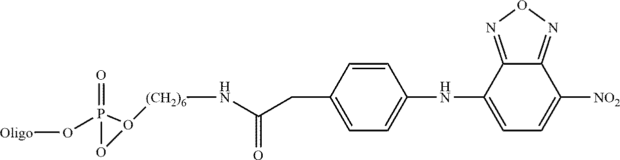 | 410-550 | 475 |
| QSY 7 | 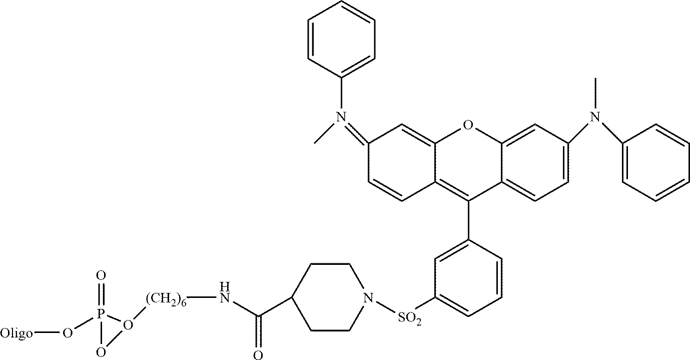 | 500-600 | 560 |
| QSY 9 | 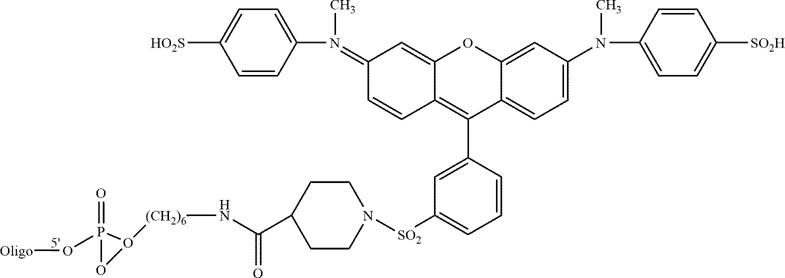 | 500-600 | 562 |
| IRDye® QC-1 | 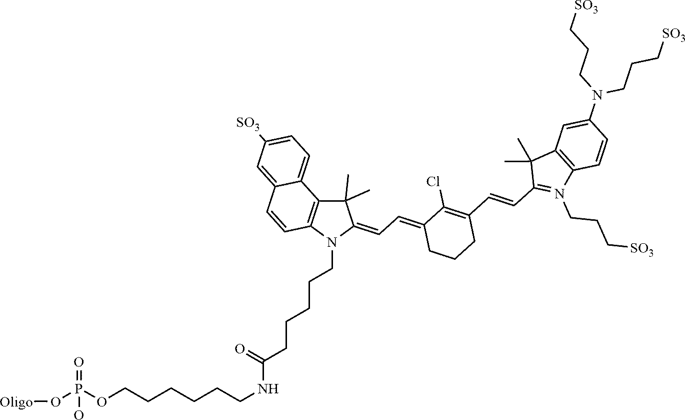 | 500-800 | 737 |

### Detection Methods

Means of detecting such reporter labels and quenchers are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label. In some embodiments of any of the aspects, the detection of a reporter and/or quencher molecule provided herein comprises fluorescence detection, luminescence detection, chemiluminescence detection, colorimetric, or immunofluorescence detection.

In some embodiments of any of the aspects, the detection method is selected from the group consisting of: lateral flow detection; hybridization with conjugated or unconjugated DNA; colorimetric assays; gel electrophoresis; a toehold-mediated strand displacement reaction; molecular beacons; fluorophore-quencher pairs; microarrays; Specific High-sensitivity Enzymatic Reporter unLOCKing (SHERLOCK); DNA endonuclease-targeted CRISPR trans reporter (DETECTR); sequencing; and quantitative polymerase chain reaction (qPCR). In some embodiments of any of the aspects, the detection method comprises a plate-based assay (e.g., SHERLOCK, DETECTR, microarray, hybridization, qPCR, sequencing, etc.).

In some embodiments of any of the aspects, the reporter molecule can be detected using lateral flow detection, also known as a lateral flow immunoassay assay (LFIA), laminar flow, the immunochromatographic assay, or strip test. LFIAs are a simple device intended to detect the presence (or absence) of antigen, e.g. a reporter molecule, in a fluid sample. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with an antibody (e.g., specific for a detectable marker on the target nucleic acid or for a detectable marker on a complementary nucleic to the target nucleic acid) or pretreated with a conjugated or unconjugated DNA as described herein. Depending upon the level of target present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick tests, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments of any of the aspects, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabeled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of lateral flow tests to detect nucleic acids have been described in the art; see e.g., U.S. Pat. Nos. 9,121,849; 9,207,236; and 9,651,549; the content of each of which is incorporated herein by reference in its entirety. The use of “dip sticks” or LFIA test strips and other solid supports have been described in the art in the context of an immunoassay for a number of targets. U.S. Pat. Nos. 4,943,522; 6,485,982; 6,187,598; 5,770,460; 5,622,871; 6,565,808, U.S. Pat. Applications Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick.

Typically, a lateral flow strip comprises: a sample pad, a conjugate pad, a detection membrane, and optionally an absorption pad. The sample pad is the first pad of the flow strip and it is the location where sample, e.g., the amplification reaction as described herein, is added. In some embodiments of any of the aspects, the sample pad comprises cellulose fiber filters and/or woven meshes. In some embodiments of any of the aspects, the sample pad further comprises a buffer. The conjugate pad is between the sample pad and the membrane; the conjugate pad comprises detector molecules, which are distributed into the membrane of the lateral flow strip after being contacted with the running buffer from the sample pad. In some embodiments of any of the aspects, the conjugate pad comprises glass fibers, cellulose fibers, and/or surface-modified polyester. In some embodiments of any of the aspects, the detection membrane is a nitrocellulose membrane, comprising the test line(s) and control lines(s). Absorbent pads, when used, are placed at the distal end of the lateral flow strip. The primary function of the absorbent pad is to increase the total volume of running buffer that enters the lateral flow strip.

In some embodiments of any of the aspects, a lateral flow strip comprises a region specific for the target amplification product or a region specific for a probe that hybridizes to the target amplification product. In some embodiments of any of the aspects, a lateral flow strip comprises a region specific to a positive control or a region specific for a probe that hybridizes to the positive control.

In some embodiments of any of the aspects, the lateral flow strip is contacted with a buffer comprising the amplicon to be detected and at least one probe; such a buffer can also be referred to herein as a running buffer or a hybridization buffer. In some embodiments of any of the aspects, the running buffer further comprises a surfactant as described further herein (e.g., SDS). In some embodiments of any of the aspects, the surfactant is added at any step described herein (e.g., amplification, exonuclease digestion, detection, etc.). In some embodiments of any of the aspects, the amplification reaction comprising a surfactant (e.g., SDS), optionally further comprising an exonuclease, are added to the running buffer. In some embodiments of any of the aspects, the amplification reaction, optionally further comprising an exonuclease, is added to the running buffer, which comprises a surfactant (e.g., SDS). In some embodiments of any of the aspects, the amplification reaction, optionally further comprising an exonuclease, is added to the running buffer, and a surfactant (e.g., SDS) is then added.

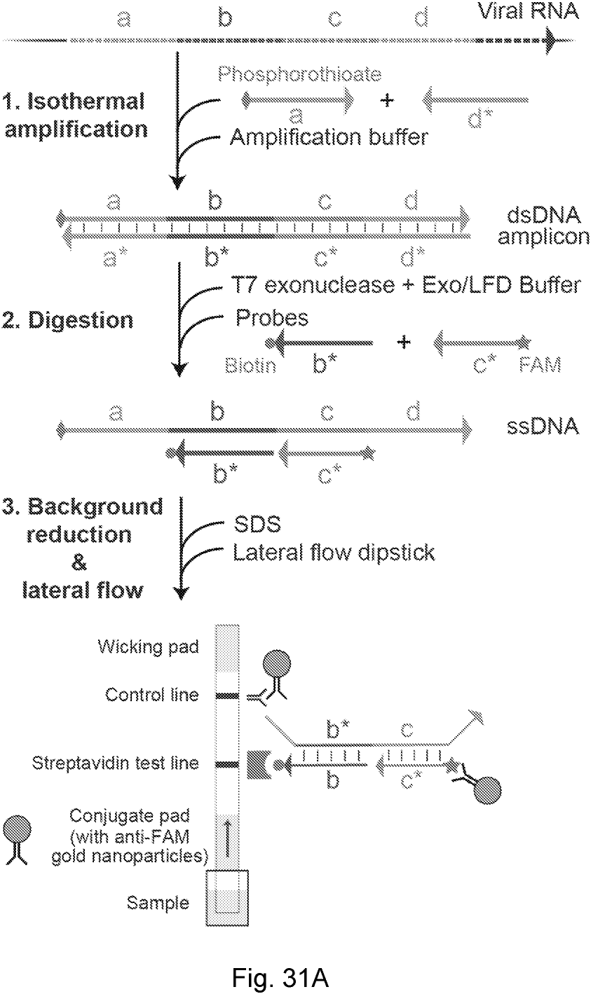

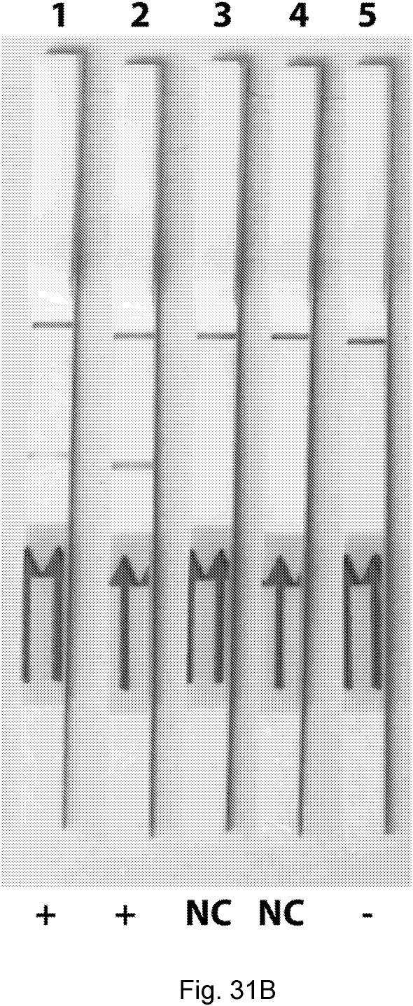

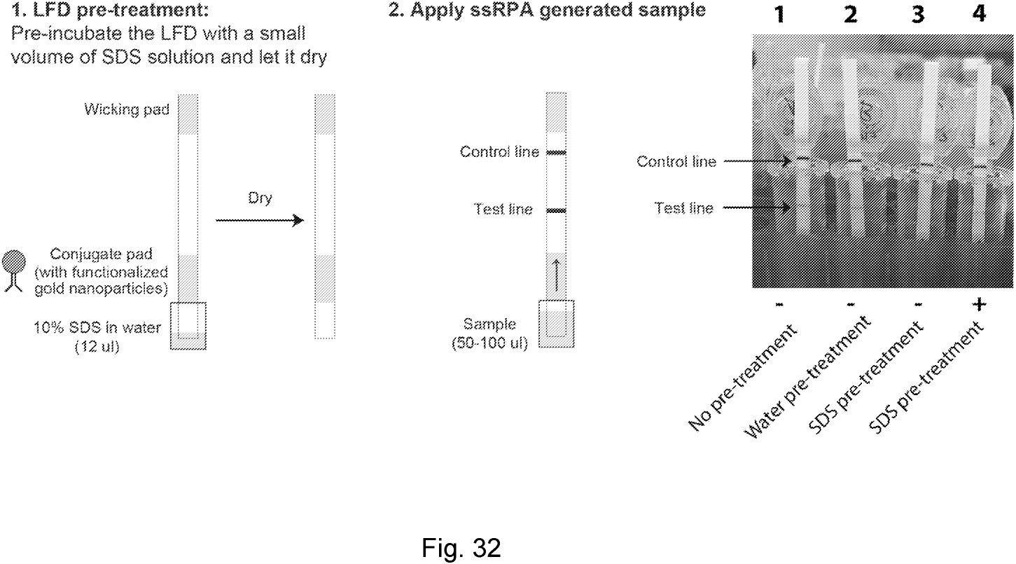

In some embodiments of any of the aspects, a lateral flow test strip of the assay is pretreated with the surfactant, e.g., SDS. In some embodiments of any of the aspects, the lateral flow strip is contacted with a surfactant prior to being contacted with the running buffer. In some embodiments of any of the aspects, the surfactant is dried onto the lateral flow strip. In some embodiments of any of the aspects, the conjugate pad of the lateral flow strip is contacted with a surfactant (e.g., SDS). In some embodiments of any of the aspects, the conjugate pad of the lateral flow strip comprises a dried surfactant (e.g., SDS). In some embodiments of any of the aspects, the detection membrane of the lateral flow strip is contacted with a surfactant (e.g., SDS). In some embodiments of any of the aspects, the detection membrane of the lateral flow strip comprises a dried surfactant (e.g., SDS). In some embodiments of any of the aspects, the sample pad of the lateral flow strip is contacted with a surfactant (e.g., SDS). In some embodiments of any of the aspects, the sample pad of the lateral flow strip comprises a dried surfactant (e.g., SDS). In some embodiments of any of the aspects, a material (e.g., a membrane) separate from the lateral flow strip is contacted with a surfactant (e.g., SDS), and the material comprising the surfactant is added to the amplification reaction or to the running buffer, prior to, at the same time, or after addition of the lateral flow strip. In some embodiments of any of the aspects, the surfactant (e.g., SDS) dried onto the material (e.g., a membrane) separate from the lateral flow strip. In some embodiments of any of the aspects, the material (e.g., a membrane) comprising a surfactant, wherein the material is separate from the lateral flow strip, is used to stir the running buffer, prior to, at the same time, or after addition of the lateral flow strip and/or amplification reaction. See e.g., FIGS. 31A-31B, FIG. 32.

The lateral flow assay can be carried out in lateral flow device (LFD), i.e., a lateral flow the test strip. The later flow device or strip comprises a test region. The test region comprises a ligand binding molecule immobilized therein. For example, a ligand binding molecule capable of binding with the reporter molecule or a moiety linked to the reporter molecule. In some embodiments, the ligand binding molecule is an antibody. In some embodiments, the later flow device or strip also comprises a control region comprising a different ligand binding molecule immobilized therein. The ligand binding molecule in the control region can bind to a ligand in the nucleic acid probe. Accordingly, in some embodiments, the nucleic acid probe comprises a lateral flow detectable moiety. Non-limiting examples of lateral flow detectable moieties include metallic moieties (e.g., metallic nanoparticle or metallic nanoshell, etc.), latex beads (including colored latex), carbon black nanoparticles, fluorophore, and the like. In some embodiments, the metallic nanoparticle or metallic nanoshell is selected from the group consisting of gold particles, silver particles, copper particles, platinum particles, cadmium particles, composite particles, gold hollow spheres, gold-coated silica nanoshells, and silica-coated gold shells.

The conditions for the detection step depend on the specific assay. In some embodiments of any of the aspects, the lateral flow detection step is performed in at most 1 minute, at most 2 minutes, at most 3 minutes, at most 4 minutes, at most 5 minutes, at most 6 minutes, at most 7 minutes, at most 8 minutes, at most 9 minutes, at most 10 minutes, at most 20 minutes, at most 30 minutes, at most 40 minutes, at most 50 minutes, or at most 60 minutes. In some embodiments of any of the aspects, the lateral flow detection step is performed in at least 5 minutes. As a non-limiting example, the lateral flow detection step can be for a period of 30 minutes or less, 25 minutes or less, 20 minutes or less, 15 minutes or less, 10 minutes or less, or 5 minutes or less. In some embodiments of any of the aspects, the lateral flow detection step is performed in at most 5 minutes. As a non-limiting example, the lateral flow detection step is performed in at most 5 minutes, at most 6 minutes, at most 7 minutes, at most 8 minutes, at most 9 minutes, at most 10 minutes, at most 15 minutes, at most 20 minutes, at most 25 minutes, at most 30 minutes, at most 40 minutes, at most 50 minutes, at most 60 minutes, at most 70 minutes, at most 80 minutes, at most 90 minutes, or at most 100 minutes.

As disclosed herein, any remaining uncleaved probes can be detected. Methods for detecting nucleic acid strands are well known in the art. For example, any remaining uncleaved probes can be detected by a sequence specific detection method. In some embodiments, said detecting the uncleaved nucleic acid probe comprises lateral flow detection.

In some embodiments, the nucleic acid probe is immobilized on a surface. In some embodiments of any of the aspects, the probe is conjugated to a lateral flow test strip as described herein. In some embodiments of any of the aspects, the probe is conjugated to a detectable marker as described herein (e.g., biotin, FAM, FITC, digoxigenin, etc.), and a lateral flow test strip comprises at least one region that is specific for the detectable marker conjugated to the probe (e.g., anti-biotin, streptavidin, anti-FAM, anti-FITC, anti-digoxigenin).

In some embodiments, at least one primer used in the amplification is immobilized on a surface. As such, each nucleic acid targets can use the same reporter molecule for detection (e.g., the same fluorophore for each different probe sequence), as the particular spatial configuration of the signal (e.g., on the immobilized surface) indicates which targets were detected. Non-limiting examples of such surfaces include a slide, a tube, a dipstick, a test strip, a diagnostic strip, a microchips, a filtration device, a membrane, a hollow-fiber reactor, or a microfluidic device, and the like.

In some embodiments of any of the aspects, the nucleic acid probe comprises a ligand for a ligand binding molecule. A ligand can be independently selected from the group consisting of organic and inorganic molecules, peptides, polypeptides, proteins, peptidomimetics, glycoproteins, lectins, nucleosides, nucleotides, monosaccharides, disaccharides, trisaccharides, oligosaccharides, polysaccharides, lipopolysaccharides, vitamins, steroids, hormones, cofactors, receptors, receptor ligands, and analogs and derivatives thereof. In some embodiments, the ligand is the reporter molecule. In some embodiments, the ligand is the quencher molecule.

In some other embodiments, the nucleic acid probe comprises a reporter molecule and a separate ligand. For example, the nucleic acid probe comprises a reporter molecule and a quencher molecule, where the quencher molecule can be a ligand for a ligand molecule.

As used herein, the term “ligand binding molecule” refers to a molecule that binds specifically to given ligand. As used herein, the terms “binds specifically”, and “binding specificity” in reference to a ligand binding molecule refers to its capacity to bind to a given target ligand preferentially over other non-target ligands. For example, if the ligand binding molecule (“molecule A”) is capable of “binding specifically” to a given target ligand (“molecule B”), molecule A has the capacity to discriminate between molecule B and any other number of potential alternative binding partners. Accordingly, when exposed to a plurality of different but equally accessible molecules as potential binding partners, molecule A will selectively bind to molecule B and other alternative potential binding partners will remain substantially unbound by molecule A. In general, molecule A will preferentially bind to molecule B at least 10-fold, preferably 50-fold, more preferably 100-fold, and most preferably greater than 100-fold more frequently than other potential binding partners. Molecule A may be capable of binding to molecules that are not molecule B at a weak, yet detectable level. This is commonly known as background binding and is readily discernible from molecule B-specific binding, for example, by use of an appropriate control.

By way of non-limiting example, the ligand binding molecules can be one member of a binding pair. For example, the ligand binding molecules can be independently selected antibodies. In some embodiments of any of the aspects, the ligand binding molecules are independently selected from the group consisting of: anti-FAM antibodies, anti-digoxigenin antibodies, anti-tetramethylrhodamine (TAMRA) antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, streptavidin and biotin.

In some embodiments of any of the aspects, the ligand and the ligand binding molecule are members of a binding pair. As used herein, the term “binding pair” refers to a pair of moieties that specifically bind each other with high affinity, generally in the low micromolar to picomolar range. When one member of a binding pair is conjugated to a first element and the other member of the pair is conjugated to a second element, the first and second elements will be brought together by the interaction of the members of the binding pair. Non-limiting examples of binding pairs include antigen:antibody (including antigen-binding fragments or derivatives thereof), biotin:avidin, biotin:streptavidin, biotin:neutravidin (or other variants of avidin that bind biotin such as ), receptor:ligand, and the like. Additional molecule for binding pair can include, neutravidin, strep-tag, strep-tactin and derivatives, and other peptide, hapten, dye-based tags-anti-Tag combinations such as SpyCatcher-SpyTag, His-Tag, Fc Tag, Digitonin, GFP, FAM, haptens, SNAP-TAG. HRP, FLAG, HA, myc, glutathione S-transferase (GST), maltose binding protein (MBP), small molecules, and the like.

In some embodiments of any of the aspects, the ligand is an antigen.

In some embodiments of any of the aspects, the ligand binding molecule is an antibody.

Some embodiments said the methods comprise sequence-specific detection of undigested probes. Methods for sequence-specific detection of nucleic acids are well known in the art. Exemplary methods for sequence-specific detection of nucleic acids include, but are not limited to, toehold-mediated strand displacement, probe-based electrochemical readout, micro-array detection, sequence-specific amplification, hybridization with conjugated or unconjugated nucleic acid strand, colorimetric assays, gel electrophoresis, molecular beacons, fluorophore-quencher pairs, microarrays, sequencing, and the like.

Modifications can be made to the nucleic acid probe provided herein to achieve enhanced sequence-specific detection. For example, a toehold can include, for example, a relatively high GC content to provide an improvement in strand displacement rate constant for hybridization to its complement relative to a sequence with lower GC content.

Acrydite modifications can be used to permit the oligonucleotides to be used in reactions with nucleophiles such as thiols (e.g., microarrays) or incorporated into gels (e.g., polyacrylamide). Accordingly, in some embodiments, the nucleic acid probe sequence comprises one or more acrydite nucleosides.

In some embodiments, the method is performed in a device comprising two or more chambers and means for irreversibly moving a fluid from a first chamber to a second chamber. In some embodiments, the means for irreversibly moving the fluid from the first to the second chamber can be actuated by a built-in spring whose potential energy is released by a solenoid trigger. In some embodiments, the device further comprises means for detecting the detectable signal from the reporter molecule.

Some embodiments of the various aspects described herein include single-stranded nucleic acid strand, e.g., single-stranded amplicons. Accordingly, in some embodiments of any one of the aspects described herein, a method described herein comprises a step of producing a single-stranded amplicon. As used herein, a “single-stranded amplicon” includes double-stranded nucleic acids having a single-stranded region.

Methods for producing single-stranded amplicons are well known in the art. For example, double-strand specific exonucleases act on the 5′-ends of double-stranded nucleic acids convert these molecules into partial duplexes whose strands are separable. Thus, in some embodiments of any of the aspects, a method descried herein comprises a step of contacting a double-stranded target nucleic acid with a 5′->3′ exonuclease, thereby producing a single-stranded region for hybridizing with the probe to produce an amplicon having single-stranded regions.

In some embodiments, the step of producing a single-stranded amplicon comprises: (a) amplifying a target nucleic acid to produce a double-stranded amplicon, wherein at least one primer for the amplification comprises a nucleic acid modification capable of inhibiting 5′->3′ cleaving activity of a 5′->3′ exonuclease; and (b) contacting the double-stranded amplicon with an exonuclease having 5′->3′ cleaving activity.

In some embodiments, the step of producing a single-stranded amplicon comprises: (a) amplifying a target nucleic acid to produce a double-stranded amplicon, wherein at least one primer for the amplification comprises one or more uridine nucleotides; and (b) contacting the double-stranded amplicon with a Uracil-DNA glycosylase (UDG) to produce an amplicon having single-stranded regions. In some further embodiments of this, at least one other primer for the amplification comprises a detectable label, e.g., at its 5′-end.

In some embodiments, the step of producing a single-stranded amplicon comprises amplifying a target nucleic acid to produce a double-stranded amplicon, wherein at least one primer for the amplification comprises a nucleic acid modification capable of inhibiting synthesis of a complementary strand by a polymerase at an internal position, and wherein the double-stranded amplicon comprises a single-stranded, e.g. a 5′ single-stranded region at one end.

In some embodiments, the step of producing a single-stranded amplicon comprises: (a) amplifying a target nucleic acid to produce a double-stranded amplicon, wherein the double-stranded amplicon comprises a single-strand, e.g., a 5′-single-stranded overhang on at least one end; and (b) contacting the double-stranded amplicon of step (a) with a nucleic acid probe comprising a sequence substantially complementary to the single-strand overhang, whereby the nucleic acid probe hybridizes with the complementary single-strand overhang and releases the non-complementary, to the probe, strand as a single-stranded amplicon.

In some embodiments, the step of preparing a single-stranded amplicon comprises: (a) amplifying a target nucleic acid to produce a double-stranded amplicon: and (b) contacting the double-stranded amplicon with a surfactant to displace one strand of the double-stranded amplicon to produce a single-stranded amplicon. In some embodiments, the surfactant is an anionic surfactant, e.g., the surfactant is sodium dodecyl sulfate (SDS).

It is noted that a single-stranded amplicon, e.g., a single-stranded amplicon produced by a method described herein can be detected using methods other than hybridizing a probe and digesting the probe to release a reporter molecule. Exemplary methods for detecting single-stranded nucleic acids, e.g., a single-stranded amplicon produced by a method described herein or uncleaved probe include, but are not limited to, fluorescence detection, luminescence detection, chemiluminescence detection, colorimetric detection, or immunofluorescence detection. In some embodiments, the method of detecting single-stranded nucleic acids, e.g., a single-stranded amplicon produced by a method described herein or uncleaved probe comprises toehold-mediated strand displacement, probe-based electrochemical readout, micro-array detection, sequence-specific amplification, hybridization with conjugated or unconjugated nucleic acid strand, colorimetric assays, gel electrophoresis, molecular beacons, fluorophore-quencher pairs, microarrays, sequencing or any combinations thereof. In some embodiments, the method of detecting single-stranded nucleic acids, e.g., a single-stranded amplicon produced by a method described herein or uncleaved probe comprises lateral flow detection.

Exemplary methods of detecting single-stranded nucleic acid strands, e.g., a single-stranded amplicon or uncleaved probes are described herein. In some embodiments, the method for detecting a single-stranded nucleic acid strand, e.g., a single-stranded amplicon or uncleaved probe comprises: hybridizing the single-stranded amplicon with a first nucleic acid probe and a second nucleic acid probe to form a complex, wherein the first nucleic acid probe comprises a first detectable label and the second nucleic acid probe comprises a ligand for a ligand binding molecule; and detecting presence of the complex, e.g., by lateral flow detection.

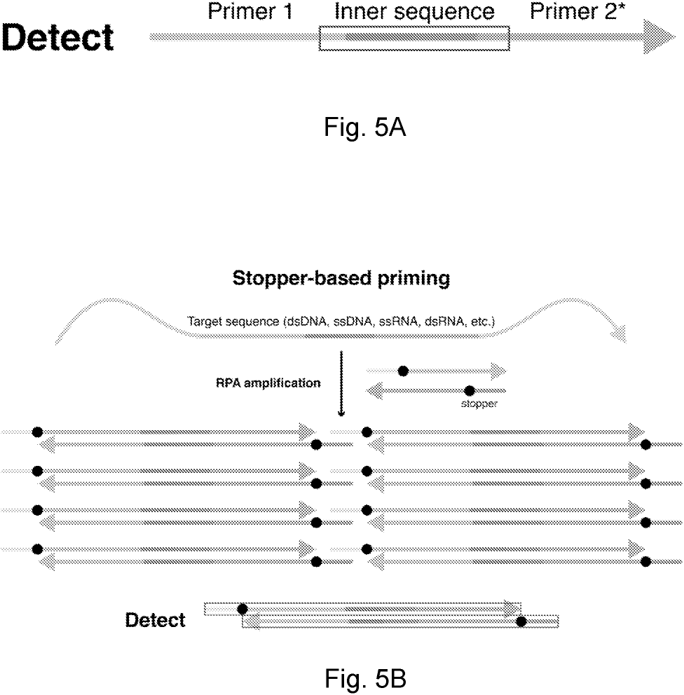

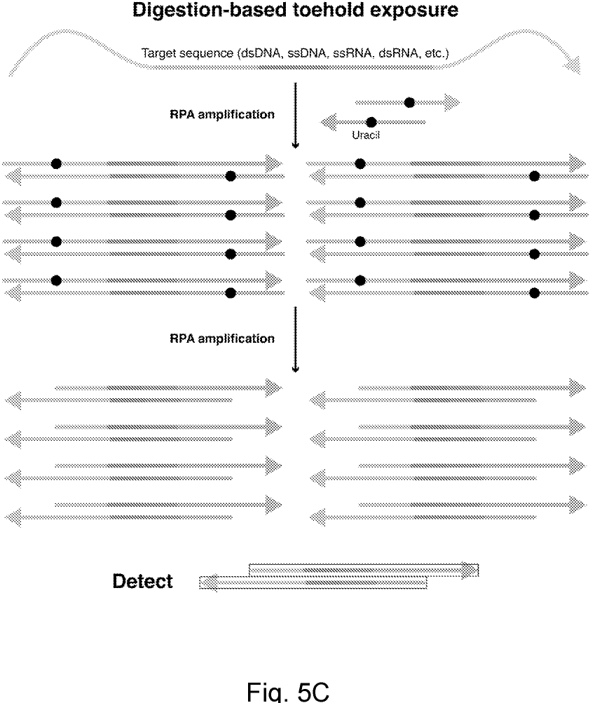

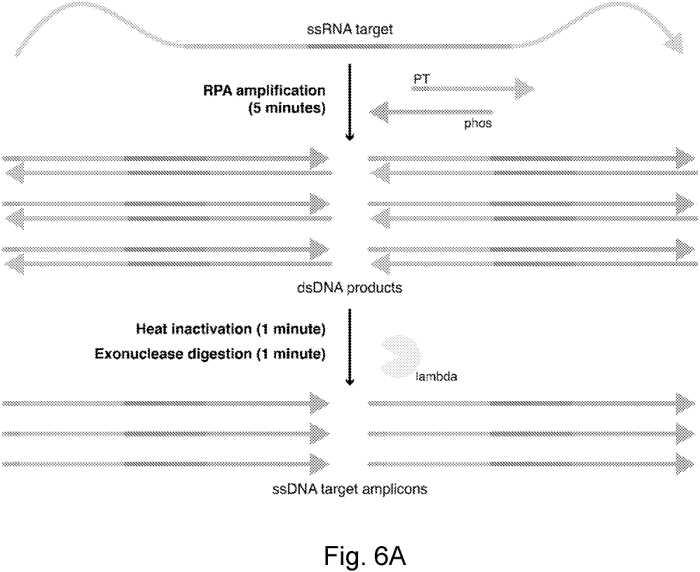

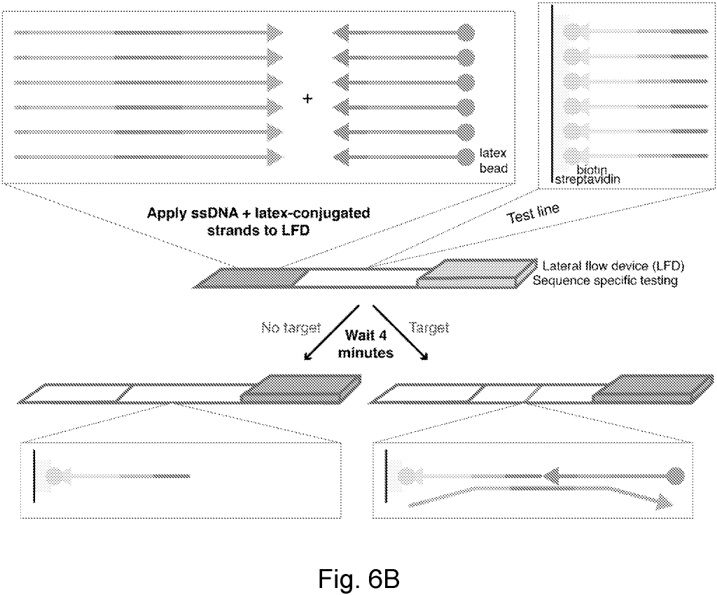

In some embodiments of any of the aspects, at least one of the first and second nucleic acid probe hybridizes at an inner region of the single-stranded amplicon. As used herein, the term “inner region” refers to a region of the amplicon that does not comprise a primer binding-site (see e.g., FIG. 5A, FIG. 6B). In some embodiments of any of the aspects, the first nucleic acid probe hybridizes at an inner region of the single-stranded amplicon. In some embodiments of any of the aspects, the second nucleic acid probe hybridizes at an inner region of the single-stranded amplicon. In some embodiments of any of the aspects, the first and second nucleic acid probes hybridize at an inner region of the single-stranded amplicon.

In some embodiments, the method for detecting a single-stranded nucleic acid strand, e.g., a single-stranded amplicon or uncleaved probe comprises: (a) contacting the single-stranded amplicon with a double-stranded probe, wherein the double-stranded probe comprises a first nucleic acid strand comprising a fluorophore and a second nucleic acid strand comprising a quencher for quenching a fluorescent emission of the fluorophore; and (b) measuring the fluorescent emission of the fluorophore, wherein the binding of the first and/or second nucleic acid strand inhibits quenching of the fluorescent emission of the fluorophore by the quencher. In some further embodiments of this, the fluorescent emission of the fluorophore is quenched when the first and second nucleic acid strands are hybridized to each other. In yet some further embodiments, the double-stranded probe comprises a single-stranded overhang at one end and the nucleic acid strand comprising the single-stranded overhang comprises a nucleotide sequence substantially complementary to a region of the single-stranded amplicon, and wherein the amplicon and the nucleic strand comprising the overhang hybridize to each other, thereby inhibiting quenching of the fluorescent emission of the fluorophore by the quencher.

In some embodiments, the method for detecting a single-stranded nucleic acid strand, e.g., a single-stranded amplicon or uncleaved probe comprises applying the single-stranded nucleic acid to a lateral flow test strip, wherein the later flow test strip comprises a test/capture region comprising a nucleic acid capture probe immobilized therein, wherein the nucleic acid capture probe comprises a toehold domain (e.g., a single-stranded region) comprising a nucleotide sequence substantially complementary to at least a part of the single-stranded nucleic acid.

In some embodiments, the method for detecting a single-stranded nucleic acid strand, e.g., a single-stranded amplicon or uncleaved probe comprises hybridizing a plurality of nucleic acid probes to the single-stranded nucleic acid strand, wherein members of the plurality comprise a nucleotide sequence substantially complementary to different regions of the strand, wherein each probe comprises a detectable label attached thereto, and wherein the detectable label undergoes a change in an optical property in response to label density, pH change and/or temperature change. In some further embodiments, said hybridizing with the plurality of nucleic acid probes is in presence of a surfactant, e.g., SDS.

In some embodiments of any of the aspects, the amplification product is detected using colorimetric assays. Colorimetric assays use reagents that undergo a measurable color change in the presence of the analyte. For example, para-Nitrophenylphosphate is converted into a yellow product by alkaline phosphatase enzyme. Coomassie Blue once bound to proteins elicits a spectrum shift, allowing quantitative dosage. A similar colorimetric assay, the Bicinchoninic acid assay, uses a chemical reaction to determine protein concentration. Enzyme linked immunoassays use enzyme-complexed-antibodies to detect antigens. Binding of the antibody is often inferred from the color change of reagents such as TMB. A colorimetric assay can be detected using a colorimeter, which is a device used to test the concentration of a solution by measuring its absorbance of a specific wavelength of light.

In some embodiments of any of the aspects, the colorimetric assay comprises nanoparticles whose optical properties change based on the particle density (see e.g., FIG. 30), e.g., plasmonic nanoparticles. For example, at least two nucleic acid probes specific to the single-stranded amplicon can each be linked to such a nanoparticle (e.g., at the 5′ end and/or 3′ end of each). In absence of amplicon binding, the diffuse nanoparticle probes cause the solution to be a first color (e.g., red). Binding to the target amplicon creates aggregation of the nanoparticles, causing the solution turn a second color (e.g., purple). The color change hence indicates the presence of the target amplicon in solution. As a non-limiting example, gold nanoparticles can exhibit color changes in solution depending on the gold nanoparticle density. In some embodiments of any of the aspects, the nanoparticles are aggregated by conjugating them or binding them to functional groups on the detection probes, e.g., during the detection step.

In some embodiments of any of the aspects, the colorimetric assay produces a color change via change of pH in a minimally buffered reaction. In some embodiments of any of the aspects, the colorimetric assay produces a color change via oxidation/reduction of a substrate (e.g., ABTS (2,2′-Azinobis [3-ethylbenzothiazoline-6-sulfonic acid]-diammonium salt) through assembly of split Horseradish Peroxidase (HRP). In some embodiments of any of the aspects, the colorimetric assay produces a color change via assembly of an enzyme or protein with optical properties (e.g., split luciferase or split GFP equivalents). In some embodiments of any of the aspects, the colorimetric assay produces a color change via DNA-intercalating dyes, e.g., cyanine dyes, TOTO, TO-PRO, SYTOX, ethidium bromide, propidium iodide, DAPI, Hoechst dye, acridine orange, 7-AAD, LDS 751, and hydroxystilbamidine. In one aspect, described herein is a method for detecting a target nucleic acid, the method comprising: (a) amplifying a target nucleic acid with a first primer and a second primer to produce a double-stranded amplicon, wherein the first primer comprises a nucleic acid modification capable of inhibiting 5′->3′ cleaving activity of a 5′->3′ exonuclease; (b) contacting the double-stranded amplicon with a 5′->3′ exonuclease to produce a single-stranded amplicon; and (c) detecting the single-stranded amplicon, wherein said detecting comprises hybridizing a plurality of nucleic acid probes to the single-stranded amplicon, wherein members of the plurality comprise a nucleotide sequence substantially complementary to different regions of the strand, wherein each probe comprises a detectable label attached thereto, and wherein the detectable label undergoes a change in an optical property in response to label density, pH change and/or temperature change, and optionally, said hybridizing is in presence of a surfactant, e.g., SDS.

In one aspect described herein is a method for detecting a target nucleic acid, the method comprising: (a) amplifying a target nucleic acid with a first primer and a second primer to produce a double-stranded amplicon, optionally, wherein the first primer comprises a nucleic acid modification capable of inhibiting 5′->3′ cleaving activity of a 5′->3′ exonuclease; and (b) detecting the double-stranded amplicon, wherein said detecting comprises hybridizing a plurality of nucleic acid probes to one strand of the double-stranded, wherein said hybridizing is in the presence of a surfactant e.g., SDS, and/or a reagent capable of localizing a single-strand nucleic acid strand to a double-stranded nucleic acid, wherein members of the plurality comprise a nucleotide sequence substantially complementary to different regions of the strand, wherein each probe comprises a detectable label attached thereto, and wherein the detectable label undergoes a change in an optical property in response to label density, pH change, and/or temperature change.

In some embodiments of any of the aspects, the reagent capable of localizing a single-strand nucleic acid strand to a double-stranded nucleic acid is recombinase, single-stranded binding protein, Cas protein, zinc finger nuclease, transcription activator-like effector nuclease (TALEN), or any combinations thereof. In some embodiments of any of the aspects, the detectable label is a nanoparticle. In some embodiments of any of the aspects, said detecting is by a lateral flow assay and wherein the lateral flow assay is in presence of a surfactant, e.g., SDS. In some embodiments of any of the aspects, a lateral flow test strip of the assay is pre-treated with the surfactant, e.g., SDS. In some embodiments of any of the aspects, the surfactant, e.g., SDS is added to a solution comprising the probe bound amplicon prior to and/or concurrently with applying the solution to a lateral flow test strip of the assay.

In some embodiments of any of the aspects, the amplification product is detected using gel electrophoresis. Gel electrophoresis is a technique used to separate DNA fragments according to their size. DNA samples are loaded into wells (indentations) at one end of a gel, and an electric current is applied to pull them through the gel. The gel electrophoresis can be performed according to methods known in the art.

In some embodiments of any of the aspects, the amplification product is detected using oligo strand displacement (OSD), also referred to as a toehold-mediated strand displacement reaction. Nucleic acid strand displacement (OSD) probes hybridize to specific sequences in amplification products and thereby generate simple yes/no readout of fluorescence, which is readable by human eye or by off-the-shelf cellphones. In some embodiments of any of the aspects, the OSD probes are short hemiduplex oligonucleotides. The single stranded ‘toehold’ regions of OSD probes bind to amplification products (e.g., LAMP amplicon loop sequences), and then signal via strand exchange that leads to separation of a fluorophore and quencher. OSDs are the functional equivalents of TaqMan probes and can specifically report single or multiplex amplicons without interference from non-specific nucleic acids or inhibitors; see e.g., Bhadra et al. bioRxiv 291849 (2018); Jiang et al. (2015) Anal Chem 87: 3314-3320; Zhang and Winfree (2009) J Am Chem Soc 131: 17303-17314; Bhadra et al. (2015) PLoS One 10: e0123126.

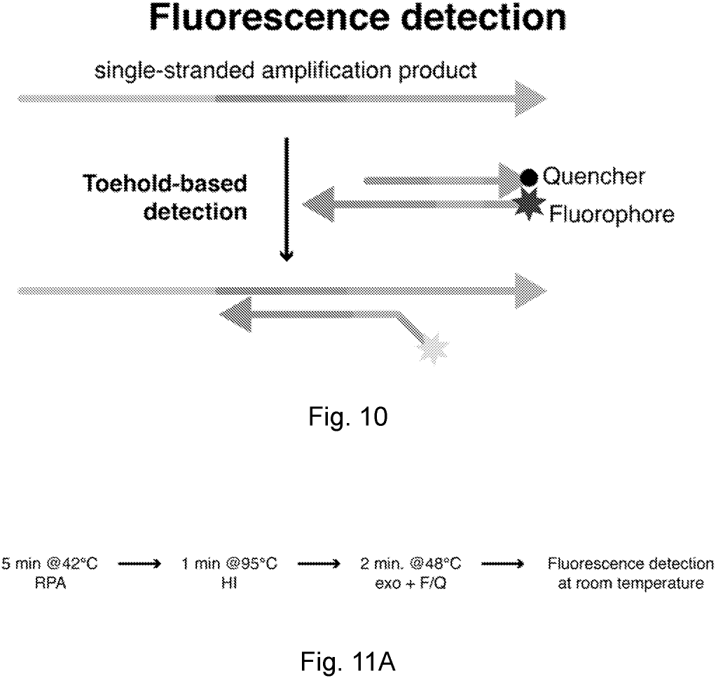

In some embodiments of the various aspects described herein, a method for detecting the single-stranded amplicon comprises toe-hold detection. For example, the single-stranded amplicon is contacted with a double-stranded probe. The probe comprises a fluorophore - quencher pair. The fluorophore and the quencher in close proximity to each other in the double-stranded probe so that a fluorescent emission of the fluorophore is quenched by the quencher. One of the strands in the double-stranded probe comprises a single-stranded region comprising a nucleotide sequence complimentary to the amplicon sequence. This single-stranded region can act as a toe-hold for the amplicon to hybridize with the strand comprising the tow-hold region, i.e., the single-stranded region. Once the amplicon hybridizes with the strand comprising the tow-hold region, the fluorophore and the quencher are no longer in close proximity to each other. The fluorescent emission of the fluorophore is no longer quenched by the quencher; thereby an increase in the fluorescent emission is seen if the single-stranded amplicon is present. An example of this method is schematically illustrated in FIG. 10.

Generally, the double-stranded probe comprises a first nucleic acid strand comprising a fluorophore and a second nucleic acid strand comprising a quencher for quenching a fluorescent emission of the fluorophore. The double-stranded probe comprises a single-stranded overhang at one end and the nucleic acid strand comprising the single-stranded overhang comprises a nucleotide sequence substantially complementary to a region of the single-stranded amplicon. It is noted that either the first nucleic acid strand with the fluorophore or the second nucleic acid strand with the quencher can comprise the single-stranded overhang. Preferably, the nucleic acid strand with the fluorophore comprises the single-stranded overhang. In some embodiments of the various aspects described herein, the first and second strands can be covalently linked to each other.

Accordingly, in one aspect, described herein is a method of detecting a single stranded amplicon (e.g., produced using a method as described herein) comprising: (a) contacting the single-stranded amplicon with a double-stranded probe, wherein the double-stranded probe comprises: (i) a first nucleic acid strand comprising a fluorophore; (ii) a second nucleic acid strand comprising a quencher for quenching a fluorescent emission of the fluorophore; and (b) measuring the fluorescent emission of the fluorophore.

In some embodiments of any of the aspects, the fluorescent emission of the fluorophore is quenched when the first and second nucleic acid strands (e.g., of the double-stranded probe) are hybridized to each other. In some embodiments of any of the aspects, the double-stranded probe comprises a single-stranded overhang at one end and the nucleic acid strand comprising the single-stranded overhang comprises a nucleotide sequence substantially complementary to a region of the single-stranded amplicon. In some embodiments of any of the aspects, the amplicon and the nucleic strand comprising the overhang hybridize to each other, thereby inhibiting quenching of the fluorescent emission of the fluorophore by the quencher.

In some embodiments of any of the aspects, the amplification product is detected using molecular beacons. Molecular beacons, or molecular beacon probes, are oligonucleotide hybridization probes that can report the presence of specific nucleic acids in homogenous solutions. Molecular beacons are hairpin-shaped molecules with an internally quenched fluorophore whose fluorescence is restored when they bind to a target nucleic acid sequence. See e.g., Tyagi S and Kramer FR (1996) Nat. Biotechnol. 14 (3): 303-8; Täpp et al. (April 2000) BioTechniques. 28 (4): 732-8; Akimitsu Okamoto (2011). Chem. Soc. Rev. 40: 5815-5828.

In some embodiments of any of the aspects, the amplification product is detected using Förster resonance energy transfer (FRET). As a non-limiting example, an amplification product can be contacted with two detection probes, wherein each probe comprises one of a FRET fluorophore pair, such that FRET occurs only when both probes bind to the amplification product. The one or more FRET pairs can comprise at least one FRET donor and at least one FRET acceptor. In some cases, the FRET donor is attached to the first probe and the FRET acceptor is attached to the second probe. In other cases, the FRET acceptor is attached to the first probe, and the FRET donor is attached to the second probe. The FRET donor and acceptor can be attached to either end (3′ or 5′) of either probe. In some cases, the FRET donor is Cy3 and the FRET acceptor is Cy5. Further non-limiting examples of FRET pairs include: Cy3 and MG; Cy3 and acetylenic MG; Cy3, Cy5 and MG; Cy3 and DIR; Cy3 and Cy5 and ICG; FITC and TRITC; EGFP and Cy3; CFP and YFP; and EGFP and YFP.

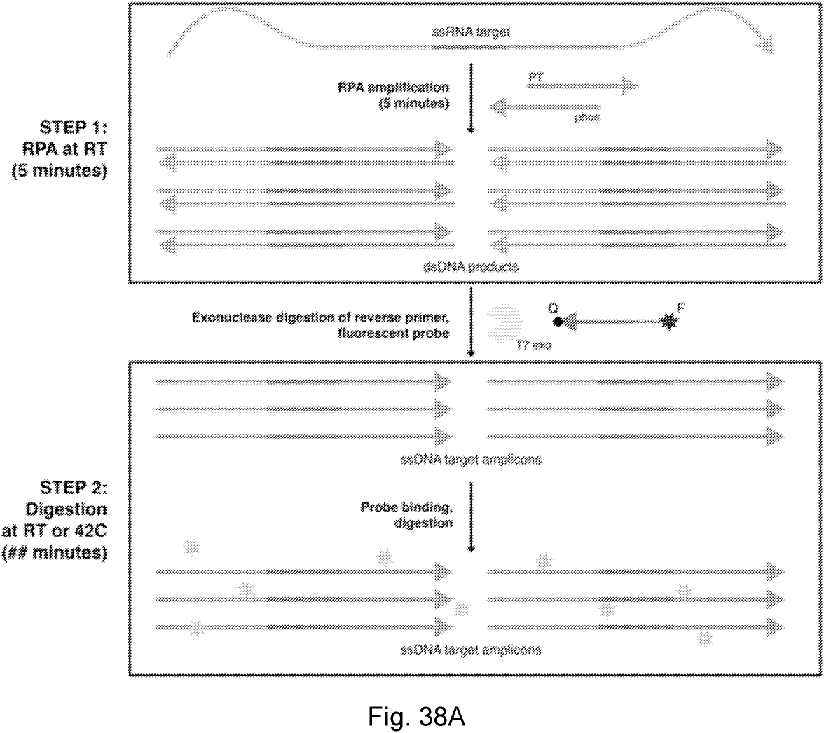

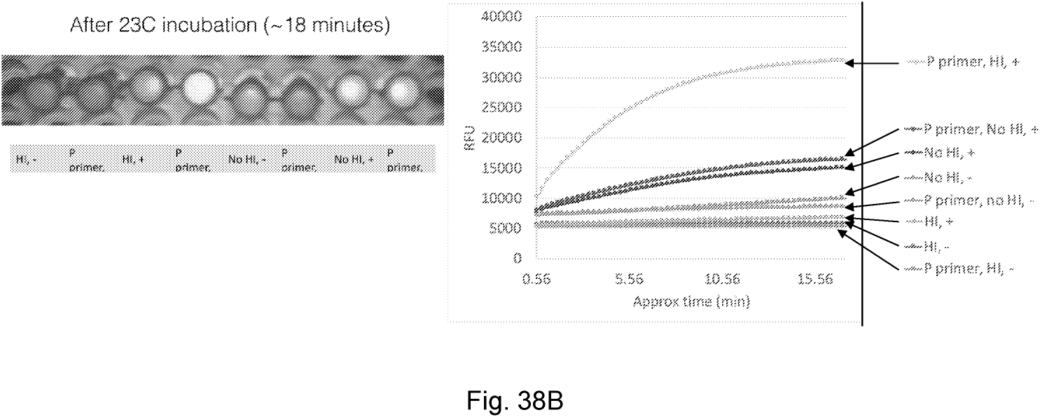

In some embodiments of any of the aspects, the amplification product is detected using fluorophore-quencher pairs. In some embodiments of any of the aspects, a detection probe comprises a fluorophore-quencher pair such that the probe generates a fluorescence signal only when it binds to its target (e.g., the amplification product of the target nucleic acid). Non-limiting examples of quenchers include: Dabcyl (quenches 400 nm-530 nm); Black Hole Quencher 1 (BHQ-1; quenches 480 nm-580 nm); Black Hole Quencher 2 (BHQ-2; quenches 550 nm-670 nm); and BlackBerry® Quencher 650 (BBQ 650; quenches 550 nm-750 nm). See e.g., Marras, Selection of fluorophore and quencher pairs for fluorescent nucleic acid hybridization probes, Methods Mol Biol. 2006;335:3-16. As a non-limiting example, the detection method comprises (a) contacting a single-stranded amplicon with a detection probe comprising a quencher and a fluorophore, wherein the quencher quenches the fluorophore in the absence of the single-stranded amplicon; (b) allowing the detection probe to bind to the single-stranded amplicon; and (c) contacting the detection probe bound to the single-stranded amplicon with a dsDNA-specific exonuclease (e.g., T7 exonuclease, lambda exonuclease, Endo IV) to release the fluorophore from the probe, leading to a detectable increase in fluorescence. See e.g., FIGS. 38A-38C.

In some embodiments of any of the aspects, the amplification product is detected using microarrays. A DNA microarray (also commonly known as DNA chip or biochip) is a collection of microscopic DNA spots attached to a solid surface. Such DNA spots comprises DNA that hybridizes to the amplification product of the at least one target nucleic acid. In some embodiments of any of the aspects, the microarray is provided on a solid support. In some embodiments of any of the aspects, the microarray is printed on a lateral flow detection strip. In some embodiments of any of the aspects, the microarray is used to detect at least 2, at least 3, at least 4, at least 5, at least 6, at least 7, at least 8, at least 9, at least 10, at least 15, at least 20, at least 30, at least 40, at least 50, at least 60, at least 70, at least 80, at least 90, or at least 100 target nucleic acids.

In some embodiments of any of the aspects, the amplification product is detected using Specific High-sensitivity Enzymatic Reporter unLOCKing (SHERLOCK). SHERLOCK is a method that can be used to detect specific RNA/DNA at low attomolar concentrations (see e.g., U.S. Pat. 10,266,886; U.S. Pat. 10,266,887; Gootenberg et al., Science. 2018 Apr 27;360(6387):439-444; Gootenberg et al., Science. 2017 Apr 28;356(6336):438-44; the content of each of which is incorporated herein by reference in its entirety). Briefly, a detection method using SHERLOCK comprises the following steps: (a) contacting amplified DNA with an RNA polymerase (e.g., T7 polymerase) to promote the production of complementary RNA; (b) contacting the RNA with: (i) a crRNA comprising a Cas enzyme scaffold and a region that hybridizes to the target RNA; (ii) a Cas enzyme (e.g., Cas13a (previously known as C2c2), Cas13b, Cas13c, Cas12a, and/or Csm6); and (iii) a detection molecule cleavable by the Cas enzyme; (c) detecting cleavage of the detection molecule, wherein said cleavage indicates presence of the target RNA.

In some embodiments of any of the aspects, the amplification product is detected using DNA endonuclease-targeted CRISPR trans reporter (DETECTR). Briefly, a detection method using DETECTR comprises the following steps: (a) contacting the amplification product with: (i) a crRNA comprising a Cas enzyme scaffold and a region that hybridizes to the amplification product; (ii) a Cas enzyme (e.g., Cas12a); and (iii) a detection molecule cleavable by the Cas enzyme; (c) detecting cleavage of the detection molecule, wherein said cleavage indicates presence of the target nucleic acid. See e.g., U.S. Pat. Application US20190241954; PCT Patent Application WO2020028729; Chen et al., CRISPR-Cas12a target binding unleashes indiscriminate single-stranded DNase activity, Science. 2018 Apr 27, 360(6387):436-439; the content of each of which is incorporated herein by reference in its entirety.

In some embodiments of any of the aspects, the level and/or sequence of an amplification product can be measured by a quantitative sequencing technology, e.g. a quantitative next-generation sequencing technology. Methods of sequencing a nucleic acid sequence are well known in the art. Briefly, a sample obtained from a subject can be contacted with one or more primers which specifically hybridize to a single-strand nucleic acid sequence (e.g., primer binding sequence) flanking the target sequence (e.g., the target nucleic acid) and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing (i.e., dideoxy chain termination), 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

In some embodiments of any of the aspects, the level and/or sequence of an amplification product can be measured using PCR. In some embodiments of any of the aspects, the amount of amplification product can be determined by quantitative PCR (QPCR) or real-time PCR methods, e.g., using a set of primers specific to the amplification product and/or SYBR® GREEN or a detectable probe. Methods of qPCR and real-time qPCR are well known in the art.

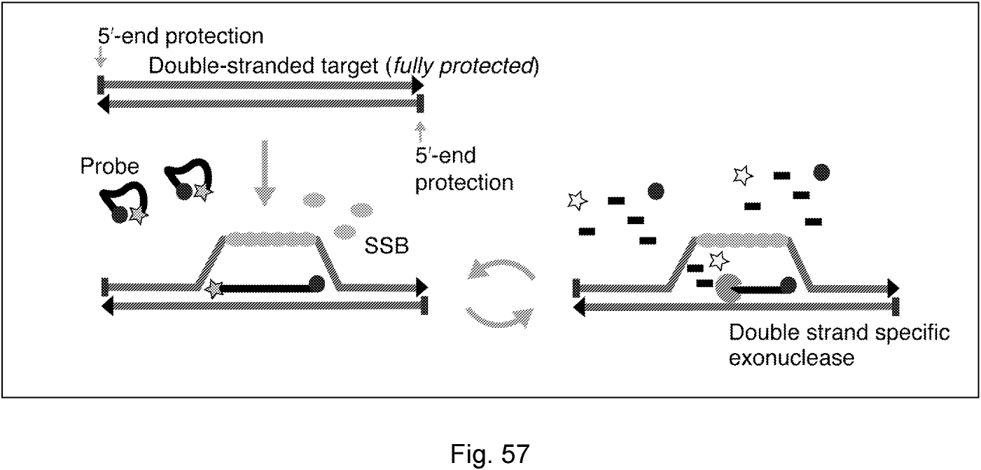

In some embodiments of any of the aspects, the detection method comprises contacting the double-stranded amplicon with a detection probe and a recombinase and/or single-stranded binding protein (SSB). Use of the recombinase and/or SSB can help localize the detection probe to the target sequence of interest in a double-stranded amplicon (see e.g., FIG. 57). In some embodiments of any of the aspects, the detection method comprises contacting the double-stranded amplicon with: (a) a detection probe; (b) a recombinase and/or single-stranded binding protein (SSB); and (c) a buffer additive. Non-limiting examples of such buffer additives include a surfactant (e.g., SDS or another detergent), a salt, a chaotropic agent (i.e., a compound which disrupts hydrogen bonding in aqueous solution), a DNA duplex destabilizer, a reducing agent, or a temperature change.

In some embodiments of any of the aspects, the detection method comprises contacting the double-stranded amplicon with a detection probe and a Cas protein (e.g., Cas9, dCas9, Cas13). In some embodiments of any of the aspects, the detection method comprises contacting the double-stranded amplicon with a detection probe and a zinc finger nuclease. In some embodiments of any of the aspects, the detection method comprises contacting the double-stranded amplicon with a detection probe and a transcription activator-like effector nuclease (TALEN). For example, the detection probe comprises a scaffold structure that is bound by the Cas, Zinc finger, or TALEN proteins. For example, the Cas, Zinc finger, or TALEN protein can guide the detection probe to the complementary region on the amplicon. In some embodiments, the Cas, zinc finger, or TALEN protein is catalytically inactive and does not cleave the amplicon target. In some embodiments, the Cas, zinc finger, or TALEN protein is catalytically active and can cleave the amplicon target. In some embodiments, the detection probe (used with the Cas, zinc finger, or TALEN protein) comprises a detectable marker that can be detected through fluorescence, colorimetric assay, LFD, or another detection assay as described herein.

In some embodiments of any of the aspects, the detection method comprises contacting the double-stranded amplicon with a detection probe that induces the formation of a non-canonical DNA structure (e.g., non-B form DNA; e.g., triplex-DNA such as H-DNA). In some embodiments of any of the aspects, the detection probe hybridizes with a GA-rich region of the double-stranded amplicon, resulting in a triplex DNA structure. Such non-canonical DNA (e.g., triplex DNA) can be detected using triplex-specific antibodies or DNA intercalating dyes with a high affinity for non-canonical DNA structures (e.g., Thiazole Orange).

In some embodiments, the radionuclide is bound to a chelating agent or chelating agent-linker attached to probe, primer or reagent. Exemplary chelating agents include, but are not limited to, diethylenetriaminepentaacetic acid (DTPA) and ethylenediaminetetraacetic acid (EDTA). Suitable radionuclides for direct conjugation include, without limitation, ³H, ¹⁸F, ¹²⁴I, ¹²⁵I, ¹³¹1, ³⁵S, ¹⁴C, ³²P, and ³³P and mixtures thereof. Suitable radionuclides for use with a chelating agent include, without limitation, ⁴⁷Sc, ⁶⁴Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi, and mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to molecules such nucleic acids.

In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromogenic substrate. Such streptavidin peroxidase detection kits are commercially available, e.g., from DAKO; Carpinteria, CA. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

In some embodiments of any of the aspects, the level of the detected amplification product can be compared to a reference. In some embodiments of any of the aspects, the reference can also be a level of expression of the target molecule in a control sample, a pooled sample of control items or a numeric value or range of values based on the same. In some embodiments of any of the aspects, the reference can be the level of a target molecule in a sample obtained from the same item at an earlier point in time.

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

### Amplification

Embodiments of the various aspects described herein comprise a step of amplifying a target nucleic acid. As used herein, “amplification” is defined as the production of additional copies of a nucleic acid sequence, i.e., for example, amplicons or amplification products. Methods of amplifying nucleic acid sequences are well known in the art. Such methods include, but are not limited to, isothermal amplification, polymerase chain reaction (PCR) and variants of PCR such as Rapid amplification of cDNA ends (RACE), ligase chain reaction (LCR), multiplex RT-PCR, immuno-PCR, SSIPA, Real Time RT-qPCR and nanofluidic digital PCR.

In some embodiments of any of the aspects, the amplification step comprises isothermal amplification reaction. As used herein, “isothermal amplification” refers to amplification that occurs at a single temperature. Isothermal amplification is an amplification process that is performed at a single temperature or where the major aspect of the amplification process is performed at a single temperature. Generally, isothermal amplification relies on the ability of a polymerase to copy the template strand being amplified to form a bound duplex. In the multi-step PCR process the product of the reaction is heated to separate the two strands such that a further primer can bind to the template repeating the process. Conversely, the isothermal amplification relies on a strand displacing polymerase in order to separate/displace the two strands of the duplex and re-copy the template. The key feature that differentiates the isothermal amplification is the method that is applied in order to initiate the reiterative process. Broadly isothermal amplification can be subdivided into those methods that rely on the replacement of a primer to initiate the reiterative template copying and those that rely on continued reuse or de novo synthesis of a single primer molecule.

Isothermal amplification permits rapid and specific amplification of a target nucleic acid at a constant temperature. In general, isothermal amplification is comprised of (i) sequence-specific hybridization of primers to sequences within a target nucleic acid, and (ii) subsequent amplification involving multiple rounds of primer annealing, elongation, and strand displacement (as a non-limiting example, using a combination of recombinase, single-stranded binding proteins, and DNA polymerase). In some embodiments of any of the aspects, the isothermal amplification produce can be detected through such methods as sequencing to confirm the identity of the amplified product or general assays such as turbidity. In some types of isothermal amplification, turbidity results from pyrophosphate byproducts produced during the reaction; these byproducts form a white precipitate that increases the turbidity of the solution. The primers used in isothermal amplification are oligonucleotides of sufficient length and appropriate sequence to provide initiation of polymerization, i.e. each primer is specifically designed to be complementary to a strand of the template (e.g., target cDNA) to be amplified. In contrast to the polymerase chain reaction (PCR) technology in which the reaction is carried out with a series of alternating temperature steps or cycles, isothermal amplification is carried out at one temperature, and does not require a thermal cycler or thermostable enzymes.

Non-limiting examples of isothermal amplification include: Loop Mediated Isothermal Amplification (LAMP), Recombinase Polymerase Amplification (RPA), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3SR), Single primer isothermal amplification (SPIA), and cross-priming amplification (CPA). See e.g., Yan et al., Isothermal amplified detection of DNA and RNA, March 2014, Molecular BioSystems 10(5), DOI: 10.1039/c3mb70304e; Piepenburg et al. PLOS Biol. 4, e204 (2006); Notomi, T. Loop-mediated isothermal amplification of DNA. Nucleic Acids Res. 28, 63e-663 (2000); Vincent et al. EMBO reports 5.8 (2004): 795-800; the contents of each which are incorporated herein by reference in their entireties.

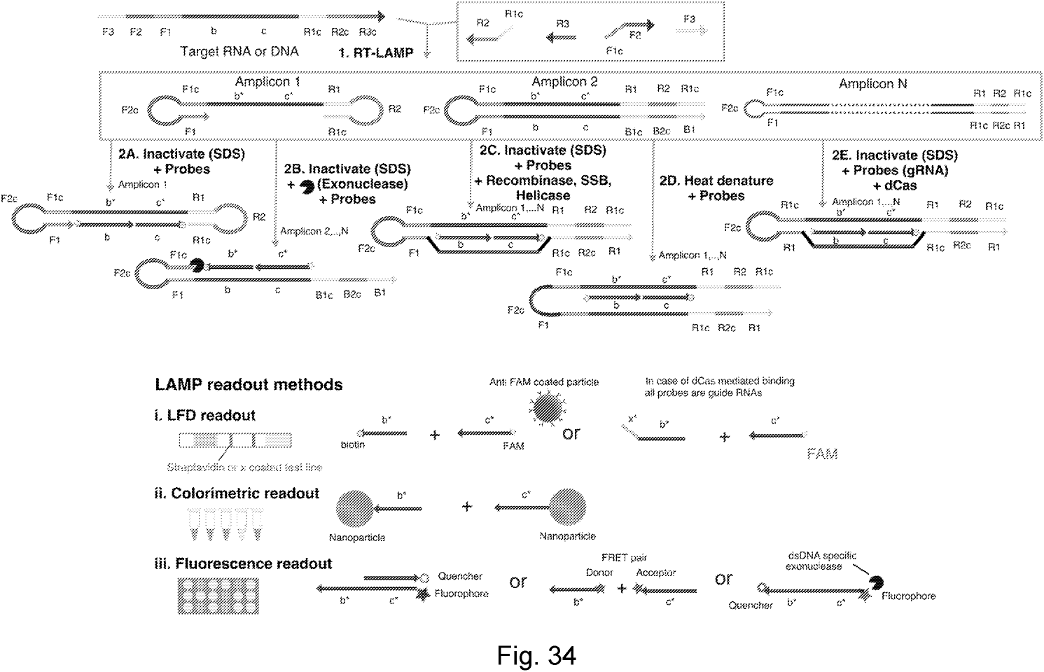

In some embodiments of any of the aspects, the isothermal amplification reaction(s) is Loop Mediated Isothermal Amplification (LAMP), i.e., i.e., the step of amplifying the target nucleic acids comprises Loop Mediated Isothermal Amplification. LAMP is a single tube technique for the amplification of DNA; LAMP uses 4-6 primers, which form loop structures to facilitate subsequent rounds of amplification. Accordingly, in some embodiments of the aspects, the amplification step comprises contacting the sample with a DNA polymerase and a set of primers, wherein the set of primers comprises 4, 5, or 6 loop-forming primers. See e.g., FIG. 34.

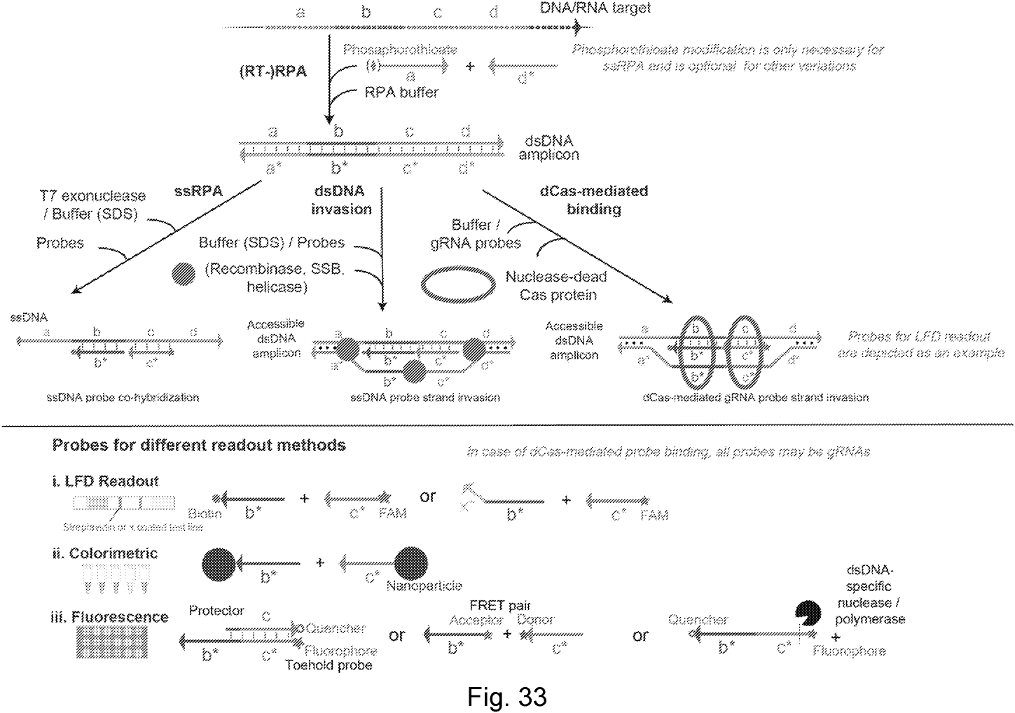

In some embodiments of any of the aspects, the isothermal amplification reaction(s) is Recombinase Polymerase Amplification (RPA), i.e., the step of amplifying the target nucleic acids comprises Recombinase Polymerase Amplification. RPA is a low temperature DNA and RNA amplification technique. The RPA process employs three core enzymes - a recombinase, a single-stranded DNA-binding protein (SSB) and strand-displacing polymerase. Recombinases are capable of pairing oligonucleotide primers with homologous sequence in duplex DNA. SSB bind to displaced strands of DNA and prevent the primers from being displaced. Finally, the strand displacing polymerase begins DNA synthesis where the primer has bound to the target DNA. By using two opposing primers, much like PCR, if the target sequence is indeed present, an exponential DNA amplification reaction is initiated. No other sample manipulation such as thermal or chemical melting is required to initiate amplification. At optimal temperatures (e.g., 37-42° C.), the RPA reaction progresses rapidly and results in specific DNA amplification from just a few target copies to detectable levels, typically within 10 minutes, for rapid detection of the target nucleic acid. In some embodiments of any of the aspects, the single-stranded DNA-binding protein is a gp32 SSB protein. In some embodiments of any of the aspects, the recombinase is a uvsX recombinase. See e.g., U.S. Pat. 7,666,598, the content of which is incorporated herein by reference in its entirety. In some embodiments of any of the aspects, RPA can also be referred to as Recombinase Aided Amplification (RAA). Accordingly, in some embodiments of any of the aspects, the amplification step comprises contacting the sample with a recombinase and single-stranded DNA binding protein. In some embodiments of any of the aspects, the amplification step comprises contacting the sample with a DNA polymerase, a set of primers, a recombinase, and single-stranded DNA binding protein. See e.g., FIG. 33.

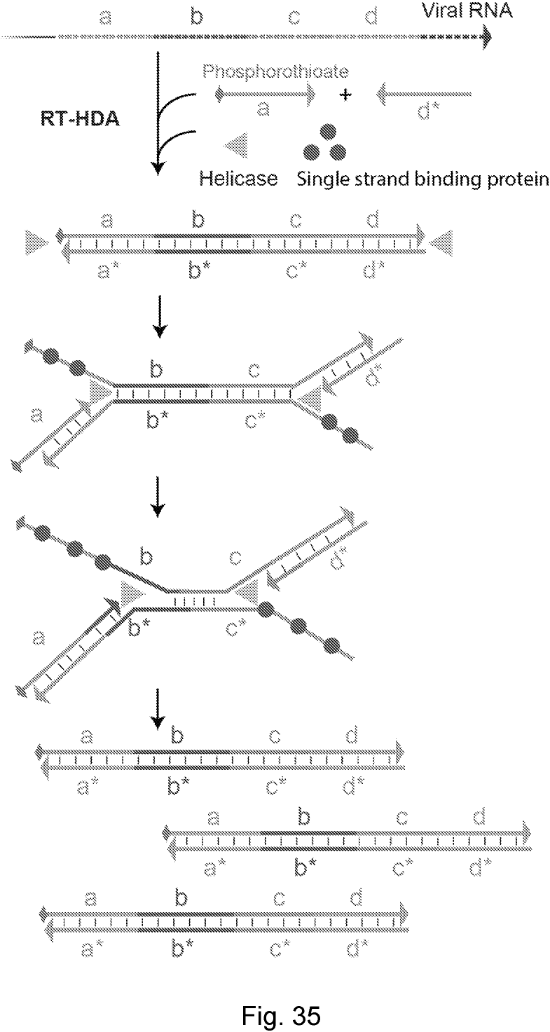

In some embodiments of any of the aspects, the isothermal amplification reaction(s) is Helicase-dependent isothermal DNA amplification (HDA). HDA uses the double-stranded DNA unwinding activity of a helicase to separate strands for in vitro DNA amplification at constant temperature. In some embodiments of any of the aspects, the helicase is a thermostable helicase, which can improve the specificity and performance of HDA; as such, the isothermal amplification reaction(s) can be thermophilic helicase-dependent amplification (tHDA). As a non-limiting example, the helicase is the thermostable UvrD helicase (Tte-UvrD), which is stable and active from 45 to 65° C. Accordingly, in some embodiments of the aspects, the amplification step comprises contacting the sample with a DNA polymerase, a set of primers, and a helicase, wherein the helicase is optionally a thermostable helicase. See e.g., FIGS. 35-37.

In some embodiments of any of the aspects, the isothermal amplification reaction(s) is Rolling Circle Amplification (RCA). RCA starts from a circular DNA template and a short DNA or RNA primer to form a long single stranded molecule. Accordingly, in some embodiments of the aspects, the amplification step comprises contacting the sample (e.g., a circular DNA) with a DNA polymerase and a set of primers, wherein the second set of primers comprises a single primer.

In some embodiments of any of the aspects, the isothermal amplification reaction(s) is Nucleic acid sequence-based amplification (NASBA), which is also known as transcription mediated amplification (TMA). NASBA is an isothermal technique predominantly used for the amplification of RNA through the cyclic formation of complimentary DNA and destruction of original RNA sequence (e.g., using RNase H). The NASBA reaction mixture contains three enzymes—reverse transcriptase (RT), RNase H, and T7 RNA polymerase—and two primers. T7 RNA Polymerase is an RNA polymerase from the T7 bacteriophage that catalyzes the formation of RNA from DNA in the 5′→ 3′ direction. Primer 1 (P1) contains a 3′ terminal sequence that is complementary to a sequence on the target nucleic acid and a 5′ terminal (+)sense sequence of a promoter that is recognized by the T7 RNA polymerase. Primer 2 (P2) contains a sequence complementary to the P1-primed DNA strand. The NASBA enzymes and primers operate in concert to amplify a specific nucleic acid sequence exponentially. NASBA results in the amplification of the target RNA to cDNA to RNA to cDNA, etc., with alternating reverse transcription (e.g., RNA to DNA) and transcription steps (e.g., DNA to RNA), and the RNA being degraded after each transcription. Accordingly, in some embodiments of the aspects, the amplification step comprises contacting the sample (e.g., a cDNA) with an RNA polymerase, a reverse transcriptase, RNaseH, and a set of primers, wherein the set of primers comprise a 5′ sequence that is recognized by the RNA polymerase.

In some embodiments of any of the aspects, the isothermal amplification reaction(s) is Strand Displacement Amplification (SDA). SDA is an isothermal, in vitro nucleic acid amplification technique based upon the ability of the restriction endonuclease HincII to nick the unmodified strand of a hemiphosphorothioate form of its recognition site, and the ability of exonuclease deficient klenow (exo-klenow) DNA polymerase to extend the 3′-end at the nick and displace the downstream DNA strand. Exponential amplification results from coupling sense and antisense reactions in which strands displaced from a sense reaction serve as target for an antisense reaction and vice versa. Accordingly, in some embodiments of the aspects, the amplification step comprises contacting the sample with a DNA polymerase (e.g., exo-klenow), a set of primers, and a restriction endonuclease (e.g., HincII).

In some embodiments of any of the aspects, the isothermal amplification reaction(s) is nicking enzyme amplification reaction (NEAR), which is a similar approach to SDA. In NEAR, DNA is amplified at a constant temperature (e.g., 55° C. to 59° C.) using a polymerase and nicking enzyme. The nicking site is regenerated with each polymerase displacement step, resulting in exponential amplification. Accordingly, in some embodiments of the aspects, the amplification step comprises contacting the sample with a DNA polymerase (e.g., exo-klenow), a set of primers, and a nicking enzyme (e.g., N.BstNBI).

In some embodiments of any of the aspects, the isothermal amplification reaction(s) is Polymerase Spiral Reaction (PSR). The PSR method employs a DNA polymerase (e.g., Bst) and a pair of primers. The forward and reverse primer sequences are reverse to each other at their 5′ end, whereas their 3′ end sequences are complementary to their respective target nucleic acid sequences. The PSR method is performed at a constant temperature 61° C.-65° C., yielding a complicated spiral structure. Accordingly, in some embodiments of the aspects, the amplification step comprises contacting the sample with a DNA polymerase (e.g., exo-klenow) and a set of primers that are reverse to each other at their 5′ end.

In some embodiments of any of the aspects, the isothermal amplification reaction(s) is polymerase cross-linking spiral reaction (PCLSR). PCLSR uses three primers (e.g., two outer-spiral primers and a cross-linking primer) to produce three independent prerequisite spiral products, which can be cross-linked into a final spiral amplification product. Accordingly, in some embodiments of the aspects, the amplification step comprises contacting the sample with a DNA polymerase and a set of primers (e.g., two outer-spiral primers and a cross-linking primer).

In some embodiments of any of the aspects, the DNA polymerase used in the amplification step is a strand-displacing polymerase. The term strand displacement describes the ability to displace downstream DNA encountered during synthesis. In some embodiments of any of the aspects, at least one (e.g. 1, 2, 3, or 4) strand-displacing DNA polymerase is selected from the group consisting of: Polymerase I Klenow fragment, Bst polymerase, Phi-29 polymerase, and Bacillus subtilis Pol I (Bsu) polymerase. In some embodiments of any of the aspects, step (c) comprising contacting the sample (e.g., cDNA) with the strand-displacing DNA polymerases Polymerase I Klenow fragment, Bst polymerase, Phi-29 polymerase, and Bacillus subtilis Pol I (Bsu) polymerase.

In some embodiments of any of the aspects, the DNA polymerase is provided (i.e., added to the reaction mixture) at a sufficient concentration to promote polymerization, e.g., 0.1 U/µL to 100 U/µL. As used herein, one unit (“U”) of DNA polymerase is defined as the amount of enzyme that will incorporate 10 nmol of dNTP into acid insoluble material in 30 minutes at 37° C.

In some embodiments of any of the aspects, the sample is contacted with at least one set of primers. In some embodiments of any of the aspects, the set of primers is specific to the target nucleic acid. In some embodiments of any of the aspects, the set of primers is specific (i.e., binds specifically through complementarity) to cDNA; in other words, the DNA produced in the RT step that is complementary to a target RNA. In some embodiments of any of the aspects, a primer comprises a detectable marker as described herein (e.g., FAM).

In some embodiments of any of the aspects, the sample is contacted with a DNA polymerase, a set of primers, and at least one of the following: reaction buffer (e.g., hydration buffer), water, and/or magnesium acetate. In some embodiments of any of the aspects, the sample is contacted with a DNA polymerase, a set of primers, a recombinase, single-stranded DNA binding protein, and at least one of the following: reaction buffer (e.g., hydration buffer), water, and/or magnesium acetate. In some embodiments of any of the aspects, the recombinase and/or ssDNA binding protein are provided in an “RPA pellet” that is dissolved with rehydration buffer and/or water.

In some embodiments of any of the aspects, a high concentration of magnesium in the amplification reaction increase the kinetics and/or yield of amplification product. In some embodiments of any of the aspects, the final magnesium concentration in the amplification reaction is 28 mM. In some embodiments of any of the aspects, the final magnesium concentration in the amplification reaction is at least 15 mM, at least 16 mM, at least 17 mM, at least 18 mM, at least 19 mM, at least 20 mM, at least 21 mM, at least 22 mM, at least 23 mM, at least 24 mM, at least 25 mM, at least 26 mM, at least 27 mM, at least 28 mM, at least 29 mM, at least 30 mM, at least 31 mM, at least 32 mM, at least 33 mM, at least 34 mM, at least 35 mM, at least 36 mM, at least 37 mM, at least 38 mM, at least 39 mM, at least 40 mM, at least 45 mM, or at least 50 mM.

In some embodiments of any of the aspects, the isothermal amplification step is performed between 12° C. and 70° C. In some embodiments of any of the aspects, the isothermal amplification step is performed at 65° C. As a non-limiting example, the isothermal amplification step is performed at a temperature of at least 12° C., at least 13° C., at least 14° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., at least 60° C., at least 61° C., at least 62° C., at least 63° C., at least 64° C., at least 65° C., at least 66° C., at least 67° C., at least 68° C., at least 69° C., or at least 70° C.

In some embodiments of any of the aspects, the isothermal amplification step is performed at a temperature of at most 12° C., at most 13° C., at most 14° C., at most 15° C., at most 16° C., at most 17° C., at most 18° C., at most 19° C., at most 20° C., at most 21° C., at most 22° C., at most 23° C., at most 24° C., at most 25° C., at most 26° C., at most 27° C., at most 28° C., at most 29° C., at most 30° C., at most 31° C., at most 32° C., at most 33° C., at most 34° C., at most 35° C., at most 36° C., at most 37° C., at most 38° C., at most 39° C., at most 40° C., at most 41° C., at most 42° C., at most 43° C., at most 44° C., at most 45° C., at most 46° C., at most 47° C., at most 48° C., at most 49° C., at most 50° C., at most 51° C., at most 52° C., at most 53° C., at most 54° C., at most 55° C., at most 56° C., at most 57° C., at most 58° C., at most 59° C., at most 60° C., at most 61° C., at most 62° C., at most 63° C., at most 64° C., at most 65° C., at most 66° C., at most 67° C., at most 68° C., at most 69° C., or at most 70° C. In some embodiments of any of the aspects, the isothermal amplification step is performed at room temperature (e.g., 20° C.-22° C.). In some embodiments of any of the aspects, the isothermal amplification step is performed at body temperature (e.g., 37° C.). In some embodiments of any of the aspects, the isothermal amplification step is performed on a heat block or an incubator set to approximately 42° C. or 65° C.

In some embodiments of any of the aspects, the isothermal amplification step is performed in at least 5 minutes. As a non-limiting example, the isothermal amplification step can be for a period of 30 minutes or less, 25 minutes or less, 20 minutes or less, 15 minutes or less, 10 minutes or less, or 5 minutes or less. In some embodiments of any of the aspects, the isothermal amplification step is performed in at most 5 minutes. As a non-limiting example, the isothermal amplification step is performed in at most 5 minutes, at most 6 minutes, at most 7 minutes, at most 8 minutes, at most 9 minutes, at most 10 minutes, at most 15 minutes, at most 20 minutes, at most 25 minutes, at most 30 minutes, at most 40 minutes, at most 50 minutes, at most 60 minutes, at most 70 minutes, at most 80 minutes, at most 90 minutes, or at most 100 minutes. In some embodiments of any of the aspects, the method further comprises a step of heating the single-stranded or double-stranded amplicon prior to detecting the amplicon. In some embodiments of any of the aspects, the heating step is performed to inactivate the enzymes (e.g., polymerase, recombinase, etc.) of the amplification reaction. As a non-limiting example, after the amplification and before detection, the amplicon is heated to at least 40° C., at least 45° C., at least 50° C., at least 55° C., at least 60° C., at least 65° C., at least 70° C., at least 75° C., at least 80° C., at least 85° C., at least 90° C., or at least 95° C. The heating step can be for a period of about 10 minutes, about 9 minutes, about 8 minutes, about 7 minutes, about 6 minutes, about 5 minutes, about 4 minutes, about 3 minutes, about 2 minutes, about 1 minute, about 45 seconds or about 30 seconds. In some embodiments of any of the aspects, the amplicon is heated for at most 1 minute. In some embodiments of any of the aspects, the amplicon is heated for at most 5 minutes. As a non-limiting example, the amplicon is heated for at most 1 minute, at most 2 minutes, at most 3 minutes, at most 4 minutes, at 5 minutes, at most 6 minutes, at most 7 minutes, at most 8 minutes, at most 9 minutes, at most 10 minutes, at most 15 minutes, at most 20 minutes, at most 25 minutes, at most 30 minutes, at most 40 minutes, at most 50 minutes, at most 60 minutes, at most 70 minutes, at most 80 minutes, at most 90 minutes, or at most 100 minutes.

### Single-Stranded Amplicon Methods

In several aspects, described herein are methods for creating single-stranded nucleic acid products from isothermal exponential amplification methods such as recombinase polymerase amplification (RPA) that can be specifically detected through lateral flow devices (LFD’s). This detection can be made specific to the target amplicon sequence, for improved specificity of detection by excluding background RPA amplicons which cause false positives. Critically, this hybridization-based sequence detection is performed directly on the LFD strip, eliminating the need for an additional long incubation step. Importantly, this step can be achieved through the use of relatively inexpensive equipment and can be performed rapidly (e.g. <15 minute turnaround time, even for detecting just a few copies of a target sequence).

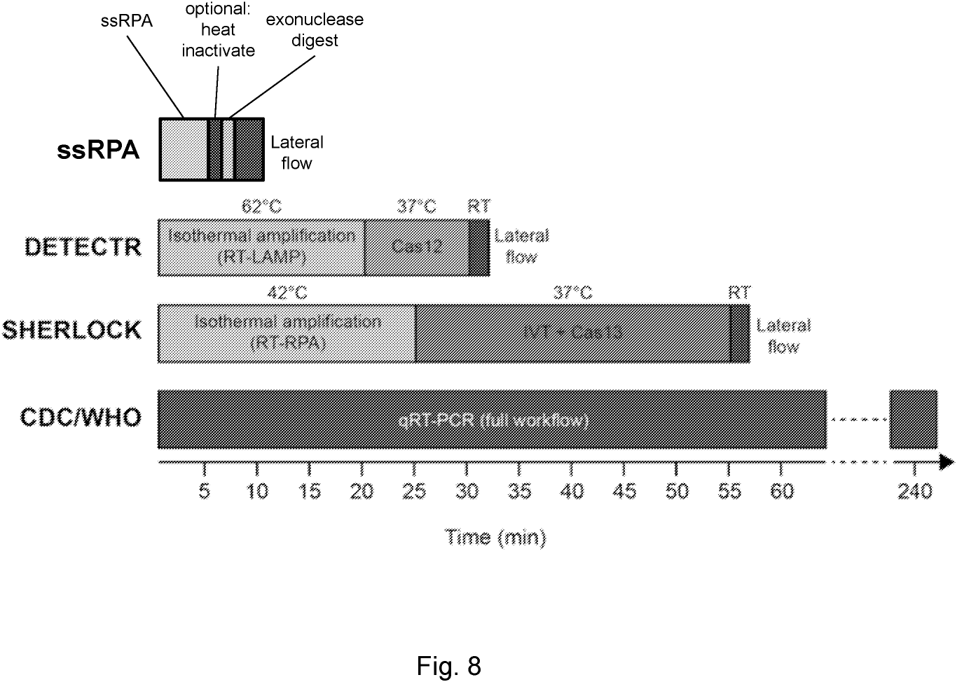

The methods described herein can be used to detect as low as 0.6 copy/uL input RNA in 8 min using lateral flow paper stick. In comparison, CDC qRT-PCR achieves 3-10 cp/uL in 120 min using expensive qPCR machine; SHERLOCK achieves 10-100 cp/uL in 60 min with a lateral flow paper stick; and Mammoth Biosciences™ DETECTR 70-300 cp/uL in 30 min with a lateral flow paper stick (see e.g., FIG. 8 and Table 1).

Table 1: Comparison of SARS-CoV-2 assay detection method. Details are shown for the present disclosure (e.g., ssRPA), DNA endonuclease-targeted CRISPR trans reporter (DETECTR), specific high-sensitivity enzymatic reporter unlocking (SHERLOCK), and the quantitative reverse transcription polymerase chain reaction (qRT-PCR) workflow used by the Centers for Disease Control and Prevention (CDC) and the World Health Organization (WHO).

| | | | | |
|---|---|---|---|---|
| | SARS-CoV-2 ssRPA | SARS-CoV-2 DETECTR | SARS-CoV-2 SHERLOCK | CDC SARS-CoV-2 qRT-PCR |
| Target | N gene or S gene | N gene & E gene (N gene gRNA compatible with CDC N2 amplicon, E gene compatible with WHO protocol) | S gene & Orflab gene | N-gene (3 amplicons) |
| Sample Control | RNase P | RNase P | None | RNase P |
| Limit of Detection | 0.6 copies/µL input | 70-300 copies/µL input | 10-100 copies/µL input | 3.16-10 copies/µL input |
| Assay Reaction Time | ~8 min | ~30 min | ~60 min | ~120 min |

In multiple aspects, described herein are methods of detecting a target nucleic acid. The target nucleic acid can be detected at the single molecular level using the methods, kits, and systems as described herein. The methods described herein generally comprise: (a) amplifying the target nucleic acid to detectable levels using a method that results in the formation of a single-stranded product and/or (b) detecting the amplified cDNA using a method as described further herein or known in the art. Accordingly, in some embodiments, the amplicon is single-stranded or partially single-stranded. In some embodiments, the method further comprises a step of preparing the single-stranded amplicon from the target nucleic acid prior to hybridizing a nucleic acid probe or set of primers as described herein with the amplicon.

In one aspect described herein is a method for preparing a single-stranded amplicon from a target nucleic acid, the method comprising: (a) amplifying a target nucleic acid with a first primer and a second primer to produce a double-stranded amplicon, wherein: (i) the first primer comprises a nucleic acid modification capable of inhibiting 5′->3′ cleaving activity of a 5′->3′ exonuclease; and (ii) the second primer optionally comprises a nucleic acid modification that enhances 5′->3′ cleaving activity of the 5′->3′ exonuclease; and (b) contacting the double-stranded amplicon from step (a) with the 5′->3′ exonuclease. In some embodiments, the nucleic acid modification capable of inhibiting 5′ -> 3′ cleaving activity of a 5′->3′ exonuclease is selected from the group consisting of modified internucleotide linkages modified nucleobase, modified sugar, and any combinations thereof.

In another aspect described herein is a method for preparing a single-stranded amplicon from a target nucleic acid, wherein the method comprises: (a) amplifying a target nucleic acid with a first primer and a second primer to produce a double-stranded amplicon, wherein the double-stranded amplicon comprises a 5′-single-stranded overhang on at least one end; and (b) contacting the double-stranded amplicon of step (a) with a nucleic acid probe comprising a sequence substantially complementary to the single-strand overhang, whereby the nucleic acid probe hybridizes with the complementary single-strand overhang and releases the non-complementary, to the probe, strand as a single-stranded amplicon.

In some embodiments of any of the aspects, at least one or both of the first or second primer comprises, at an internal position, a nucleic acid modification capable of inhibiting 5′->3′ cleaving activity of a 5′->3′ exonuclease. In some embodiments of any of the aspects, the method further comprises contacting the double-stranded amplicon with the 5′->3′ exonuclease prior to contacting with the nucleic acid probe. In some embodiments of any of the aspects, at least one or both of the first or second primer comprises a nucleic acid modification capable of inhibiting synthesis of a complementary strand by a polymerase. In some embodiments of any of the aspects, at least one or both of the first or second primer comprises a secondary structure that inhibits synthesis of a complementary strand by a polymerase. In some embodiments, the nucleic acid modification capable of inhibiting synthesis of a complementary strand by a polymerase is a non-canonical base or a spacer. In some embodiments, at least one or both of the first or second primer comprises a secondary structure that inhibits synthesis of a complementary strand by a polymerase.

In one aspect described herein is a method for detecting a nucleic acid target, wherein the method comprises: (a) asymmetrically amplifying a target nucleic acid to produce a single-stranded amplicon; and (b) detecting presence of the single-stranded amplicon.

In some embodiments, the method further comprises a step of adding a surfactant to the double-stranded amplicon. In some embodiments, preparing a single-stranded amplicon from the target nucleic acid comprises: (a) amplifying a target nucleic acid with a first primer and a second primer to produce a double-stranded amplicon: and (b) contacting the double-stranded amplicon from step (a) with a surfactant to displace the single-stranded amplicon. In some embodiments, the surfactant is an anionic surfactant. In some embodiments, the surfactant is sodium dodecyl sulfate (SDS).

In some embodiments, said amplification further comprises amplifying a target nucleic acid to produce a double-stranded amplicon. In some embodiments, the method further comprises hybridizing at least one nucleic acid probe to one strand of the double-stranded amplicon to form a complex comprising the at least one probe hybridized to one strand of the double-stranded amplicon, wherein said hybridizing is in the presence of a surfactant e.g., SDS, and/or a reagent capable of hybridizing/localizing a single-strand nucleic acid strand to a double-stranded nucleic acid. In some embodiments, the reagent capable of localizing a single-strand nucleic acid strand to a double-stranded nucleic acid is recombinase, single-stranded binding protein, Cas protein, zinc finger nuclease, transcription activator-like effector nuclease (TALEN), or any combinations thereof.

### Exonuclease

Described herein are methods comprising contacting a double-stranded amplicon with a 5′->3′ exonuclease. Exonucleases are enzymes that work by cleaving nucleotides one at a time from the end (exo) of a polynucleotide chain. A hydrolyzing reaction that breaks phosphodiester bonds at either the 3′ or the 5′ end occurs. Its close relative is the endonuclease, which cleaves phosphodiester bonds in the middle (endo) of a polynucleotide chain.

In some embodiments of any of the aspects, the exonuclease can be T7 exonuclease, Exonuclease VIII, lambda exonuclease, T5 exonuclease, RecJf, or any combinations thereof. In some embodiments, two or more, e.g., 3, 4, or 5 different exonucleases can be used.

In some embodiments of any of the aspects, the exonuclease is lambda exonuclease. Lambda exonuclease can also be referred to as Exodeoxyribonuclease (lambda-induced), EC 3.1.11.3, phage lambda-induced exonuclease, Escherichia coli exonuclease IV, E.coli exonuclease IV, exodeoxyribonuclease IV, and exonuclease IV. Lambda exonuclease has preference for double-stranded DNA (dsDNA), meaning that it degrades a single strand of dsDNA, primarily any strand which has a phosphate at its 5′ end. Lambda exonuclease catalyzes the removal of nucleotides from linear or nicked double-stranded DNA in the 5′ to 3′ direction. Lambda exonuclease exhibits highly processive degradation of double-stranded DNA from the 5′ end. The preferred substrate of Lambda exonuclease is 5′-phosphorylated double-stranded DNA, although non-phosphorylated substrates are degraded at a greatly reduced rate. In some embodiments of any of the aspects, Lambda Exonuclease can be used for conversion of linear double-stranded DNA to single-stranded DNA via preferred activity on 5′-phosphorylated ends. In some embodiments of any of the aspects, the Lambda exonuclease is isolated or derived from an E.coli strain that carries the cloned Lambda exonuclease gene (nfo) from Escherichia coli.

In some embodiments of any of the aspects, the exonuclease is T7 exonuclease. T7 exonuclease is a double-stranded DNA specific exonuclease. T7 exonuclease can also be referred to as Exonuclease gp6, Gene product 6 (EC:3.1.11.3), or Gp6. T7 exonuclease initiates at the 5′ termini of linear or nicked double-stranded DNA. T7 exonuclease catalyzes the removal of nucleotides from linear or nicked double-stranded DNA in the 5′ to 3′ direction. T7 Exonuclease can be used for site-directed mutagenesis or nick-site extension. In some embodiments of any of the aspects, the T7 exonuclease is isolated or derived from an E.coli strain that carries the cloned T7 exonuclease gene (gene 6) from Escherichia phage T7 (Bacteriophage T7).

In some embodiments of any of the aspects, the exonuclease is Exonuclease VIII. Exonuclease VIII is a double-stranded DNA specific exonuclease that initiates at the 5′ termini of linear double-stranded DNA and catalyzes the removal of nucleotides from linear double-stranded DNA in the 5′ to 3′ direction. In some embodiments of any of the aspects, the exonuclease is T5 exonuclease. T5 exonuclease is a double-stranded DNA specific exonuclease and single-stranded DNA endonuclease that initiates at the 5′ termini of linear or nicked double-stranded DNA and cleaves linear or nicked double-stranded DNA in the 5′ to 3′ direction. In some embodiments of any of the aspects, the exonuclease is RecJf. RecJf is a DNA specific exonuclease that catalyzes the removal of nucleotides from linear single-stranded DNA in the 5′ to 3′ direction. The preferred substrate of RecJf is double-stranded DNA with 5′ single stranded overhangs > 6 nucleotides long.

In some embodiments of any of the aspects, the exonuclease is provided (i.e., added to the reaction mixture) at a concentration of 0.1 U/µL to 5 U/µL. As used herein one unit (e.g., of Lambda exonuclease) is defined as the amount of enzyme required to produce 10 nmol of acid-soluble deoxyribonucleotide from double-stranded substrate in a total reaction volume of 50 µl in 30 minutes at 37° C. in 1X Lambda Exonuclease Reaction Buffer with 1 µg sonicated duplex [³H]-DNA; or one unit (e.g., of T7 exonuclease) is defined as the amount of enzyme required to produce 1 nmol of acid-soluble deoxyribonucleotide in a total reaction volume of 50 µl in 30 minutes at 37° C. in 1X NEBuffer 4 with 0.15 mM sonicated duplex [³H]-DNA.

As a non-limiting example, the exonuclease (e.g., Lambda exonuclease or T7 exonuclease) is provided at a concentration of at least 0.1 U/µL, at least 0.2 U/µL, at least 0.3 U/µL, at least 0.4 U/µL, at least 0.5 U/µL, at least 0.6 U/µL, at least 0.7 U/µL, at least 0.8 U/µL, at least 0.9 U/µL, at least 1.0 U/µL, at least 1.1 U/µL, at least 1.2 U/µL, at least 1.3 U/µL, at least 1.4 U/µL, at least 1.5 U/µL, at least 1.6 U/µL, at least 1.7 U/µL, at least 1.8 U/µL, at least 1.9 U/µL, at least 2.0 U/µL, at least 2.1 U/µL, at least 2.2 U/µL, at least 2.3 U/µL, at least 2.4 U/µL, at least 2.5 U/µL, at least 2.6 U/µL, at least 2.7 U/µL, at least 2.8 U/µL, at least 2.9 U/µL, at least 3.0 U/µL, at least 3.1 U/µL, at least 3.2 U/µL, at least 3.3 U/µL, at least 3.4 U/µL, at least 3.5 U/µL, at least 3.6 U/µL, at least 3.7 U/µL, at least 3.8 U/µL, at least 3.9 U/µL, at least 4.0 U/µL, at least 4.1 U/µL, at least 4.2 U/µL, at least 4.3 U/µL, at least 4.4 U/µL, at least 4.5 U/µL, at least 4.6 U/µL, at least 4.7 U/µL, at least 4.8 U/µL, at least 4.9 U/µL, at least 5.0 U/µL, at least 5.1 U/µL, at least 5.2 U/µL, at least 5.3 U/µL, at least 5.4 U/µL, at least 5.5 U/µL, at least 5.6 U/µL, at least 5.7 U/µL, at least 5.8 U/µL, at least 5.9 U/µL, at least 6.0 U/µL, at least 6.1 U/µL, at least 6.2 U/µL, at least 6.3 U/µL, at least 6.4 U/µL, at least 6.5 U/µL, at least 6.6 U/µL, at least 6.7 U/µL, at least 6.8 U/µL, at least 6.9 U/µL, at least 7.0 U/µL, at least 7.1 U/µL, at least 7.2 U/µL, at least 7.3 U/µL, at least 7.4 U/µL, at least 7.5 U/µL, at least 7.6 U/µL, at least 7.7 U/µL, at least 7.8 U/µL, at least 7.9 U/µL, at least 8.0 U/µL, at least 8.1 U/µL, at least 8.2 U/µL, at least 8.3 U/µL, at least 8.4 U/µL, at least 8.5 U/µL, at least 8.6 U/µL, at least 8.7 U/µL, at least 8.8 U/µL, at least 8.9 U/µL, at least 9.0 U/µL, at least 9.1 U/µL, at least 9.2 U/µL, at least 9.3 U/µL, at least 9.4 U/µL, at least 9.5 U/µL, at least 9.6 U/µL, at least 9.7 U/µL, at least 9.8 U/µL, at least 9.9 U/µL, at least 10 U/µL, at least 20 U/µL, at least 30 U/µL, at least 40 U/µL, or at least 50 U/µL.

In some embodiments of any of the aspects, the exonuclease step is performed between 12° C. and 45° C. As a non-limiting example, the exonuclease step is performed at a temperature of at least 12° C., at least 13° C., at least 14° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C.

In some embodiments of any of the aspects, the exonuclease step is performed at a temperature of at most 12° C., at most 13° C., at most 14° C., at most 15° C., at most 16° C., at most 17° C., at most 18° C., at most 19° C., at most 20° C., at most 21° C., at most 22° C., at most 23° C., at most 24° C., at most 25° C., at most 26° C., at most 27° C., at most 28° C., at most 29° C., at most 30° C., at most 31° C., at most 32° C., at most 33° C., at most 34° C., at most 35° C., at most 36° C., at most 37° C., at most 38° C., at most 39° C., at most 40° C., at most 41° C., at most 42° C., at most 43° C., at most 44° C., at most 45° C. In some embodiments of any of the aspects, the exonuclease step is performed at ambient or room temperature (e.g., 20° C.-22° C.). In some embodiments of any of the aspects, the exonuclease step is performed at body temperature (e.g., 37° C.). In some embodiments of any of the aspects, the exonuclease step is performed on a heat block set to approximately 42° C.

Treatment with exonuclease can be for a period of about 10 minutes, about 9 minutes, about 8 minutes, about 7 minutes, about 6 minutes, about 5 minutes, about 4 minutes, about 3 minutes, about 2 minutes, about 1 minute, about 45 seconds or about 30 seconds. In some embodiments of any of the aspects, the exonuclease step is performed at most 1 minute. In some embodiments of any of the aspects, the exonuclease step is performed at most 5 minutes. As a non-limiting example, the exonuclease step is performed in at most 1 minute, at most 2 minutes, at most 3 minutes, at most 4 minutes, at 5 minutes, at most 6 minutes, at most 7 minutes, at most 8 minutes, at most 9 minutes, at most 10 minutes, at most 15 minutes, at most 20 minutes, at most 25 minutes, at most 30 minutes, at most 40 minutes, at most 50 minutes, at most 60 minutes, at most 70 minutes, at most 80 minutes, at most 90 minutes, or at most 100 minutes.

In some embodiments of any of the aspects, the exonuclease (e.g., Lambda exonuclease) is provided with a reaction buffer (e.g., Lambda Exonuclease Reaction Buffer), comprising, for example, Glycine-KOH, MgCl₂, and Bovine Serum Albumin (BSA).

In some embodiments of any of the aspects, the exonuclease (e.g., T7 exonuclease) is provided with a reaction buffer (e.g., NEBuffer 4), comprising, for example, Potassium Acetate, Trisacetate, Magnesium Acetate, and/or DTT.

In some embodiments of any of the aspects, the method further comprises a step of heating the double-stranded amplicon prior to contacting with the 5′->3′ exonuclease. In some embodiments of any of the aspects, the heating step is performed to inactivate the enzymes (e.g., polymerase, recombinase, etc.) of the isothermal amplification reaction. As a non-limiting example, after the amplification and before contacting with a 5′->3′ exonuclease, the double-stranded amplicon is heated to at least 40° C., at least 45° C., at least 50° C., at least 55° C., at least 60° C., at least 65° C., at least 70° C., at least 75° C., at least 80° C., at least 85° C., at least 90° C., or at least 95° C. The heating step can be for a period of about 10 minutes, about 9 minutes, about 8 minutes, about 7 minutes, about 6 minutes, about 5 minutes, about 4 minutes, about 3 minutes, about 2 minutes, about 1 minute, about 45 seconds or about 30 seconds. In some embodiments of any of the aspects, the double-stranded amplicon is heated for at most 1 minute. In some embodiments of any of the aspects, the double-stranded amplicon is heated for at most 5 minutes. As a non-limiting example, the double-stranded amplicon is heated for at most 1 minute, at most 2 minutes, at most 3 minutes, at most 4 minutes, at 5 minutes, at most 6 minutes, at most 7 minutes, at most 8 minutes, at most 9 minutes, at most 10 minutes, at most 15 minutes, at most 20 minutes, at most 25 minutes, at most 30 minutes, at most 40 minutes, at most 50 minutes, at most 60 minutes, at most 70 minutes, at most 80 minutes, at most 90 minutes, or at most 100 minutes.

In some embodiments of any of the aspects, the method does not comprise a step of heating the double-stranded amplicon prior to contacting with the 5′->3′ exonuclease. In one aspect, described herein is a method for preparing a single-stranded amplicon from a target nucleic acid, the method comprising: (a) amplifying a target nucleic acid with a first primer and a second primer to produce a double-stranded amplicon, wherein: (i) the first primer comprises a nucleic acid modification capable of inhibiting 5′->3′ cleaving activity of a 5′->3′ exonuclease; and (ii) the second primer optionally comprises a nucleic acid modification that enhances 5′->3′ cleaving activity of the 5′->3′ exonuclease; and (b) contacting the double-stranded amplicon from step (a) with a T7 exonuclease, wherein the method does not comprise a step of heating the double-stranded amplicon prior to contacting with the exonuclease.

### Asymmetric Amplification

In one aspect described herein is a method for detecting a nucleic acid target, wherein the method comprises: (a) asymmetrically amplifying a target nucleic acid to produce a single-stranded amplicon; and (b) detecting presence of the single-stranded amplicon. As used herein the term “asymmetric amplification” refers to an amplification reaction in which a specific ssDNA product is produced. In some embodiments of any of the aspects, the amplification comprises isothermal amplification. In some embodiments of any of the aspects, the amplification comprises recombinase polymerase amplification.

In one aspect described herein is a method for detecting a nucleic acid target, wherein the method comprises: (a) asymmetrically amplifying a target nucleic acid to produce a single-stranded amplicon, wherein the amplification comprises isothermal amplification; and (b) detecting presence of the single-stranded amplicon. In one aspect described herein is a method for detecting a nucleic acid target, wherein the method comprises: (a) asymmetrically amplifying a target nucleic acid to produce a single-stranded amplicon, wherein the amplification comprises recombinase polymerase amplification (RPA); and (b) detecting presence of the single-stranded amplicon.

In some embodiments of any of the aspects, asymmetric amplification is due to an increased ratio of one primer (e.g., first or second) compared to the other primer (e.g. second or first). As a non-limiting example, an asymmetric amplification reaction can comprise an at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 250%, at least 300%, at least 350%, at least 400%, at least 450%, or at least 500% increase of one primer (e.g., first or second) compared to the other primer (e.g. second or first). In some embodiments of any of the aspects, amplification with the more abundant primer results in an increased abundance of that primer’s ssDNA extension product. In some embodiments of any of the aspects, amplification with the less abundant primer results in dsDNA product and little to none of that primer’s ssDNA extension product.

In some embodiments of any of the aspects, the products of an asymmetric comprise a mixture of dsDNA and ssDNA. In some embodiments of any of the aspects, the dsDNA product of the asymmetric amplification reaction is degraded using a dsDNA-specific nuclease (e.g., dsDNase, T5 exonuclease).

In some embodiments of any of the aspects, one or both of the primers for the asymmetric amplification reaction are modified to reduce or prevent further spurious extension of the ssDNA product. In some embodiments of any of the aspects, the 5′ end of the less abundant primer is modified to reduce or prevent further spurious extension of the ssDNA product. In some embodiments of any of the aspects, the modification to one or both amplification primers comprises dideoxynucleotides, which are chain-elongating inhibitors of DNA polymerase (e.g., ddGTP, ddATP, ddTTP, ddCTP).

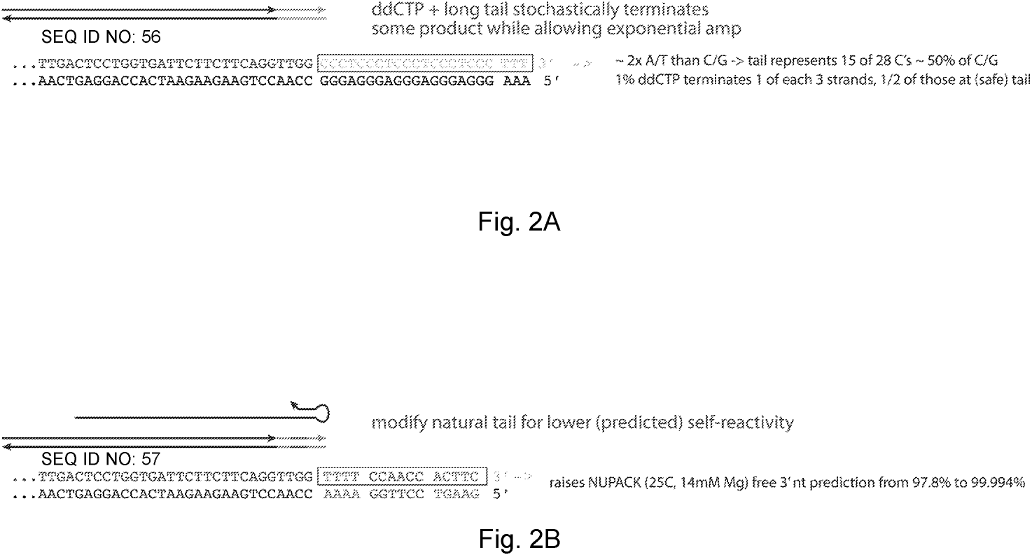

In some embodiments of any of the aspects, the modification to one or both amplification primers comprises a tail, e.g., comprising a repeating nucleotide motif with an increased abundance of at least one nucleotide. In some embodiments of any of the aspects, the amplification reaction mixture comprises at least one type of dideoxynucleotide (e.g., ddGTP, ddATP, ddTTP, ddCTP) that base pairs with the abundant nucleotide in the tail, which stochastically terminates some product while dNTPs present in the reaction mixture allow exponential amplification. In some embodiments of any of the aspects, the tail comprises an increased ratio of C/G to A/T and the amplification reaction mixture comprises ddCTP or ddGTP. In some embodiments of any of the aspects, the tail comprises an increased ratio of A/T to C/G and the amplification reaction mixture comprises ddATP or ddTTP. In some embodiments of any of the aspects, the tail comprises the motif GGGA, e.g., repeated 1, 2, 3, 4, 5, or more times, and the amplification reaction mixture comprises ddCTP. As a non-limiting example, the tail comprises at least two times more C/G than A/T, such that the tail is over 50% C/G; with 1% ddCTP in the reaction mixture, extension is terminated at 1 of each 3 strands, predominantly at the less abundant primer (see e.g., FIG. 2A).

In some embodiments of any of the aspects, one or both of the primers for the asymmetric amplification reaction are modified to reduce or prevent self-reactivity. In some embodiments of any of the aspects, the 5′ end of the less abundant primer is modified to reduce or prevent self-reactivity. As used herein, “self-reactivity” refers to the propensity of a primer to hybridize with itself, thus creating a hairpin structure that can cause self-annealing and aberrant extension of the ssDNA product. As a non-limiting example, the primer can be designed using analysis software (e.g., NUPACK), such that the free 3′ nt prediction is greater than 97.8%, e.g., at least 97.9%, at least 98.0%, at least 98.1%, at least 98.2%, at least 98.3%, at least 98.4%, at least 98.5%, at least 98.6%, at least 98.7%, at least 98.8%, at least 98.9%, at least 99.0%, at least 99.1%, at least 99.2%, at least 99.3%, at least 99.4%, at least 99.5%, at least 99.6%, at least 99.7%, at least 99.8%, at least 99.9%, at least 99.95%, at least 99.99%, or at least 99.994%.

### Stopper-Based Priming

In one aspect described herein is a method for preparing a single-stranded amplicon from a target nucleic acid, wherein the method comprises: (a) amplifying a target nucleic acid with a first primer and a second primer to produce a double-stranded amplicon, wherein the double-stranded amplicon comprises a 5′-single-stranded overhang on at least one end; and (b) contacting the double-stranded amplicon of step (a) with a nucleic acid probe comprising a sequence substantially complementary to the single-strand overhang, whereby the nucleic acid probe hybridizes with the complementary single-strand overhang and releases the non-complementary, to the probe, strand as a single-stranded amplicon. In some embodiments of any of the aspects, the amplification comprises isothermal amplification. In some embodiments of any of the aspects, the amplification comprises recombinase polymerase amplification.

In some embodiments of any of the aspects, at least one or both of the first or second primer comprises a nucleic acid modification capable of inhibiting synthesis of a complementary strand by a polymerase. In some embodiments of any of the aspects, the first primer comprises a nucleic acid modification capable of inhibiting synthesis of a complementary strand by a polymerase. In some embodiments of any of the aspects, the second primer comprises a nucleic acid modification capable of inhibiting synthesis of a complementary strand by a polymerase. In some embodiments of any of the aspects, the first and second primer each comprises a nucleic acid modification capable of inhibiting synthesis of a complementary strand by a polymerase, which can be the same or different modification.

In some embodiments of any of the aspects, the nucleic acid modification capable of inhibiting synthesis of a complementary strand by a polymerase is a non-canonical base, as described further herein. In some embodiments of any of the aspects, the non-canonical bases is isocytosine (iso-dC). In some embodiments of any of the aspects, the non-canonical bases is isoguanosine (iso-dG). In some embodiments of any of the aspects, the nucleic acid modification capable of inhibiting synthesis of a complementary strand by a polymerase is a spacer. In some embodiments of any of the aspects, the spacer is located at an internal location of one or both primers. Non-limiting examples of spacers include the C3 spacer (phosphoramidite); 1′,2′-Dideoxyribose (dSpacer); PC (Photo-Cleavable) Spacer; Spacer 9 (a triethylene glycol spacer); and Spacer 18 (an 18-atom hexa-ethyleneglycol spacer).

In some embodiments of any of the aspects, at least one or both of the first or second primer comprises a secondary structure that inhibits synthesis of a complementary strand by a polymerase. In some embodiments of any of the aspects, the first primer comprises a secondary structure that inhibits synthesis of a complementary strand by a polymerase. In some embodiments of any of the aspects, the second primer comprises a secondary structure that inhibits synthesis of a complementary strand by a polymerase. In some embodiments of any of the aspects, the first and second primer each comprises a secondary structure that inhibits synthesis of a complementary strand by a polymerase, which can be the same or different secondary structure.

### Toehold Exposure

In one aspect described herein is a method for preparing a single-stranded amplicon from a target nucleic acid, wherein the method comprises: (a) amplifying a target nucleic acid with a first primer and a second primer to produce a double-stranded amplicon, wherein the double-stranded amplicon comprises a 5′-single-stranded overhang on at least one end; and (b) contacting the double-stranded amplicon of step (a) with a nucleic acid probe comprising a sequence substantially complementary to the single-strand overhang, whereby the nucleic acid probe hybridizes with the complementary single-strand overhang and releases the non-complementary, to the probe, strand as a single-stranded amplicon. In some embodiments of any of the aspects, the amplification comprises isothermal amplification. In some embodiments of any of the aspects, the amplification comprises recombinase polymerase amplification.

In some embodiments of any of the aspects, at least one or both of the first or second primer comprises, at an internal position, a nucleic acid modification capable of inhibiting 5′->3′ cleaving activity of a 5′->3′ exonuclease. In some embodiments of any of the aspects, the first primer comprises, at an internal position, a nucleic acid modification capable of inhibiting 5′->3′ cleaving activity of a 5′->3′ exonuclease. In some embodiments of any of the aspects, the second primer comprises, at an internal position, a nucleic acid modification capable of inhibiting 5′->3′ cleaving activity of a 5′->3′ exonuclease. In some embodiments of any of the aspects, the first and second primer each comprises, at an internal position, a nucleic acid modification capable of inhibiting 5′->3′ cleaving activity of a 5′->3′ exonuclease, which can be the same or different modification. In some embodiments of any of the aspects, the method further comprises contacting the double-stranded amplicon with the 5′->3′ exonuclease prior to contacting with the nucleic acid probe.

In some embodiments of any of the aspects, at least one or both of the first or second primer comprises, at an internal position, a ribonucleotide (e.g., uracil) as opposed to a deoxynucleotide. Non-limiting examples of ribonucleotides include uracil, thymine ribonucleotide, cytosine ribonucleotide, adenine ribonucleotide, and guanine ribonucleotide. In some embodiments of any of the aspects, the first primer comprises, at an internal position, a ribonucleotide (e.g., uracil). In some embodiments of any of the aspects, the second primer comprises, at an internal position, a ribonucleotide (e.g., uracil). In some embodiments of any of the aspects, the first and second primer each comprises, at an internal position, a ribonucleotide (e.g., uracil), which can be the same or different ribonucleotide. In some embodiments of any of the aspects, a nucleic acid described herein (e.g., one or both primers) comprises 1, 2, 3, 4, 5, 6, or more ribonucleotides (e.g., uracil), e.g., at an internal position.

In some embodiments of any of the aspects, the method further comprises contacting the double-stranded amplicon with a ribonucleotide-specific endonuclease prior to contacting with the nucleic acid probe. Contacting the double-stranded amplicon with a ribonucleotide-specific endonuclease introduces an internal cut in one strand of the amplicon, such that at the incubation temperature the short ssDNA fragment is removed, creating a single-stranded overhang. In some embodiments of any of the aspects, the method further comprises contacting the double-stranded amplicon with a uracil-specific endonuclease prior to contacting with the nucleic acid probe. In some embodiments of any of the aspects, the uracil-specific endonuclease is USER™ (Uracil-Specific Excision Reagent) enzyme. USER Enzyme generates a single nucleotide gap at the location of a uracil. USER Enzyme is a mixture of Uracil DNA glycosylase (UDG) and the DNA glycosylase-lyase Endonuclease VIII. UDG catalyzes the excision of a uracil base, forming an abasic (apyrimidinic) site while leaving the phosphodiester backbone intact. The lyase activity of Endonuclease VIII breaks the phosphodiester backbone at the 3′ and 5′ sides of the abasic site so that base-free deoxyribose is released.

In some embodiments of any of the aspects, the method further comprises a step of heating the double-stranded amplicon after contacting with a ribonucleotide-specific endonuclease and prior to contacting with a nucleic acid probe. In some embodiments of any of the aspects, the heating step is performed to expose the single-stranded overhang(s). As a non-limiting example, after contacting with a ribonucleotide-specific endonuclease and prior to contacting with a nucleic acid probe, the double-stranded amplicon is heated to at least 40° C., at least 45° C., at least 50° C., at least 55° C., at least 60° C., at least 65° C., at least 70° C., at least 75° C., at least 80° C., at least 85° C., at least 90° C., or at least 95° C. The heating step can be for a period of about 10 minutes, about 9 minutes, about 8 minutes, about 7 minutes, about 6 minutes, about 5 minutes, about 4 minutes, about 3 minutes, about 2 minutes, about 1 minute, about 45 seconds or about 30 seconds. In some embodiments of any of the aspects, the double-stranded amplicon is heated for at most 1 minute. In some embodiments of any of the aspects, the double-stranded amplicon is heated for at most 5 minutes. As a non-limiting example, the double-stranded amplicon is heated for at most 1 minute, at most 2 minutes, at most 3 minutes, at most 4 minutes, at 5 minutes, at most 6 minutes, at most 7 minutes, at most 8 minutes, at most 9 minutes, at most 10 minutes, at most 15 minutes, at most 20 minutes, at most 25 minutes, at most 30 minutes, at most 40 minutes, at most 50 minutes, at most 60 minutes, at most 70 minutes, at most 80 minutes, at most 90 minutes, or at most 100 minutes.

In some embodiments of any of the aspects, a double-stranded amplicon comprising two single-stranded overhangs is contacted with two nucleic acid probes, wherein the first probe comprises a sequence substantially complementary to the first single-strand overhang, and wherein the second probe comprises a sequence substantially complementary to the second single-strand overhang. In some embodiments of any of the aspects, a double-stranded amplicon comprising two single-stranded overhangs is contacted with 2, 3, 4, 5, 6, or more nucleic acid probes. In some embodiments of any of the aspects, the two or more nucleic acid probe hybridizes with the complementary single-strand overhang and releases the non-complementary, to the probes, strand as a single-stranded amplicon. In some embodiments of any of the aspects, at least one of the probes comprises a detectable marker and/or a ligand, as described further herein.

In one aspect described herein is a method for detecting a target nucleic acid, the method comprising: (a) amplifying a target nucleic acid with a first primer and a second primer to produce a double-stranded amplicon, wherein the first primer comprises a detectable label at its 5′-end; (b) contacting the double-stranded amplicon with a 5′->3′ exonuclease to produce an amplicon having a single-stranded region (e.g., a single-stranded amplicon); and (c) detecting the amplicon having a single-stranded region, wherein said detecting comprises applying amplicon having a single-stranded region to a lateral flow test strip, wherein the later flow test strip comprises: a test/capture region comprising a nucleic acid capture probe immobilized therein, wherein the nucleic acid capture probe comprises a toehold domain (e.g., a single-stranded region) comprising a nucleotide sequence substantially complementary to at least a part of the single-stranded amplicon.

In one aspect described herein is a method for detecting a target nucleic acid, the method comprising: (a) amplifying a target nucleic acid with a first primer and a second primer to produce a double-stranded amplicon, wherein: (i) the first primer comprises a detectable label at its 5′-end; (ii) the second primer comprises one or more uridine nucleotides; and (b) contacting the double-stranded amplicon from step (a) with Uracil-DNA glycosylase (UDG) to produce an amplicon having a single-stranded region (e.g., a single-stranded amplicon); and (c) detecting the amplicon having the single-stranded region, wherein said detecting comprises applying the amplicon having the single-stranded region to a lateral flow test strip, wherein the later flow test strip comprises: a test/capture region comprising a nucleic acid capture probe immobilized therein, wherein the nucleic acid capture probe comprises a toehold domain (e.g., a single-stranded region) comprising a nucleotide sequence substantially complementary to at least a part of the single-stranded region of the amplicon.

In one aspect described herein is a method for detecting a target nucleic acid, the method comprising: (a) amplifying a target nucleic acid with a first primer and a second primer to produce a double-stranded amplicon, wherein the first primer comprises a detectable label at its 5′-end and a nucleic acid modification capable of inhibiting synthesis of a complementary strand by a polymerase at an internal position, and wherein the double-stranded amplicon comprises a 5′ single-stranded region at one end; and (b) detecting the amplicon having the 5′ single-stranded region, wherein said detecting comprises applying the amplicon to a lateral flow test strip, wherein the later flow test strip comprises: a test/capture region comprising a nucleic acid capture probe immobilized therein, wherein a first region/domain of the nucleic acid capture probe comprises a toehold domain comprising a nucleotide sequence substantially complementary to at least a part of the single-stranded amplicon.

In some embodiments of any of the aspects, the method further comprises a step of contacting the double-stranded amplicon with a surfactant, e.g., SDS.

### Buffer Additives

In some embodiments of any of the aspects, the method further comprises a step of adding a buffer additive to at least one of the reactions as described herein. As non-limiting examples, the buffer additive can be added to the amplification reaction, the exonuclease reaction, and/or to the detection reaction (e.g. LFD). Addition of a buffer additive can improve the accuracy of the LFD output. Non-limiting examples of buffer modifications include surfactant (e.g., SDS, LDS, alkyl sulfates, alkyl sulfonates or other detergents), bile salts, ionic salt, chaotropic agents (i.e., a compound which disrupts hydrogen bonding in aqueous solution), formamide, DNA duplex destabilizers, or reducing agents. In some embodiments of any of the aspects, the buffer additive is a surfactant.

In some embodiments of any of the aspects, the detection step is carried out in presence of a surfactant, bile salt, ionic salt, chaotropic agent (i.e., a compound which disrupts hydrogen bonding in aqueous solution), DNA duplex destabilizer, reducing agent, or any combinations thereof. In some embodiments of any of the aspects, the surfactant, bile salt, ionic salt, chaotropic agent, formamide, DNA duplex destabilizer, and/or reducing agent is present in the lateral flow assay (e.g., in a running buffer) at a concentration ranging from 0.5% to 20%. For example, the surfactant, bile salt, ionic salt, chaotropic agent, formamide, DNA duplex destabilizer, and/or reducing agent is present in the lateral flow assay (e.g., in a running buffer) at a concentration of about 5% to about 15%, about 7.5% to about 12.5%. In some embodiments, the surfactant, bile salt, ionic salt, chaotropic agent, formamide, DNA duplex destabilizer, and/or reducing agent is present in the lateral flow assay (e.g., in a running buffer) at a concentration of about 10%.

In some embodiments of any of the aspects, the method further comprises a step of adding a surfactant, bile salt, ionic salt, chaotropic agent, formamide, DNA duplex destabilizer, and/or reducing agent to the double-stranded amplicon. In some embodiments of any of the aspects, the surfactant is added to the double-stranded amplicon prior to contacting with an exonuclease as described herein. In some embodiments of any of the aspects, the surfactant, bile salt, ionic salt, chaotropic agent, formamide, DNA duplex destabilizer, and/or reducing agent is added to the double-stranded amplicon after contacting with an exonuclease as described herein. In some embodiments of any of the aspects, the surfactant is added to the double-stranded amplicon prior to contacting with a detection probe. In some embodiments of any of the aspects, the surfactant is added at the start, middle, or end of the amplification reaction. In some embodiments of any of the aspects, the surfactant is added at the start of the amplification reaction. In some embodiments of any of the aspects, the surfactant is added with the detection probe. In some embodiments of any of the aspects, the surfactant, bile salt, ionic salt, chaotropic agent, formamide, DNA duplex destabilizer, and/or reducing agent is added to the lateral flow device, as described further herein.

The surfactant can act to make a single strand of the double-stranded amplicon more accessible, e.g., to the exonuclease or detection probe. The surfactant can be in ionic surfactant or a non-ionic surfactant. In some embodiments of any of the aspects, the surfactant can allow for a one-pot reaction. In some embodiments of any of the aspects, the surfactant reduces the rate of false positives (e.g., by at least 5%, at least 10%, at least 20%, at least 30%, at least 40%, at least 50%, at least 60%, at least 70%, at least 80%, at least 90%, or at least 100%).

Accordingly, in one aspect described herein is a method for preparing a single-stranded amplicon from a target nucleic acid, the method comprising: (a) amplifying a target nucleic acid with a first primer and a second primer to produce a double-stranded amplicon: and (b) contacting the double-stranded amplicon from step (a) with a surfactant to displace the single-stranded amplicon. A double-stranded amplicon produced by any of the methods described herein can be contacted with a surfactant, e.g., to prepare a single-stranded amplicon for detection.

For example, the surfactant can anionic, cationic or zwitterionic. Exemplary anionic surfactants include, but are not limited to, alkyl sulfate, alkyl ether sulfate, alkyl sulfonate, alkylaryl sulfonate, alkyl succinate, alkyl sulfobutane Diacid salt, N-alkylfluorenyl sarcosinate, fluorenyl taurate, fluorenyl isethionate, alkyl phosphate, alkyl ether phosphate, alkyl ether carboxylate, α- Olefin sulfonates and alkali metal salts and alkaline earth metal salts and ammonium salts with their triethanolamine salts. Specific exemplary anionic surfactants include, but are not limited to, ammonium laurylsulfosuccinate, sodium lauryl sulfate, sodium lauryl ether sulfate, ammonium lauryl ether sulfate, triethanolamine dodecylbenzenesulfonate, Sodium lauryl sarcosinate, ammonium lauryl sulfate, sodium oleyl succinate, sodium lauryl sulfate and sodium dodecylbenzenesulfonate. Exemplary cationic surfactants include, but are not limited to, cetylpyridinium chloride, cetyltrimethylammonium bromide (CTAB; Calbiochem™ #B22633 or Aldrich™ #85582-0), cetyltrimethylammonium chloride (CTACl; Aldrich™ #29273-7), dodecyltrimethylammonium bromide (DTAB, Sigma #D-8638), dodecyltrimethylammonium chloride (DTACl), octyl trimethyl ammonium bromide, tetradecyltrimethylammonium bromide (TTAB), tetradecyltrimethylammonium chloride (TTACl), dodecylethyidimethylammonium bromide (DEDTAB), decyltrimethylammonium bromide (DlOTAB), dodecyltriphenylphosphonium bromide (DTPB), octadecylyl trimethyl ammonium bromide, stearoalkonium chloride, olealkonium chloride, cetrimonium chloride, alkyl trimethyl ammonium methosulfate, palmitamidopropyl trimethyl chloride, quaternium 84 (Mackemium™ NLE; McIntyre Group™, Ltd.), and wheat lipid epoxide (Mackernium WLE™; McIntyre Group™, Ltd.), octyldimethylamine, decyidimethylamine, dodecyidimethylamine, tetradecyldimethylamine, hexadecyidimethylamine, octyldecyldimethylamine, octyidecylmethylamine, didecylmethylamine, dodecylmethylamine, triacetylammonium chloride, cetrimonium chloride, and alkyl dimethyl benzyl ammonium chloride. Additional classes of cationic surfactants include, but are not limited to, phosphonium, imidzoline, and ethylated amine groups.

In some embodiments of the various aspects, the surfactant is an anionic surfactant.

In some preferred embodiments of any of the aspects, the surfactant is selected from the group consisting of: sodium dodecyl sulfate (SDS); lithium dodecyl sulfate (LDS); an alkyl sulfate; or an alkyl sulfonate. In some preferred embodiments of any of the aspects, the surfactant is sodium dodecyl sulfate (SDS).

The surfactant, bile salt, ionic salt, chaotropic agent, formamide, DNA duplex destabilizer, and/or reducing agent can be added to any desired amount. For example, the surfactant can be added to a final concentration of about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 2 mM, about 3 mM, about 4 mM, about 5 mM, about 6 mM, about 7 mM, about 8 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM.

In some embodiments of the various aspects, the surfactant, bile salt, ionic salt, chaotropic agent, formamide, DNA duplex destabilizer, and/or reducing agent is present in a solution (e.g., amplification reaction, exonuclease reaction, LFD running buffer) at a concentration ranging from 0.5% to 20%. In some embodiments of the various aspects, the surfactant, bile salt, ionic salt, chaotropic agent, formamide, DNA duplex destabilizer, and/or reducing agent is present in a solution at a concentration of at least 0.5%. In some embodiments of the various aspects, the surfactant, bile salt, ionic salt, chaotropic agent, formamide, DNA duplex destabilizer, and/or reducing agent is present in a solution at a concentration of about 5%. In some embodiments of the various aspects, the surfactant, bile salt, ionic salt, chaotropic agent, formamide, DNA duplex destabilizer, and/or reducing agent is present in a solution at a concentration of about 10%. In some embodiments of the various aspects, the surfactant, bile salt, ionic salt, chaotropic agent, formamide, DNA duplex destabilizer, and/or reducing agent is present in a solution at a concentration of at most 20%. In some embodiments of the various aspects, the surfactant, bile salt, ionic salt, chaotropic agent, formamide, DNA duplex destabilizer, and/or reducing agent is present in a solution at a concentration of at least 0.5%, at least 1%, at least 1.5%, at least 2%, at least 2.5%, at least 3%, at least 3.5%, at least 4%, at least 4.5%, at least 5%, at least 5.5%, at least 6%, at least 6.5%, at least 7%, at least 7.5%, at least 8%, at least 8.5%, at least 9%, at least 9.5%, at least 10%, at least 10.5%, at least 11%, at least 11.5%, at least 12%, at least 12.5%, at least 13%, at least 13.5%, at least 14%, at least 14.5%, at least 15%, at least 15.5%, at least 16%, at least 16.5%, at least 17%, at least 17.5%, at least 18%, at least 18.5%, at least 19%, at least 19.5%, or at least 20%. In some embodiments of the various aspects, the surfactant, bile salt, ionic salt, chaotropic agent, formamide, DNA duplex destabilizer, and/or reducing agent is present in a solution at a concentration of at most 0.5%, at most 1%, at most 1.5%, at most 2%, at most 2.5%, at most 3%, at most 3.5%, at most 4%, at most 4.5%, at most 5%, at most 5.5%, at most 6%, at most 6.5%, at most 7%, at most 7.5%, at most 8%, at most 8.5%, at most 9%, at most 9.5%, at most 10%, at most 10.5%, at most 11%, at most 11.5%, at most 12%, at most 12.5%, at most 13%, at most 13.5%, at most 14%, at most 14.5%, at most 15%, at most 15.5%, at most 16%, at most 16.5%, at most 17%, at most 17.5%, at most 18%, at most 18.5%, at most 19%, at most 19.5%, or at most 20%.

In some embodiments of the various aspects, the surfactant, bile salt, ionic salt, chaotropic agent, formamide, DNA duplex destabilizer, and/or reducing agent is added to a solution at a volume of at most 20 uL. In some embodiments of the various aspects, the surfactant, bile salt, ionic salt, chaotropic agent, formamide, DNA duplex destabilizer, and/or reducing agent is added to a solution at a volume of at most 20 uL. In some embodiments of the various aspects, the surfactant, bile salt, ionic salt, chaotropic agent, formamide, DNA duplex destabilizer, and/or reducing agent is added to a solution at a volume of at most 1 uL, at most 2 uL, at most 3 uL, at most 4 uL, at most 5 uL, at most 6 uL, at most 7 uL, at most 8 uL, at most 9 uL, at most 10 uL, at most 11 uL, at most 12 uL, at most 13 uL, at most 14 uL, at most 15 uL, at most 16 uL, at most 17 uL, at most 18 uL, at most 19 uL, or at most 20 uL

In one aspect described herein is a method for detecting a target nucleic acid, the method comprising: (a) amplifying a target nucleic acid to produce a double-stranded amplicon; and (b) hybridizing a first nucleic acid probe and a second nucleic acid probe to one strand of the double-stranded amplicon to form a complex comprising the first and second probes hybridized to one strand of the double-stranded amplicon, wherein said hybridizing is in the presence of a surfactant e.g., SDS, and/or a reagent capable of hybridizing/localizing a single-strand nucleic acid strand to a double-stranded nucleic acid, wherein the first nucleic acid probe comprises a first detectable label and the second nucleic acid probe comprises a ligand for a ligand binding molecule; and (c) detecting the complex, e.g., by a lateral flow assay/device.

In some embodiments of any of the aspects, the method further comprises a step of adding a crowding agent to at least one of the reactions as described herein. As non-limiting examples, the crowding additive can be added to the amplification reaction, the exonuclease reaction, and/or to the detection reaction (e.g. LFD). Non-limiting examples of crowding agents include PEG, PEG8000, dextran of different molecular weights, dextran sulfate, ficoll, or glycerol.

In some embodiments of any of the aspects, the method further comprises a step of adding a blocking agent to at least one of the reactions as described herein. As non-limiting examples, the blocking additive can be added to the amplification reaction, the exonuclease reaction, and/or to the detection reaction (e.g. LFD). In some embodiments, the blocking agent is added to a detection reaction as described herein. Non-limiting examples of blocking agents include BSA, IgGs, tRNA, single stranded excess DNA or RNA, excess orthogonal or random primers, double-stranded excess DNA, and the like.

In some embodiments of any of the aspects, after the amplification reaction, the double-stranded amplicon is contacted with at least one detection probe. Several methods can be used to increase invasion of the detection probe into the double-stranded amplicon. In some embodiments of any of the aspects, the concentration of a recombinase, single-strand-binding protein (SSB), and/or a helicase is modulated to improve detection probe invasion. In some embodiments of any of the aspects, the concentration of a recombinase, single-strand-binding protein (SSB), and/or a helicase is increased to improve detection probe invasion. In some embodiments of any of the aspects, the concentration of a recombinase is increased to improve detection probe invasion. In some embodiments of any of the aspects, the concentration of SSB is increased to improve detection probe invasion. In some embodiments of any of the aspects, the concentration of a helicase is increased to improve detection probe invasion. Such modulation of the concentration of a recombinase, single-strand-binding protein (SSB), and/or a helicase can also be performed in the presence of a buffer additive, as described further herein.

In some embodiments of any of the aspects, after the amplification reaction, the double-stranded amplicon is contacted with at least one detection probe and a sequence guided endonuclease, e.g., that lacks endonuclease activity. In some embodiments, the sequence guidance endonuclease is a CRISPR-Cas protein. Generally, the sequence guided endonuclease that lacks any endonuclease activity, and can be referred to herein as a dCas. For example, the sequence guided endonuclease is catalytically inactive. In other words, the sequence guided endonuclease lacks nuclease, e.g., endonuclease activity of the parent CRISPR-Cas protein. In embodiments comprising a sequence guided endonuclease, the at least one detection probe further comprises a scaffold region for binding to the sequence guided endonuclease.

In some embodiments of the various aspects described herein, the sequence guided endonuclease comprises a CRISPR-Cas protein selected from the group consisting of C2c1, C2c3, Cas1, Cas100, Cas12a, Cas12b, Cas12c, Cas12d, Cas12e, Cas13a, Cas13b, Cas13c, Cas1B, Cas2, Cas3, Cas4, Cas5, Cas6, Cas7, Cas8, Cas9 (also known as Csnl and Csx12), Casl, CaslB, CaslO, Cmr1, Cmr3, Cmr4, Cmr5, Cmr6, Cpf1, Csa5, Csa5, CsaX, Csb1, Csb2, Csb3, Csc1, Csc2, Cse1, Cse2, Csf1, Csf2, Csf3, Csf4, Csm2, Csm3, Csm4, Csm5, Csm6, Csn2, Csx1, Csx10, Csx14, Csx15, Csx16, Csx17, Csx3, Csy1, Csy2, Csy3, and homologues thereof, or modified versions thereof. It is noted that the sequence guided endonuclease can be from an analog or variant of a known CRISPR-Cas protein. In some embodiments of the various aspects described herein, the sequence guided endonuclease is dCas9, dCas 12, or dCas 13.

### Target Nucleic Acid

Described herein are methods, kits, and systems that can be used to detect a target nucleic acid. Furthermore, the compositions provided herein can further comprise the target nucleic acid. In some embodiments of any of the aspects, the target nucleic acid is a target DNA, which can also be referred to as “an DNA of interest” or a “gene of interest.” In some embodiments of any of the aspects, the target DNA can be any DNA sequence or any gene. In some embodiments of any of the aspects, the target DNA is single-stranded DNA (ssDNA). In some embodiments of any of the aspects, the target DNA is double-stranded DNA (dsDNA).

The methods and compositions provided herein can be used to detect, e.g., disease biomarkers, microbial nucleic acid sequences, viral nucleic acid sequences, and the like. In some embodiments, the methods and compositions provided herein can be used to diagnose, prevent, or treat a disease (e.g., an infection). In some embodiments, the methods, compositions, and kits provided herein can be used to identify the presence of SAR-CoV2 in a sample. In some embodiments, the methods, compositions, and kits provided herein can be used to diagnose a subject with an infection. In some embodiments, the infection is COVID 19.

In some embodiments of any of the aspects, the target nucleic acid is a target RNA, which can also be referred to as “an RNA of interest.” In some embodiments of any of the aspects, the target nucleic acid is a target RNA is single-stranded DNA (ssRNA). Ribonucleic acid (RNA) is a polymeric nucleic acid molecule essential in various biological roles in coding, decoding, regulation and expression of genes. Each nucleotide in RNA contains a ribose sugar, with carbons numbered 1′ through 5′. A base is attached to the 1′ position, in general, adenine (A), cytosine (C), guanine (G), or uracil (U). A phosphate group is attached to the 3′ position of one ribose and the 5′ position of the next. The phosphate groups have a negative charge each, making RNA a charged molecule (polyanion). An important structural component of RNA that distinguishes it from DNA is the presence of a hydroxyl group at the 2′ position of the ribose sugar. In some embodiments of any of the aspects, the target RNA can be any known type of RNA. In some embodiments of any of the aspects, the target RNA comprises an RNA selected from Table 2.

TABLE 2

Non-limiting Examples of Target RNAs
RNAs involved in protein synthesis

| | | | |
|---|---|---|---|
| Type | Abbr. | Function | Distribution |
| Messenger RNA | mRNA | Codes for protein | All organisms |
| Ribosomal RNA | rRNA | Translation | All organisms |
| Signal recognition particle RNA | 7SL RNA or SRP RNA | Membrane integration | All organisms |
| Transfer RNA | tRNA | Translation | All organisms |
| Transfer-messenger RNA | tmRNA | Rescuing stalled ribosomes | Bacteria |
| RNAs involved in post-transcriptional modification or DNA replication | | | |
| Type | Abbr. | Function | Distribution |
| Small nuclear RNA | snRNA | Splicing and other functions | Eukaryotes and archaea |
| Small nucleolar RNA | snoRNA | Nucleotide modification of RNAs | Eukaryotes and archaea |
| SmY RNA | SmY | mRNA trans-splicing | Nematodes |
| Small Cajal body-specific RNA | scaRNA | Type of snoRNA; Nucleotide modification of RNAs | |
| Guide RNA | gRNA | mRNA nucleotide modification | Kinetoplastid mitochondria |
| Ribonuclease P | RNase P | tRNA maturation | All organisms |
| Ribonuclease MRP | RNase MRP | rRNA maturation, DNA replication | Eukaryotes |
| Y RNA | | RNA processing, DNA replication | Animals |
| Telomerase RNA Component | TERC | Telomere synthesis | Most eukaryotes |
| Spliced Leader RNA | SL RNA | mRNA trans-splicing, RNA processing | |
| Antisense RNA | aRNA, asRNA | Transcriptional attenuation / mRNA degradation / mRNA stabilisation / Translation block | All organisms |
| Cis-natural antisense transcript | cis-NAT | Gene regulation | |
| CRISPR RNA | crRNA | Resistance to parasites, by targeting their DNA | Bacteria and archaea |
| Long noncoding RNA | lncRNA | Regulation of gene transcription, epigenetic regulation | Eukaryotes |
| MicroRNA | miRNA | Gene regulation | Most eukaryotes |
| Piwi-interacting RNA | piRNA | Transposon defense, maybe other functions | Most animals |
| Small interfering RNA | siRNA | Gene regulation | Most eukaryotes |
| Short hairpin RNA | shRNA | Gene regulation | Most eukaryotes |
| Trans-acting siRNA | tasiRNA | Gene regulation | Land plants |
| Repeat associated siRNA | rasiRNA | Type of piRNA; transposon defense | Drosophila |
| 7SK RNA | 7SK | negatively regulating CDK9/cyclin T complex | |
| Enhancer RNA | eRNA | Gene regulation | |
| Parasitic RNAs | | | |
| Type | | Function | Distribution |
| Retrotransposon | | Self-propagating | Eukaryotes and some bacteria |
| Viral genome | | Information carrier | Double-stranded RNA viruses, positive-sense RNA viruses, negative-sense RNA viruses, many satellite viruses and reverse transcribing viruses |
| Viroid | | Self-propagating | Infected plants |
| Satellite RNA | | Self-propagating | Infected cells |
| Other RNAs | | | |
| Type | Abbr. | Function | Distribution |
| Vault RNA | vRNA, vtRNA | Expulsion of xenobiotics (conjectured) | |

In some embodiments of any of the aspects, the target nucleic acid can be detected at single molecular level. In some embodiments of any of the aspects, less than 10 molecules of the target nucleic acid can be detected using the methods, kits, and systems described herein. As a non-limiting example, at least 1 molecule, at least 2 molecules, at least 3 molecules, at least 4 molecules, at least 5 molecules, at least 6 molecules, at least 7 molecules, at least 8 molecules, at least 9 molecules, at least 10 molecules, at least 20 molecules, at least 30 molecules, at least 40 molecules, at least 50 molecules, at least 60 molecules, at least 70 molecules, at least 80 molecules, at least 90 molecules, at least 10 molecules, at least 10² molecules, at least 10³ molecules, at least 10⁴ molecules, or at least 10⁵ molecules of the target nucleic acid can be detected using the methods, kits, or systems described herein.

In some embodiments of any of the aspects, at least 0.6 molecules of target nucleic acid per microliter of sample input (molecules/µL or copies/µL) can be detected using the methods, kits, and systems described herein. As a non-limiting example, at least 0.1 copies/µL, at least 0.2 copies/µL, at least 0.3 copies/µL, at least 0.4 copies/µL, at least 0.5 copies/µL, at least 0.6 copies/µL, at least 0.7 copies/µL, at least 0.8 copies/µL, at least 0.9 copies/µL, at least 1 copy/µL, at least 2 copies/µL, at least 3 copies/µL, at least 4 copies/µL, at least 5 copies/µL, at least 6 copies/µL, at least 7 copies/µL, at least 8 copies/µL, at least 9 copies/µL, at least 10 copies/µL, at least 20 copies/µL, at least 30 copies/µL, at least 40 copies/µL, at least 50 copies/µL, at least 60 copies/µL, at least 70 copies/µL, at least 80 copies/µL, at least 90 copies/µL, at least 10 copies/µL, at least 10² copies/µL, at least 10³ copies/µL, or at least 10⁴ copies/µLof target nucleic acid can be detected using the methods, kits, or systems described herein.

In some embodiments of any of the aspects, the target RNA can be a viral RNA. Accordingly, in one aspect described herein is a method of detecting an RNA virus in a sample from a subject, comprising: (a) isolating viral RNA from the subject; and (b) performing the methods as described herein (e.g., Digest-LAMP and/or ssRPA and detection).

As used herein, the term “RNA virus” refers to a virus comprising an RNA genome. In some embodiments of any of the aspects, the RNA virus is a double-stranded RNA virus, a positive-sense RNA virus, a negative-sense RNA virus, or a reverse transcribing virus (e.g., retrovirus).

In some embodiments of any of the aspects, the RNA virus is a Group III (i.e., double stranded RNA (dsRNA)) virus. In some embodiments of any of the aspects, the Group III RNA virus belongs to a viral family selected from the group consisting of: Amalgaviridae, Birnaviridae, Chrysoviridae, Cystoviridae, Endomaviridae, Hypoviridae, Megabirnaviridae, Partitiviridae, Picobirnaviridae, Reoviridae (e.g., Rotavirus), Totiviridae, Quadriviridae. In some embodiments of any of the aspects, the Group III RNA virus belongs to the Genus Botybirnavirus. In some embodiments of any of the aspects, the Group III RNA virus is an unassigned species selected from the group consisting of: Botrytis porri RNA virus 1, Circulifer tenellus virus 1, Colletotrichum camelliae filamentous virus 1, Cucurbit yellows associated virus, Sclerotinia sclerotiorum debilitation-associated virus, and Spissistilus festinus virus 1.

In some embodiments of any of the aspects, the RNA virus is a Group IV (i.e., positive-sense single stranded (ssRNA)) virus. In some embodiments of any of the aspects, the Group IV RNA virus belongs to a viral order selected from the group consisting of: Nidovirales, Picomavirales, and Tymovirales. In some embodiments of any of the aspects, the Group IV RNA virus belongs to a viral family selected from the group consisting of: Arteriviridae, Coronaviridae (e.g., Coronavirus, SARS-CoV), Mesoniviridae, Roniviridae, Dicistroviridae, Iflaviridae, Marnaviridae, Picornaviridae (e.g., Poliovirus, Rhinovirus (a common cold virus), Hepatitis A virus), Secoviridae (e.g., sub Comovirinae), Alphaflexiviridae, Betaflexiviridae, Gammaflexiviridae, Tymoviridae, Alphatetraviridae, Alvernaviridae, Astroviridae, Barnaviridae, Benyviridae, Bromoviridae, Caliciviridae (e.g., Norwalk virus), Carmotetraviridae, Closteroviridae, Flaviviridae (e.g., Yellow fever virus, West Nile virus, Hepatitis C virus, Dengue fever virus, Zika virus), Fusariviridae, Hepeviridae, Hypoviridae, Leviviridae, Luteoviridae (e.g., Barley yellow dwarf virus), Polycipiviridae, Narnaviridae, Nodaviridae, Permutotetraviridae, Potyviridae, Sarthroviridae, Statovirus, Togaviridae (e.g., Rubella virus, Ross River virus, Sindbis virus, Chikungunya virus), Tombusviridae, and Virgaviridae.. In some embodiments of any of the aspects, the Group IV RNA virus belongs to a viral genus selected from the group consisting of: Bacillariornavirus, Dicipivirus, Labyrnavirus, Sequiviridae, Blunervirus, Cilevirus, Higrevirus, Idaeovirus, Negevirus, Ourmiavirus, Polemovirus, Sinaivirus, and Sobemovirus. In some embodiments of any of the aspects, the Group IV RNA virus is an unassigned species selected from the group consisting of: Acyrthosiphon pisum virus, Bastrovirus, Blackford virus, Blueberry necrotic ring blotch virus, Cadicistrovirus, Chara australis virus, Extra small virus, Goji berry chlorosis virus, Hepelivirus, Jingmen tick virus, Le Blanc virus, Nedicistrovirus, Nesidiocoris tenuis virus 1, Niflavirus, Nylanderia fulva virus 1, Orsay virus, Osedax japonicus RNA virus 1, Picalivirus, Plasmopara halstedii virus, Rosellinia necatrix fusarivirus 1, Santeuil virus, Secalivirus, Solenopsis invicta virus 3, Wuhan large pig roundworm virus. In some embodiments of any of the aspects, the Group IV RNA virus is a satellite virus selected from the group consisting of: Family Sarthroviridae, Genus Albetovirus, Genus Aumaivirus, Genus Papanivirus, Genus Virtovirus, and Chronic bee paralysis virus.

In some embodiments of any of the aspects, the RNA virus is a Group V (i.e., negative-sense ssRNA) virus. In some embodiments of any of the aspects, the Group V RNA virus belongs to a viral phylum or subphylum selected from the group consisting of: Negarnaviricota, Haploviricotina, and Polyploviricotina. In some embodiments of any of the aspects, the Group V RNA virus belongs to a viral class selected from the group consisting of: Chunqiuviricetes, Ellioviricetes, Insthoviricetes, Milneviricetes, Monjiviricetes, and Yunchangviricetes. In some embodiments of any of the aspects, the Group V RNA virus belongs to a viral order selected from the group consisting of: Articulavirales, Bunyavirales, Goujianvirales, Jingchuvirales, Mononegavirales, Muvirales, and Serpentovirales. In some embodiments of any of the aspects, the Group V RNA virus belongs to a viral family selected from the group consisting of: Amnoonviridae (e.g., Taastrup virus), Arenaviridae (e.g., Lassa virus), Aspiviridae, Bornaviridae (e.g., Borna disease virus), Chuviridae, Cruliviridae, Feraviridae, Filoviridae (e.g., Ebola virus, Marburg virus), Fimoviridae, Hantaviridae, Jonviridae, Mymonaviridae, Nairoviridae, Nyamiviridae, Orthomyxoviridae (e.g., Influenza viruses), Paramyxoviridae (e.g., Measles virus, Mumps virus, Nipah virus, Hendra virus, and NDV), Peribunyaviridae, Phasmaviridae, Phenuiviridae, Pneumoviridae (e.g., RSV and Metapneumovirus), Qinviridae, Rhabdoviridae (e.g., Rabies virus), Sunviridae, Tospoviridae, and Yueviridae. In some embodiments of any of the aspects, the Group V RNA virus belongs to a viral genus selected from the group consisting of: Anphevirus, Arlivirus, Chengtivirus, Crustavirus, Tilapineviridae, Wastrivirus, and Deltavirus (e.g., Hepatitis D virus).

In some embodiments of any of the aspects, the RNA virus is a Group VI RNA virus, which comprise a virally encoded reverse transcriptase. In some embodiments of any of the aspects, the Group VI RNA virus belongs to the viral order Ortervirales. In some embodiments of any of the aspects, the Group VI RNA virus belongs to a viral family or subfamily selected from the group consisting of: Belpaoviridae, Caulimoviridae, Metaviridae, Pseudoviridae, Retroviridae (e.g., Retroviruses, e.g. HIV), Orthoretrovirinae, and Spumaretrovirinae. In some embodiments of any of the aspects, the Group VI RNA virus belongs to a viral genus selected from the group consisting of: Alpharetrovirus (e.g., Avian leukosis virus; Rous sarcoma virus), Betaretrovirus (e.g., Mouse mammary tumour virus), Bovispumavirus (e.g., Bovine foamy virus), Deltaretrovirus (e.g., Bovine leukemia virus; Human T-lymphotropic virus), Epsilonretrovirus (e.g., Walleye dermal sarcoma virus), Equispumavirus (e.g., Equine foamy virus), Felispumavirus (e.g., Feline foamy virus), Gammaretrovirus (e.g., Murine leukemia virus; Feline leukemia virus), Lentivirus (e.g., Human immunodeficiency virus 1; Simian immunodeficiency virus; Feline immunodeficiency virus), Prosimiispumavirus (e.g., Brown greater galago prosimian foamy virus), and Simiispumavirus (e.g., Eastern chimpanzee simian foamy virus). In some embodiments of any of the aspects, the virus is an endogenous retrovirus (ERV; e.g., endogenous retrovirus group W envelope member 1 (ERVWE1); HCP5 (HLA Complex P5); Human teratocarcinoma-derived virus), which are endogenous viral elements in the genome that closely resemble and can be derived from retroviruses.

In some embodiments of any of the aspects, the target nucleic acid comprises viral DNA or RNA produced by a virus with a DNA genome, i.e., a DNA virus. As a non-limiting example the DNA virus is a Group I (dsDNA) virus, a Group II (ssDNA) virus, or a Group VII (dsDNA-RT) virus. In some embodiments of any of the aspects, the DNA produced by a DNA virus comprises the DNA genome or fragments thereof. In some embodiments of any of the aspects, the RNA produced by a DNA virus comprises an RNA transcript of the DNA genome.

In some embodiments of any of the aspects, the DNA virus is a Group I (i.e., dsDNA) virus. In some embodiments of any of the aspects, the Group I dsDNA virus belongs to a viral order selected from the group consisting of: Caudovirales; Herpesvirales; and Ligamenvirales. In some embodiments of any of the aspects, the Group I dsDNA virus belongs to a viral family selected from the group consisting of: Adenoviridae (e.g., adenoviruses), Alloherpesviridae, Ampullaviridae, Ascoviridae, Asfarviridae (e.g., African swine fever virus), Baculoviridae, Bicaudaviridae, Clavaviridae, Corticoviridae, Fuselloviridae, Globuloviridae, Guttaviridae, Herpesviridae (e.g., human herpesviruses, Varicella Zoster virus), Hytrosaviridae, Iridoviridae, Lavidaviridae, Lipothrixviridae, Malacoherpesviridae, Marseilleviridae, Mimiviridae, Myoviridae (e.g., Enterobacteria phage T4), Nimaviridae, Nudiviridae, Pandoraviridae, Papillomaviridae, Phycodnaviridae, Plasmaviridae, Podoviridae (e.g., Enterobacteria phage T7), Polydnaviruses, Polyomaviridae (e.g., Simian virus 40, JC virus, BK virus), Poxviridae (e.g., Cowpox virus, smallpox), Rudiviridae, Siphoviridae (e.g., Enterobacteria phage λ), Sphaerolipoviridae, Tectiviridae, Tristromaviridae, and Turriviridae. In some embodiments of any of the aspects, the Group I dsDNA virus belongs to a viral genus selected from the group consisting of: Dinodnavirus, Rhizidiovirus, and Salterprovirus. In some embodiments of any of the aspects, the Group I dsDNA virus belongs to an unassigned viral species selected from the group consisting of: Abalone shriveling syndrome-associated virus, Apis mellifera filamentous virus, Bandicoot papillomatosis carcinomatosis virus, Cedratvirus, Kaumoebavirus, KIs-V, Lentille virus, Leptopilina boulardi filamentous virus, Megavirus, Metallosphaera turreted icosahedral virus, Methanosarcina spherical virus, Mollivirus sibericum virus, Orpheovirus IHUMI-LCC2, Phaeocystis globosa virus, and Pithovirus. In some embodiments of any of the aspects, the Group I dsDNA virus is a virophage selected from the group consisting of: Organic Lake virophage, Ace Lake Mavirus virophage, Dishui Lake virophage 1, Guarani virophage, Phaeocystis globosa virus virophage, Rio Negro virophage, Sputnik virophage 2, Yellowstone Lake virophage 1, Yellowstone Lake virophage 2, Yellowstone Lake virophage 3, Yellowstone Lake virophage 4, Yellowstone Lake virophage 5, Yellowstone Lake virophage 6, Yellowstone Lake virophage 7, and Zamilon virophage 2.

In some embodiments of any of the aspects, the DNA virus is a Group II (i.e., ssDNA) virus. In some embodiments of any of the aspects, the Group II ssDNA virus belongs to a viral family selected from the group consisting of: Anelloviridae, Bacilladnaviridae, Bidnaviridae, Circoviridae, Geminiviridae, Genomoviridae, Inoviridae, Microviridae, Nanoviridae, Parvoviridae, Smacoviridae, and Spiraviridae.

In some embodiments of any of the aspects, the DNA virus is a Group VII (i.e., dsDNA-RT) virus. In some embodiments of any of the aspects, the Group VII dsDNA-RT virus belongs to the Ortervirales order. In some embodiments of any of the aspects, the Group VII dsDNA-RT virus belongs to the Caulimoviridae family or to the Hepadnaviridae family (e.g., Hepatitis B virus). In some embodiments of any of the aspects, the Group VII dsDNA-RT virus belongs to a viral genus selected from the group consisting of: Badnavirus, Caulimovirus, Cavemovirus, Petuvirus, Rosadnavirus, Solendovirus, Soymovirus, Tungrovirus, Avihepadnavirus, and Orthohepadnavirus.

In some embodiments of any of the aspects, the target nucleic acid is from a coronavirus. The scientific name for coronavirus is Orthocoronavirinae or Coronavirinae. Coronaviruses belong to the family of Coronaviridae, order Nidovirales, and realm Riboviria. They are divided into alphacoronaviruses and betacoronaviruses which infect mammals - and gammacoronaviruses and deltacoronaviruses which primarily infect birds. Non limiting examples of alphacoronaviruses include: Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512, and Feline Infectious Peritonitis Virus (FIPV, also referred to as Feline Infectious Hepatitis Virus). Non limiting examples of betacoronaviruses include: Betacoronavirus 1 (e.g., Bovine Coronavirus, Human coronavirus OC43), Human coronavirus HKU1, Murine coronavirus (also known as Mouse hepatitis virus (MHV)), Pipistrellus bat coronavirus HKU5, Rousettus bat coronavirus HKU9, Severe acute respiratory syndrome-related coronavirus (e.g., SARS-CoV, SARS-CoV-2), Tylonycteris bat coronavirus HKU4, Middle East respiratory syndrome (MERS)-related coronavirus, and Hedgehog coronavirus 1 (EriCoV). Non limiting examples of gammacoronaviruses include: Beluga whale coronavirus SW1, and Infectious bronchitis virus. Non limiting examples of deltacoronaviruses include: Bulbul coronavirus HKU11, and Porcine coronavirus HKU15.

In some embodiments of any of the aspects, the target nucleic acid is from a coronavirus selected from the group consisting of: severe acute respiratory syndrome-associated coronavirus (SARS-CoV); severe acute respiratory syndrome-associated coronavirus 2 (SARS-CoV-2); Middle East respiratory syndrome-related coronavirus (MERS-CoV); HCoV-NL63; and HCoV-HKu1. In some embodiments of any of the aspects, the target nucleic acid is from severe acute respiratory syndrome coronavirus 2 (SARS-CoV-2), which causes coronavirus disease of 2019 (COVID19 or simply COVID). In some embodiments of any of the aspects, the target nucleic acid is from severe acute respiratory syndrome coronavirus (SARS-CoV), which causes SARS. In some embodiments of any of the aspects, the target nucleic acid is from Middle East respiratory syndrome-related coronavirus (MERS-CoV), which causes MERS. In some embodiments of any of the aspects, the target nucleic acid is from is any known RNA or DNA virus.

In some embodiments of any of the aspects, at least one viral RNA is a SARS-CoV-2 RNA. In some embodiments of any of the aspects, the target nucleic acid comprises at least a portion of Severe acute respiratory syndrome coronavirus 2 isolate SARS-CoV-2, (see e.g., complete genome, SARS-CoV-2 January 2020/NC_045512.2 Assembly (wuhCorl)). In some embodiments of any of the aspects, the target nucleic acid comprises any gene from SARS-CoV-2, such as the N gene, the S gene, or the ORF lab gene. In some embodiments of any of the aspects, the target nucleic acid comprises SEQ ID NO: 1 (Severe acute respiratory syndrome coronavirus 2 isolate SARS-CoV-2, N gene). In some embodiments of any of the aspects, the target nucleic acid comprises SEQ ID NO: 2 (Severe acute respiratory syndrome coronavirus 2 isolate SARS-CoV-2, S gene). In some embodiments of any of the aspects, the target nucleic acid comprises SEQ ID NO: 3 (Severe acute respiratory syndrome coronavirus 2 isolate SARS-CoV-2, ORF1ab gene). In some embodiments of any of the aspects, the target nucleic acid comprises SEQ ID NO: 58 (Severe acute respiratory syndrome coronavirus 2 isolate SARS-CoV-2, E gene). In some embodiments of any of the aspects, the target nucleic acid comprises one of SEQ ID NOs: 1-3 or 58, or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NO: 1-3 or 58 that maintains the same function or a codon-optimized version of one of SEQ ID NOs: 1-3 or 58. In some embodiments of any of the aspects, the target nucleic acid comprises one of SEQ ID NOs: 1-3 or 58, or a nucleic acid sequence that is at least 95% identical to one of SEQ ID NOs: 1-3 or 58 that maintains the same function.

In some embodiments, the target nucleic acid comprises one of SEQ ID NOs: 1-4, 20, 58 or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 1-4, 20, or 58 that maintains the same function or a functional fragment thereof.

SEQ ID NO: 1, Severe acute respiratory syndrome coronavirus 2 isolate Wuhan-Hu-1, N nucleocapsid phosphoprotein, Gene ID: 43740575, 1260 bp ss-RNA, NC_045512 REGION: 28274-29533

ATGTCTGATAATGGACCCCAAAATCAGCGAAATGCACCCCGCATTACGTTTGGTGGACCC
TCAGATTCAACTGGCAGTAACCAGAATGGAGAACGCAGTGGGGCGCGATCAAAACAACG
TCGGCCCCAAGGTTTACCCAATAATACTGCGTCTTGGTTCACCGCTCTCACTCAACATGG
CAAGGAAGACCTTAAATTCCCTCGAGGACAAGGCGTTCCAATTAACACCAATAGCAGTC
CAGATGACCAAATTGGCTACTACCGAAGAGCTACCAGACGAATTCGTGGTGGTGACGGT
AAAATGAAAGATCTCAGTCCAAGATGGTATTTCTACTACCTAGGAACTGGGCCAGAAGC
TGGACTTCCCTATGGTGCTAACAAAGACGGCATCATATGGGTTGCAACTGAGGGAGCCTT
GAATACACCAAAAGATCACATTGGCACCCGCAATCCTGCTAACAATGCTGCAATCGTGCT
ACAACTTCCTCAAGGAACAACATTGCCAAAAGGCTTCTACGCAGAAGGGAGCAGAGGCG
GCAGTCAAGCCTCTTCTCGTTCCTCATCACGTAGTCGCAACAGTTCAAGAAATTCAACTC
CAGGCAGCAGTAGGGGAACTTCTCCTGCTAGAATGGCTGGCAATGGCGGTGATGCTGCT
CTTGCTTTGCTGCTGCTTGACAGATTGAACCAGCTTGAGAGCAAAATGTCTGGTAAAGGC
CAACAACAACAAGGCCAAACTGTCACTAAGAAATCTGCTGCTGAGGCTTCTAAGAAGCC
TCGGCAAAAACGTACTGCCACTAAAGCATACAATGTAACACAAGCTTTCGGCAGACGTG
GTCCAGAACAAACCCAAGGAAATTTTGGGGACCAGGAACTAATCAGACAAGGAACTGAT
TACAAACATTGGCCGCAAATTGCACAATTTGCCCCCAGCGCTTCAGCGTTCTTCGGAATG
TCGCGCATTGGCATGGAAGTCACACCTTCGGGAACGTGGTTGACCTACACAGGTGCCATC
AAATTGGATGACAAAGATCCAAATTTCAAAGATCAAGTCATTTTGCTGAATAAGCATATT
GACGCATACAAAACATTCCCACCAACAGAGCCTAAAAAGGACAAAAAGAAGAAGGCTG
ATGAAACTCAAGCCTTACCGCAGAGACAGAAGAAACAGCAAACTGTGACTCTTCTTCCT
GCTGCAGATTTGGATGATTTCTCCAAACAATTGCAACAATCCATGAGCAGTGCTGACTCA
ACTCAGGCCTAA

SEQ ID NO: 2, Severe acute respiratory syndrome coronavirus 2 isolate Wuhan-Hu-1, S surface glycoprotein, Gene ID: 43740568, 3822 bp ss-RNA, NC_045512 REGION: 21563-25384

ATGTTTGTTTTTCTTGTTTTATTGCCACTAGTCTCTAGTCAGTGTGTTAATCTTACAACCAG
AACTCAATTACCCCCTGCATACACTAATTCTTTCACACGTGGTGTTTATTACCCTGACAAA
GTTTTCAGATCCTCAGTTTTACATTCAACTCAGGACTTGTTCTTACCTTTCTTTTCCAATGT
TACTTGGTTCCATGCTATACATGTCTCTGGGACCAATGGTACTAAGAGGTTTGATAACCC
TGTCCTACCATTTAATGATGGTGTTTATTTTGCTTCCACTGAGAAGTCTAACATAATAAGA
GGCTGGATTTTTGGTACTACTTTAGATTCGAAGACCCAGTCCCTACTTATTGTTAATAACG
CTACTAATGTTGTTATTAAAGTCTGTGAATTTCAATTTTGTAATGATCCATTTTTGGGTGT
TTATTACCACAAAAACAACAAAAGTTGGATGGAAAGTGAGTTCAGAGTTTATTCTAGTGC
GAATAATTGCACTTTTGAATATGTCTCTCAGCCTTTTCTTATGGACCTTGAAGGAAAACA
GGGTAATTTCAAAAATCTTAGGGAATTTGTGTTTAAGAATATTGATGGTTATTTTAAAAT
ATATTCTAAGCACACGCCTATTAATTTAGTGCGTGATCTCCCTCAGGGTTTTTCGGCTTTA
GAACCATTGGTAGATTTGCCAATAGGTATTAACATCACTAGGTTTCAAACTTTACTTGCTT
TACATAGAAGTTATTTGACTCCTGGTGATTCTTCTTCAGGTTGGACAGCTGGTGCTGCAG
CTTATTATGTGGGTTATCTTCAACCTAGGACTTTTCTATTAAAATATAATGAAAATGGAA
CCATTACAGATGCTGTAGACTGTGCACTTGACCCTCTCTCAGAAACAAAGTGTACGTTGA
AATCCTTCACTGTAGAAAAAGGAATCTATCAAACTTCTAACTTTAGAGTCCAACCAACAG
AATCTATTGTTAGATTTCCTAATATTACAAACTTGTGCCCTTTTGGTGAAGTTTTTAACGC
CACCAGATTTGCATCTGTTTATGCTTGGAACAGGAAGAGAATCAGCAACTGTGTTGCTGA
TTATTCTGTCCTATATAATTCCGCATCATTTTCCACTTTTAAGTGTTATGGAGTGTCTCCTA
CTAAATTAAATGATCTCTGCTTTACTAATGTCTATGCAGATTCATTTGTAATTAGAGGTGA
TGAAGTCAGACAAATCGCTCCAGGGCAAACTGGAAAGATTGCTGATTATAATTATAAAT
TACCAGATGATTTTACAGGCTGCGTTATAGCTTGGAATTCTAACAATCTTGATTCTAAGG
TTGGTGGTAATTATAATTACCTGTATAGATTGTTTAGGAAGTCTAATCTCAAACCTTTTGA
GAGAGATATTTCAACTGAAATCTATCAGGCCGGTAGCACACCTTGTAATGGTGTTGAAGG
TTTTAATTGTTACTTTCCTTTACAATCATATGGTTTCCAACCCACTAATGGTGTTGGTTAC
CAACCATACAGAGTAGTAGTACTTTCTTTTGAACTTCTACATGCACCAGCAACTGTTTGT
GGACCTAAAAAGTCTACTAATTTGGTTAAAAACAAATGTGTCAATTTCAACTTCAATGGT
TTAACAGGCACAGGTGTTCTTACTGAGTCTAACAAAAAGTTTCTGCCTTTCCAACAATTT
GGCAGAGACATTGCTGACACTACTGATGCTGTCCGTGATCCACAGACACTTGAGATTCTT
GACATTACACCATGTTCTTTTGGTGGTGTCAGTGTTATAACACCAGGAACAAATACTTCT
AACCAGGTTGCTGTTCTTTATCAGGATGTTAACTGCACAGAAGTCCCTGTTGCTATTCATG
CAGATCAACTTACTCCTACTTGGCGTGTTTATTCTACAGGTTCTAATGTTTTTCAAACACG
TGCAGGCTGTTTAATAGGGGCTGAACATGTCAACAACTCATATGAGTGTGACATACCCAT
TGGTGCAGGTATATGCGCTAGTTATCAGACTCAGACTAATTCTCCTCGGCGGGCACGTAG
TGTAGCTAGTCAATCCATCATTGCCTACACTATGTCACTTGGTGCAGAAAATTCAGTTGC
TTACTCTAATAACTCTATTGCCATACCCACAAATTTTACTATTAGTGTTACCACAGAAATT
CTACCAGTGTCTATGACCAAGACATCAGTAGATTGTACAATGTACATTTGTGGTGATTCA
ACTGAATGCAGCAATCTTTTGTTGCAATATGGCAGTTTTTGTACACAATTAAACCGTGCTT
TAACTGGAATAGCTGTTGAACAAGACAAAAACACCCAAGAAGTTTTTGCACAAGTCAAA
CAAATTTACAAAACACCACCAATTAAAGATTTTGGTGGTTTTAATTTTTCACAAATATTA
CCAGATCCATCAAAACCAAGCAAGAGGTCATTTATTGAAGATCTACTTTTCAACAAAGTG
ACACTTGCAGATGCTGGCTTCATCAAACAATATGGTGATTGCCTTGGTGATATTGCTGCT
AGAGACCTCATTTGTGCACAAAAGTTTAACGGCCTTACTGTTTTGCCACCTTTGCTCACA
GATGAAATGATTGCTCAATACACTTCTGCACTGTTAGCGGGTACAATCACTTCTGGTTGG
ACCTTTGGTGCAGGTGCTGCATTACAAATACCATTTGCTATGCAAATGGCTTATAGGTTT
AATGGTATTGGAGTTACACAGAATGTTCTCTATGAGAACCAAAAATTGATTGCCAACCAA
TTTAATAGTGCTATTGGCAAAATTCAAGACTCACTTTCTTCCACAGCAAGTGCACTTGGA
AAACTTCAAGATGTGGTCAACCAAAATGCACAAGCTTTAAACACGCTTGTTAAACAACTT
AGCTCCAATTTTGGTGCAATTTCAAGTGTTTTAAATGATATCCTTTCACGTCTTGACAAAG
TTGAGGCTGAAGTGCAAATTGATAGGTTGATCACAGGCAGACTTCAAAGTTTGCAGACA
TATGTGACTCAACAATTAATTAGAGCTGCAGAAATCAGAGCTTCTGCTAATCTTGCTGCT
ACTAAAATGTCAGAGTGTGTACTTGGACAATCAAAAAGAGTTGATTTTTGTGGAAAGGG
CTATCATCTTATGTCCTTCCCTCAGTCAGCACCTCATGGTGTAGTCTTCTTGCATGTGACT
TATGTCCCTGCACAAGAAAAGAACTTCACAACTGCTCCTGCCATTTGTCATGATGGAAAA
GCACACTTTCCTCGTGAAGGTGTCTTTGTTTCAAATGGCACACACTGGTTTGTAACACAA
AGGAATTTTTATGAACCACAAATCATTACTACAGACAACACATTTGTGTCTGGTAACTGT
GATGTTGTAATAGGAATTGTCAACAACACAGTTTATGATCCTTTGCAACCTGAATTAGAC
TCATTCAAGGAGGAGTTAGATAAATATTTTAAGAATCATACATCACCAGATGTTGATTTA
GGTGACATCTCTGGCATTAATGCTTCAGTTGTAAACATTCAAAAAGAAATTGACCGCCTC
AATGAGGTTGCCAAGAATTTAAATGAATCTCTCATCGATCTCCAAGAACTTGGAAAGTAT
GAGCAGTATATAAAATGGCCATGGTACATTTGGCTAGGTTTTATAGCTGGCTTGATTGCC
ATAGTAATGGTGACAATTATGCTTTGCTGTATGACCAGTTGCTGTAGTTGTCTCAAGGGC
TGTTGTTCTTGTGGATCCTGCTGCAAATTTGATGAAGACGACTCTGAGCCAGTGCTCAAA
GGAGTCAAATTACATTACACATAA

SEQ ID NO: 3, ORF 1ab polyprotein, Severe acute respiratory syndrome coronavirus 2, isolate Wuhan-Hu-1, NCBI Reference Sequence: NC_045512.2 region: 266-21555, 21290 nt

atggagagccttgtccctggtttcaacgagaaaacacacgtccaactcagtttgcctgttttacaggttcgcgacgtgctcgtacgtggct
ttggagactccgtggaggaggtcttatcagaggcacgtcaacatcttaaagatggcacttgtggcttagtagaagttgaaaaaggcgttt
tgcctcaacttgaacagccctatgtgttcatcaaacgttcggatgctcgaactgcacctcatggtcatgttatggttgagctggtagcaga
actcgaaggcattcagtacggtcgtagtggtgagacacttggtgtccttgtccctcatgtgggcgaaataccagtggcttaccgcaagg
ttcttcttcgtaagaacggtaataaaggagctggtggccatagttacggcgccgatctaaagtcatttgacttaggcgacgagcttggca
ctgatccttatgaagattttcaagaaaactggaacactaaacatagcagtggtgttacccgtgaactcatgcgtgagcttaacggagggg
catacactcgctatgtcgataacaacttctgtggccctgatggctaccctcttgagtgcattaaagaccttctagcacgtgctggtaaagct
tcatgcactttgtccgaacaactggactttattgacactaagaggggtgtatactgctgccgtgaacatgagcatgaaattgcttggtaca
cggaacgttctgaaaagagctatgaattgcagacaccttttgaaattaaattggcaaagaaatttgacaccttcaatggggaatgtccaa
attttgtatttcccttaaattccataatcaagactattcaaccaagggttgaaaagaaaaagcttgatggctttatgggtagaattcgatctgt
ctatccagttgcgtcaccaaatgaatgcaaccaaatgtgcctttcaactctcatgaagtgtgatcattgtggtgaaacttcatggcagacg
ggcgattttgttaaagccacttgcgaattttgtggcactgagaatttgactaaagaaggtgccactacttgtggttacttaccccaaaatgc
tgttgttaaaatttattgtccagcatgtcacaattcagaagtaggacctgagcatagtcttgccgaataccataatgaatctggcttgaaaa
ccattcttcgtaagggtggtcgcactattgcctttggaggctgtgtgttctcttatgttggttgccataacaagtgtgcctattgggttccacg
tgctagcgctaacataggttgtaaccatacaggtgttgttggagaaggttccgaaggtcttaatgacaaccttcttgaaatactccaaaaa
gagaaagtcaacatcaatattgttggtgactttaaacttaatgaagagatcgccattattttggcatctttttctgcttccacaagtgcttttgt
ggaaactgtgaaaggtttggattataaagcattcaaacaaattgttgaatcctgtggtaattttaaagttacaaaaggaaaagctaaaaaa
ggtgcctggaatattggtgaacagaaatcaatactgagtcctctttatgcatttgcatcagaggctgctcgtgttgtacgatcaattttctcc
cgcactcttgaaactgctcaaaattctgtgcgtgttttacagaaggccgctataacaatactagatggaatttcacagtattcactgagact
cattgatgctatgatgttcacatctgatttggctactaacaatctagttgtaatggcctacattacaggtggtgttgttcagttgacttcgcagt
ggctaactaacatctttggcactgtttatgaaaaactcaaacccgtccttgattggcttgaagagaagtttaaggaaggtgtagagtttctt
agagacggttgggaaattgttaaatttatctcaacctgtgcttgtgaaattgtcggtggacaaattgtcacctgtgcaaaggaaattaagg
agagtgttcagacattctttaagcttgtaaataaatttttggctttgtgtgctgactctatcattattggtggagctaaacttaaagccttgaatt
taggtgaaacatttgtcacgcactcaaagggattgtacagaaagtgtgttaaatccagagaagaaactggcctactcatgcctctaaaag
ccccaaaagaaattatcttcttagagggagaaacacttcccacagaagtgttaacagaggaagttgtcttgaaaactggtgatttacaac
cattagaacaacctactagtgaagctgttgaagctccattggttggtacaccagtttgtattaacgggcttatgttgctcgaaatcaaagac
acagaaaagtactgtgcccttgcacctaatatgatggtaacaaacaataccttcacactcaaaggcggtgcaccaacaaaggttactttt
ggtgatgacactgtgatagaagtgcaaggttacaagagtgtgaatatcacttttgaacttgatgaaaggattgataaagtacttaatgaga
agtgctctgcctatacagttgaactcggtacagaagtaaatgagttcgcctgtgttgtggcagatgctgtcataaaaactttgcaaccagt
atctgaattacttacaccactgggcattgatttagatgagtggagtatggctacatactacttatttgatgagtctggtgagtttaaattggctt
cacatatgtattgttctttctaccctccagatgaggatgaagaagaaggtgattgtgaagaagaagagtttgagccatcaactcaatatga
gtatggtactgaagatgattaccaaggtaaacctttggaatttggtgccacttctgctgctcttcaacctgaagaagagcaagaagaaga
ttggttagatgatgatagtcaacaaactgttggtcaacaagacggcagtgaggacaatcagacaactactattcaaacaattgttgaggtt
caacctcaattagagatggaacttacaccagttgttcagactattgaagtgaatagttttagtggttatttaaaacttactgacaatgtataca
ttaaaaatgcagacattgtggaagaagctaaaaaggtaaaaccaacagtggttgttaatgcagccaatgtttaccttaaacatggagga
ggtgttgcaggagccttaaataaggctactaacaatgccatgcaagttgaatctgatgattacatagctactaatggaccacttaaagtgg
gtggtagttgtgttttaagcggacacaatcttgctaaacactgtcttcatgttgtcggcccaaatgttaacaaaggtgaagacattcaacttc
ttaagagtgcttatgaaaattttaatcagcacgaagttctacttgcaccattattatcagctggtatttttggtgctgaccctatacattctttaa
gagtttgtgtagatactgttcgcacaaatgtctacttagctgtctttgataaaaatctctatgacaaacttgtttcaagctttttggaaatgaag
agtgaaaagcaagttgaacaaaagatcgctgagattcctaaagaggaagttaagccatttataactgaaagtaaaccttcagttgaaca
gagaaaacaagatgataagaaaatcaaagcttgtgttgaagaagttacaacaactctggaagaaactaagttcctcacagaaaacttgtt
actttatattgacattaatggcaatcttcatccagattctgccactcttgttagtgacattgacatcactttcttaaagaaagatgctccatatat
agtgggtgatgttgttcaagagggtgttttaactgctgtggttatacctactaaaaaggctggtggcactactgaaatgctagcgaaagct
ttgagaaaagtgccaacagacaattatataaccacttacccgggtcagggtttaaatggttacactgtagaggaggcaaagacagtgct
taaaaagtgtaaaagtgccttttacattctaccatctattatctctaatgagaagcaagaaattcttggaactgtttcttggaatttgcgagaa
atgcttgcacatgcagaagaaacacgcaaattaatgcctgtctgtgtggaaactaaagccatagtttcaactatacagcgtaaatataag
ggtattaaaatacaagagggtgtggttgattatggtgctagattttacttttacaccagtaaaacaactgtagcgtcacttatcaacacactt
aacgatctaaatgaaactcttgttacaatgccacttggctatgtaacacatggcttaaatttggaagaagctgctcggtatatgagatctct
caaagtgccagctacagtttctgtttcttcacctgatgctgttacagcgtataatggttatcttacttcttcttctaaaacacctgaagaacattt
tattgaaaccatctcacttgctggttcctataaagattggtcctattctggacaatctacacaactaggtatagaatttcttaagagaggtgat
aaaagtgtatattacactagtaatcctaccacattccacctagatggtgaagttatcacctttgacaatcttaagacacttctttctttgagag
aagtgaggactattaaggtgtttacaacagtagacaacattaacctccacacgcaagttgtggacatgtcaatgacatatggacaacagt
ttggtccaacttatttggatggagctgatgttactaaaataaaacctcataattcacatgaaggtaaaacattttatgttttacctaatgatgac
actctacgtgttgaggcttttgagtactaccacacaactgatcctagttttctgggtaggtacatgtcagcattaaatcacactaaaaagtg
gaaatacccacaagttaatggtttaacttctattaaatgggcagataacaactgttatcttgccactgcattgttaacactccaacaaataga
gttgaagtttaatccacctgctctacaagatgcttattacagagcaagggctggtgaagctgctaacttttgtgcacttatcttagcctactg
taataagacagtaggtgagttaggtgatgttagagaaacaatgagttacttgtttcaacatgccaatttagattcttgcaaaagagtcttga
acgtggtgtgtaaaacttgtggacaacagcagacaacccttaagggtgtagaagctgttatgtacatgggcacactttcttatgaacaatt
taagaaaggtgttcagataccttgtacgtgtggtaaacaagctacaaaatatctagtacaacaggagtcaccttttgttatgatgtcagcac
cacctgctcagtatgaacttaagcatggtacatttacttgtgctagtgagtacactggtaattaccagtgtggtcactataaacatataactt
ctaaagaaactttgtattgcatagacggtgctttacttacaaagtcctcagaatacaaaggtcctattacggatgttttctacaaagaaaac
agttacacaacaaccataaaaccagttacttataaattggatggtgttgtttgtacagaaattgaccctaagttggacaattattataagaaa
gacaattcttatttcacagagcaaccaattgatcttgtaccaaaccaaccatatccaaacgcaagcttcgataattttaagtttgtatgtgata
atatcaaatttgctgatgatttaaaccagttaactggttataagaaacctgcttcaagagagcttaaagttacatttttccctgacttaaatggt
gatgtggtggctattgattataaacactacacaccctcttttaagaaaggagctaaattgttacataaacctattgtttggcatgttaacaatg
caactaataaagccacgtataaaccaaatacctggtgtatacgttgtctttggagcacaaaaccagttgaaacatcaaattcgtttgatgta
ctgaagtcagaggacgcgcagggaatggataatcttgcctgcgaagatctaaaaccagtctctgaagaagtagtggaaaatcctacca
tacagaaagacgttcttgagtgtaatgtgaaaactaccgaagttgtaggagacattatacttaaaccagcaaataatagtttaaaaattaca
gaagaggttggccacacagatctaatggctgcttatgtagacaattctagtcttactattaagaaacctaatgaattatctagagtattaggt
ttgaaaacccttgctactcatggtttagctgctgttaatagtgtcccttgggatactatagctaattatgctaagccttttcttaacaaagttgtt
agtacaactactaacatagttacacggtgtttaaaccgtgtttgtactaattatatgccttatttctttactttattgctacaattgtgtacttttact
agaagtacaaattctagaattaaagcatctatgccgactactatagcaaagaatactgttaagagtgtcggtaaattttgtctagaggcttc
atttaattatttgaagtcacctaatttttctaaactgataaatattataatttggtttttactattaagtgtttgcctaggttctttaatctactcaacc
gctgctttaggtgttttaatgtctaatttaggcatgccttcttactgtactggttacagagaaggctatttgaactctactaatgtcactattgca
acctactgtactggttctataccttgtagtgtttgtcttagtggtttagattctttagacacctatccttctttagaaactatacaaattaccatttc
atcttttaaatgggatttaactgcttttggcttagttgcagagtggtttttggcatatattcttttcactaggtttttctatgtacttggattggctgc
aatcatgcaattgtttttcagctattttgcagtacattttattagtaattcttggcttatgtggttaataattaatcttgtacaaatggccccgattt
cagctatggttagaatgtacatcttctttgcatcattttattatgtatggaaaagttatgtgcatgttgtagacggttgtaattcatcaacttgtat
gatgtgttacaaacgtaatagagcaacaagagtcgaatgtacaactattgttaatggtgttagaaggtccttttatgtctatgctaatggag
gtaaaggcttttgcaaactacacaattggaattgtgttaattgtgatacattctgtgctggtagtacatttattagtgatgaagttgcgagaga
cttgtcactacagtttaaaagaccaataaatcctactgaccagtcttcttacatcgttgatagtgttacagtgaagaatggttccatccatctt
tactttgataaagctggtcaaaagacttatgaaagacattctctctctcattttgttaacttagacaacctgagagctaataacactaaaggtt
cattgcctattaatgttatagtttttgatggtaaatcaaaatgtgaagaatcatctgcaaaatcagcgtctgtttactacagtcagcttatgtgt
caacctatactgttactagatcaggcattagtgtctgatgttggtgatagtgcggaagttgcagttaaaatgtttgatgcttacgttaatacgt
tttcatcaacttttaacgtaccaatggaaaaactcaaaacactagttgcaactgcagaagctgaacttgcaaagaatgtgtccttagacaa
tgtcttatctacttttatttcagcagctcggcaagggtttgttgattcagatgtagaaactaaagatgttgttgaatgtcttaaattgtcacatca
atctgacatagaagttactggcgatagttgtaataactatatgctcacctataacaaagttgaaaacatgacaccccgtgaccttggtgctt
gtattgactgtagtgcgcgtcatattaatgcgcaggtagcaaaaagtcacaacattgctttgatatggaacgttaaagatttcatgtcattgt
ctgaacaactacgaaaacaaatacgtagtgctgctaaaaagaataacttaccttttaagttgacatgtgcaactactagacaagttgttaat
gttgtaacaacaaagatagcacttaagggtggtaaaattgttaataattggttgaagcagttaattaaagttacacttgtgttcctttttgttgc
tgctattttctatttaataacacctgttcatgtcatgtctaaacatactgacttttcaagtgaaatcataggatacaaggctattgatggtggtgt
cactcgtgacatagcatctacagatacttgttttgctaacaaacatgctgattttgacacatggtttagccagcgtggtggtagttatactaa
tgacaaagcttgcccattgattgctgcagtcataacaagagaagtgggttttgtcgtgcctggtttgcctggcacgatattacgcacaact
aatggtgactttttgcatttcttacctagagtttttagtgcagttggtaacatctgttacacaccatcaaaacttatagagtacactgactttgc
aacatcagcttgtgttttggctgctgaatgtacaatttttaaagatgcttctggtaagccagtaccatattgttatgataccaatgtactagaa
ggttctgttgcttatgaaagtttacgccctgacacacgttatgtgctcatggatggctctattattcaatttcctaacacctaccttgaaggttc
tgttagagtggtaacaacttttgattctgagtactgtaggcacggcacttgtgaaagatcagaagctggtgtttgtgtatctactagtggta
gatgggtacttaacaatgattattacagatctttaccaggagttttctgtggtgtagatgctgtaaatttacttactaatatgtttacaccactaa
ttcaacctattggtgctttggacatatcagcatctatagtagctggtggtattgtagctatcgtagtaacatgccttgcctactattttatgagg
tttagaagagcttttggtgaatacagtcatgtagttgcctttaatactttactattccttatgtcattcactgtactctgtttaacaccagtttactc
attcttacctggtgtttattctgttatttacttgtacttgacattttatcttactaatgatgtttcttttttagcacatattcagtggatggttatgttca
cacctttagtacctttctggataacaattgcttatatcatttgtatttccacaaagcatttctattggttctttagtaattacctaaagagacgtgt
agtctttaatggtgtttcctttagtacttttgaagaagctgcgctgtgcacctttttgttaaataaagaaatgtatctaaagttgcgtagtgatgt
gctattacctcttacgcaatataatagatacttagctctttataataagtacaagtattttagtggagcaatggatacaactagctacagaga
agctgcttgttgtcatctcgcaaaggctctcaatgacttcagtaactcaggttctgatgttctttaccaaccaccacaaacctctatcacctc
agctgttttgcagagtggttttagaaaaatggcattcccatctggtaaagttgagggttgtatggtacaagtaacttgtggtacaactacac
ttaacggtctttggcttgatgacgtagtttactgtccaagacatgtgatctgcacctctgaagacatgcttaaccctaattatgaagatttact
cattcgtaagtctaatcataatttcttggtacaggctggtaatgttcaactcagggttattggacattctatgcaaaattgtgtacttaagctta
aggttgatacagccaatcctaagacacctaagtataagtttgttcgcattcaaccaggacagactttttcagtgttagcttgttacaatggtt
caccatctggtgtttaccaatgtgctatgaggcccaatttcactattaagggttcattccttaatggttcatgtggtagtgttggttttaacata
gattatgactgtgtctctttttgttacatgcaccatatggaattaccaactggagttcatgctggcacagacttagaaggtaacttttatggac
cttttgttgacaggcaaacagcacaagcagctggtacggacacaactattacagttaatgttttagcttggttgtacgctgctgttataaat
ggagacaggtggtttctcaatcgatttaccacaactcttaatgactttaaccttgtggctatgaagtacaattatgaacctctaacacaaga
ccatgttgacatactaggacctctttctgctcaaactggaattgccgttttagatatgtgtgcttcattaaaagaattactgcaaaatggtatg
aatggacgtaccatattgggtagtgctttattagaagatgaatttacaccttttgatgttgttagacaatgctcaggtgttactttccaaagtg
cagtgaaaagaacaatcaagggtacacaccactggttgttactcacaattttgacttcacttttagttttagtccagagtactcaatggtcttt
gttcttttttttgtatgaaaatgcctttttaccttttgctatgggtattattgctatgtctgcttttgcaatgatgtttgtcaaacataagcatgcattt
ctctgtttgtttttgttaccttctcttgccactgtagcttattttaatatggtctatatgcctgctagttgggtgatgcgtattatgacatggttgga
tatggttgatactagtttgtctggttttaagctaaaagactgtgttatgtatgcatcagctgtagtgttactaatccttatgacagcaagaactg
tgtatgatgatggtgctaggagagtgtggacacttatgaatgtcttgacactcgtttataaagtttattatggtaatgctttagatcaagccat
ttccatgtgggctcttataatctctgttacttctaactactcaggtgtagttacaactgtcatgtttttggccagaggtattgtttttatgtgtgttg
agtattgccctattttcttcataactggtaatacacttcagtgtataatgctagtttattgtttcttaggctatttttgtacttgttactttggcctcttt
tgtttactcaaccgctactttagactgactcttggtgtttatgattacttagtttctacacaggagtttagatatatgaattcacagggactactc
ccacccaagaatagcatagatgccttcaaactcaacattaaattgttgggtgttggtggcaaaccttgtatcaaagtagccactgtacagt
ctaaaatgtcagatgtaaagtgcacatcagtagtcttactctcagttttgcaacaactcagagtagaatcatcatctaaattgtgggctcaat
gtgtccagttacacaatgacattctcttagctaaagatactactgaagcctttgaaaaaatggtttcactactttctgttttgctttccatgcag
ggtgctgtagacataaacaagctttgtgaagaaatgctggacaacagggcaaccttacaagctatagcctcagagtttagttcccttcca
tcatatgcagcttttgctactgctcaagaagcttatgagcaggctgttgctaatggtgattctgaagttgttcttaaaaagttgaagaagtctt
tgaatgtggctaaatctgaatttgaccgtgatgcagccatgcaacgtaagttggaaaagatggctgatcaagctatgacccaaatgtata
aacaggctagatctgaggacaagagggcaaaagttactagtgctatgcagacaatgcttttcactatgcttagaaagttggataatgatg
cactcaacaacattatcaacaatgcaagagatggttgtgttcccttgaacataatacctcttacaacagcagccaaactaatggttgtcata
ccagactataacacatataaaaatacgtgtgatggtacaacatttacttatgcatcagcattgtgggaaatccaacaggttgtagatgcag
atagtaaaattgttcaacttagtgaaattagtatggacaattcacctaatttagcatggcctcttattgtaacagctttaagggccaattctgct
gtcaaattacagaataatgagcttagtcctgttgcactacgacagatgtcttgtgctgccggtactacacaaactgcttgcactgatgaca
atgcgttagcttactacaacacaacaaagggaggtaggtttgtacttgcactgttatccgatttacaggatttgaaatgggctagattccct
aagagtgatggaactggtactatctatacagaactggaaccaccttgtaggtttgttacagacacacctaaaggtcctaaagtgaagtatt
tatactttattaaaggattaaacaacctaaatagaggtatggtacttggtagtttagctgccacagtacgtctacaagctggtaatgcaaca
gaagtgcctgccaattcaactgtattatctttctgtgcttttgctgtagatgctgctaaagcttacaaagattatctagctagtgggggacaa
ccaatcactaattgtgttaagatgttgtgtacacacactggtactggtcaggcaataacagttacaccggaagccaatatggatcaagaa
tcctttggtggtgcatcgtgttgtctgtactgccgttgccacatagatcatccaaatcctaaaggattttgtgacttaaaaggtaagtatgtac
aaatacctacaacttgtgctaatgaccctgtgggttttacacttaaaaacacagtctgtaccgtctgcggtatgtggaaaggttatggctgt
agttgtgatcaactccgcgaacccatgcttcagtcagctgatgcacaatcgtttttaaacgggtttgcggtgtaagtgcagcccgtcttac
accgtgcggcacaggcactagtactgatgtcgtatacagggcttttgacatctacaatgataaagtagctggttttgctaaattcctaaaa
actaattgttgtcgcttccaagaaaaggacgaagatgacaatttaattgattcttactttgtagttaagagacacactttctctaactaccaac
atgaagaaacaatttataatttacttaaggattgtccagctgttgctaaacatgacttctttaagtttagaatagacggtgacatggtaccac
atatatcacgtcaacgtcttactaaatacacaatggcagacctcgtctatgctttaaggcattttgatgaaggtaattgtgacacattaaaag
aaatacttgtcacatacaattgttgtgatgatgattatttcaataaaaaggactggtatgattttgtagaaaacccagatatattacgcgtata
cgccaacttaggtgaacgtgtacgccaagctttgttaaaaacagtacaattctgtgatgccatgcgaaatgctggtattgttggtgtactg
acattagataatcaagatctcaatggtaactggtatgatttcggtgatttcatacaaaccacgccaggtagtggagttcctgttgtagattct
tattattcattgttaatgcctatattaaccttgaccagggctttaactgcagagtcacatgttgacactgacttaacaaagccttacattaagt
gggatttgttaaaatatgacttcacggaagagaggttaaaactctttgaccgttattttaaatattgggatcagacataccacccaaattgtg
ttaactgtttggatgacagatgcattctgcattgtgcaaactttaatgttttattctctacagtgttcccacctacaagttttggaccactagtga
gaaaaatatttgttgatggtgttccatttgtagtttcaactggataccacttcagagagctaggtgttgtacataatcaggatgtaaacttaca
tagctctagacttagttttaaggaattacttgtgtatgctgctgaccctgctatgcacgctgcttctggtaatctattactagataaacgcact
acgtgcttttcagtagctgcacttactaacaatgttgcttttcaaactgtcaaacccggtaattttaacaaagacttctatgactttgctgtgtc
taagggtttctttaaggaaggaagttctgttgaattaaaacacttcttctttgctcaggatggtaatgctgctatcagcgattatgactactat
cgttataatctaccaacaatgtgtgatatcagacaactactatttgtagttgaagttgttgataagtactttgattgttacgatggtggctgtatt
aatgctaaccaagtcatcgtcaacaacctagacaaatcagctggttttccatttaataaatggggtaaggctagactttattatgattcaatg
agttatgaggatcaagatgcacttttcgcatatacaaaacgtaatgtcatccctactataactcaaatgaatcttaagtatgccattagtgca
aagaatagagctcgcaccgtagctggtgtctctatctgtagtactatgaccaatagacagtttcatcaaaaattattgaaatcaatagccg
ccactagaggagctactgtagtaattggaacaagcaaattctatggtggttggcacaacatgttaaaaactgtttatagtgatgtagaaaa
ccctcaccttatgggttgggattatcctaaatgtgatagagccatgcctaacatgcttagaattatggcctcacttgttcttgctcgcaaaca
tacaacgtgttgtagcttgtcacaccgtttctatagattagctaatgagtgtgctcaagtattgagtgaaatggtcatgtgtggcggttcact
atatgttaaaccaggtggaacctcatcaggagatgccacaactgcttatgctaatagtgtttttaacatttgtcaagctgtcacggccaatg
ttaatgcacttttatctactgatggtaacaaaattgccgataagtatgtccgcaatttacaacacagactttatgagtgtctctatagaaatag
agatgttgacacagactttgtgaatgagttttacgcatatttgcgtaaacatttctcaatgatgatactctctgacgatgctgttgtgtgtttca
atagcacttatgcatctcaaggtctagtggctagcataaagaactttaagtcagttctttattatcaaaacaatgtttttatgtctgaagcaaa
atgttggactgagactgaccttactaaaggacctcatgaattttgctctcaacatacaatgctagttaaacagggtgatgattatgtgtacct
tccttacccagatccatcaagaatcctaggggccggctgttttgtagatgatatcgtaaaaacagatggtacacttatgattgaacggttc
gtgtctttagctatagatgcttacccacttactaaacatcctaatcaggagtatgctgatgtctttcatttgtacttacaatacataagaaagct
acatgatgagttaacaggacacatgttagacatgtattctgttatgcttactaatgataacacttcaaggtattgggaacctgagttttatga
ggctatgtacacaccgcatacagtcttacaggctgttggggcttgtgttctttgcaattcacagacttcattaagatgtggtgcttgcatacg
tagaccattcttatgttgtaaatgctgttacgaccatgtcatatcaacatcacataaattagtcttgtctgttaatccgtatgtttgcaatgctcc
aggttgtgatgtcacagatgtgactcaactttacttaggaggtatgagctattattgtaaatcacataaaccacccattagttttccattgtgt
gctaatggacaagtttttggtttatataaaaatacatgtgttggtagcgataatgttactgactttaatgcaattgcaacatgtgactggacaa
atgctggtgattacattttagctaacacctgtactgaaagactcaagctttttgcagcagaaacgctcaaagctactgaggagacatttaa
actgtcttatggtattgctactgtacgtgaagtgctgtctgacagagaattacatctttcatgggaagttggtaaacctagaccaccacttaa
ccgaaattatgtctttactggttatcgtgtaactaaaaacagtaaagtacaaataggagagtacacctttgaaaaaggtgactatggtgat
gctgttgtttaccgaggtacaacaacttacaaattaaatgttggtgattattttgtgctgacatcacatacagtaatgccattaagtgcaccta
cactagtgccacaagagcactatgttagaattactggcttatacccaacactcaatatctcagatgagttttctagcaatgttgcaaattatc
aaaaggttggtatgcaaaagtattctacactccagggaccacctggtactggtaagagtcattttgctattggcctagctctctactaccct
tctgctcgcatagtgtatacagcttgctctcatgccgctgttgatgcactatgtgagaaggcattaaaatatttgcctatagataaatgtagt
agaattatacctgcacgtgctcgtgtagagtgttttgataaattcaaagtgaattcaacattagaacagtatgtcttttgtactgtaaatgcatt
gcctgagacgacagcagatatagttgtctttgatgaaatttcaatggccacaaattatgatttgagtgttgtcaatgccagattacgtgcta
agcactatgtgtacattggcgaccctgctcaattacctgcaccacgcacattgctaactaagggcacactagaaccagaatatttcaattc
agtgtgtagacttatgaaaactataggtccagacatgttcctcggaacttgtcggcgttgtcctgctgaaattgttgacactgtgagtgcttt
ggtttatgataataagcttaaagcacataaagacaaatcagctcaatgctttaaaatgttttataagggtgttatcacgcatgatgtttcatct
gcaattaacaggccacaaataggcgtggtaagagaattccttacacgtaaccctgcttggagaaaagctgtctttatttcaccttataattc
acagaatgctgtagcctcaaagattttgggactaccaactcaaactgttgattcatcacagggctcagaatatgactatgtcatattcactc
aaaccactgaaacagctcactcttgtaatgtaaacagatttaatgttgctattaccagagcaaaagtaggcatactttgcataatgtctgat
agagacctttatgacaagttgcaatttacaagtcttgaaattccacgtaggaatgtggcaactttacaagctgaaaatgtaacaggactctt
taaagattgtagtaaggtaatcactgggttacatcctacacaggcacctacacacctcagtgttgacactaaattcaaaactgaaggtttat
gtgttgacatacctggcatacctaaggacatgacctatagaagactcatctctatgatgggttttaaaatgaattatcaagttaatggttacc
ctaacatgtttatcacccgcgaagaagctataagacatgtacgtgcatggattggcttcgatgtcgaggggtgtcatgctactagagaag
ctgttggtaccaatttacctttacagctaggtttttctacaggtgttaacctagttgctgtacctacaggttatgttgatacacctaataataca
gatttttccagagttagtgctaaaccaccgcctggagatcaatttaaacacctcataccacttatgtacaaaggacttccttggaatgtagt
gcgtataaagattgtacaaatgttaagtgacacacttaaaaatctctctgacagagtcgtatttgtcttatgggcacatggctttgagttgac
atctatgaagtattttgtgaaaataggacctgagcgcacctgttgtctatgtgatagacgtgccacatgcttttccactgcttcagacactta
tgcctgttggcatcattctattggatttgattacgtctataatccgtttatgattgatgttcaacaatggggttttacaggtaacctacaaagca
accatgatctgtattgtcaagtccatggtaatgcacatgtagctagttgtgatgcaatcatgactaggtgtctagctgtccacgagtgctttg
ttaagcgtgttgactggactattgaatatcctataattggtgatgaactgaagattaatgcggcttgtagaaaggttcaacacatggttgtta
aagctgcattattagcagacaaattcccagttcttcacgacattggtaaccctaaagctattaagtgtgtacctcaagctgatgtagaatgg
aagttctatgatgcacagccttgtagtgacaaagcttataaaatagaagaattattctattcttatgccacacattctgacaaattcacagat
ggtgtatgcctattttggaattgcaatgtcgatagatatcctgctaattccattgtttgtagatttgacactagagtgctatctaaccttaacttg
cctggttgtgatggtggcagtttgtatgtaaataaacatgcattccacacaccagcttttgataaaagtgcttttgttaatttaaaacaattac
catttttctattactctgacagtccatgtgagtctcatggaaaacaagtagtgtcagatatagattatgtaccactaaagtctgctacgtgtat
aacacgttgcaatttaggtggtgctgtctgtagacatcatgctaatgagtacagattgtatctcgatgcttataacatgatgatctcagctgg
ctttagcttgtgggtttacaaacaatttgatacttataacctctggaacacttttacaagacttcagagtttagaaaatgtggcttttaatgttgt
aaataagggacactttgatggacaacagggtgaagtaccagtttctatcattaataacactgtttacacaaaagttgatggtgttgatgtag
aattgtttgaaaataaaacaacattacctgttaatgtagcatttgagctttgggctaagcgcaacattaaaccagtaccagaggtgaaaat
actcaataatttgggtgtggacattgctgctaatactgtgatctgggactacaaaagagatgctccagcacatatatctactattggtgtttg
ttctatgactgacatagccaagaaaccaactgaaacgatttgtgcaccactcactgtcttttttgatggtagagttgatggtcaagtagactt
atttagaaatgcccgtaatggtgttcttattacagaaggtagtgttaaaggtttacaaccatctgtaggtcccaaacaagctagtcttaatgg
agtcacattaattggagaagccgtaaaaacacagttcaattattataagaaagttgatggtgttgtccaacaattacctgaaacttactttac
tcagagtagaaatttacaagaatttaaacccaggagtcaaatggaaattgatttcttagaattagctatggatgaattcattgaacggtata
aattagaaggctatgccttcgaacatatcgtttatggagattttagtcatagtcagttaggtggtttacatctactgattggactagctaaacg
ttttaaggaatcaccttttgaattagaagattttattcctatggacagtacagttaaaaactatttcataacagatgcgcaaacaggttcatct
aagtgtgtgtgttctgttattgatttattacttgatgattttgttgaaataataaaatcccaagatttatctgtagtttctaaggttgtcaaagtgac
tattgactatacagaaatttcatttatgctttggtgtaaagatggccatgtagaaacattttacccaaaattacaatctagtcaagcgtggca
accgggtgttgctatgcctaatctttacaaaatgcaaagaatgctattagaaaagtgtgaccttcaaaattatggtgatagtgcaacattac
ctaaaggcataatgatgaatgtcgcaaaatatactcaactgtgtcaatatttaaacacattaacattagctgtaccctataatatgagagtta
tacattttggtgctggttctgataaaggagttgcaccaggtacagctgttttaagacagtggttgcctacgggtacgctgcttgtcgattca
gatcttaatgactttgtctctgatgcagattcaactttgattggtgattgtgcaactgtacatacagctaataaatgggatctcattattagtga
tatgtacgaccctaagactaaaaatgttacaaaagaaaatgactctaaagagggttttttcacttacatttgtgggtttatacaacaaaagct
agctcttggaggttccgtggctataaagataacagaacattcttggaatgctgatctttataagctcatgggacacttcgcatggtggaca
gcctttgttactaatgtgaatgcgtcatcatctgaagcatttttaattggatgtaattatcttggcaaaccacgcgaacaaatagatggttatg
tcatgcatgcaaattacatattttggaggaatacaaatccaattcagttgtcttcctattctttatttgacatgagtaaatttccccttaaattaag
gggtactgctgttatgtctttaaaagaaggtcaaatcaatgatatgattttatctcttcttagtaaaggtagacttataattagagaaaacaac
agagttgttatttctagtgatgttcttgttaacaactaa

SEQ ID NO: 58, Severe acute respiratory syndrome coronavirus 2 isolate Wuhan-Hu-1, E envelope protein, Gene ID: 43740570, 228 bp ss-RNA, NC_045512 region 26245-26472

ATGTACTCATTCGTTTCGGAAGAGACAGGTACGTTAATAGTTAATAGCGTACTTCTTTTTC
TTGCTTTCGTGGTATTCTTGCTAGTTACACTAGCCATCCTTACTGCGCTTCGATTGTGTGC
GTACTGCTGCAATATTGTTAACGTGAGTCTTGTAAAACCTTCTTTTTACGTTTACTCTCGT
GTTAAAAATCTGAATTCTTCTAGAGTTCCTGATCTTCTGGTCTAA

In some embodiments of any of the aspects, the target nucleic acid is a synthetic sequence. In some embodiments of any of the aspect, the synthetic sequence comprises canonical bases. In some embodiments of any of the aspect, the synthetic sequence (e.g., synthetic target nucleic acid and/or one or both primers) comprises non-canonical bases. A nucleic acid can also include nucleobase (often referred to in the art simply as “base”) modifications or substitutions.

As used herein, “unmodified” or “natural” or “canonical” nucleobases include the purine bases adenine (A) and guanine (G), and the pyrimidine bases thymine (T), cytosine (C) and uracil (U). Modified or non-canonical nucleobases can include other synthetic and natural nucleobases including but not limited to as inosine, isocytosine, isoguanine, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications. In some embodiments of any of the aspects, modified nucleobases can include d5SICS and dNAM, which are a non-limiting example of unnatural nucleobases that can be used separately or together as base pairs (see e.g., Leconte et. al. J. Am. Chem. Soc.2008, 130, 7, 2336-2343; Malyshev et. al. PNAS. 2012. 109 (30) 12005-12010). In some embodiments of any of the aspects, the nucleic acid comprises any modified nucleobases known in the art, i.e., any nucleobase that is modified from an unmodified and/or natural nucleobase. In some embodiments of any of the aspects, the target nucleic acid is left-handed DNA, right-handed DNA, RNA, a chimera (e.g., of DNA and RNA), or another nucleic acid structure.

In some embodiments of any of the aspects, the target nucleic acid is attached covalently or non-covalently to an antibody, protein, lipid, surface, or other substrate. Non-limiting examples of a substrate include: a lateral flow strip; a nucleic acid scaffold; a protein scaffold; a lipid scaffold; a dendrimer; a microparticle; a microbead; a magnetic microbead; a paramagnetic microbead; medical apparatuses (e.g., needles or catheters) or medical implants; a microtiter plate; a microporous membrane; a microchip; a hollow fiber; a hollow fiber reactor or cartridge; a fluid filtration membrane; a fluid filtration device; a membrane; a diagnostic strip; a dipstick; an extracorporeal device; a mixing element (e.g., a spiral mixer); a microscopic slide; a flow device; a microfluidic device; a living cell; an extracellular matrix of a biological tissue or organ; or any combination thereof. The solid substrate can be made of any material, including, but not limited to, metal, metal alloy, polymer, plastic

In some embodiments of any of the aspects, the target nucleic acid has been previously cleaved from such a substrate. In some embodiments of any of the aspects, the sequence of the target nucleic acid represents or encodes or designates the identity of the substrate (e.g. protein) to which the target nucleic acid is attached. In some embodiments of any of the aspects, the sequence of the target nucleic acid represents or encodes or designates the identity of another element in which it has formed a complex (e.g. designating the antigen of the antibody to which the target nucleic acid is attached).

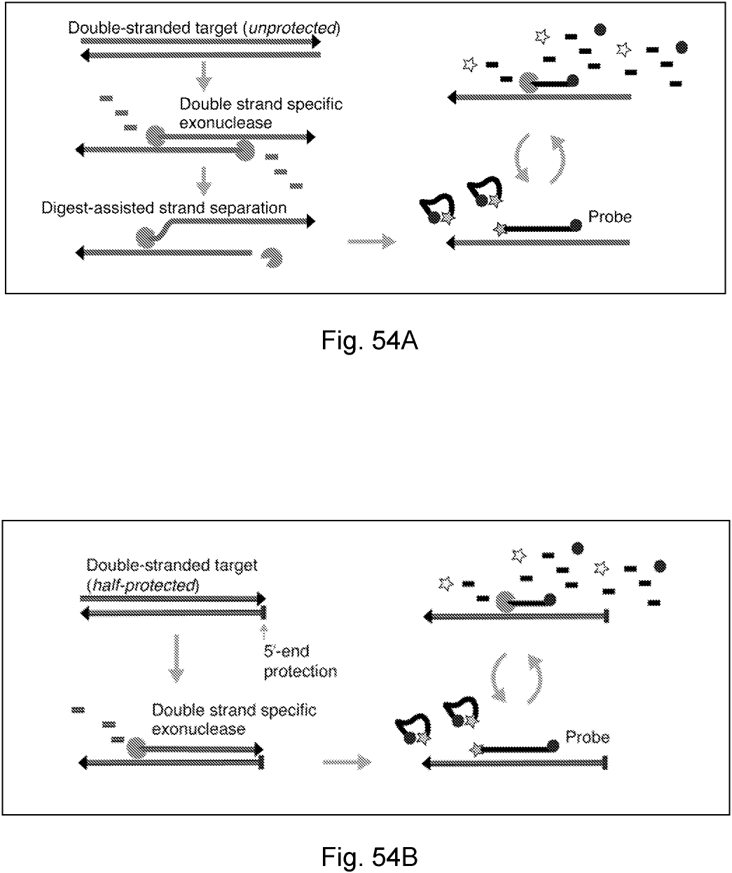

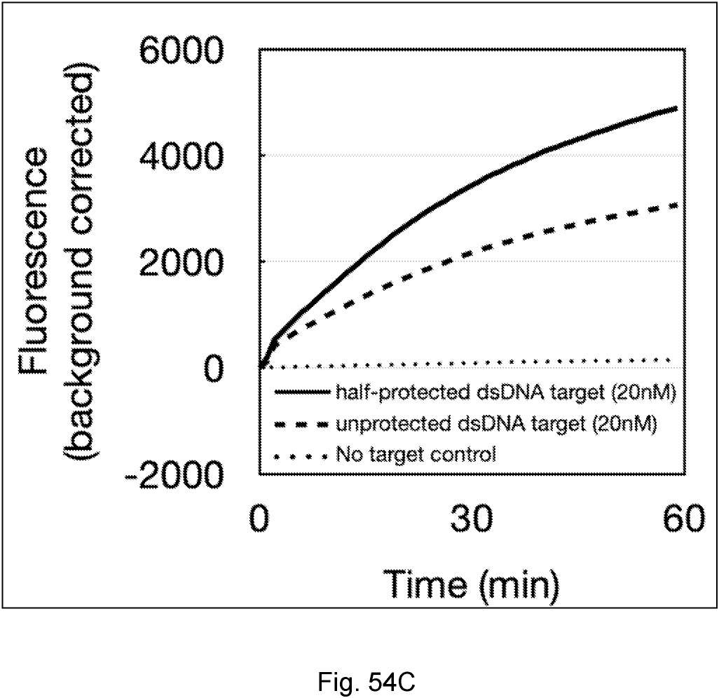

In some embodiments of any of the aspects, at least one strand of the target nucleic acid comprises a nucleic acid modification known in the art. As a non-limiting example, the non-target strand of a double-stranded target nucleic acid (i.e., the strand not bound by a probe as described herein) comprises a nucleic acid modification (see e.g., FIGS. 54A-54C). In some embodiments of any of the aspects, at least one strand of the target nucleic acid comprises a nucleic acid modification that can inhibit 5′->3′ cleaving activity of a 5′->3′ exonuclease. Nucleic acid modifications that can inhibit 5′-> 3′ cleaving activity of a 5′->3′ exonuclease are known in the art, such as modified internucleotide linkages, modified nucleobase, modified sugar, and any combinations thereof. Exemplary modifications include, but are not limited to 1, 2, 3, 4, 5, 6 or more modified internucleotide linkages, such as phosphorothioates; an inverted nucleoside or 5′->5′ internucleotide linkage; a 3′->3′ internucleotide linkage; a 2′-OH or a 2′-modified nucleoside; a 5′-modified nucleotide; a 2′->5′ linkage; an abasic nucleoside; an acyclic nucleoside; nucleotides with non-canonical nucleobases; replacement of 5′-OH group; or any combinations thereof.

The modification capable of inhibiting 5′->3′ cleaving activity can be present anywhere in the target nucleic acid. For example, it can be at the 5′-end or terminus, at an internal position, or at a position within the 5′-terminal, e.g., within positions within 1, 2, 3, 4, 5, 6, 7, 8, 9, 10, 11, 12, 13, 14 or 15 from the 5′-end. In some embodiments of any of the aspects, the nucleic acid modification is located at the 5′-end of the target nucleic acid. In some embodiments of any of the aspects, the modification is a phosphorothioate base, a spacer modification, 2′-O-Methyl RNA, 5′ inverted dideoxy-dT base, and/ or 2′ Fluoro bases.

It will be apparent to those skilled in the art that various modifications and variations can be made to improve the stability of the target nucleic acid.

### Sample Preparation

Described herein are methods, kits, and systems permitting detection of a target nucleic acid from a sample. The term “sample” or “test sample” as used herein denotes a sample taken or isolated from a biological organism, e.g., a subject in need of testing. In some embodiments of any of the aspects, the technology described herein encompasses several examples of a biological sample, including but not limited to a sputum sample, a pharyngeal sample, or a nasal sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

In some embodiments of any of the aspects, the sample is contacted with a transport media, such a viral transport media (VTM). In some embodiments of any of the aspects, transport media preserves the target nucleic acid between the time of sample collection and detection of the target nucleic acid. The constituents of suitable viral transport media are designed to provide an isotonic solution containing protective protein, antibiotics to control microbial contamination, and one or more buffers to control the pH. Isotonicity, however, is not an absolute requirement; some highly successful transport media contain hypertonic solutions of sucrose. Liquid transport media are used primarily for transporting swabs or materials released into the medium from a collection swab. Liquid media may be added to other specimens when inactivation of the viral agent is likely and when the resultant dilution is acceptable. A suitable VTM for use in collecting throat and nasal swabs from human patients is prepared as follows: (1) add 10 g veal infusion broth and 2 g bovine albumin fraction V to sterile distilled water (to 400 ml); (2) add 0.8 ml gentamicin sulfate solution (50 mg/ml) and 3.2 ml amphotericin B (250 µg/ml); and (3) sterilize by filtration. Additional non-limiting examples of viral transport media include COPAN Universal Transport Medium; Eagle Minimum Essential Medium (E-MEM); Transport medium 199; and PBS-Glycerol transport medium. see e.g., Johnson, Transport of Viral Specimens, CLINICAL MICROBIOLOGY REVIEWS, April 1990, p. 120-131; Collecting, preserving and shipping specimens for the diagnosis of avian influenza A(H5N1) virus infection, Guide for field operations, October 2006. In some embodiments of any of the aspects, viral transport media does not inhibit the methods (Digest-LAMP and/or ssRPA) as described herein.

In some embodiments of any of the aspects, the target nucleic acids are isolated from the sample. In some embodiments of any of the aspects, RNA isolation can be performed using standard RNA extraction methods or kits. Non-limiting examples of standard RNA extraction methods include: (1) organic extraction, such as phenol-Guanidine Isothiocyanate (GITC)-based solutions (e.g., TRIZOL and TRI reagent); (2) silica-membrane based spin column technology (e.g., RNeasy and its variants); (3) paramagnetic particle technology (e.g., DYNABEADS mRNA DIRECT MICRO); (4) density gradient centrifugation using cesium chloride or cesium trifluoroacetate; (5) lithium chloride and urea isolation; (6) oligo(dt)-cellulose column chromatography; and (7) non-column poly (A)+ purification/isolation. In some embodiments of any of the aspects, DNA isolation can be performed using standard DNA extraction methods or kits. Non-limiting examples of standard DNA extraction methods include: organic extraction, CHELEX 100 extraction, and solid phase extraction.

Target nucleic acid molecules can be isolated from a particular biological sample using any of a number of procedures, which are known in the art, the particular isolation procedure chosen being appropriate for the particular biological sample. For example, freeze-thaw and alkaline lysis procedures can be useful for obtaining nucleic acid molecules from solid materials (Roiff, A et al. PCR: Clinical Diagnostics and Research, Springer (1994)).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, homogenization, sonication, filtration, thawing, purification, and any combinations thereof. In some embodiments of any of the aspects, the test sample can be treated with a chemical and/or biological reagent. Chemical and/or biological reagents can be employed, for example, to protect and/or maintain the stability of the sample, including biomolecules (e.g., nucleic acid and protein) therein, during processing. The skilled artisan is well aware of methods and processes appropriate for pre-processing of biological samples required for detection of a nucleic acid as described herein.

### Reverse Transcription

In embodiments where the target nucleic acid is an RNA, the target RNA can be reverse transcribed to a complementary DNA (cDNA) that is thereafter amplified and detected. Accordingly, the methods described herein can further comprise a step of contacting the sample with a reverse transcriptase and a set of primers. The methods described herein can further comprise a step of reverse transcribing a target RNA prior to amplification and hybridizing with the probe.

In some embodiments of any of the aspects, the reverse transcription step and amplification step(s) are performed simultaneously in the same reaction.

The term “reverse transcriptase” (RT) refers to an RNA-dependent DNA polymerase used to generate complementary DNA (cDNA) from an RNA template. In some embodiments of any of the aspects, the cDNA is single-stranded DNA (ssDNA) or double-stranded DNA (dsDNA). Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Reverse transcriptases are also used in the synthesis of extrachromosomal DNA/RNA chimeric elements called multicopy single-stranded DNA (msDNA) in bacteria. Retroviral RT has three sequential biochemical activities: RNA-dependent DNA polymerase activity, ribonuclease H (RNAse H), and/or DNA-dependent DNA polymerase activity. Collectively, these activities permit the enzyme to convert single-stranded RNA into double-stranded cDNA.

In some embodiments of any of the aspects, the reverse transcriptase can be any enzyme that can produce cDNA from an RNA transcript. In some embodiments of any of the aspects, the reverse transcriptase comprises a HIV-1 reverse transcriptase from human immunodeficiency virus type 1. In some embodiments of any of the aspects, the reverse transcriptase comprises M-MuLV reverse transcriptase from the Moloney murine leukemia virus (referred to as M-MuLV, M-MLV, or MMLV). In some embodiments of any of the aspects, the reverse transcriptase comprises AMV reverse transcriptase from the avian myeloblastosis virus (AVM). In some embodiments of any of the aspects, the reverse transcriptase comprises telomerase reverse transcriptase that maintains the telomeres of eukaryotic chromosomes. In some embodiments of any of the aspects, the reverse transcriptase is selected from those expressed by any Group VI or Group VII virus. In some embodiments of any of the aspects, the reverse transcriptase is a naturally occurring RT selected from the group consisting of: an M-MLV RT, an AMV RT, a retrotransposon RT, a telomerase reverse transcriptase, and an HIV-1 reverse transcriptase.

In some embodiments of any of the aspects, the reverse transcriptase is an engineered or recombinant version of an M-MuLV RT, AMV RT, or another naturally occurring RT as described herein. In some embodiments of any of the aspects, the reverse transcriptase is ProtoScript® II Reverse Transcriptase, which is also referred to herein as ProtoScript® II RT or Protoscriptase II. ProtoScript® II RT is a recombinant Moloney Murine Leukemia Virus (M-MuLV) reverse transcriptase, e.g., a fusion of the Escherichia coli trpE gene with the central region of the M-MuLV pol gene.

In some embodiments of any of the aspects, the reverse transcriptase is selected from the group consisting of: Maxima® RT, Omniscript® RT, PowerScript® RT, Sensiscript® RT (SES), SuperScript® II (SSII or SS2), SuperScript® III (SSIII or SS3), SuperScript® IV (SSIV), Accuscript® RT (ACC), a recombinant HIV RT, imProm-II® (IP2) RT, M-MLV RT (MML), Protoscript® RT (PRS), Smart MMLV (SML) RT, ThermoScript® (TSR) RT (see e.g., Levesque-Sergerie et al., BMC Molecular Biology volume 8, Article number: 93 (2007); Okello et al., PLoS One. 2010 Nov 10;5(11):e13931). Non limiting examples of RTs derived from MMLV include PowerScript®, ACC, MML, SML, SS2, and SS3. Non limiting examples of RTs derived from AMV include PRS and TSR. Non limiting examples of RTs derived proprietary sources include IP2, SES, Omniscript®. In some embodiments of any of the aspects, reverse transcriptase exhibits increased thermostability (e.g., up to 48° C.) compared to the wild type RT.

As used herein, one unit (“U”) of reverse transcriptase (e.g., ProtoScript® II RT) is defined as is defined as the amount of enzyme that will incorporate 1 nmol of dTTP into acid-insoluble material in a total reaction volume of 50 µl in 10 minutes at 37° C. using poly(rA)•oligo(dT)₁₈ as template. In some embodiments of any of the aspects, the reverse transcriptase is provided at a concentration of at least 1 U/µL, at least 2 U/µL, at least 3 U/µL, at least 4 U/µL, at least 5 U/µL, at least 6 U/µL, at least 7 U/µL, at least 8 U/µL, at least 9 U/µL, at least 10 U/µL, at least 20 U/µL, at least 30 U/µL, at least 40 U/µL, at least 50 U/µL, at least 60 U/µL, at least 70 U/µL, at least 80 U/µL, at least 90 U/µL, at least 100 U/µL, at least 110 U/µL, at least 120 U/µL, at least 130 U/µL, at least 140 U/µL, at least 150 U/µL, at least 160 U/µL, at least 170 U/µL, at least 180 U/µL, at least 190 U/µL, at least 200 U/µL, at least 210 U/µL, at least 220 U/µL, at least 230 U/µL, at least 240 U/µL, at least 250 U/µL, at least 260 U/µL, at least 270 U/µL, at least 280 U/µL, at least 290 U/µL, at least 300 U/µL, at least 310 U/µL, at least 320 U/µL, at least 330 U/µL, at least 340 U/µL, at least 350 U/µL, at least 360 U/µL, at least 370 U/µL, at least 380 U/µL, at least 390 U/µL, at least 400 U/µL, at least 410 U/µL, at least 420 U/µL, at least 430 U/µL, at least 440 U/µL, at least 450 U/µL, at least 460 U/µL, at least 470 U/µL, at least 480 U/µL, at least 490 U/µL, or at least 500 U/µL. In some embodiments of any of the aspects, the reverse transcriptase is provided at a concentration of 20 U/µL. In some embodiments of any of the aspects, the reverse transcriptase is provided at a concentration of 200 U/µL.

In some embodiments of any of the aspects, the sample is contacted with a first set of primers. In some embodiments of any of the aspects, the first set of primers comprises primers that bind to target RNA and non-target RNA in the sample, i.e., “general” primers. In some embodiments of any of the aspects, the first set of primers comprises random hexamers, i.e., a mixture of oligonucleotides representing all possible hexamer sequences. In some embodiments of any of the aspects, the first set of primers comprises oligo(dT) primer, which bind to the polyA tails of mRNAs or viral transcripts.

In some embodiments of any of the aspects, the first set of primers is specific to the target RNA. In some embodiments of any of the aspects, the first set of primers comprises the reverse primer of the second set of primers (e.g., used in the amplification step). In embodiments comprising a one-pot reaction, the first set of primers can comprise the second set of primers, or the second set of primers can comprise the first set of primers. In some embodiments of any of the aspects, the RT step comprises one round of polymerization, wherein the target RNA is reverse-transcribed into a single-stranded cDNA.

In some embodiments of any of the aspects, the reverse transcription step comprises contacting the sample with a reverse transcriptase, a first set of primers, and at least one of the following: a reaction buffer, water, magnesium acetate (or another magnesium compound such as magnesium chloride) dNTPs, DTT, and/or an RNase inhibitor. In some embodiments of any of the aspects, the reaction buffer maintains the reaction at specific optimal pH (e.g., 8.1) and can include such components as Tris(pH8.1), KCl, MgCl2, and other buffers or salts. Magnesium ions (Mg2+) can function as a cofactor for polymerases, increasing their activity. Deoxynucleoside triphosphate (dNTPs) are free nucleoside triphosphates comprising deoxyribose as the sugar (e.g., dATP, dGTP, dCTP, and dTTP) that are used in the polymerization of the cDNA. Dithiothreitol (DTT) is a redox reagent used to stabilize proteins which possess free sulfhydryl groups (e.g., RT). In some embodiments of any of the aspects, the RNase inhibitor specifically inhibits RNases A, B and C, which specifically cleave ssRNA or dsRNA. RNase A and RNase B are an endoribonuclease that specifically degrades single-stranded RNA at C and U residues. RNase C recognizes dsRNA and cleaves it at specific targeted locations to transform them into mature RNAs.

In some embodiments of any of the aspects, the RT step is performed between 12° C. and 45° C. As a non-limiting example, the RT step is performed at a temperature of at least 12° C., at least 13° C., at least 14° C., at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C.

In some embodiments of any of the aspects, the RT step is performed at a temperature of at most 12° C., at most 13° C., at most 14° C., at most 15° C., at most 16° C., at most 17° C., at most 18° C., at most 19° C., at most 20° C., at most 21° C., at most 22° C., at most 23° C., at most 24° C., at most 25° C., at most 26° C., at most 27° C., at most 28° C., at most 29° C., at most 30° C., at most 31° C., at most 32° C., at most 33° C., at most 34° C., at most 35° C., at most 36° C., at most 37° C., at most 38° C., at most 39° C., at most 40° C., at most 41° C., at most 42° C., at most 43° C., at most 44° C., at most 45° C. In some embodiments of any of the aspects, the RT step is performed at room temperature (e.g., 20° C.-22° C.). In some embodiments of any of the aspects, the RT step is performed at body temperature (e.g., 37° C.). In some embodiments of any of the aspects, the RT step is performed on a heat block set to approximately 42° C.

In some embodiments of any of the aspects, the RT step is performed in at most 1 minute. In some embodiments of any of the aspects, the RT step is performed in at most 5 minutes. In some embodiments of any of the aspects, the RT step is performed in at most 20 minutes. As a non-limiting example, the RT step is performed in at most 1 minute, 2 minutes, 3 minutes, 4 minutes, 5 minutes, at most 6 minutes, at most 7 minutes, at most 8 minutes, at most 9 minutes, at most 10 minutes, at most 15 minutes, at most 20 minutes, at most 25 minutes, at most 30 minutes, at most 40 minutes, at most 50 minutes, at most 60 minutes, at most 70 minutes, at most 80 minutes, at most 90 minutes, or at most 100 minutes.

### Compositions

In another aspect, provided herein are compositions useful in detecting a target nucleic acid. The composition may comprise any of the reagents discussed herein. In one aspect, the composition comprises: (a) an exonuclease having 5′->3′ cleaving activity; (b) a primer set for amplifying a target nucleic acid; and (c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set. In some embodiments, said amplification is LAMP and the primer set comprises a forward outer primer (F3), a reverse outer primer (R3), a forward inner primer (FIP), and a reverse inner primer (RIP). In some embodiments, the primer set further comprises a forward loop primer (LF), and a reverse loop primer (LR).

In some embodiments of any of the aspects, the nucleic acid probe comprises further comprises a quencher molecule. In some embodiments, the quencher molecule quenches the detectable signal from the reporter molecule when the nucleic acid probe is not hybridized to a complementary nucleic acid strand. In some embodiments, the quencher molecule quenches the detectable signal from the reporter molecule when the nucleic acid probe is hybridized to a complementary nucleic acid strand. In some embodiments, the nucleic acid probe further comprises at least one additional quencher molecule.

In some embodiments of any of the aspects, the nucleic acid probe comprises a plurality of reporter molecules. In some embodiments, at least two reporter molecules in the plurality of reporter molecules are different. In some embodiments, the nucleic acid probe comprises at least one nucleic acid modification capable of increasing a melting temperature (Tm) of the nucleic acid probe for hybridizing with a complementary strand relative to a nucleic acid probe lacking said modification. In some embodiments, the nucleic acid probe comprises at least one nucleic acid modification capable of inhibiting extension by a polymerase.

In some embodiments, the nucleic acid probe comprises a nucleotide sequence substantially complementary to a primer used in the amplification of the target nucleic acid. In some embodiments, the nucleic acid probe comprises a nucleotide sequence substantially identical to a primer used in the amplification of the target nucleic acid. In some embodiments, the nucleic acid probe comprises a nucleotide sequence substantially complementary to a nucleotide sequence at an internal position of the amplicon.

In some embodiments, the nucleic acid probe comprises a first nucleic acid strand and a second nucleic acid strand, wherein the first strand comprises a region that is substantially complementary to a region in the second strand. In some embodiments, the first and second strand are linked to each other. In some embodiments, the nucleic acid probe forms a hairpin structure when hybridized to a complementary nucleic acid.

In some embodiments, the composition further comprises a reference or control nucleic acid. In some embodiments, the composition further comprises the target nucleic acid. In some embodiments, the composition further comprises reagents for preparing a double-stranded amplicon from the target nucleic acid. In some embodiments, the composition further comprises a double-stranded amplicon produced from the target nucleic acid. In some embodiments, the composition further comprises reagents for preparing a single-stranded amplicon from the target nucleic acid. In some embodiments, the composition further comprises a single-stranded amplicon produced from the target nucleic acid.

In one aspect, the composition comprises one or more of the following: (i) an exonuclease; (ii) a polymerase; (iii) a recombinase; (iv) single-stranded binding protein; (v) a first primer and optionally a second primer for amplification; (vi) one or more reagents for nucleic acid amplification; and (vii) an amplified nucleic acid. It is noted that a composition can comprise any one, two, three, four, five, six, or all seven of the components listed above. In one aspect, the composition comprises: (i) an exonuclease; (ii) a polymerase; (iii) a first primer and optionally a second primer for amplification; (iv) one or more reagents for nucleic acid amplification; and (v) an amplified nucleic acid.

In one aspect described herein is a composition comprising a first primer and a second primer for amplifying a target nucleic acid. In some embodiments of any of the aspects, the first primer comprises a nucleic acid modification capable of inhibiting 5′->3′ cleaving activity of a 5′->3′ exonuclease. In some embodiments of any of the aspects, the second primer comprises a nucleic acid modification that enhances 5′->3′ cleaving activity of the 5′->3′ exonuclease. Accordingly, in one aspect described herein is a composition comprising a first primer and a second primer for amplifying a target nucleic acid, wherein the first primer comprises a nucleic acid modification capable of inhibiting 5′->3′ cleaving activity of a 5′->3′ exonuclease, and the second primer optionally comprises a nucleic acid modification that enhances 5′->3′ cleaving activity of the 5′->3′ exonuclease.

In some embodiments of any of the aspects, the first primer comprises a nucleic acid modification capable of inhibiting 5′->3′ cleaving activity of a 5′->3′ exonuclease. In some embodiments of any of the aspects, the second primer comprises a nucleic acid modification capable of inhibiting 5′->3′ cleaving activity of a 5′->3′ exonuclease. In some embodiments of any of the aspects, the first and second primer independently comprises a nucleic acid modification capable of inhibiting 5′->3′ cleaving activity of a 5′->3′ exonuclease, which can be the same or different nucleic acid modification. In one aspect described herein is a composition comprising a first primer and a second primer for amplifying a target nucleic acid, wherein each of the first primer and second primer independently comprises a nucleic acid modification capable of inhibiting 5′->3′ cleaving activity of a 5′->3′ exonuclease.

In some embodiments of any of the aspects, the nucleic acid modification capable of inhibiting 5′-> 3′ cleaving activity of a 5′->3′ exonuclease is present at the 5′-end (e.g., of the first and/or second primer). In some embodiments of any of the aspects, the nucleic acid modification capable of inhibiting 5′-> 3′ cleaving activity of a 5′->3′ exonuclease is present at the 3′-end (e.g., of the first and/or second primer). In some embodiments of any of the aspects, the nucleic acid modification capable of inhibiting 5′-> 3′ cleaving activity of a 5′->3′ exonuclease is present at the 5′-end and the 3′ end (e.g., of the first and/or second primer), which can be the same or different nucleic acid modification. In some embodiments of any of the aspects, the nucleic acid modification capable of inhibiting 5′-> 3′ cleaving activity of a 5′->3′ exonuclease is present at an internal position (e.g., of the first and/or second primer). Non-limiting examples of such nucleic acid modifications are described further herein.

In some embodiments of any of the aspects, the composition further comprises one or more reagents for nucleic acid amplification. In some embodiments, the composition further comprises a DNA polymerase having strand displacement activity. In some embodiments, the composition further comprises dNTPs. In some embodiments, the composition further comprises a buffer. In some embodiments, the composition is in lyophilized form. In some embodiments, the composition further comprises at least one of the following: a reverse transcriptase, reaction buffer, diluent, water, magnesium salt (such as magnesium acetate or magnesium chloride) dNTPs, reducing agent (such as DTT), and/or an RNase inhibitor.

In some embodiments of any of the aspects, the composition further comprises a 5′->3′ exonuclease. In some embodiments of any of the aspects, the exonuclease is T7 exonuclease, lambda exonuclease, Exonuclease VIII, T5 exonuclease, RecJf, or any combinations thereof, as described further herein.

In some embodiments of any of the aspects, the composition further comprises a target nucleic acid for amplification. In some embodiments of any of the aspects, the target nucleic acid is a reference nucleic acid (e.g., a positive control such as a known nucleic acid sequence). In some embodiments of any of the aspects, the target nucleic acid is a target nucleic acid as described further herein, such as a viral RNA or a viral DNA.

In some embodiments of any of the aspects, the composition further comprises an amplicon produced by amplification of a target nucleic acid. In some embodiments of any of the aspects, the amplicon is double-stranded. In some embodiments of any of the aspects, the amplicon comprises a 5′-single-stranded overhang on at least one end. In some embodiments of any of the aspects, the amplicon comprises a 5′-single-stranded overhang on one end. In some embodiments of any of the aspects, the amplicon comprises a 5′-single-stranded overhang on both ends. Such a 5′-single-stranded overhang can be produced using methods as described further herein (e.g., stopper-based priming, digestion-based toehold exposure).

In some embodiments of any of the aspects, the amplicon is single stranded. Such a single stranded amplicon can be produced using methods as described further herein (e.g., 5′->3′ exonuclease digestion, asymmetrical amplification).

In one aspect described herein is a double-stranded nucleic acid comprising: (a) a first nucleic acid strand comprising a detectable label; and (b) a second nucleic acid probe comprising a ligand for a ligand binding molecule. In some embodiments of any of the aspects, the first nucleic acid strand and the second nucleic acid strands are substantially complementary to each other. Accordingly, in one aspect, described herein is a double-stranded nucleic acid comprising: (a) a first nucleic acid strand comprising a detectable label; and (b) a second nucleic acid probe comprising a ligand for a ligand binding molecule, wherein the first nucleic acid strand and the second nucleic acid strands are substantially complementary to each other. In some embodiments of any of the aspects, the first nucleic acid strand comprising a detectable label is produced using a method as described herein. Non-limiting examples of such detectable labels and ligands are described further herein. In one aspect described herein is a composition comprising a double-stranded nucleic acid as described herein.

In some embodiments of any of the aspects, the composition further comprises a ligand binding molecule capable of binding with the ligand. In some embodiments of any of the aspects, the ligand binding molecule is an antibody. In some embodiments of any of the aspects, the ligand binding molecule is an antibody that specifically binds to a ligand is selected from the group consisting of organic and inorganic molecules, peptides, polypeptides, proteins, peptidomimetics, glycoproteins, lectins, nucleosides, nucleotides, monosaccharides, disaccharides, trisaccharides, oligosaccharides, polysaccharides, lipopolysaccharides, vitamins, steroids, hormones, cofactors, receptors, receptor ligands, and analogs and derivatives thereof. In some embodiments of any of the aspects, the ligand is biotin, and the ligand-binding molecule is avidin or streptavidin. In some embodiments of any of the aspects, a ligand as described herein is used as a ligand-binding molecule, and a ligand binding molecule as described herein is used as a ligand.

In some embodiments of any of the aspects, the ligand and ligand-binding molecule are members of an affinity pair. In some embodiments of any of the aspects, the ligand and ligand-binding molecule are members of an affinity pair, selected from the group consisting of: a haptenic or antigenic compound in combination with a corresponding antibody or binding portion or fragment thereof; digoxigenin and anti-digoxigenin; mouse immunoglobulin and goat anti-mouse immunoglobulin; a non-immunological binding pair; biotin and avidin; biotin and streptavidin; a hormone and a hormone-binding protein; thyroxine and cortisol-hormone binding protein; a receptor and a receptor agonist; a receptor and a receptor antagonist; acetylcholine receptor and acetylcholine or an analog thereof; IgG and protein A; lectin and carbohydrate; an enzyme and an enzyme cofactor; an enzyme and an enzyme inhibitor; complementary oligonucleotide pairs capable of forming nucleic acid duplexes; and a first molecule that is negatively charged and a second molecule that is positively charged.

In some embodiments of any of the aspects, the ligand binding molecule can be immobilized or conjugated to a surface of various substrates. In some embodiments of any of the aspects, a composition as described herein further comprises such a substrate. Accordingly, a further aspect provided herein is a “nucleic acid detection substrate” or product for targeting or binding an amplicon of a target nucleic acid as described herein, comprising a substrate and at least one ligand binding molecule described herein, wherein the substrate comprises on its surface at least one, including at least two, at least three, at least four, at least five, at least ten, at least 25, at least 50, at least 100, at least 250, at least 500, or more ligand binding molecules. In some embodiments, the substrate can be conjugated or coated with at least one ligand binding molecules described herein, using any of conjugation methods described herein or any other art-recognized methods.

The solid substrate can be made from a wide variety of materials and in a variety of formats. For example, the solid substrate can be utilized in the form of beads (including polymer microbeads, magnetic microbeads, and the like), filters, fibers, screens, mesh, tubes, hollow fibers, scaffolds, plates, channels, other substrates commonly utilized in assay formats, and any combinations thereof. Non-limiting examples of a substrate include: a lateral flow strip; a nucleic acid scaffold; a protein scaffold; a lipid scaffold; a dendrimer; a microparticle; a microbead; a magnetic microbead; a paramagnetic microbead; medical apparatuses (e.g., needles or catheters) or medical implants; a microtiter plate; a microporous membrane; a microchip; a hollow fiber; a hollow fiber reactor or cartridge; a fluid filtration membrane; a fluid filtration device; a membrane; a diagnostic strip; a dipstick; an extracorporeal device; a mixing element (e.g., a spiral mixer); a microscopic slide; a flow device; a microfluidic device; a living cell; an extracellular matrix of a biological tissue or organ; or any combination thereof. The solid substrate can be made of any material, including, but not limited to, metal, metal alloy, polymer, plastic, paper, glass, fabric, packaging material, biological material such as cells, tissues, hydrogels, proteins, peptides, nucleic acids, and any combinations thereof.

In some embodiments of any of the aspects, the composition further comprises means for detecting the detectable label. In some embodiments of any of the aspects, said means for detecting the detectable label comprises lateral flow detection. In some embodiments of any of the aspects, said means for detecting the detectable label comprises LFIA. In some embodiments of any of the aspects, said means for detecting the detectable label comprises a detection method selected from: lateral flow detection; hybridization with conjugated or unconjugated DNA; colorimetric assays; gel electrophoresis; a toehold-mediated strand displacement reaction; molecular beacons; fluorophore-quencher pairs; microarrays; Specific High-sensitivity Enzymatic Reporter unLOCKing (SHERLOCK); DNA endonuclease-targeted CRISPR trans reporter (DETECTR); sequencing; and quantitative polymerase chain reaction (qPCR).

In some embodiments, one or more components of the composition is disposed in a device comprising two or more chambers and means for irreversibly moving a fluid from a first chamber to a second chamber. In some embodiments, the means for irreversibly moving the fluid from the first to the second chamber can be actuated by a built-in spring whose potential energy is released by a solenoid trigger. In some embodiments, the device further comprises means for detecting the detectable signal from the reporter molecule.

In some embodiments of any of the aspects, a composition described herein is in form of a kit.

### Kits

Another aspect of the technology described herein relates to kits for detecting a target nucleic acid. Described herein are kit components that can be included in one or more of the kits described herein. The kit can comprise any of the compositions provided herein and packaging and materials therefore.

In one aspect, the kit comprises a) an exonuclease having 5′->3′ cleaving activity; b) a primer set for amplifying a target nucleic acid; and c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set. In some embodiments, said amplification is LAMP and the primer set comprises a forward outer primer (F3), a reverse outer primer (R3), a forward inner primer (FIP), and a reverse inner primer (RIP). In some embodiments, the primer set further comprises a forward loop primer (LF), and a reverse loop primer (LR). In some embodiments, the nucleic acid probe(s) in the kit are selected from SEQ ID NOs: 51-55.

In another aspect, the kit comprises: (a) an exonuclease having 5′->3′ cleaving activity; (b) a primer set for amplifying a target nucleic acid by LAMP and wherein the primer set comprises a forward outer primer (F3), a reverse outer primer (R3), a forward inner primer (FIP), and a reverse inner primer (RIP); and (c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set.

In some embodiments of any of the aspects, the nucleic acid probe comprises further comprises a quencher molecule. In some embodiments, the quencher molecule quenches the detectable signal from the reporter molecule when the nucleic acid probe is not hybridized to a complementary nucleic acid strand. In some embodiments, the quencher molecule quenches the detectable signal from the reporter molecule when the nucleic acid probe is hybridized to a complementary nucleic acid strand. In some embodiments, the nucleic acid probe further comprises at least one additional quencher molecule.

In some embodiments of any of the aspects, the nucleic acid probe comprises a plurality of reporter molecules. In some embodiments, at least two reporter molecules in the plurality of reporter molecules are different. In some embodiments, the nucleic acid probe comprises at least one nucleic acid modification capable of increasing a melting temperature (Tm) of the nucleic acid probe for hybridizing with a complementary strand relative to a nucleic acid probe lacking said modification. In some embodiments, the nucleic acid probe comprises at least one nucleic acid modification capable of inhibiting extension by a polymerase.

In some embodiments, the nucleic acid probe comprises a nucleotide sequence substantially complementary to a primer in the primer set. In some embodiments, the nucleic acid probe comprises a nucleotide sequence substantially identical to a primer in the primer set. In some embodiments, the nucleic acid probe comprises a nucleotide sequence substantially complementary to a nucleotide sequence at an internal position of an amplicon prepared using the primer set.

In some embodiments, the nucleic acid probe comprises a first nucleic acid strand and a second nucleic acid strand, wherein the first strand comprises a region that is substantially complementary to a region in the second strand. In some embodiments, the first and second strand are linked to each other. In some embodiments, the nucleic acid probe forms a hairpin structure when hybridized to a complementary nucleic acid.

In some embodiments, the kit further comprises a reference or control nucleic acid. In some embodiments, the kit further comprises a lateral flow device for detecting the reporter molecule. In some embodiments, the kit further comprises means for detecting a detectable signal from the reporter molecule. In some embodiments, the kit further comprises a DNA polymerase having strand displacement activity.

In some embodiments of any of the aspects, the kit or compositions provided herein comprises one or more reaction mixture. In some embodiments of any of the aspects, the reaction mixture further comprises nucleotide triphosphates (NTPs) or deoxynucleotide triphosphates (dNTPs). In some embodiments, the reaction mixture further comprises a buffer. It is contemplated that buffer used in the reaction mixture is chosen that permit the stability of the nucleic acid probe and/or primers provided herein. Methods of choosing such buffers are known in the art and can also be chosen for their properties in various conditions including pH or temperature of the reaction being performed.

In one aspect, described herein is a kit for detecting a target nucleic acid in a sample, comprising: (a) an exonuclease; and (b) a DNA polymerase.

In one aspect, described herein is a kit for detecting a target nucleic acid in a sample, comprising: (a) an exonuclease; (b) a DNA polymerase; and (c) a first set of primers. In another aspect, described herein is a kit for detecting a target nucleic acid comprising (a) an exonuclease; (b) a DNA polymerase; (c) a first set of primers; (d) a recombinase; and (e) single-stranded DNA binding protein.

In some embodiments of any of the aspects, the kit is used to produce a target isothermal amplification product from the target nucleic acid and the first set of primers using an isothermal amplification reaction. In some embodiments, the kit further comprises reagents for preparing a double-stranded amplicon from the target nucleic acid. In some embodiments, the kit further comprises reagents for preparing a single-stranded amplicon from the target nucleic acid. In some embodiments of any of the aspects, the kit is used to produce a single stranded amplification product using the exonuclease.

In some embodiments of any of the aspects, the DNA polymerase is a strand-displacing DNA polymerase. In some embodiments of any of the aspects, the strand-displacing DNA polymerase is selected from the group consisting of: Polymerase I Klenow fragment, Bst polymerase, Phi-29 polymerase, and Bacillus subtilis Pol I (Bsu) polymerase. In some embodiments of any of the aspects, the kit comprises a sufficient amount of Polymerase I Klenow fragment, Bst polymerase, Phi-29 polymerase, and Bacillus subtilis Pol I (Bsu) polymerase. In some embodiments of any of the aspects, the DNA polymerase(s) is provided at a sufficient amount to be added to the reaction mixture.

In some embodiments of any of the aspects, the kit comprises at least one set of primers for isothermal amplification. In some embodiments of any of the aspects, the set of amplification primers is specific to the target RNA. In some embodiments of any of the aspects, the set of amplification primers is specific (i.e., binds specifically through complementarity) to cDNA, in other words, the DNA produced in the RT step that is complementary to the target RNA.

In some embodiments of any of the aspects, the kit further comprises a set of reverse transcription (RT) primers. In some embodiments of any of the aspects, the set of RT primers comprises primers that bind to target RNA and non-target RNA in the sample, i.e., “general” primers. In some embodiments of any of the aspects, the set of RT primers comprises random hexamers, i.e., a mixture of oligonucleotides representing all possible hexamer sequences. In some embodiments of any of the aspects, the set of RT primers comprises oligo(dT) primer, which bind to the polyA tails of mRNAs or viral transcripts.

In some embodiments of any of the aspects, the set of RT primers is specific to the target RNA. In some embodiments of any of the aspects, the set of RT primers comprises the reverse primer from the set of amplification primers. In some embodiments of any of the aspects, set of RT primers can comprise the set of amplification primers, or the set of amplification primers can comprise the set of RT primers.

In some embodiments of any of the aspects, the primers and/or probe(s) are provided at a sufficient concentration, e.g., 0.2 uM to 1.6 uM, e.g., 5 uM to 35 uM, to be added to reaction mixture. As a non-limiting example, the primers and/or probe(s) are provided at a concentration of at least 0.05 uM, at least 0.1 uM, at least 0.2 uM, at least 0.3 uM, at least 0.4 uM, at least 0.5 uM, at least 0.6 uM, at least 0.7 uM, at least 0.8 uM, at least 0.9 uM, at least 1 uM, at least 2 uM, at least 3 uM, at least 4 uM, at least 5 uM, at least 6 uM, at least 7 uM, at least 8 uM, at least 9 uM, at least 10 uM, at least 11 uM, at least 12 uM, at least 13 uM, at least 14 uM, at least 15 uM, at least 16 uM, at least 17 uM, at least 18 uM, at least 19 uM, at least 20 uM, at least 21 uM, at least 22 uM, at least 23 uM, at least 24 uM, at least 25 uM, at least 26 uM, at least 27 uM, at least 28 uM, at least 29 uM, at least 30 uM, at least 35 uM, at least 40 uM, at least 45 uM, at least or at least 50 uM.

In some embodiments of any of the aspects, the kit further comprises a recombinase and single-stranded DNA binding (SSB) protein. In some embodiments of any of the aspects, the single-stranded DNA-binding protein is a gp32 SSB protein. In some embodiments of any of the aspects, the recombinase is a uvsX recombinase. In some embodiments of any of the aspects, the recombinase and single-stranded DNA binding proteins are provided at a sufficient amount to be added to the reaction mixture. In some embodiments of any of the aspects, the kit comprises RPA pellets comprising RPA reagents (e.g., DNA polymerase, helicase, SSB) at a sufficient concentration. See e.g., U.S. Pat. 7,666,598, the content of which is incorporated herein by reference in its entirety.

In some embodiments of any of the aspects, the kit further comprises a reverse transcriptase. In some embodiments of any of the aspects, the kit is used to reverse transcribe target RNA into DNA, and to amplify the DNA to a detectable amplification product. In some embodiments of any of the aspects, the reverse transcriptase is selected from the group consisting of: a Moloney murine leukemia virus (M-MLV) reverse transcriptase (RT), an avian myeloblastosis virus (AMV) RT, a retrotransposon RT, a telomerase reverse transcriptase, an HIV-1 reverse transcriptase, or a recombinant version thereof. In some embodiments of any of the aspects, the reverse transcriptase is provided at a sufficient amount, such that at least 200 U/µL can be added to the reaction mixture.

In some embodiments of any of the aspects, the kit further comprises at least one of the following: reaction buffer, diluent, water, magnesium acetate (or another magnesium compound such as magnesium chloride) dNTPs, DTT, and/or an RNase inhibitor. In some embodiments of any of the aspects, the kit comprises a composition as described herein, e.g., a nucleic acid composition.

In some embodiments of any of the aspects, the kit further comprises reagents for isolating nucleic acid from the sample. In some embodiments of any of the aspects, the kit further comprises reagents for isolating DNA from the sample. In some embodiments of any of the aspects, the kit further comprises reagents for isolating RNA from the sample. In some embodiments of any of the aspects, the kit further comprises detergent, e.g., for lysing the sample. In some embodiments of any of the aspects, the kit further comprises a sample collection device, such a swab. In some embodiments of any of the aspects, the kit further comprises a sample collection container, optionally containing transport media.

In some embodiments of any of the aspects, the kit further comprises reagents for detecting the amplification product(s), comprising reagents appropriate for a detection method selected from: lateral flow detection; hybridization with conjugated or unconjugated DNA; colorimetric assays; gel electrophoresis; a toehold-mediated strand displacement reaction; molecular beacons; fluorophore-quencher pairs; microarrays; Specific High-sensitivity Enzymatic Reporter unLOCKing (SHERLOCK); DNA endonuclease-targeted CRISPR trans reporter (DETECTR); sequencing; and quantitative polymerase chain reaction (qPCR). In some embodiments of any of the aspects, the kit further comprises an additional set of primers and/or a detectable probe (e.g., for detection using qPCR, sequencing).

In some embodiments of any of the aspects, the kit further comprises reagents for amplifying and/or detecting a control. Non-limiting examples of negative controls for SARS-CoV-2 include MERS, SARS, 229e, NL63, and hKu1, which can be detected using specific primers. In some embodiments of any of the aspects, the kit further comprises one or more lateral flow strips specific for the target amplification product and/or at least one positive control. In some embodiments of any of the aspects, the kit further comprises a set of probes for detection through hybridization with a target amplification product.

In some embodiments, the kit further comprises a device comprising two or more chambers and means for irreversibly moving a fluid from a first chamber to a second chamber. In some embodiments, at least one component of the kit is disposed in a device comprising two or more chambers and means for irreversibly moving a fluid from a first chamber to a second chamber. In some embodiments, the means for irreversibly moving the fluid from the first to the second chamber can be actuated by a built-in spring whose potential energy is released by a solenoid trigger. In some embodiments, the device further comprises means for detecting the detectable signal from the reporter molecule, e.g., fluorescence detection, luminescence detection, chemiluminescence detection, colorimetric, or immunofluorescence detection.

In some embodiments, the kit comprises an effective amount of the reagents as described herein. As will be appreciated by one of skill in the art, the reagents can be supplied in a lyophilized form or a concentrated form that can diluted or suspended in liquid prior to use. The kit reagents described herein can be supplied in aliquots or in unit doses.

In some embodiments, the components described herein can be provided singularly or in any combination as a kit. Such a kit includes the components described herein and packaging materials thereof. In addition, a kit optionally comprises informational material.

In some embodiments, the compositions in a kit can be provided in a watertight or gas tight container which in some embodiments is substantially free of other components of the kit. For example, the reagents described herein can be supplied in more than one container, e.g., it can be supplied in a container having sufficient reagent for a predetermined number of applications, e.g., 1, 2, 3 or greater. One or more components as described herein can be provided in any form, e.g., liquid, dried or lyophilized form. Liquids or components for suspension or solution of the reagents can be provided in sterile form and should not contain microorganisms or other contaminants. When the components described herein are provided in a liquid solution, the liquid solution preferably is an aqueous solution.

The informational material can be descriptive, instructional, marketing or other material that relates to the methods described herein. The informational material of the kits is not limited in its form. In some embodiments, the informational material can include information about production of the reagents, concentration, date of expiration, batch or production site information, and so forth. In some embodiments, the informational material relates to methods for using or administering the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

In some embodiments of any of the aspects, the kit can further comprise a detection device. As a non-limiting example, a detection device can comprise a light-emitting diode (LED) light source and/or a filter (e.g., plastic filter specific for the emitting wavelength of a detectable marker). In some embodiments of any of the aspects, the kit and/or the detection device is field-deployable, i.e., transportable, non-refrigerated, and/or inexpensive. In some embodiments of any of the aspects, a detection device further comprises a wireless device (e.g., a cell phone, a personal digital assistant (PDA), a tablet).

### Systems

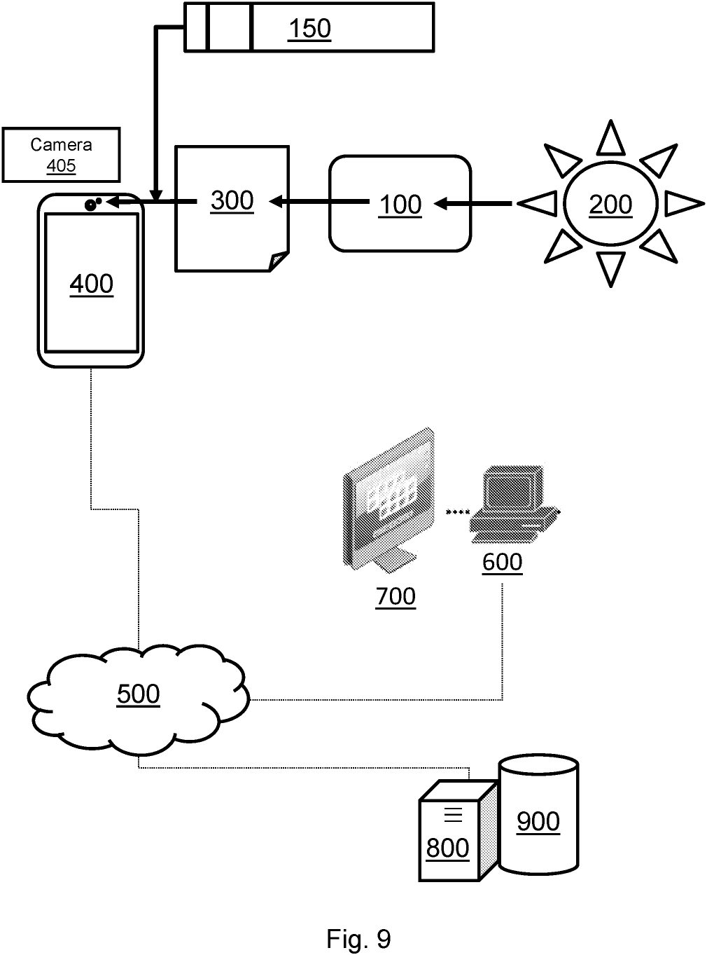

FIG. 9 shows an exemplary schematic of a system as described herein. As a non-limiting example, the amplification product as described herein can be detected using a plate-based assay 100 as described herein (e.g., SHERLOCK, DETECTR, microarray, hybridization, qPCR, sequencing, etc.). In embodiments where the assay is detected using detectable markers such as fluorophores, the results of the assay can be detected by exposing the detection assay 100 to a light source 200 (according to the specific excitation wavelength of a detection molecule in the assay) and a filter 300 (according to the specific emission wavelength of a detection molecule in the assay). The emitted wavelength of the detection molecule in the assay can be detected by the camera 405 of a portable computing device 400 (e.g., a mobile phone) or any other device comprising a camera 405. In some embodiments of any of the aspects, the amplification product is detected using a test strip 150 (e.g., using lateral flow detection and/or conjugated or unconjugated DNA). The colorimetric signals of the test strip 150 can be detected by the camera 405 of a portable computing device 400 (e.g., a mobile phone) or any other device comprising a camera 405.

The portable computing device 400 can be connected to a network 500. In some embodiments, the network 500 can be connected to another computing device 600 and/or a server 800. The network 500 can be connected to various other devices, servers, or network equipment for implementing the present disclosure. A computing device 600 can be connected to a display 700. Computing device 400 or 600 can be any suitable computing device, including a desktop computer, server (including remote servers), mobile device, or any other suitable computing device. In some examples, programs for implementing the system can be stored in database 900 and run on server 800. Additionally, data and data processed or produced by said programs can be stored in database 900.

It should initially be understood that the methods and systems described herein can be implemented with any type of hardware and/or software, and can include use of a pre-programmed general purpose computing device. For example, the system can be implemented using a server, a personal computer, a portable computer, a thin client, or any suitable device or devices. The kits, methods and/or components for the performance thereof can include the use of a single device at a single location, or multiple devices at a single, or multiple, locations that are connected together using any appropriate communication protocols over any communication medium such as electric cable, fiber optic cable, or in a wireless manner.

It should also be noted that the systems as described herein can be arranged or used in a format having a plurality of modules which perform particular functions. It should be understood that these modules are merely schematically illustrated based on their function for clarity purposes only, and do not necessary represent specific hardware or software. In this regard, these modules can be hardware and/or software implemented to substantially perform the particular functions discussed. Moreover, the modules can be combined together within the disclosure, or divided into additional modules based on the particular function desired. Thus, the disclosure should not be construed to limit the present technology as disclosed herein, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be performed in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of these. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program can, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA or an ASIC as noted above.

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from, or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

Various embodiments described herein comprise a single-stranded overhang. By a “single-stranded overhang” is meant that the strand extended beyond the 3′-end of the complementary strand. The single-strand overhang can be of any desired length. For example, each overhang independently can be 5 or more nucleotides in length, from about 5 nucleotides to about 20 nucleotides in length, from about 5 nucleotides to about 15 nucleotides in length, from about 10 nucleotides to about 25 nucleotides in length, from about 10 nucleotides to about 20 nucleotides in length, from about 15 nucleotides to about 25 nucleotides in length, or from about 15 nucleotides to about 20 nucleotides in length. In some embodiments, each overhang independently is 5, 6, 7, 8, 9, 10, 11, 12, 13, 14, 15, 16, 17, 18, 19, 20, 21, 22, 23, 24, 25 or more nucleotides in length.

When a single-strand overhang is present at both ends, they can be of same length or different length. For example, a first single strand overhang can be 5 or more nucleotides in length, from about 5 nucleotides to about 20 nucleotides in length, from about 5 nucleotides to about 15 nucleotides in length, from about 10 nucleotides to about 25 nucleotides in length, from about 10 nucleotides to about 20 nucleotides in length, from about 15 nucleotides to about 25 nucleotides in length, or from about 15 nucleotides to about 20 nucleotides in length. In some embodiments of the various aspects describe herein, the first overhang is 5, 6, 7, 8, 9, 10, 11, 12, 13, 14, 15, 16, 17, 18, 19, 20, 21, 22, 23, 24, 25 or more nucleotides in length.

Similarly, the second single strand overhang can be 5 or more nucleotides in length, from about 5 nucleotides to about 20 nucleotides in length, from about 5 nucleotides to about 15 nucleotides in length, from about 10 nucleotides to about 25 nucleotides in length, from about 10 nucleotides to about 20 nucleotides in length, from about 15 nucleotides to about 25 nucleotides in length, or from about 15 nucleotides to about 20 nucleotides in length. In some embodiments of the various aspects describe herein, the second overhang is 5, 6, 7, 8, 9, 10, 11, 12, 13, 14, 15, 16, 17, 18, 19, 20, 21, 22, 23, 24, 25 or more nucleotides in length.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of viral infection. A subject can be male or female.

A subject can be one who has been previously diagnosed with or identified as suffering from or having a condition in need of treatment (e.g. a viral infection) or one or more complications related to such a condition, and optionally, have already undergone treatment for a viral infection or the one or more complications related to a viral infection. Alternatively, a subject can also be one who has not been previously diagnosed as having a viral infection or one or more complications related to a viral infection. For example, a subject can be one who exhibits one or more risk factors for a viral infection or one or more complications related to a viral infection or a subject who does not exhibit risk factors. A “subj ect in need” of testing for a particular condition can be a subject having that condition, diagnosed as having that condition, or at risk of developing that condition.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested confirm that a desired activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide’s activity according to the assays described herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan to generate and test artificial variants.

A variant DNA or amino acid sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments, the methods described herein relate to measuring, detecting, or determining the level of at least one target, e.g., the target nucleic acid. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation. Sequence determination, e.g., that indicates or confirms the presence of a given sequence element, e.g., a barcode element or region thereof, is a form of detecting.

In some embodiments of any of the aspects, a polypeptide, nucleic acid, cell, or microorganism as described herein can be engineered. As used herein, “engineered” refers to the aspect of having been manipulated by the hand of man. For example, a polynucleotide is considered to be “engineered” when at least one aspect of the polynucleotide, e.g., its sequence, has been manipulated by the hand of man to differ from the aspect as it exists in nature.

As used herein, “contacting” refers to any suitable means for delivering, or exposing, an agent to at least one component as described herein (e.g., sample, a target nucleic acid, target RNA, cDNA, amplification product, etc.). In some embodiments, contacting comprises physical human activity, e.g., an injection; an act of dispensing, mixing, and/or decanting; and/or manipulation of a delivery device or machine.

As used herein, the term “hybridizing”, “hybridize”, “hybridization”, “annealing”, or “anneal” are used interchangeably in reference to the pairing of complementary nucleic acids using any process by which a strand of nucleic acid joins with a complementary strand through base pairing to form a hybridization complex. In other words, the term “hybridization” refers to the process in which two single-stranded polynucleotides bind non-covalently to form a stable double-stranded polynucleotide. Furthermore, the term “hybridize” refers to the phenomenon of a single-stranded nucleic acid or region thereof forming hydrogen-bonded base pair interactions with either another single stranded nucleic acid or region thereof (intermolecular hybridization) or with another single-stranded region of the same nucleic acid (intramolecular hybridization). Hybridization is governed by the base sequences involved, with complementary nucleobases forming hydrogen bonds, and the stability of any hybrid being determined by the identity of the base pairs (e.g., G:C base pairs being stronger than A:T base pairs) and the number of contiguous base pairs, with longer stretches of complementary bases forming more stable hybrids. The term “hybridization” may also refer to triple-stranded hybridization. The resulting (usually) double-stranded polynucleotide is a “hybrid” or “duplex.”

In some embodiments of the various aspects described herein, the step of hybridizing the probe with the amplified product comprises heating and/or cooling. For example, a reaction comprising the amplified product and the probe can be heated and then cooled to promote hybridization.

It is noted that the hybridization step can be carried out in the same reaction vessel used for preparing the amplified product. Alternatively, the amplified product can be isolated or purified from the amplification reaction prior to the hybridization step. In other words, the amplification step and the hybridization steps are in different reaction vessels.

“Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tm for the specific sequence at a defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5 × SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1st Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

The term “substantially identical” means two or more nucleotide sequences have at least 65%, 70%, 80%, 85%, 90%, 95%, or 97% identical nucleotides. In some embodiments, “substantially identical” means two or more nucleotide sequences have the same identical nucleotides.

The term “substantial complementary” or “substantially complementary” as used herein refers both to complete complementarity of binding nucleic acids, in some cases referred to as an identical sequence, as well as complementarity sufficient to achieve the desired binding of nucleic acids. Correspondingly, the term “complementary hybrids” encompasses substantially complementary hybrids.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50 oC or 70 oC for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

As used herein, the term “complementary,” in the context of an oligonucleotide (i.e., a sequence of nucleotides such as an oligonucleotide primers or a target nucleic acid) refers to standard Watson/Crick base pairing rules. For example, the sequence “5′-A-G-T-C-3′” is complementary to the sequence “3′-T-C-A-G-5′.” Certain nucleotides not commonly found in natural nucleic acids or chemically synthesized may be included in the nucleic acids described herein; these include but not limited to base and sugar modified nucleosides, nucleotides, and nucleic acids, such as inosine, isocytosine and isoguanine. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogsteen base pairing. In other words, complementarity need not be perfect; stable duplexes may contain mismatched base pairs, degenerative, or unmatched nucleotides. Those skilled in the art of nucleic acid technology can determine duplex stability empirically considering a number of variables including, for example, the length of the oligonucleotide, base composition and sequence of the oligonucleotide, incidence of mismatched base pairs, ionic strength, other hybridization buffer components and conditions.

Complementarity may be partial in which only some of the nucleotide bases of two nucleic acid strands are matched according to the base pairing rules. Complementarity may be complete or total where all of the nucleotide bases of two nucleic acid strands are matched according to the base pairing rules. Complementarity may be absent where none of the nucleotide bases of two nucleic acid strands are matched according to the base pairing rules. In some embodiments of any of the aspects, two nucleic acid strands are at least 50%, at least 55%, at least 60%, at least 65%, at least 70%, at least 75%, at least 80%, at least 85%, at least 90%, at least 95%, at least 96%, at least 97%, at least 98%, at least 99%, or 100% complementary. The degree of complementarity between nucleic acid strands has significant effects on the efficiency and strength of hybridization between nucleic acid strands. This is of particular importance in detection methods that depend upon binding between nucleic acids.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third non-target entity. A reagent specific for a given target is one that exhibits specific binding for that target under the conditions of the assay being utilized.

As used herein, the term “oligonucleotide” is intended to include, but is not limited to, a single-stranded DNA or RNA molecule, typically prepared by synthetic means. Nucleotides of the present invention will typically be the naturally-occurring nucleotides such as nucleotides derived from adenosine, guanosine, uridine, cytidine and thymidine. When oligonucleotides are referred to as “double-stranded,” it is understood by those of skill in the art that a pair of oligonucleotides exists in a hydrogen-bonded, helical array typically associated with, for example, DNA. In addition to the 100% complementary form of double-stranded oligonucleotides, the term “double-stranded” as used herein is also meant to include those form which include such structural features as bulges and loops (see Stryer, Biochemistry, Third Ed. (1988), incorporated herein by reference in its entirety for all purposes).

The term “statistically significant” or “significantly” refers to statistical significance and generally means a two standard deviations (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%. In some embodiments of any of the aspects, the term “about” when used in connection with percentages can mean ±5% (e.g., ±4%, ±3%, ±2%, ±1%) of the value being referred to.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Where a range of values is provided, each numerical value between the upper and lower limits of the range is contemplated and disclosed herein.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway’s Immunobiology, Kenneth Murphy, Allan Mowat, Casey Weaver (eds.), W. W. Norton & Company, 2016 (ISBN 0815345054, 978-0815345053); Lewin’s Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It will be apparent to those skilled in the relevant art that various modifications, additions, substitutions, and the like can be performed without altering the spirit or scope of the invention, and such modifications and variations are encompassed within the scope of the invention as defined in the claims which follow.

Some embodiments of the technology described herein can be defined according to any of the following numbered paragraphs:

1. A method for preparing a single-stranded amplicon from a target nucleic acid, the method comprising: (a) amplifying a target nucleic acid with a first primer and a second primer to produce a double-stranded amplicon, wherein: (i) the first primer comprises a nucleic acid modification capable of inhibiting 5′->3′ cleaving activity of a 5′->3′ exonuclease; and (ii) the second primer optionally comprises a nucleic acid modification that enhances 5′->3′ cleaving activity of the 5′->3′ exonuclease; and (b) contacting the double-stranded amplicon from step (a) with the 5′->3′ exonuclease.

2. The method of paragraph 1, wherein the nucleic acid modification capable of inhibiting 5′-> 3′ cleaving activity of a 5′->3′ exonuclease is selected from the group consisting of modified internucleotide linkages modified nucleobase, modified sugar, and any combinations thereof.

3. The method of paragraph 1 or 2, wherein the first primer comprises: (a) 1, 2, 3, 4, 5, 6 or more modified internucleotide linkages; (b) an inverted nucleoside or 5′->5′ internucleotide linkage; (c) a 2′-OH or a 2′-modified nucleoside; (d) a 5′-modified nucleotide and/or a 3′ modified nucleotide; (e) a 2′->5′ linkage; (f) an abasic nucleoside; (g) an acyclic nucleoside; (h) a spacer; (i) left-handed DNA; and (j) any combinations of (a)-(j).

4. The method of paragraph 3, wherein said modified internucleotide linkages are selected from the group consisting of phosphorothioates, phosphorodithioates, phosphotriesters, alkylphosphonates, phosphoramidate, phosphoroselenates, borano phosphates, borano phosphate esters, hydrogen phosphonates, alkyl or aryl phosphonates, bridged phosphoroamidates, bridged phosphorothioates, bridged alkylenephosphonates, methylenemethylimino (—CH2—N(CH3)—O—CH2—), thiodiester (—O—C(O)—S—), thionocarbamate (—O—C(O)(NH)—S—), siloxane (—O—Si(H)2—O—), and N,N′-dimethylhydrazine (—CH2—N(CH3)—N(CH3)—), amide-3 (3′—CH₂—C(═O)—N(H)—5′), amide-4 (3′—CH₂—N(H)—C(═O)—5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′—S—CH₂—O—5′), formacetal (3′—O—CH₂—O—5′), oxime, methyleneimino, methykenecarbonylamino, methylenemethylimino (MMI, 3′—CH₂—N(CH₃)—O—5′), methylenehydrazo, methylenedimethylhydrazo, methyleneoxymethylimino, ethers (C3′—O—C5′), thioethers (C3′—S—C5′), thioacetamido (C3′—N(H)—C(═O)—CH₂—S—C5′, C3′—O—P(O)—O—SS—C5′, C3′—CH₂—NH—NH—C5′, 3′—NHP(O)(OCH₃)—O—5′ and 3′—NHP(O)(OCH₃)—O—5′.

5. The method of paragraph 4, wherein said modified internucleotide linkages are phosphorothioate.

6. The method of any one of paragraphs 3-5, wherein said 2′-modified nucleoside comprises a modification selected from the group consisting of 2′-halo (e.g., 2′-fluoro), 2′-alkoxy (e.g., 2′-Omethyl, 2′-Omethylmethoxy and 2′-Omethylethoxy), 2′-aryloxy, 2′-O-amine or 2′-O-alkylamine (amine NH₂; alkylamino, dialkylamino, heterocyclyl, arylamino, diaryl amino, heteroaryl amino, dihet.eroaryl amino, ethylene diamine or polyamino), O-CH₂CH₂(NCH₂CH₂NMe₂)₂, methyleneoxy (4′—CH₂—O—2′) LNA, ethyleneoxy (4′—(CH₂)₂—O—2′) ENA, 2′-amino (e.g. 2′-NH₂, 2′-alkylamino, 2′-dialkylamino, 2′-heterocyclylamino, 2′-arylamino, 2′-diaryl amino, 2′-heteroaryl amino, 2′-diheteroaryl amino, and 2′-amino acid); NH(CH₂CH₂NH)ₙCH₂CH₂-AMINE (AMINE = NH₂, alkylamino, dialkylamino, heterocyclyl, arylamino, diaryl amino, heteroaryl amino, or diheteroaryl amino), —NHC(O)R (R = alkyl, cycloalkyl, aryl, aralkyl, heteroaryl or sugar), 2′-cyano, 2′-mercapto, 2′-alkyl-thio-alkyl, 2′-thioalkoxy, 2′-thioalkyl, 2′-alkyl, 2′-cycloalkyl, 2′-aryl, 2′-alkenyl and 2′-alkynyl.

7. The method of any one of paragraphs 3-6, wherein the inverted nucleoside is dT.

8. The method of any one of paragraphs 3-7, wherein the 5′-modified nucleotide comprises a 5′-modification selected from the group consisting of 5′-monothiophosphate (phosphorothioate), 5′-monodithiophosphate (phosphorodithioate), 5′-phosphorothiolate, 5′-alpha-thiotriphosphate, 5′-beta-thiotriphosphate, 5′-gamma-thiotriphosphate, 5′-phosphoramidates, 5′-alkylphosphonate, 5′-alkyletherphosphonate, a detectable label, and a ligand; or the 3′-modified nucleotide comprises a 3′-modification selected from the group consisting of 3′-monothiophosphate (phosphorothioate), 3′-monodithiophosphate (phosphorodithioate), 3′-phosphorothiolate, 3′-alpha-thiotriphosphate, 3′-beta-thiotriphosphate, 3′-gamma-thiotriphosphate, 3′-phosphoramidates, 3′-alkylphosphonate, 3′-alkyletherphosphonate, a detectable label, and a ligand.

9. The method of any one of paragraphs 3-8, wherein the 5′-modified nucleotide comprises a detectable label at the 5′-end.

10. The method of any one of paragraphs 1-9, wherein the second primer comprises a 5′-OH or a phosphate group at the 5′-end.

11. The method of any of paragraphs 1-10, wherein the second primer comprises a 5′-monophosphate; 5′-diphosphate or a 5′-triphosphate at the 5′-end.

12. The method of any one of paragraphs 1-11, wherein the exonuclease is T7 exonuclease, lambda exonuclease, Exonuclease VIII, T5 exonuclease, RecJf, or any combinations thereof.

13. The method of any one of paragraphs 1-12, wherein said amplifying of step (a) comprises isothermal amplification, selected from the group consisting of: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3SR), Single primer isothermal amplification (SPIA), and cross-priming amplification (CPA).

14. The method of any one of paragraphs 1-13, wherein said amplifying of step (a) comprises recombinase polymerase amplification (RPA), Loop Mediated Isothermal Amplification (LAMP), or Helicase-dependent isothermal DNA amplification (HDA).

15. The method of any one of paragraphs 1-14, wherein the target nucleic acid is single-stranded.

16. The method of any one of paragraphs 1-14, wherein the target nucleic acid is double-stranded.

17. The method of any one of paragraphs 1-16, wherein the target nucleic acid is RNA.

18. The method of any one of paragraphs 1-17, wherein the target nucleic acid is a viral RNA.

19. The method of any one of paragraphs 1-15, wherein the target nucleic acid is DNA.

20. The method of any one of paragraphs 1-19, wherein the target nucleic acid is a viral DNA.

21. The method of any one paragraphs 1-20, further comprising a step of heating the double-stranded amplicon prior to contacting with the exonuclease.

22. The method of any one of paragraphs 1-21, further comprising a step of detecting the single-stranded amplicon after step (b).

23. The method of paragraph 22, wherein said detection is selected from the group consisting of: lateral flow detection; hybridization with conjugated or unconjugated DNA; colorimetric assays; gel electrophoresis; a toehold-mediated strand displacement reaction; molecular beacons; fluorophore-quencher pairs; microarrays; sequencing; and quantitative polymerase chain reaction (qPCR).

24. The method of paragraph 22, wherein said detecting comprises: (a) hybridizing the single-stranded amplicon with a first nucleic acid probe and a second nucleic acid probe to form a complex, wherein: (i) the first nucleic acid probe comprises a first detectable label; and (ii) the second nucleic acid probe comprises a ligand for a ligand binding molecule; and (b) detecting presence of the complex.

25. The method of paragraph 24, wherein at least one of the first and second nucleic acid probe hybridizes at an inner region of the single-stranded amplicon.

26. The method of paragraph 24 or 25, wherein the detectable label is selected from the group consisting of a light-absorbing dye, a fluorescent dye, a luminescent or bioluminescent molecule, a quantum dot, a radiolabel, an enzyme, a colorimetric label.

27. The method of any one of paragraphs 24-26, wherein the detectable label is colorimetric label selected from the group consisting of colloidal gold, colored glass or plastic beads, and any combinations thereof.

28. The method of paragraph 27, wherein the detectable label is a gold nanoparticle or a latex bead.

29. The method of any one of paragraphs 24-28, wherein the ligand is selected from the group consisting of organic and inorganic molecules, peptides, polypeptides, proteins, peptidomimetics, glycoproteins, lectins, nucleosides, nucleotides, monosaccharides, disaccharides, trisaccharides, oligosaccharides, polysaccharides, lipopolysaccharides, vitamins, steroids, hormones, cofactors, receptors, receptor ligands, and analogs and derivatives thereof.

30. The method of any one of paragraphs 24-29, wherein the ligand is biotin.

31. The method of any one of paragraphs 24-30, wherein the ligand binding molecule is an antibody.

32. The method of any one of paragraphs 24-31, wherein said detecting is by lateral flow detection.

33. A method for preparing a single-stranded amplicon from a target nucleic acid, wherein the method comprises: (a) amplifying a target nucleic acid with a first primer and a second primer to produce a double-stranded amplicon, wherein the double-stranded amplicon comprises a 5′-single-stranded overhang on at least one end; and (b) contacting the double-stranded amplicon of step (a) with a nucleic acid probe comprising a sequence substantially complementary to the single-strand overhang, whereby the nucleic acid probe hybridizes with the complementary single-strand overhang and releases the non-complementary, to the probe, strand as a single-stranded amplicon.

34. The method of paragraph 33, wherein at least one or both of the first or second primer comprises, at an internal position, a nucleic acid modification capable of inhibiting 5′->3′ cleaving activity of a 5′->3′ exonuclease and the method further comprises contacting the double-stranded amplicon with the 5′->3′ exonuclease prior to contacting with the nucleic acid probe.

35. The method of paragraph 34, wherein the nucleic acid modification capable of inhibiting 5′-> 3′ cleaving activity of a 5′->3′ exonuclease is selected from the group consisting of modified internucleotide linkages modified nucleobase, modified sugar, and any combinations thereof.

36. The method of any one of paragraphs 33-35, wherein at least one or both of the first or second primer comprises, at an internal position: (a) a modified internucleotide linkage; (b)an inverted nucleoside, a 5′->5′ internucleotide linkage or a 3′->3′ internucleotide linkage; (c) a 2′-OH or a 2′-modified nucleoside; (d) a 2′->5′ linkage; (e) an abasic nucleoside; (f) an acyclic nucleoside; (g) a spacer; and (h) any combinations of (a)-(g).

37. The method of paragraph 36, wherein said modified internucleotide linkage is selected from the group consisting of phosphorothioates, phosphorodithioates, phosphotriesters, alkylphosphonates, phosphoramidate, phosphoroselenates, borano phosphates, borano phosphate esters, hydrogen phosphonates, alkyl or aryl phosphonates, bridged phosphoroamidates, bridged phosphorothioates, bridged alkylenephosphonates, methylenemethylimino (—CH2—N(CH3)—O—CH2—), thiodiester (—O—C(O)—S—), thionocarbamate (—O—C(O)(NH)—S—), siloxane (—O—Si(H)2—O—), and N,N′-dimethylhydrazine (—CH2—N(CH3)—N(CH3)—), amide-3 (3′—CH₂—C(═O)—N(H)—5′), amide-4 (3′—CH₂—N(H)—C(═O)—5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′—S—CH₂—O—5′), formacetal (3′—O—CH₂—O—5′), oxime, methyleneimino, methykenecarbonylamino, methylenemethylimino (MMI, 3′—CH₂—N(CH₃)—O—5′), methylenehydrazo, methylenedimethylhydrazo, methyleneoxymethylimino, ethers (C3′—O—C5′), thioethers (C3′—S—C5′), thioacetamido (C3′—N(H)—C(═O)—CH₂—S—C5′, C3′—O—P(O)—O—SS—C5′, C3′—CH₂—NH—NH—C5′, 3′—NHP(O)(OCH₃)—O—5′ and 3′—NHP(O)(OCH₃)—O—5′.

38. The method of paragraph 37, wherein said modified internucleotide linkage is phosphorothioate.

39. The method of any one of paragraphs 33-38, wherein at least one or both of the first or second primer independently comprises a 2′-OH nucleoside or a 2′-modified nucleoside comprising a modification selected from the group consisting of 2′-halo (e.g., 2′-fluoro), 2′-alkoxy (e.g., 2′-Omethyl, 2′-Omethylmethoxy and 2′-Omethylethoxy), 2′-aryloxy, 2′-O-amine or 2′-O-alkylamine (amine NH2; alkylamino, dialkylamino, heterocyclyl, arylamino, diaryl amino, heteroaryl amino, dihet.eroaryl amino, ethylene diamine or polyamino), O-CH₂CH₂(NCH₂CH₂NMe₂)₂, methyleneoxy (4′-CH₂-O-2′) LNA, ethyleneoxy (4′-(CH₂)₂-O-2′) ENA, 2′-amino (e.g. 2′-NH₂, 2′-alkylamino, 2′-dialkylamino, 2′-heterocyclylamino, 2′-arylamino, 2′-diaryl amino, 2′-heteroaryl amino, 2′-diheteroaryl amino, and 2′-amino acid); NH(CH₂CH₂NH)ₙCH₂CH₂-AMINE (AMINE = NH₂, alkylamino, dialkylamino, heterocyclyl, arylamino, diaryl amino, heteroaryl amino, or diheteroaryl amino), —NHC(O)R (R = alkyl, cycloalkyl, aryl, aralkyl, heteroaryl or sugar), 2′-cyano, 2′-mercapto, 2′-alkyl-thio-alkyl, 2′-thioalkoxy, 2′-thioalkyl, 2′-alkyl, 2′-cycloalkyl, 2′-aryl, 2′-alkenyl and 2′-alkynyl.

40. The method of paragraph 39, wherein at least one or both of the first or second primer comprises a 2′-OH nucleoside.

41. The method of any one of paragraphs 33-40, wherein at least one or both of the first or second primer comprises a 5′-OH or a phosphate group at the 5′-end.

42. The method of any of paragraphs 33-41, wherein at least one or both of the first or second primer comprises a 5′-monophosphate; 5′-diphosphate or a 5′-triphosphate at the 5′-end.

43. The method of any one of paragraphs 33-42, wherein the exonuclease is T7 exonuclease, lambda exonuclease, Exonuclease VIII, T5 exonuclease, RecJf, or any combinations thereof.

44. The method of any one of paragraphs 33-43, wherein said amplifying of step (a) comprises isothermal amplification, selected from the group consisting of: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3SR), Single primer isothermal amplification (SPIA), and cross-priming amplification (CPA).

45. The method of any one of paragraphs 33-44, wherein said amplifying of step (a) comprises recombinase polymerase amplification (RPA), Loop Mediated Isothermal Amplification (LAMP), or Helicase-dependent isothermal DNA amplification (HDA).

46. The method of any one of paragraphs 33-45, wherein the target nucleic acid is single-stranded.

47. The method of any one of paragraphs 33-46, wherein the target nucleic acid is double-stranded.

48. The method of any one of paragraphs 33-47, wherein the target nucleic acid is RNA.

49. The method of any one of paragraphs 33-48, wherein the target nucleic acid is a viral RNA.

50. The method of any one of paragraphs 33-47, wherein the target nucleic acid is DNA.

51. The method of any one of paragraphs 33-47, wherein the target nucleic acid is a viral DNA.

52. The method of any one paragraphs 33-51, further comprising a step of heating the double-stranded amplicon prior to contacting with the exonuclease.

53. The method of any one of paragraphs 33-52, further comprising a step of detecting the single-stranded amplicon after step (b).

54. The method of paragraph 53, wherein said detection is selected from the group consisting of: lateral flow detection; hybridization with conjugated or unconjugated DNA; colorimetric assays; gel electrophoresis; a toehold-mediated strand displacement reaction; molecular beacons; fluorophore-quencher pairs; microarrays; sequencing; and quantitative polymerase chain reaction (qPCR)

55. The method of paragraph 53, wherein said detecting comprises: (a) hybridizing the single-stranded amplicon with a first nucleic acid probe and a second nucleic acid probe to form a complex, wherein: (i) the first nucleic acid probe comprises a first detectable label; and (ii) the second nucleic acid probe comprises a ligand for a ligand binding molecule; and (b) detecting presence of the complex.

56. The method of paragraph 55, wherein at least one of the first and second nucleic acid probe hybridizes at an inner region of the single-stranded amplicon.

57. The method of paragraph 55 or 56, wherein the detectable label is selected from the group consisting of a light-absorbing dye, a fluorescent dye, a luminescent or bioluminescent molecule, a quantum dot, a radiolabel, an enzyme, a calorimetric label.

58. The method of any one of paragraphs 55-57, wherein the detectable label is colorimetric label selected from the group consisting of colloidal gold, colored glass or plastic beads, and any combinations thereof.

59. The method of paragraph 58, wherein the detectable label is a gold nanoparticle or a latex bead.

60. The method of any one of paragraphs 55-59, wherein the ligand is selected from the group consisting of organic and inorganic molecules, peptides, polypeptides, proteins, peptidomimetics, glycoproteins, lectins, nucleosides, nucleotides, monosaccharides, disaccharides, trisaccharides, oligosaccharides, polysaccharides, lipopolysaccharides, vitamins, steroids, hormones, cofactors, receptors, receptor ligands, and analogs and derivatives thereof.

61. The method of any one of paragraphs 55-60, wherein the ligand is biotin.

62. The method of any one of paragraphs 55-61, wherein the ligand binding molecule is an antibody.

63. The method of any one of paragraphs 55-62, wherein said detecting is by lateral flow detection.

64. A method for preparing a single-stranded amplicon from a target nucleic acid, wherein the method comprises: (a) amplifying a target nucleic acid with a first primer and a second primer to produce a double-stranded amplicon, wherein the double-stranded amplicon comprises a 5′-single-stranded overhang on at least one end; and (b) contacting the double-stranded amplicon of step (a) with a nucleic acid probe comprising a sequence substantially complementary to the single-strand overhang, whereby the nucleic acid probe hybridizes with the complementary single-strand overhang and releases the non-complementary, to the probe, strand as a single-stranded amplicon.

65. The method of paragraph 64, wherein at least one or both of the first or second primer comprises a nucleic acid modification capable of inhibiting synthesis of a complementary strand by a polymerase.

66. The method of paragraph 65, wherein the nucleic acid modification capable of inhibiting synthesis of a complementary strand by a polymerase is a non-canonical base or a spacer.

67. The method of paragraph 64 or 65, wherein at least one or both of the first or second primer comprises a secondary structure that inhibits synthesis of a complementary strand by a polymerase.

68. The method of any one of paragraphs 64-67, wherein said amplifying of step (a) comprises isothermal amplification, selected from the group consisting of: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3SR), Single primer isothermal amplification (SPIA), and cross-priming amplification (CPA).

69. The method of any one of paragraphs 64-68, wherein said amplifying of step (a) comprises recombinase polymerase amplification (RPA), Loop Mediated Isothermal Amplification (LAMP), or Helicase-dependent isothermal DNA amplification (HDA).

70. The method of any one of paragraphs 64-69, wherein the target nucleic acid is single-stranded.

71. The method of any one of paragraphs 64-70, wherein the target nucleic acid is double-stranded.

72. The method of any one of paragraphs 64-71, wherein the target nucleic acid is RNA.

73. The method of any one of paragraphs 64-72, wherein the target nucleic acid is a viral RNA.

74. The method of any one of paragraphs 64-71, wherein the target nucleic acid is DNA.

75. The method of any one of paragraphs 64-71, wherein the target nucleic acid is a viral DNA.

76. The method of any one of paragraphs 64-75, further comprising a step of detecting the single-stranded amplicon after step (b).

77. The method of paragraph 76, wherein said detection is selected from the group consisting of: lateral flow detection; hybridization with conjugated or unconjugated DNA; colorimetric assays; gel electrophoresis; a toehold-mediated strand displacement reaction; molecular beacons; fluorophore-quencher pairs; microarrays; sequencing; and quantitative polymerase chain reaction (qPCR)

78. The method of paragraph 76, wherein said detecting comprises: (a) hybridizing the single-stranded amplicon with a first nucleic acid probe and a second nucleic acid probe to form a complex, wherein: (i) the first nucleic acid probe comprises a first detectable label; and (ii) the second nucleic acid probe comprises a ligand for a ligand binding molecule; and (b) detecting presence of the complex.

79. The method of paragraph 77, wherein at least one of the first and second nucleic acid probe hybridizes at an inner region of the single-stranded amplicon.

80. The method of paragraph 77 or 78, wherein the detectable label is selected from the group consisting of a light-absorbing dye, a fluorescent dye, a luminescent or bioluminescent molecule, a quantum dot, a radiolabel, an enzyme, a colorimetric label.

81. The method of any one of paragraphs 77-80, wherein the detectable label is colorimetric label selected from the group consisting of colloidal gold, colored glass or plastic beads, and any combinations thereof.

82. The method of paragraph 81, wherein the detectable label is a gold nanoparticle or a latex bead.

83. The method of any one of paragraphs 76-82, wherein the ligand is selected from the group consisting of organic and inorganic molecules, peptides, polypeptides, proteins, peptidomimetics, glycoproteins, lectins, nucleosides, nucleotides, monosaccharides, disaccharides, trisaccharides, oligosaccharides, polysaccharides, lipopolysaccharides, vitamins, steroids, hormones, cofactors, receptors, receptor ligands, and analogs and derivatives thereof.

84. The method of any one of paragraphs 76-83, wherein the ligand is biotin.

85. The method of any one of paragraphs 76-84, wherein the ligand binding molecule is an antibody.

86. The method of any one of paragraphs 76-85, wherein said detecting is by lateral flow detection.

87. A method for detecting a nucleic acid target, wherein the method comprises: (a) asymmetrically amplifying a target nucleic acid to produce a single-stranded amplicon; and (b) detecting presence of the single-stranded amplicon.

88. The method of paragraph 87, wherein said asymmetrically amplifying of step (a) comprises isothermal amplification, selected from the group consisting of: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3SR), Single primer isothermal amplification (SPIA), and cross-priming amplification (CPA).

89. The method of paragraph 87 or 88, wherein said asymmetrically amplifying of step (a) comprises recombinase polymerase amplification (RPA), Loop Mediated Isothermal Amplification (LAMP), or Helicase-dependent isothermal DNA amplification (HDA).

90. The method of any one of paragraphs 87-89, wherein said detection is selected from the group consisting of: lateral flow detection; hybridization with conjugated or unconjugated DNA; colorimetric assays; gel electrophoresis; a toehold-mediated strand displacement reaction; molecular beacons; fluorophore-quencher pairs; microarrays; sequencing; and quantitative polymerase chain reaction (qPCR)

91. The method of any one of paragraphs 87-90, wherein said detecting comprises: (a) hybridizing the single-stranded amplicon with a first nucleic acid probe and a second nucleic acid probe to form a complex, wherein: (i) the first nucleic acid probe comprises a first detectable label; and (ii) the second nucleic acid probe comprises a ligand for a ligand binding molecule; and (b) detecting presence of the complex.

92. The method of paragraph 91, wherein at least one of the first and second nucleic acid probe hybridizes at an inner region of the single-stranded amplicon.

93. The method of paragraph 91 or 92, wherein the detectable label is selected from the group consisting of a light-absorbing dye, a fluorescent dye, a luminescent or bioluminescent molecule, a quantum dot, a radiolabel, an enzyme, a calorimetric label.

94. The method of any one of paragraphs 91-93, wherein the detectable label is calorimetric label selected from the group consisting of colloidal gold, colored glass or plastic beads, and any combinations thereof.

95. The method of paragraph 94, wherein the detectable label is a gold nanoparticle or a latex bead.

96. The method of any one of paragraphs 91-95, wherein the ligand is selected from the group consisting of organic and inorganic molecules, peptides, polypeptides, proteins, peptidomimetics, glycoproteins, lectins, nucleosides, nucleotides, monosaccharides, disaccharides, trisaccharides, oligosaccharides, polysaccharides, lipopolysaccharides, vitamins, steroids, hormones, cofactors, receptors, receptor ligands, and analogs and derivatives thereof.

97. The method of any one of paragraphs 91-96, wherein the ligand is biotin.

98. The method of any one of paragraphs 91-97, wherein the ligand binding molecule is an antibody.

99. The method of any one of paragraphs 91-98, wherein said detecting is by lateral flow detection.

100. The method of any one of paragraphs 91-99, wherein the target nucleic acid is single-stranded.

101. The method of any one of paragraphs 87-100, wherein the target nucleic acid is double-stranded.

102. The method of any one of paragraphs 87-101, wherein the target nucleic acid is RNA.

103. The method of any one of paragraphs 87-102, wherein the target nucleic acid is a viral RNA.

104. The method of any one of paragraphs 87-103, wherein the target nucleic acid is DNA.

105. The method of any one of paragraphs 87-104, wherein the target nucleic acid is a viral DNA.

106. A method of detecting a target nucleic acid, wherein the method comprises: (a) hybridizing the target nucleic acid with a first nucleic acid probe and a second nucleic acid probe to form a complex, wherein: (i) the first nucleic acid probe comprises a first detectable label; and (ii) the second nucleic acid probe comprises a ligand for a ligand binding molecule; and (b) detecting presence of the complex,
wherein the target nucleic acid is a single-stranded.

107. The method of paragraph 106, wherein at least one of the first and second nucleic acid probe hybridizes at an inner region of the target nucleic acid.

108. The method of paragraph 106 or 107, wherein the detectable label is selected from the group consisting of a light-absorbing dye, a fluorescent dye, a luminescent or bioluminescent molecule, a quantum dot, a radiolabel, an enzyme, a colorimetric label.

109. The method of any one of paragraphs 106-108, wherein the detectable label is colorimetric label selected from the group consisting of colloidal gold, colored glass or plastic beads, and any combinations thereof.

110. The method of paragraph 109, wherein the detectable label is a gold nanoparticle or a latex bead.

111. The method of any one of paragraphs 106-110, wherein the ligand is selected from the group consisting of organic and inorganic molecules, peptides, polypeptides, proteins, peptidomimetics, glycoproteins, lectins, nucleosides, nucleotides, monosaccharides, disaccharides, trisaccharides, oligosaccharides, polysaccharides, lipopolysaccharides, vitamins, steroids, hormones, cofactors, receptors, receptor ligands, and analogs and derivatives thereof.

112. The method of any one of paragraphs 106-111, wherein the ligand is biotin.

113. The method of any one of paragraphs 106-112, wherein the ligand binding molecule is an antibody.

114. The method of any one of paragraphs 106-113, wherein said detecting is by lateral flow detection.

115. The method of any one of paragraphs 106-114, wherein the target nucleic acid is RNA.

116. The method of any one of paragraphs 106-115, wherein the target nucleic acid is a viral RNA.

117. The method of any one of paragraphs 106-114, wherein the target nucleic acid is DNA.

118. The method of any one of paragraphs 106-114, wherein the target nucleic acid is a viral DNA.

119. The method of any one of paragraphs 106-118, wherein the target nucleic acid is a single-stranded amplicon.

120. The method of paragraph 119, wherein the method further comprises preparing the single-stranded amplicon prior to the detecting step (a).

121. A composition comprising a first primer and a second primer for amplifying a target nucleic acid, wherein the first primer comprises a nucleic acid modification capable of inhibiting 5′->3′ cleaving activity of a 5′->3′ exonuclease, and the second primer optionally comprises a nucleic acid modification that enhances 5′->3′ cleaving activity of the 5′->3′ exonuclease.

122. A composition comprising a first primer and a second primer for amplifying a target nucleic acid, wherein each of the first primer and second primer independently comprises a nucleic acid modification capable of inhibiting 5′->3′ cleaving activity of a 5′->3′ exonuclease.

123. The composition of paragraph 121 or 122, wherein the nucleic acid modification capable of inhibiting 5′-> 3′ cleaving activity of a 5′->3′ exonuclease is present at the 5′-end.

124. The composition of paragraph 121 or 122, wherein the nucleic acid modification capable of inhibiting 5′-> 3′ cleaving activity of a 5′->3′ exonuclease is present at an internal position.

125. The composition any one of paragraphs 121-124, wherein the nucleic acid modification capable of inhibiting 5′-> 3′ cleaving activity of a 5′->3′ exonuclease is selected from the group consisting of modified internucleotide linkages modified nucleobase, modified sugar, and any combinations thereof.

126. A composition comprising a first primer and a second primer for amplifying a target nucleic acid, wherein at least one or both of the first or second primers comprises a nucleic acid modification capable of inhibiting synthesis of a complementary strand by a polymerase.

127. The composition of any one of paragraphs 122-126, wherein at least one of the primers comprises a modification selected from the group consisting of: (a) modified internucleotide linkages; (b) inverted nucleosides or 5′->5′ internucleotide linkages; (c) 2′-OH or 2′-modified nucleosides; (d) 5′-modified nucleotides and/or 3′-modified nucleotides; (e) 2′->5′ linkages; (f) abasic nucleosides; (g) acyclic nucleosides; (h) spacers; (i) left-handed DNA; and (j) any combinations of (a)-(i).

128. The composition of paragraph 127, wherein said modified internucleotide linkages are selected from the group consisting of phosphorothioates, phosphorodithioates, phosphotriesters, alkylphosphonates, phosphoramidate, phosphoroselenates, borano phosphates, borano phosphate esters, hydrogen phosphonates, alkyl or aryl phosphonates, bridged phosphoroamidates, bridged phosphorothioates, bridged alkylenephosphonates, methylenemethylimino (—CH2—N(CH3)—O—CH2—), thiodiester (—O—C(O)—S—), thionocarbamate (—O—C(O)(NH)—S—), siloxane (—O—Si(H)2—O—), and N,N′-dimethylhydrazine (—CH2—N(CH3)—N(CH3)—), amide-3 (3′—CH₂—C(═O)—N(H)—5′), amide-4 (3′—CH₂—N(H)—C(═O)—5′)), hydroxylamino, siloxane (dialkylsiloxane), carboxamide, carbonate, carboxymethyl, carbamate, carboxylate ester, thioether, ethylene oxide linker, sulfide, sulfonate, sulfonamide, sulfonate ester, thioformacetal (3′—S—CH₂—O—5′), formacetal (3 ′—O—CH₂—O—5′), oxime, methyleneimino, methykenecarbonylamino, methylenemethylimino (MMI, 3′-CH₂—N(CH₃)—O—5′), methylenehydrazo, methylenedimethylhydrazo, methyleneoxymethylimino, ethers (C3′—O—C5′), thioethers (C3′-S-C5′), thioacetamido (C3′-N(H)—C(═O)—CH₂—S—C5′, C3′—O—P(O)—O—SS—C5′, C3′—CH₂—NH—NH—C5′, 3′—NHP(O)(OCH₃)—O—5′ and 3′—NHP(O)(OCH₃)—O—5′.

129. The composition of paragraph 128, wherein said modified internucleotide linkages are phosphorothioate.

130. The composition of any one of paragraphs 127-129, wherein said 2′-modified nucleoside comprises a modification selected from the group consisting of 2′-halo (e.g., 2′-fluoro), 2′-alkoxy (e.g., 2′-Omethyl, 2′-Omethylmethoxy and 2′-Omethylethoxy), 2′-aryloxy, 2′-O-amine or 2′-O-alkylamine (amine NH₂; alkylamino, dialkylamino, heterocyclyl, arylamino, diaryl amino, heteroaryl amino, dihet.eroaryl amino, ethylene diamine or polyamino), O-CH₂CH₂(NCH₂CH₂NMe₂)₂, methyleneoxy (4′-CH₂-O-2′) LNA, ethyleneoxy (4′-(CH₂)₂-O-2′) ENA, 2′-amino (e.g. 2′-NH₂, 2′-alkylamino, 2′-dialkylamino, 2′-heterocyclylamino, 2′-arylamino, 2′-diaryl amino, 2′-heteroaryl amino, 2′-diheteroaryl amino, and 2′-amino acid); NH(CH₂CH₂NH)ₙCH₂CH₂-AMINE (AMINE = NH₂, alkylamino, dialkylamino, heterocyclyl, arylamino, diaryl amino, heteroaryl amino, or diheteroaryl amino), -NHC(O)R (R = alkyl, cycloalkyl, aryl, aralkyl, heteroaryl or sugar), 2′-cyano, 2′-mercapto, 2′-alkyl-thio-alkyl, 2′-thioalkoxy, 2′-thioalkyl, 2′-alkyl, 2′-cycloalkyl, 2′-aryl, 2′-alkenyl and 2′-alkynyl.

131. The composition of any one of paragraphs 127-130, wherein the inverted nucleoside is dT.

132. The composition of any one of paragraphs 127-131, wherein the 5′-modified nucleotide comprises a 5′-modification selected from the group consisting of 5′-monothiophosphate (phosphorothioate), 5′-monodithiophosphate (phosphorodithioate), 5′-phosphorothiolate, 5′-alpha-thiotriphosphate, 5′-beta-thiotriphosphate, 5′-gamma-thiotriphosphate, 5′-phosphoramidates, 5′-alkylphosphonate, 5′-alkyletherphosphonate, a detectable label, and a ligand; or the 3′-modified nucleotide comprises a 3′-modification selected from the group consisting of 3′-monothiophosphate (phosphorothioate), 3′-monodithiophosphate (phosphorodithioate), 3′-phosphorothiolate, 3′-alpha-thiotriphosphate, 3′-beta-thiotriphosphate, 3′-gamma-thiotriphosphate, 3′-phosphoramidates, 3′-alkylphosphonate, 3′-alkyletherphosphonate, a detectable label, and a ligand.

133. The composition of any one of paragraphs 127-132, wherein the 5′-modified nucleotide comprises a detectable label at the 5′-end.

134. The composition of any one of paragraphs 121-127, wherein one of the first or second primer comprises a 5′-OH or a phosphate group at the 5′-end.

135. The composition of any one of paragraphs 121-134, wherein one of the first or second primer comprises a 5′-monophosphate; 5′-diphosphate or a 5′-triphosphate at the 5′-end.

136. The composition of any one of paragraphs 121-135, wherein the composition further comprises one or more reagents for nucleic acid amplification.

137. The composition of any one of paragraphs 121-136, wherein the composition further comprises a 5′->3′ exonuclease.

138. The composition of paragraph 137, wherein the exonuclease is T7 exonuclease, lambda exonuclease, Exonuclease VIII, T5 exonuclease, RecJf, or any combinations thereof.

139. The composition of any one of paragraphs 121-138, wherein the composition further comprises a target nucleic acid for amplification.

140. The composition of paragraph 139, wherein the target nucleic acid is a reference nucleic acid.

141. The composition of any one of paragraphs 121-140, wherein the composition further comprises an amplicon produced by amplification of a target nucleic acid.

142. The composition of paragraph 141, wherein the amplicon is double-stranded.

143. The composition of paragraph 142, wherein the amplicon comprises a 5′-single-stranded overhang on at least one end.

144. The composition of paragraph 141, wherein the amplicon is single stranded.

145. The composition of any one of paragraphs 121-144, wherein the composition is in form of a kit.

146. A double-stranded nucleic acid comprising:
a. a first nucleic acid strand comprising a detectable label; and
b. a second nucleic acid probe comprising a ligand for a ligand binding molecule; and wherein the first nucleic acid strand and the second nucleic acid strands are substantially complementary to each other.

147. The double-stranded nucleic acid of paragraph 146, wherein the detectable label is selected from the group consisting of a light-absorbing dye, a fluorescent dye, a luminescent or bioluminescent molecule, a quantum dot, a radiolabel, an enzyme, a colorimetric label.

148. The double-stranded nucleic acid of paragraph 146 or 147, wherein the detectable label is a colorimetric label selected from the group consisting of colloidal gold, colored glass or plastic beads, and any combinations thereof.

149. The double-stranded nucleic acid of paragraph 148, wherein the detectable label is a gold nanoparticle or a latex bead.

150. The double-stranded nucleic acid of any one of paragraphs 146-149, wherein the ligand is selected from the group consisting of organic and inorganic molecules, peptides, polypeptides, proteins, peptidomimetics, glycoproteins, lectins, nucleosides, nucleotides, monosaccharides, disaccharides, trisaccharides, oligosaccharides, polysaccharides, lipopolysaccharides, vitamins, steroids, hormones, cofactors, receptors, receptor ligands, and analogs and derivatives thereof.

151. The double-stranded nucleic acid of any one of paragraphs 146-150, wherein the ligand is biotin.

152. A composition comprising a double-stranded nucleic acid of any one of paragraphs 146-151.

153. The composition of paragraph 152, wherein the composition further comprises a ligand binding molecule capable of binding with the ligand.

154. The composition of paragraph 152, wherein the ligand binding molecule is an antibody.

155. The composition of any one of paragraphs 152-154, wherein the composition further comprises means for detecting the detectable label.

156. The composition of paragraph 155, wherein said means for detecting the detectable label comprises lateral flow detection.

157. The composition of paragraph 155, wherein said means for detecting the detectable label comprises LFIA.

158. The composition of any one of paragraphs 152-157, wherein the composition is in form of a kit.

159. The method of any one of paragraphs 22, 53, 76, or 87, wherein said detecting the single-stranded amplicon comprises: (a) contacting the single-stranded amplicon with a double-stranded probe, wherein the double-stranded probe comprises: (i) a first nucleic acid strand comprising a fluorophore; (ii) a second nucleic acid strand comprising a quencher for quenching a fluorescent emission of the fluorophore; and (b) measuring the fluorescent emission of the fluorophore, wherein the fluorescent emission of the fluorophore is quenched when the first and second nucleic acid strands are hybridized to each other, wherein the double-stranded probe comprises a single-stranded overhang at one end and the nucleic acid strand comprising the single-stranded overhang comprises a nucleotide sequence substantially complementary to a region of the single-stranded amplicon, and wherein the amplicon and the nucleic strand comprising the overhang hybridize to each other, thereby inhibiting quenching of the fluorescent emission of the fluorophore by the quencher.

160. The method of paragraph 159, wherein the first nucleic acid strand comprises the single-stranded overhang.

161. The method of paragraph 159 or 160, wherein the first and second nucleic acid strands are covalently linked to each other.

162. The method of any one of paragraphs 1-105, further comprising a step of adding a surfactant to the double-stranded amplicon.

163. A method for preparing a single-stranded amplicon from a target nucleic acid, the method comprising: (a) amplifying a target nucleic acid with a first primer and a second primer to produce a double-stranded amplicon: and (b) contacting the double-stranded amplicon from step (a) with a surfactant to displace the single-stranded amplicon.

164. The method of paragraph 162 or 163, wherein the surfactant is an anionic surfactant.

165. The method of any one of paragraphs 162-164, wherein the surfactant is sodium dodecyl sulfate (SDS).

166. A method for detecting a target nucleic acid, the method comprising: amplifying a target nucleic acid with a first primer and a second primer to produce a double-stranded amplicon, wherein the first primer comprises a detectable label at its 5′-end; (b) contacting the double-stranded amplicon with a 5′->3′ exonuclease to produce an amplicon having a single-stranded region (e.g., a single-stranded amplicon); and (c) detecting the amplicon having a single-stranded region, wherein said detecting comprises applying amplicon having a single-stranded region to a lateral flow test strip, wherein the later flow test strip comprises: a test/capture region comprising a nucleic acid capture probe immobilized therein, wherein the nucleic acid capture probe comprises a toehold domain (e.g., a single-stranded region) comprising a nucleotide sequence substantially complementary to at least a part of the single-stranded amplicon.

167. A method for detecting a target nucleic acid, the method comprising: (a) amplifying a target nucleic acid with a first primer and a second primer to produce a double-stranded amplicon, wherein: (i) the first primer comprises a detectable label at its 5′-end; (ii) the second primer comprises one or more uridine nucleotides; and (b) contacting the double-stranded amplicon from step (a) with Uracil-DNA glycosylase (UDG) to produce an amplicon having a single-stranded region (e.g., a single-stranded amplicon); and (c) detecting the amplicon having the single-stranded region, wherein said detecting comprises applying the amplicon having the single-stranded region to a lateral flow test strip, wherein the later flow test strip comprises: a test/capture region comprising a nucleic acid capture probe immobilized therein, wherein the nucleic acid capture probe comprises a toehold domain (e.g., a single-stranded region) comprising a nucleotide sequence substantially complementary to at least a part of the single-stranded region of the amplicon.

168. A method for detecting a target nucleic acid, the method comprising: (a) amplifying a target nucleic acid with a first primer and a second primer to produce a double-stranded amplicon, wherein the first primer comprises a detectable label at its 5′-end and a nucleic acid modification capable of inhibiting synthesis of a complementary strand by a polymerase at an internal position, and wherein the double-stranded amplicon comprises a 5′ single-stranded region at one end; and (b) detecting the amplicon having the 5′ single-stranded region, wherein said detecting comprises applying the amplicon to a lateral flow test strip, wherein the lateral flow test strip comprises: a test/capture region comprising a nucleic acid capture probe immobilized therein, wherein a first region/domain of the nucleic acid capture probe comprises a toehold domain comprising a nucleotide sequence substantially complementary to at least a part of the single-stranded amplicon.

169. The method of any one of paragraphs 166-168, further comprising a step of contacting the double-stranded amplicon with a surfactant, e.g., SDS.

170. A method for detecting a target nucleic acid, the method comprising: (a) amplifying a target nucleic acid to produce a double-stranded amplicon; (b) hybridizing a first nucleic acid probe and a second nucleic acid probe to one strand of the double-stranded amplicon to form a complex comprising the first and second probes hybridized to one strand of the double-stranded amplicon, wherein said hybridizing is in the presence of a surfactant e.g., SDS, and/or a reagent capable of hybridizing/localizing a single-strand nucleic acid strand to a double-stranded nucleic acid, wherein the first nucleic acid probe comprises a first detectable label and the second nucleic acid probe comprises a ligand for a ligand binding molecule; and (c) detecting the complex, e.g., by a lateral flow assay/device.

171. A method for detecting a target nucleic acid, the method comprising: (a) amplifying a target nucleic acid with a first primer and a second primer to produce a double-stranded amplicon, wherein the first primer comprises a nucleic acid modification capable of inhibiting 5′->3′ cleaving activity of a 5′->3′ exonuclease; (b) contacting the double-stranded amplicon with a 5′->3′ exonuclease to produce a single-stranded amplicon; and (c) detecting the single-stranded amplicon, wherein said detecting comprises hybridizing a plurality of nucleic acid probes to the single-stranded amplicon, wherein members of the plurality comprise a nucleotide sequence substantially complementary to different regions of the strand, wherein each probe comprises a detectable label attached thereto, and wherein the detectable label undergoes a change in an optical property in response to label density, pH change and/or temperature change, and optionally, said hybridizing is in presence of a surfactant, e.g., SDS.

172. A method for detecting a target nucleic acid, the method comprising: (a) amplifying a target nucleic acid with a first primer and a second primer to produce a double-stranded amplicon, optionally, wherein the first primer comprises a nucleic acid modification capable of inhibiting 5′->3′ cleaving activity of a 5′->3′ exonuclease; and (b) detecting the double-stranded amplicon, wherein said detecting comprises hybridizing a plurality of nucleic acid probes to one strand of the double-stranded, wherein said hybridizing is in the presence of a surfactant e.g., SDS, and/or a reagent capable of localizing a single-strand nucleic acid strand to a double-stranded nucleic acid, wherein members of the plurality comprise a nucleotide sequence substantially complementary to different regions of the strand, wherein each probe comprises a detectable label attached thereto, and wherein the detectable label undergoes a change in an optical property in response to label density, pH change, and/or temperature change.

173. The method of paragraph 171 or 172, wherein the reagent capable of localizing a single-strand nucleic acid strand to a double-stranded nucleic acid is recombinase, single-stranded binding protein, Cas protein, zinc finger nuclease, transcription activator-like effector nuclease (TALEN), or any combinations thereof.

174. The method of any one of paragraphs 166-173, wherein the detectable label is a nanoparticle.

175. The method of any one of the preceding paragraphs, wherein said detecting is by a lateral flow assay and wherein the lateral flow assay is in presence of a surfactant, bile salt, ionic salt, chaotropic agent, formamide, DNA duplex destabilizer, and/or reducing agent.

176. The method of paragraph 175, wherein the surfactant, bile salt, ionic salt, chaotropic agent, formamide, DNA duplex destabilizer, and/or reducing agent is at a concentration ranging from 0.5% to 20%.

177. The method of paragraph 178, wherein the surfactant, bile salt, ionic salt, chaotropic agent, formamide, DNA duplex destabilizer, and/or reducing agent is at a concentration of about 10%.

178. The method of any one of paragraphs 175-176, wherein the surfactant, bile salt, ionic salt, chaotropic agent, formamide, DNA duplex destabilizer, and/or reducing agent is in a buffer, e.g., running buffer for the lateral flow assay.

179. The method of any one of paragraphs 175-178, wherein the surfactant, bile salt, ionic salt, chaotropic agent, formamide, DNA duplex destabilizer, and/or reducing agent is added to a solution comprising the probe bound amplicon prior to and/or concurrently with applying the solution to a lateral flow test strip of the assay.

180. The method of any one of paragraphs 175-179, wherein a lateral flow test strip of the assay is pre-treated with the surfactant, bile salt, ionic salt, chaotropic agent, formamide, DNA duplex destabilizer, and/or reducing agent.

Some embodiments of the technology described herein can be defined according to any of the following numbered paragraphs:

1. A method for detecting a target nucleic acid in a sample, the method comprising: (a) hybridizing a nucleic acid probe to an amplicon from amplification of a target nucleic acid, wherein the nucleic acid probe comprises a nucleotide sequence substantially complementary or identical to a nucleotide sequence of the target nucleic acid or a primer in used in the amplification of the target nucleic acid, wherein the nucleic acid probe comprises a reporter molecule capable of producing a detectable signal, and wherein said amplification is Loop-mediated Isothermal Amplification (LAMP); (b) cleaving the hybridized nucleic acid probe with a double-strand specific exonuclease having 5′ to 3′ exonuclease activity; and (c) detecting the reporter molecule from the cleaved nucleic acid probe or detecting with a sequence specific method any remaining uncleaved nucleic acid probe.

2. The method of paragraph 1, wherein said hybridizing the nucleic acid probe or cleaving the hybridized nucleic acid probe is simultaneous with the amplification of the target nucleic acid.

3. The method of paragraph 1 or 2, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, and ferromagnetic metals.

4. The method of any one of paragraphs 1-3, wherein the nucleic acid probe further comprises a quencher molecule.

5. The method of paragraph 4, wherein the quencher molecule quenches the detectable signal from the reporter molecule when the nucleic acid probe is not hybridized to the amplicon.

6. The method of paragraph 4 or 5, wherein the quencher molecule quenches the detectable signal from the reporter molecule when the nucleic acid probe is hybridized to the amplicon.

7. The method of any one of paragraphs 4-6, wherein the nucleic acid probe further comprises at least one additional quencher molecule.

8. The method of any one of paragraphs 1-7, wherein the nucleic acid probe comprises a plurality of reporter molecules.

9. The method of any one paragraphs 1-8, wherein at least one primer used in the amplification comprises a nucleic acid modification capable of inhibiting the 5′->3′ exonuclease activity of the exonuclease.

10. The method of any one of paragraphs 1-9, wherein the nucleic acid probe comprises at least one nucleic acid modification capable of increasing a melting temperature (Tm) of the nucleic acid probe for hybridizing with a complementary strand relative to a nucleic acid probe lacking said modification.

11. The method of any one of paragraphs 1-10, wherein the nucleic acid probe comprises at least one nucleic acid modification capable of inhibiting extension by a polymerase.

12. The method of any one of paragraphs 1-11, wherein the exonuclease lacks polymerase activity.

13. The method of any one of paragraphs 1-12, wherein the exonuclease has polymerase activity.

14. The method of any one of paragraphs 1-13, wherein the exonuclease is selected from the group consisting of Bst Full Length, Taq DNA polymerase, T7 Exonuclease, Exonuclease VIII, Exonuclease VIII truncated, Lambda exonuclease, T5 Exonuclease, RecJf, and any combination thereof.

15. The method of any one of paragraphs 1-14, wherein said detecting the reporter molecule comprises detecting a detectable signal produced by the reporter molecule.

16. The method of paragraph 1-15, wherein said detecting the reporter molecule comprises fluorescence detection, luminescence detection, chemiluminescence detection, or immunofluorescence detection.

17. The method of any one of paragraphs 1-16, wherein said detecting the reporter molecule comprises a lateral flow assay.

18. The method of any one of paragraphs 1-17, wherein the nucleic acid probe comprises a ligand for a ligand binding molecule.

19. The method of any one of paragraphs 1-18, wherein said sequence-specific detection comprises toehold-mediated strand displacement, probe-based electrochemical readout, micro-array detection, sequence-specific amplification or any combinations thereof.

20. The method of any one of paragraphs 1-19, wherein the nucleic acid probe comprises a nucleotide sequence substantially complementary to a primer used in the amplification of the target nucleic acid.

21. A kit for detecting a target nucleic acid in a sample, the kit comprising: (a) an exonuclease having 5′->3′ cleaving activity; (b) a primer set for amplifying a target nucleic acid by LAMP and wherein the primer set comprises a forward outer primer (F3), a reverse outer primer (R3), a forward inner primer (FIP), and a reverse inner primer (RIP); and (c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set.

22. The kit of paragraph 21, wherein the primer set further comprises a forward loop primer (LF), and a reverse loop primer (LR).

23. The kit of paragraph 21 or 23, wherein the nucleic acid probe comprises further comprises a quencher molecule.

24. The kit of paragraph 23, wherein the quencher molecule quenches the detectable signal from the reporter molecule when the nucleic acid probe is not hybridized to a complementary nucleic acid strand.

25. The kit of any one of paragraphs 21-24, wherein the kit further comprises a reference nucleic acid.

26. The kit of any one of paragraphs 21-25, wherein the kit further comprises a lateral flow device for detecting the reporter molecule.

27. The kit of any one of paragraphs 21-26, wherein the kit further comprises means for detecting a detectable signal from the reporter molecule.

28. The kit of any one of paragraphs 21-27, wherein the kit further comprises a DNA polymerase having strand displacement activity.

29. The kit of any one of paragraphs 21-28, wherein the kit further comprises dNTPs.

30. The kit of any one of paragraphs 21-29, wherein the kit further comprises a buffer.

31. A composition comprising: (a) an exonuclease having 5′->3′ cleaving activity; (b) a primer set for amplifying a target nucleic acid via LAMP and wherein and the primer set comprises a forward outer primer (F3), a reverse outer primer (R3), a forward inner primer (FIP), and a reverse inner primer (RIP); and (c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set.

32. The composition of paragraph 31, wherein the primer set further comprises a forward loop primer (LF), and a reverse loop primer (LR).

33. The composition of paragraph 31 or 32, wherein the nucleic acid probe further comprises a quencher molecule.

34. The composition of paragraph 33, wherein the quencher molecule quenches the detectable signal from the reporter molecule when the nucleic acid probe is not hybridized to a complementary strand.

35. The composition of any one of paragraphs 31-34, wherein the composition further comprises the target nucleic acid.

36. The composition of any one of paragraphs 31-35, wherein the composition further comprises a DNA polymerase having strand displacement activity.

37. The composition of any one of paragraphs 31-36, wherein the composition further comprises dNTPs.

38. The composition of any one of paragraphs 31-37, wherein the composition further comprises a buffer.

39. The composition of any one of paragraphs 31-38, wherein the composition is in lyophilized form.

40. The composition of any one of paragraphs 31-39, wherein one or more components of the composition is disposed in a device comprising two or more chambers and means for irreversibly moving a fluid from a first chamber to a second chamber.

41. The kit of any one of paragraphs 21-30, wherein the kit further comprises a device comprising two or more chambers and means for irreversibly moving a fluid from a first chamber to a second chamber.

42. The kit of any one of paragraphs 21-30 or 41, wherein at least one component of the kit is disposed in a device comprising two or more chambers and means for irreversibly moving a fluid from a first chamber to a second chamber.

43. The method of any one of paragraphs 1-20, wherein the method is performed in a device comprising two or more chambers and means for irreversibly moving a fluid from a first chamber to a second chamber.

44. The composition, kit, or method of any one of paragraphs 40-43, wherein the means for irreversibly moving the fluid from the first to the second chamber can be actuated by a built-in spring whose potential energy is released by a solenoid trigger.

45. The composition, kit, or method of any one of paragraphs 40-44, wherein the device further comprises means for detecting the detectable signal from the reporter molecule.

Some embodiments of the technology described herein can be defined according to any of the following numbered paragraphs:

1. A method for detecting an amplicon from amplification of a target nucleic acid in a sample, the method comprising:
hybridizing a nucleic acid probe to an amplicon from amplification of a target nucleic acid, wherein the nucleic acid probe comprises a nucleotide sequence substantially complementary or identical to a nucleotide sequence of the target nucleic acid or a primer in used in the amplification of the target nucleic acid, wherein the nucleic acid probe comprises a reporter molecule capable of producing a detectable signal, and, optionally, the detectable signal from the reporter molecule is partially quenched when the nucleic acid probe is hybridized to the amplicon;
cleaving the hybridized nucleic acid probe with a double-strand specific exonuclease having 5′ to 3′ exonuclease activity; and
detecting the reporter molecule from the cleaved nucleic acid probe or detecting any remaining uncleaved nucleic acid probe.

2. The method of paragraph 1, wherein said hybridizing the nucleic acid probe or cleaving the hybridized nucleic acid probe is simultaneous with the amplification of the target nucleic acid.

3. The method of paragraph 1, wherein said hybridizing the nucleic acid probe or cleaving the hybridized nucleic acid probe is after the amplification of the target nucleic acid.

4. The method of any one of paragraphs 1-3, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, and ferromagnetic metals.

5. The method of any one of paragraphs 1-4, wherein the nucleic acid probe further comprises a quencher molecule.

6. The method of paragraph 5, wherein the quencher molecule quenches the detectable signal from the reporter molecule when the nucleic acid probe is not hybridized to the amplicon.

7. The method of paragraph 5 or 6, wherein the quencher molecule quenches the detectable signal from the reporter molecule when the nucleic acid probe is hybridized to the amplicon.

8. The method of any one of paragraphs 5-7, wherein the nucleic acid probe further comprises at least one additional quencher molecule.

9. The method of any one of paragraphs 1-8, wherein the nucleic acid probe comprises a plurality of reporter molecules.

10. The method of paragraph 9, wherein at least two reporter molecules in the plurality of reporter molecules are different.

11. The method of any one paragraphs 1-10, wherein at least one primer used in the amplification comprises a nucleic acid modification capable of inhibiting the 5′->3′ exonuclease activity of the exonuclease.

12. The method of any one of paragraphs 1-11, wherein the nucleic acid probe comprises at least one nucleic acid modification.

13. The method of any one of paragraphs 1-12, wherein the nucleic acid probe comprises at least one nucleic acid modification capable of increasing a melting temperature (Tm) of the nucleic acid probe for hybridizing with a complementary strand relative to a nucleic acid probe lacking said modification.

14. The method of any one of paragraphs 1-13, wherein the nucleic acid probe comprises at least one nucleic acid modification capable of inhibiting extension by a polymerase.

15. The method of any one of paragraphs 1-14, wherein the exonuclease lacks polymerase activity.

16. The method of any one of paragraphs 1-15, wherein the exonuclease has polymerase activity.

17. The method of any one of paragraphs 1-16, wherein the exonuclease is selected from the group consisting of Bst Full Length, Taq DNA polymerase, T7 Exonuclease, Exonuclease VIII, Exonuclease VIII truncated, Lambda exonuclease, T5 Exonuclease, RecJf, and any combination thereof.

18. The method of any one of paragraphs 1-17, wherein said amplification is isothermal amplification.

19. The method of any one of paragraphs 1-18, wherein said amplification is selected from the group consisting of: Loop Mediated Isothermal Amplification (LAMP), Recombinase Polymerase Amplification (RPA), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3SR), Single primer isothermal amplification (SPIA), and cross-priming amplification (CPA).

20. The method of any one of paragraphs 1-19, wherein said amplification is Loop-mediated Isothermal Amplification (LAMP).

21. The method of any one of paragraphs 1-20, wherein the amplicon is single-stranded.

22. The method of paragraph 21, wherein the method further comprises a step of preparing the single-stranded amplicon from the target nucleic acid prior to hybridizing the nucleic acid probe with the amplicon.

23. The method of any one of paragraphs 1-22, wherein said detecting the reporter molecule comprises detecting a detectable signal produced by the reporter molecule.

24. The method of paragraph 1-23, wherein said detecting the reporter molecule comprises fluorescence detection, luminescence detection, chemiluminescence detection, colorimetric detection, or immunofluorescence detection.

25. The method of any one of paragraphs 1-24, wherein said detecting the reporter molecule comprises a lateral flow assay.

26. The method of any one of paragraphs 1-25, wherein the nucleic acid probe comprises a ligand for a ligand binding molecule.

27. The method of any one of paragraphs 1-26, wherein the nucleic acid probe comprises a lateral flow detectable moiety.

28. The method of any one of paragraphs 1-27, wherein said detecting the uncleaved nucleic acid probe comprises sequence-specific detection.

29. The method of paragraph 28, wherein said sequence-specific detection comprises toehold-mediated strand displacement, probe-based electrochemical readout, micro-array detection, sequence-specific amplification, hybridization with conjugated or unconjugated nucleic acid strand, colorimetric assays, gel electrophoresis, molecular beacons, fluorophore-quencher pairs, microarrays, sequencing or any combinations thereof.

30. The method of any one of paragraphs 1-29, wherein said detecting the uncleaved nucleic acid probe comprises lateral flow detection.

31. The method of any one of paragraphs 1-30, wherein the nucleic acid probe is immobilized on a surface.

32. The method of any one of paragraphs 1-31, wherein at least one primer used in the amplification is immobilized on a surface.

33. The method of any one of paragraphs 1-32, wherein the nucleic acid probe comprises a nucleotide sequence substantially complementary to a primer used in the amplification of the target nucleic acid.

34. The method of any one of paragraphs 1-33, wherein the nucleic acid probe comprises a nucleotide sequence substantially identical to a primer used in the amplification of the target nucleic acid.

35. The method of any one of paragraphs 1-34, wherein the nucleic acid probe comprises a nucleotide sequence substantially complementary to a nucleotide sequence at an internal position of the amplicon.

36. The method of any one of paragraphs 1-35, wherein the nucleic acid probe comprises a first nucleic acid strand and a second nucleic acid strand, wherein the first strand comprises a region that is substantially complementary to a region in the second strand.

37. The method of paragraph 36, wherein the first and second strands are linked to each other.

38. The method of any one of paragraphs 1-37, wherein the nucleic acid probe forms a hairpin structure when hybridized to the amplicon.

39. The method of any one of paragraphs 1-38, wherein the nucleic acid probe comprises a single-stranded region when hybridized to the amplicon.

40. The method any one of paragraphs 1-39, wherein said detection is multiplexed detection of at least two target nucleic acids.

41. The method of any one of paragraphs 1-40, wherein the method is performed in a device comprising two or more chambers and means for irreversibly moving a fluid from a first chamber to a second chamber.

42. The method of paragraph 41, wherein the means for irreversibly moving the fluid from the first to the second chamber can be actuated by a built-in spring whose potential energy is released by a solenoid trigger.

43. The method of paragraph 42, wherein the device further comprises means for detecting the detectable signal from the reporter molecule.

44. A kit for detecting a target nucleic acid in a sample, the kit comprising
a) an exonuclease having 5′->3′ cleaving activity;
b) a primer set for amplifying a target nucleic acid; and
c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set.

45. The kit of paragraph 44, wherein said amplification is LAMP and the primer set comprises a forward outer primer (F3), a reverse outer primer (R3), a forward inner primer (FIP), and a reverse inner primer (RIP).

46. The kit of paragraph 45, wherein the primer set further comprises a forward loop primer (LF), and a reverse loop primer (LR).

47. The kit of any one of paragraphs 44-46, wherein the nucleic acid probe comprises further comprises a quencher molecule.

48. The kit of paragraph 47, wherein the quencher molecule quenches the detectable signal from the reporter molecule when the nucleic acid probe is not hybridized to a complementary nucleic acid strand.

49. The kit of paragraph 47, wherein the quencher molecule quenches the detectable signal from the reporter molecule when the nucleic acid probe is hybridized to a complementary nucleic acid strand

50. The kit of any one of paragraphs 47-49, wherein the nucleic acid probe further comprises at least one additional quencher molecule.

51. The kit of any one of paragraphs 44-50, wherein the nucleic acid probe comprises a plurality of reporter molecules.

52. The kit of paragraph 51, wherein at least two reporter molecules in the plurality of reporter molecules are different.

53. The kit of any one of paragraphs 44-52, wherein the nucleic acid probe comprises at least one nucleic acid modification capable of increasing a melting temperature (Tm) of the nucleic acid probe for hybridizing with a complementary strand relative to a nucleic acid probe lacking said modification.

54. The kit of any one of paragraphs 44-53, wherein the nucleic acid probe comprises at least one nucleic acid modification capable of inhibiting extension by a polymerase.

55. The kit of any one of paragraphs 44-54, wherein the kit further comprises a reference nucleic acid.

56. The kit of any one of paragraphs 44-55, wherein the kit further comprises a lateral flow device for detecting the reporter molecule.

57. The kit of any one of paragraphs 44-56, wherein the kit further comprises means for detecting a detectable signal from the reporter molecule.

58. The kit of any one of paragraphs 44-57, further comprising reagents for preparing a double-stranded amplicon from the target nucleic acid.

59. The kit of any one of paragraphs 44-58, further comprising reagents for preparing a single-stranded amplicon from the target nucleic acid.

60. The kit of any one of paragraphs 44-59, wherein the kit further comprises a DNA polymerase having strand displacement activity.

61. The kit of any one of paragraphs 44-60, wherein the kit further comprises dNTPs.

62. The kit of any one of paragraphs 44-61, wherein the kit further comprises a buffer.

63. The kit of any one of paragraphs 44-62, wherein the kit further comprises a device comprising two or more chambers and means for irreversibly moving a fluid from a first chamber to a second chamber.

64. The kit of any one of paragraphs 44-63, wherein at least one component of the kit is disposed in a device comprising two or more chambers and means for irreversibly moving a fluid from a first chamber to a second chamber.

65. The kit of paragraph 63 or 64 wherein the means for irreversibly moving the fluid from the first to the second chamber can be actuated by a built-in spring whose potential energy is released by a solenoid trigger.

66. The kit of any one of paragraphs 63-65, wherein the device further comprises means for detecting the detectable signal from the reporter molecule.

67. The kit of any one of paragraphs 44-66, wherein the nucleic acid probe comprises a nucleotide sequence substantially complementary to a primer in the primer set.

68. The kit of any one of paragraphs 44-67, wherein the nucleic acid probe comprises a nucleotide sequence substantially identical to a primer in the primer set.

69. The kit of any one of paragraphs 44-68, wherein the nucleic acid probe comprises a nucleotide sequence substantially complementary to a nucleotide sequence at an internal position of an amplicon prepared using the primer set.

70. The kit of any one of paragraphs 44-69, wherein the nucleic acid probe comprises a first nucleic acid strand and a second nucleic acid strand, wherein the first strand comprises a region that is substantially complementary to a region in the second strand.

71. The kit of paragraph 70, wherein the first and second strand are linked to each other.

72. The kit of any one of paragraphs 44-71, wherein the nucleic acid probe forms a hairpin structure when hybridized to a complementary nucleic acid.

73. A composition comprising:
a) an exonuclease having 5′->3′ cleaving activity;
b) a primer set for amplifying a target nucleic acid; and
c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set.

74. The composition of paragraph 73, wherein said amplification is LAMP and the primer set comprises a forward outer primer (F3), a reverse outer primer (R3), a forward inner primer (FIP), and a reverse inner primer (RIP).

75. The composition of paragraph 74, wherein the primer set further comprises a forward loop primer (LF), and a reverse loop primer (LR).

76. The composition of any one of paragraphs 73-75, wherein the nucleic acid probe further comprises a quencher molecule.

77. The composition of paragraph 76, wherein the quencher molecule quenches the detectable signal from the reporter molecule when the nucleic acid probe is not hybridized to a complementary strand.

78. The composition of paragraph 76, wherein the quencher molecule quenches the detectable signal from the reporter molecule when the nucleic acid probe is hybridized to a complementary nucleic acid strand

79. The composition of any one of paragraphs 73-78, wherein the nucleic acid probe further comprises at least one additional quencher molecule.

80. The composition of any one of paragraphs 73-79, wherein the nucleic acid probe comprises a plurality of reporter molecules.

81. The composition of paragraph 80, wherein at least two reporter molecules in the plurality of reporter molecules are different.

82. The composition of any one of paragraphs 73-81, wherein the nucleic acid probe comprises at least one nucleic acid modification capable of increasing a melting temperature (Tm) of the nucleic acid probe for hybridizing with a complementary strand relative to a nucleic acid probe lacking said modification.

83. The composition of any one of paragraphs 73-82, wherein the nucleic acid probe comprises at least one nucleic acid modification capable of inhibiting extension by a polymerase.

84. The composition of any one of paragraphs 73-83, wherein the composition further comprises a reference nucleic acid.

85. The composition of any one of paragraphs 73-84, wherein the composition further comprises the target nucleic acid.

86. The composition of any one of paragraphs 73-85, further comprising reagents for preparing a double-stranded amplicon from the target nucleic acid.

87. The composition of any one of paragraphs 73-86, further comprising reagents for preparing a single-stranded amplicon from the target nucleic acid.

88. The composition of any one of paragraphs 73-87, wherein the composition further comprises a DNA polymerase having strand displacement activity.

89. The composition of any one of paragraphs 73-88, wherein the composition further comprises dNTPs.

90. The composition of any one of paragraphs 73-89, wherein the composition further comprises a buffer.

91. The composition of any one of paragraphs 73-90, wherein the composition is in lyophilized form.

92. The composition of any one of paragraphs 73-91, wherein one or more components of the composition is disposed in a device comprising two or more chambers and means for irreversibly moving a fluid from a first chamber to a second chamber.

93. The composition of paragraph 92, wherein the means for irreversibly moving the fluid from the first to the second chamber can be actuated by a built-in spring whose potential energy is released by a solenoid trigger.

94. The composition of paragraph 92 or 93, wherein the device further comprises means for detecting the detectable signal from the reporter molecule.

95. The composition of any one of paragraphs 73-94, wherein the nucleic acid probe comprises a nucleotide sequence substantially complementary to a primer used in the amplification of the target nucleic acid

96. The composition of any one of paragraphs 73-95, wherein the nucleic acid probe comprises a nucleotide sequence substantially identical to a primer used in the amplification of the target nucleic acid.

97. The composition of any one of paragraphs 73-96, wherein the nucleic acid probe comprises a nucleotide sequence substantially complementary to a nucleotide sequence at an internal position of the amplicon.

98. The composition of any one of paragraphs 73-97, wherein the nucleic acid probe comprises a first nucleic acid strand and a second nucleic acid strand, wherein the first strand comprises a region that is substantially complementary to a region in the second strand.

99. The composition of paragraph 98, wherein the first and second strand are linked to each other.

100. The composition of any one of paragraphs 73-99, wherein the nucleic acid probe forms a hairpin structure when hybridized to a complementary nucleic acid.

101. The composition of any one of paragraphs 73-100, further comprising a single-stranded amplicon produced from the target nucleic acid.

102. The composition of any one of paragraphs 73-101, further comprising a double-stranded amplicon produced from the target nucleic acid.

103. A kit for detecting a target nucleic acid in a sample, the kit comprising a nucleic acid probe and wherein the nucleic acid probe comprises a nucleotide sequence selected from the group consisting of SEQ ID NOs: 51-55.

104. The kit of paragraph 103, wherein the kit further comprises an exonuclease having 5′->3′ cleaving activity

105. The kit of paragraph 103 or 104, wherein the kit further comprise a primer set for amplifying a target nucleic acid.

106. The kit of paragraph 105, wherein said amplification is LAMP and the primer set comprises a forward outer primer (F3), a reverse outer primer (R3), a forward inner primer (FIP), and a reverse inner primer (RIP).

107. The kit of paragraph 106, wherein the primer set further comprises a forward loop primer (LF), and a reverse loop primer (LR).

108. The kit of any one of paragraphs 103-107, wherein the kit further comprises a reference nucleic acid.

109. The kit of any one of paragraphs 103-108, wherein the kit further comprises a lateral flow device.

110. The kit of any one of paragraphs 103-109, wherein the kit further comprises means for detecting a detectable signal from the nucleic acid probe.

111. The kit of any one of paragraphs 103-110, further comprising reagents for preparing a double-stranded amplicon from the target nucleic acid.

112. The kit of any one of paragraphs 103-111, further comprising reagents for preparing a single-stranded amplicon from the target nucleic acid.

113. The kit of any one of paragraphs 103-112, wherein the kit further comprises a DNA polymerase having strand displacement activity.

114. The kit of any one of paragraphs 103-113, wherein the kit further comprises dNTPs.

115. The kit of any one of paragraphs 103-114, wherein the kit further comprises a buffer.

116. The kit of any one of paragraphs 103-115, wherein the kit further comprises a device comprising two or more chambers and means for irreversibly moving a fluid from a first chamber to a second chamber.

117. The kit of any one of paragraphs 103-116, wherein at least one component of the kit is disposed in a device comprising two or more chambers and means for irreversibly moving a fluid from a first chamber to a second chamber.

118. The kit of paragraph 116 or 117 wherein the means for irreversibly moving the fluid from the first to the second chamber can be actuated by a built-in spring whose potential energy is released by a solenoid trigger.

119. The kit of any one of paragraphs 116-118, wherein the device further comprises means for detecting the detectable signal from the nucleic acid probe.

120. The kit of any one of paragraphs 105-119, wherein a primer in the primer set comprise a nucleotide sequence substantially complementary to the nucleic acid probe.

121. The kit of any one of paragraphs 105-120, wherein a primer in the primer set comprise a nucleotide sequence substantially identical to the nucleic acid probe.

122. The kit of any one of paragraphs 103-121, wherein an internal position of an amplicon prepared using the primer set comprises a nucleotide sequence substantially complementary to the nucleic acid probe.

### EXAMPLES

### Example 1: High-Specificity Detection of Nucleic Acids Using Recombinase Polymerase Amplification (RPA) and Sequence-Specific Lateral Flow Devices (LFD)

### Introduction

Recent innovations in isothermal amplification of specific target analyte sequences, paired with visual readout of the result have brought the prospect of highly sensitive point-of-care (POC) diagnostics that are fast, cheap, and use readily accessible equipment.

One isothermal amplification method of particular interest is Recombinase Polymerase Amplification (RPA), which allows rapid, exponential amplification of target nucleic acid sequences (DNA, RNA) (see e.g., Piepenburg 2006). Like PCR, it utilizes a pair of primers corresponding to opposite strands of the target sequence, which makes the amplification very specific due to the need to detect two independent sequences to form a successful amplicon. Unlike PCR, however, the reaction occurs isothermally, so there is no need for expensive thermocycling machines. This also allows the reaction to occur very quickly (typically less than 30 minutes) compared to standard PCR protocols (see e.g., Piepenburg 2006, Tsaloglou 2018).

While a number of visual diagnostic readout assays have been developed, Lateral Flow Device (LFD) readout has a number of key advantages. LFD’s, which use capillary action to transport reactants or reagents along a series of membranes to generate signal at a test line only in the presence of a target analyte, have been utilized to detect a wide variety of targets (e.g. proteins, antibodies, nucleic acids, drug concentrations) through several different types of readout (e.g. fluorescence, chromogenic, colorimetric). Importantly, they use unidirectional reagent flow allows multiple lines of reagents to be printed in series. This allows multiplexed detection assays through the use of multiple test lines. This aspect also allows just a single assay to perform both test and control experiments in a single assay through the use of printed control lines that can be built into the device to can check validity or stability of reagents.

LFD readouts can be very specific, and when paired with prior amplification of target-dependent signal, the detection can also be extremely sensitive. A number of demonstrations have shown the potential for combining RPA amplification with LFD-based readout. However, many RPA-amplified DNA detection schemes with LFD readout rely on non-DNA signals such as fluorophores or biotin, initially on separate primers but brought together during amplification. These have intrinsically limited specificity, since RPA is error prone, and primer ‘dimers’ or other non-specific connections result in positive signals on LFD. There have been several demonstrations the application of RPA products to Lateral Flow Devices (LFD’s) for rapid visual detection of target amplicons, but they lack the capability of checking the target amplicon in a sequence specific way which would eliminate the problem of false positives from RPA background amplicons. One recent technology follows up the RPA step with a CRISPR-based detection step to achieve sequence-specific checking of the amplicons. By checking the amplicon in a sequence-specific way via CRISPR binding to the target amplicon, this approach can significantly improve the specificity of detection through reduction in false positive signals from RPA. However, this technique requires an extra heated incubation step (typically 30 minutes) and extra enzymatic reagents prior to LFD readout to achieve this post-RPA sequence-specific checking (see e.g., FIG. 8).

Non-LFD readouts may also make good use of single-stranded products, allowing ‘testing’ for sequence by hybridization to a complementary test strand either directly or through toehold-mediated strand displacement. Examples include hybridization to microarrays, or any other system where melting duplexed products is precluded.

Described herein are methods for creating single-stranded nucleic acid products from isothermal exponential amplification methods such as RPA that can be specifically detected through lateral flow devices (LFD’s). This detection can be made specific to the target amplicon sequence, for improved specificity of detection by excluding background RPA amplicons which cause false positives. Critically, this hybridization-based sequence detection is performed directly on the LFD strip, eliminating the need for an additional long incubation step. Importantly, this step can be achieved through the use of relatively inexpensive equipment and can be performed rapidly (e.g. <15 minute turnaround time, even for detecting just a few copies of a target sequence).

### Strategies for Creating ssDNA Product

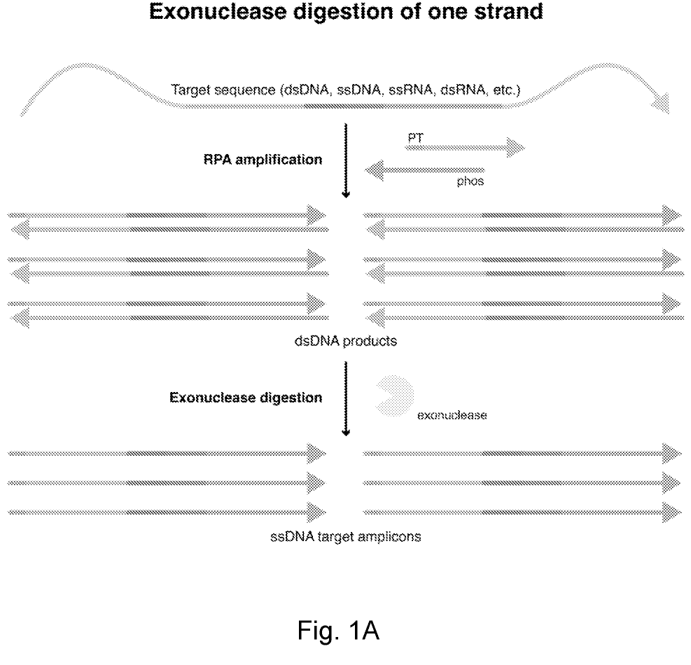

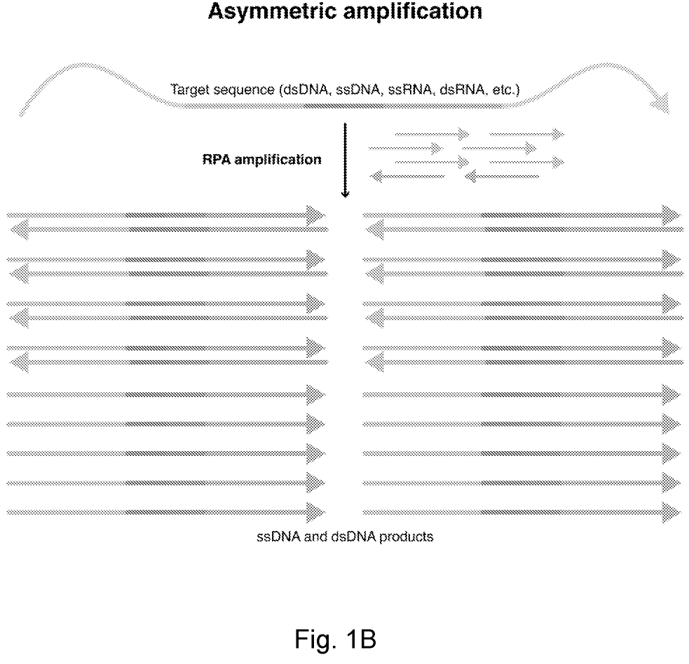

There are several strategies that can be utilized to produce a single-stranded RPA amplicon sequence. One strategy uses an exonuclease, (e.g. T7 exonuclease, lambda exonuclease, Exonuclease VIII, T5 exonuclease, RecJf, or any combinations thereof) to digest just one of the two strands of the double-stranded amplicon (see e.g., FIG. 1A). The primer being digested can be phosphorylated on its 5′ end to ensure better digestion, and the remaining primer can be protected on its 5′ end (e.g. with a series of phosphorothioate (PS) bonds) to reduce exonuclease digestion.

Another strategy for producing single-stranded DNA is to include a higher quantity of one primer versus the other, so that an asymmetric RPA reaction produces both double-stranded amplicons and single-stranded amplicons (see e.g., FIG. 1B). In some cases, there may be a possibility of the single-stranded products undergoing further spurious extension. In this case, a number of strategies can be deployed to protect the end from extending further (either on itself or on another strand). The 3′ ends of the product can be protected through a couple of strategies that modify or add additional bases to the 5′ of the opposite primer (see e.g., FIGS. 2A-2B).

A strategy to permit hybridization-based detection of RPA amplification can be to detect a product of transcription (e.g. T7-based transcription; see e.g., U.S. Pat. 10,266,886; U.S. Pat. 10,266,887; Gootenberg et al., Science. 2018 Apr 27;360(6387):439-444; Gootenberg et al., Science. 2017 Apr 28;356(6336):438-44), which produces single-stranded RNA that can be detected through any of the single-stranded sequence readouts described.

While a few steps at elevated temperatures enable the method to proceed rapidly, the amplification can also take place at lower temperatures such as room temperature more slowly thus requiring no special incubation equipment for amplification.

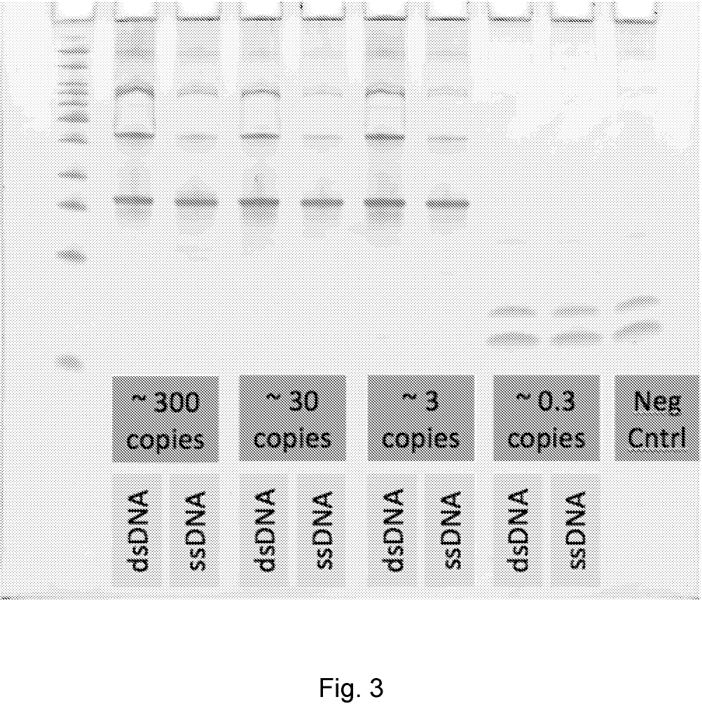

RPA amplification was performed using different copy numbers of RNA (starting material). Gel electrophoresis data indicates that RPA can successfully amplify product down to approximately 3 copies. As control, negative control (with no RNA template or starting material) was run on the same gel. “dsDNA” indicates post-RPA samples, when amplicons remain in double-stranded product. “ssDNA” indicates post-exo treatment, in which double-stranded product is digested to only leave single-stranded target band (see e.g., FIG. 3).

### Additional Strategies

In addition to exonuclease-digestion, asymmetric amplification, and/or stopper-based priming to expose single-stranded amplicon, any of the following three strategies can be used.

1) Use of recombinase and/or single-stranded binding protein (SSB) to localize probe(s) to the target sequence of interest in a double-stranded amplicon.

2) Use of Cas-family proteins (Cas9, dCas9, Cas13) or zinc finger nucleases or TALEN’s, which may be mutated to have non cleavage effects or be programmed to have activated specific or nuclease activity upon binding the target sequence, to localize guide RNA or DNA probes to amplicon sequence. These probes may be functionalized as previously described for fluorescence, colorimetric, LFD, or other readout.

3) Use of non-canonical (e.g. non B-form) DNA structure formation for detection of duplex DNA, such as by localizing a single-stranded probe to a GA-rich region of amplicon sequence via the formation of a triplex structure.

### Lateral Flow Device Readout

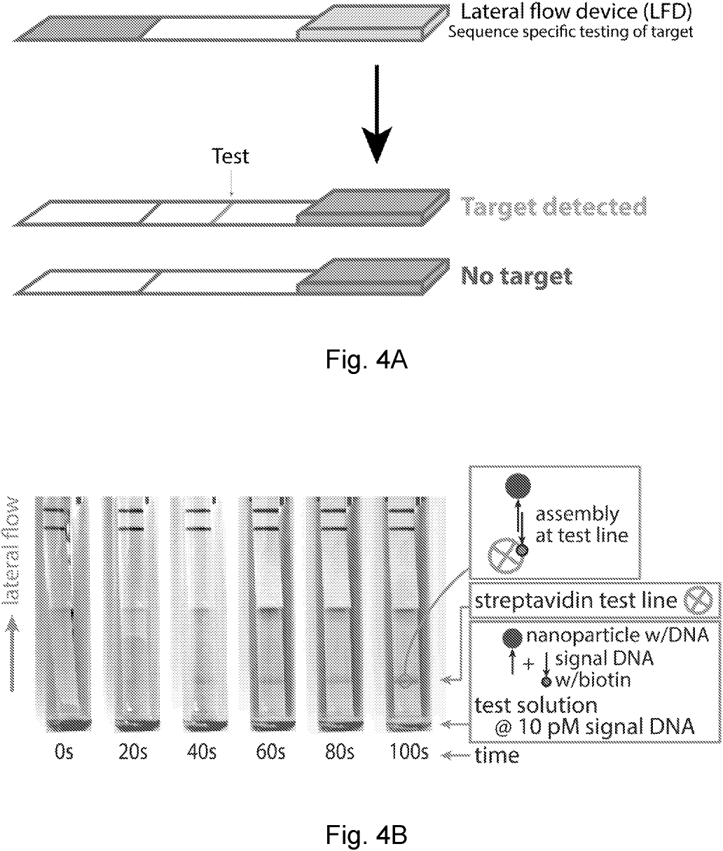

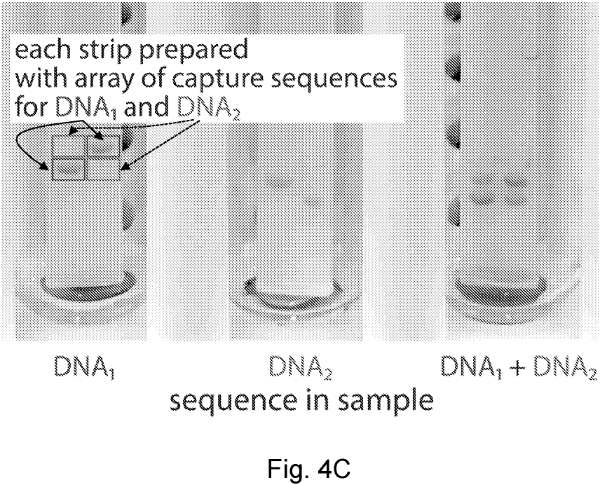

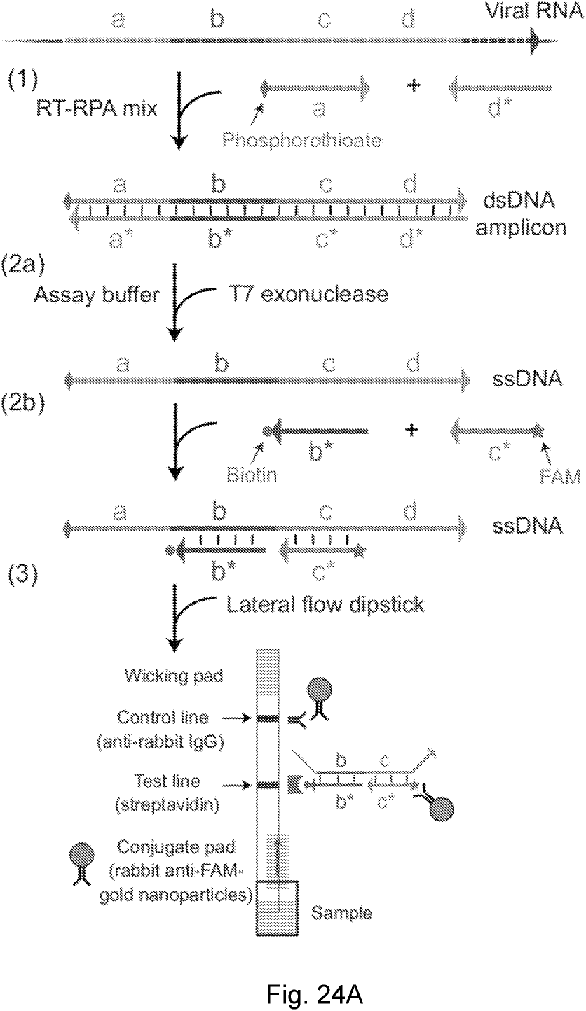

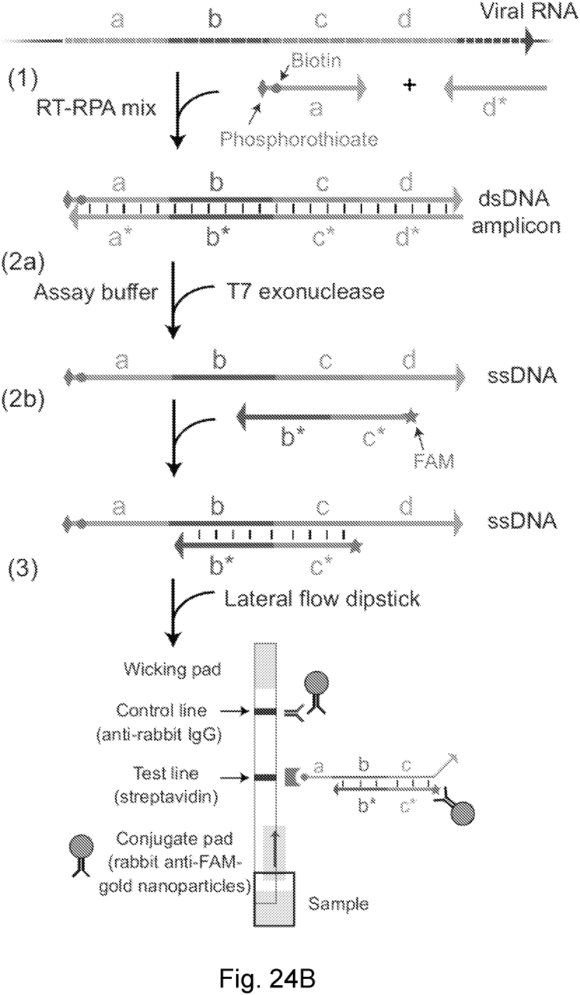

Sequence-specific LFD detection of target amplicons can be performed through a number of hybridization-based strategies. One strategy of single-stranded target detection utilizes the target directly labeled with biotin (see e.g., FIGS. 4A-4C). For example, a biotinylated, protected primer and a simple, ‘normal’ primer act to produce amplicons with a biotin. These cannot by themselves activate an LFD test line, but digestion (or other conversion) to ssDNA labeled with biotin allows a FAM probe to hybridize. This strategy provides a double check on sequence of amplicon (see e.g., FIGS. 24A-24B). Using this type of single-stranded nucleic acid detection, a signal DNA was detected at 10pM sensitivity in less than 1 minute (e.g., 45 seconds) with (see e.g., FIG. 4B).

A splint strategy whereby the target strand specifically tethers a signal strand (e.g. a sequence conjugated to a colored latex bead) to the test line (e.g. via a biotin-streptavidin interaction) can also be utilized, such as demonstrated in FIGS. 6A-6B. This strategy with two hybridization probes provides a triple check on the sequence of the amplicon.

Alternative designs utilize a primer with a pad-tethering moiety (e.g. a biotin that binds a streptavidin test line) or a primer directly attached to a signal moiety (e.g. latex bead, gold nanoparticle, or an agent that associates with other reagents to tether the signal). Importantly, due to the programmability of nucleic acids, further strands can be incorporated into the tethered signal complex, such as a bridge strand that binds to the sequence on the signal strand and projects a distinct single-stranded domain to allow the same signal conjugates to be utilized for multiple target sequences.

Toehold-mediated strand displacement (see e.g., Yurke 2000) can also be used to read out the amplicon sequence. This can be done through the use of one of the aforementioned strategies for creating single-stranded products followed by toehold-based detection of part or all of the amplicon sequence between the primers (see e.g., FIG. 5A), or through the use of strategies to expose parts of primer sequences or their complements that can act as toeholds (see e.g., FIGS. 5B-5C). The latter strategy is suitable for use with standard symmetrical RPA, where primers are included at equimolar concentrations and primarily double-stranded amplicon products are produced. The use of toehold-mediated strand displacement, rather than purely hybridization-based associations to detect target sequences, can further improve specificity to single-based detection (see e.g., Zhang 2012). Instead of toehold-mediated strand displacement readout of these amplicons, molecular beacons (see e.g., Tyagi 1996) can instead be utilized.

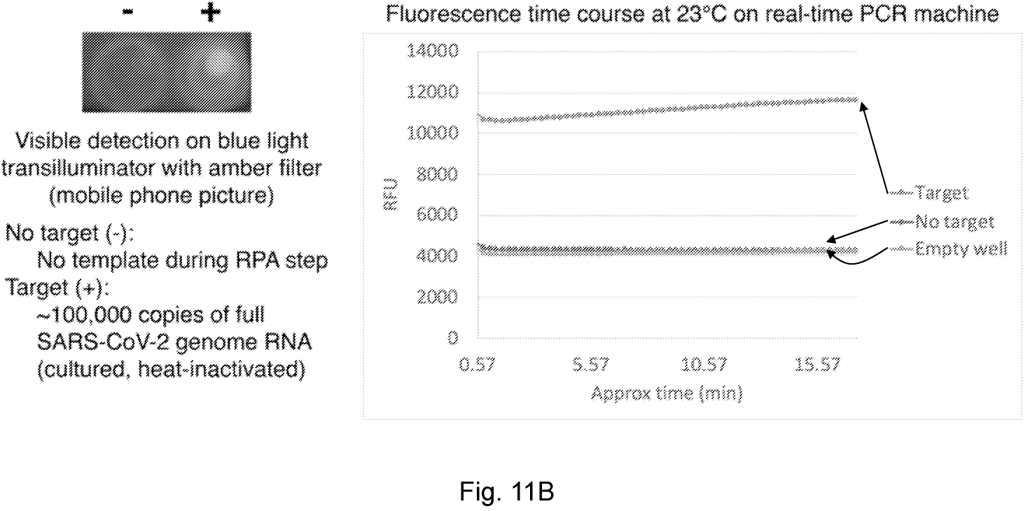

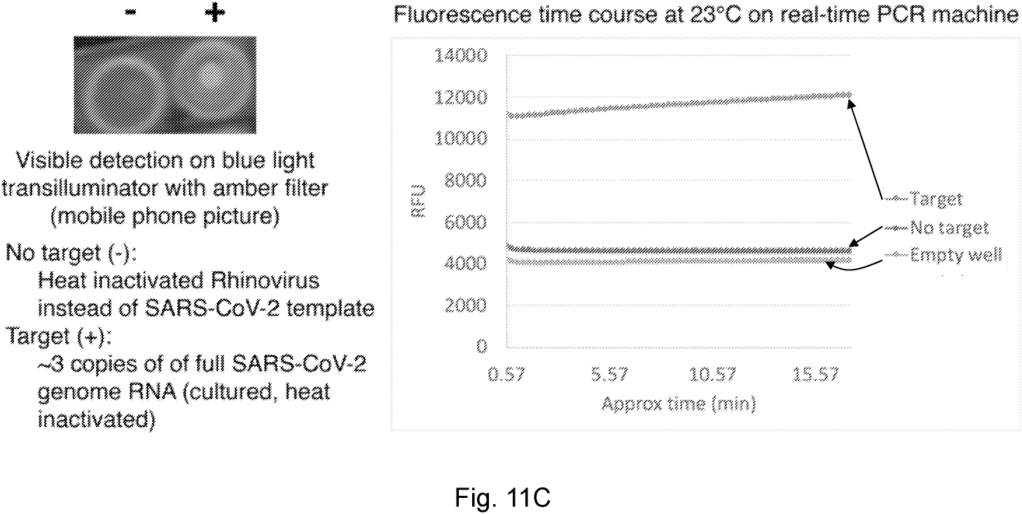

Toehold-mediated strand displacement can be used to specifically detect the single-stranded amplicon through a fluorescence assay (see e.g., FIG. 10 and e.g. Zhang et al. 2012 Nature chemistry 4.3 (2012): 208). A fluorophore-labeled strand and quencher strand are assembled together so that the fluorescence is quenched in the absence of the target amplicon, but when in the presence of the target, the fluorescent strand can become displaced from the quencher strand and produce fluorescence. This fluorescence can be detected by eye, for example, with the use of appropriate lighting, or through fluorescence scanners, fluorescence plate readers, or real-time PCR machines (see e.g., FIGS. 11A-11C).

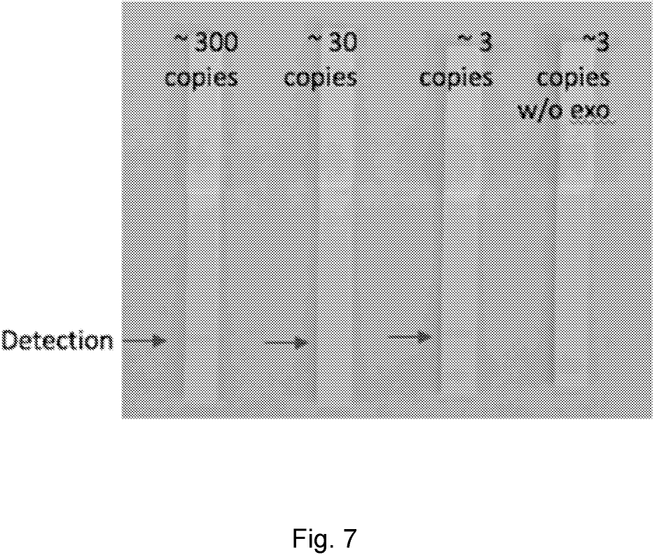

The full workflow was tested with the strategy depicted in FIGS. 6A-6B. LFD can detect amplified product of ~ 3 copies of RNA. LFD strips show a red test line that indicate presence of target (at red arrow that says “Detection”). RPA product without exonuclease treatment (still remaining in double-stranded product) cannot be detected on LFD. Therefore, only when ssRPA is applied (RPA+exo) can single-stranded target be detected (see e.g., FIG. 7).

Even higher sensitivity detection of the single-stranded amplicon readout can be achieved through the use of secondary amplification steps, such as an HRP-mediated chromogenic precipitate reaction. However, these also require additional components and complexity. In addition to visible latex bead readouts, gold nanoparticles, chemiluminescent, fluorescent, or other visual readout strategies can be utilized. LFD’s can be further paired with a digital reader device for accurate quantification of results.

Alternative readout strategies can also be deployed, such as fluorescent readout of the single-stranded products in solution with fluorophore-quencher pairs, microarrays printed on LFD’s or other surfaces, or through sequencing of products.

### Conclusion

Described herein are methods for preparing single-stranded DNA products from RPA followed by rapid detection with Lateral Flow Devices (LFD’s). Importantly, the readout mechanism checks that the correct sequence has been amplified to ensure that background amplicons from the RPA step (e.g. primer dimers, incorrect products) are filtered out and therefore do not result in false positives. The strategy is flexible to a variety of target types (single-stranded RNA, single-stranded DNA, double-stranded DNA, etc.) and for arbitrary sequences, thus making it a general strategy for combined high-sensitivity and high-specificity detection of target sequences.

### Example 2: Single-Strand RPA for Rapid and Sensitive Detection of SARS-CoV-2 RNA

Described herein is the single-strand Recombinase Polymerase Amplification (ssRPA) method, which merges the fast isothermal amplification of RPA with subsequent rapid conversion of the double-strand DNA amplicon to single strands, and hence permits facile hybridization based high specificity read out. Demonstrated herein is the utility of ssRPA for sensitive (e.g., 10 copies per reaction) and rapid (e.g., 8 min reaction time post extraction) visual detection of SARS-CoV-2 RNA spiked samples, as well as clinical nasopharyngeal swabs in viral transport media (VTM) or water, and saliva, on lateral flow devices. ssRPA offers rapid, sensitive, and accessible RNA detection to facilitate mass testing for the COVID-19 pandemic.

### Introduction

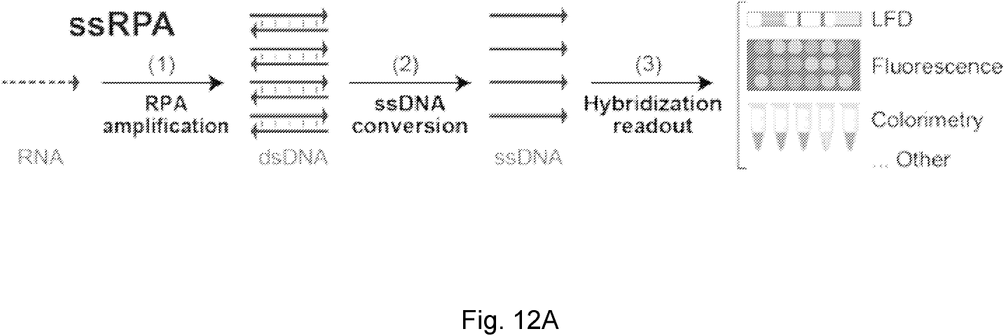

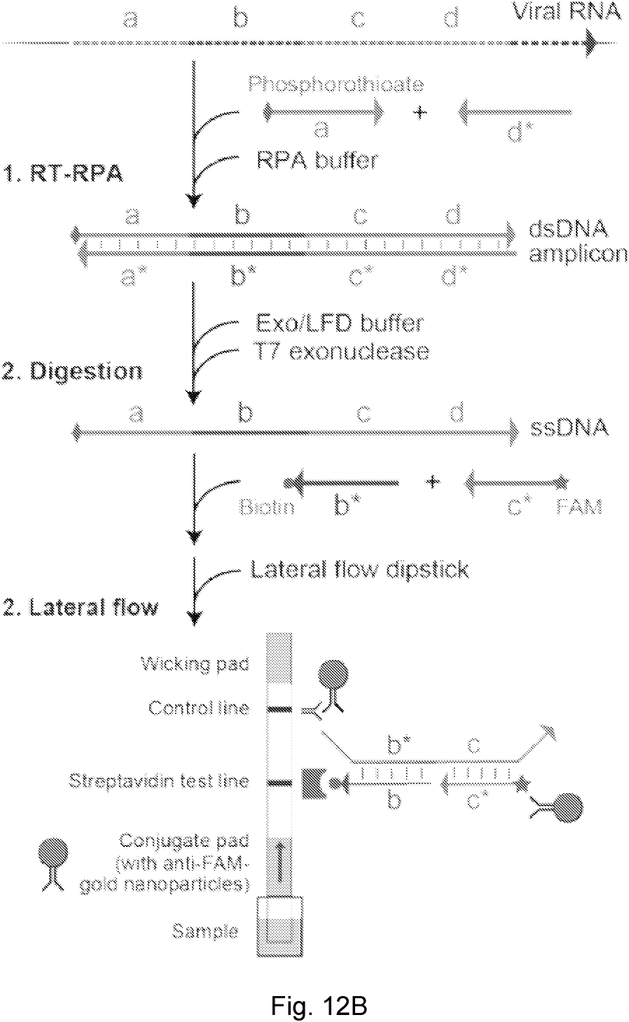

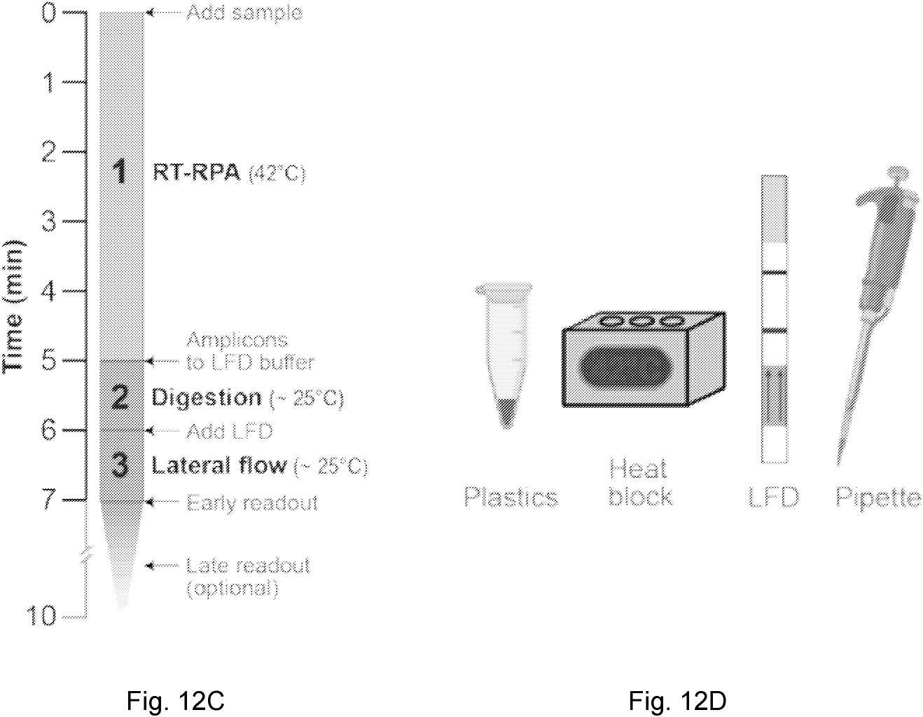

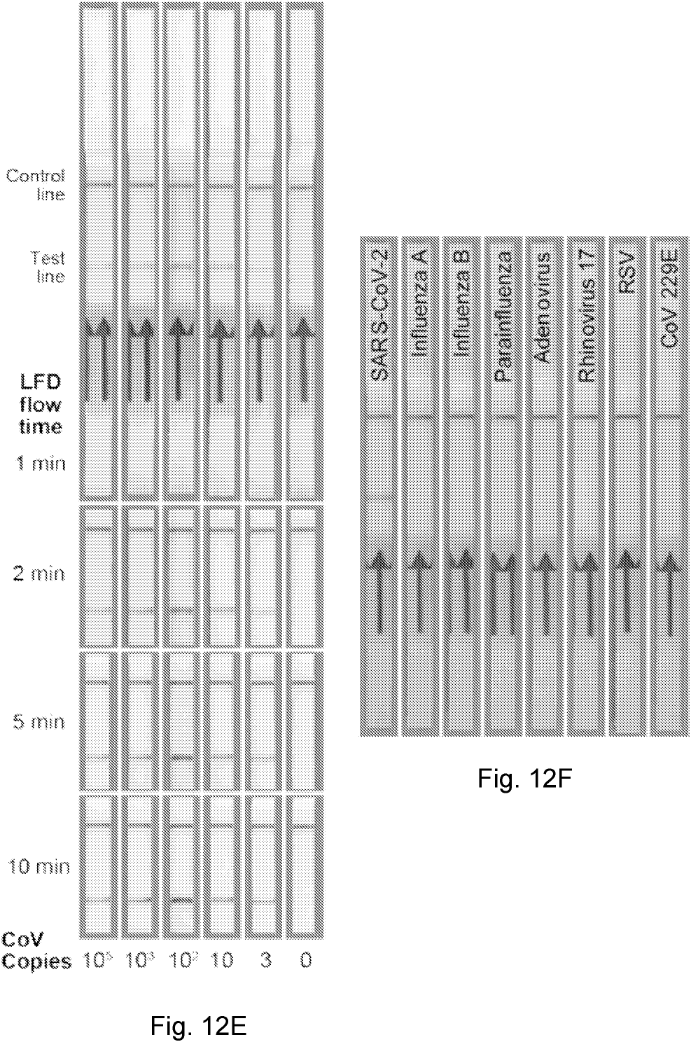

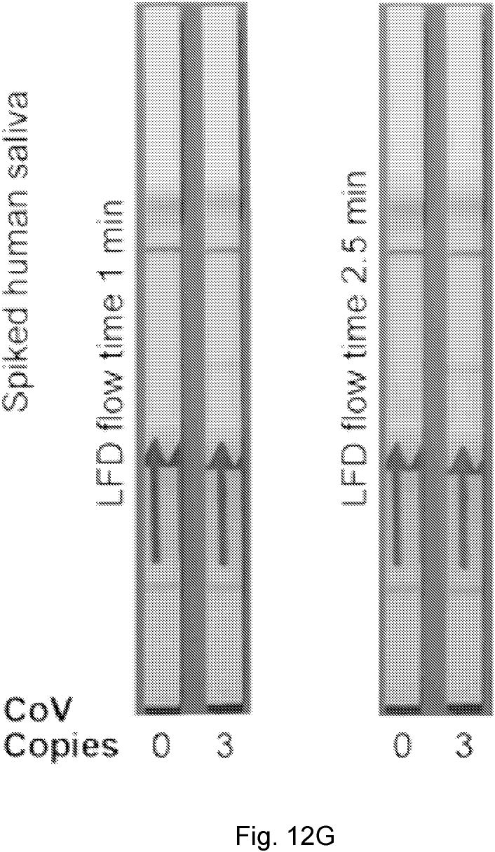

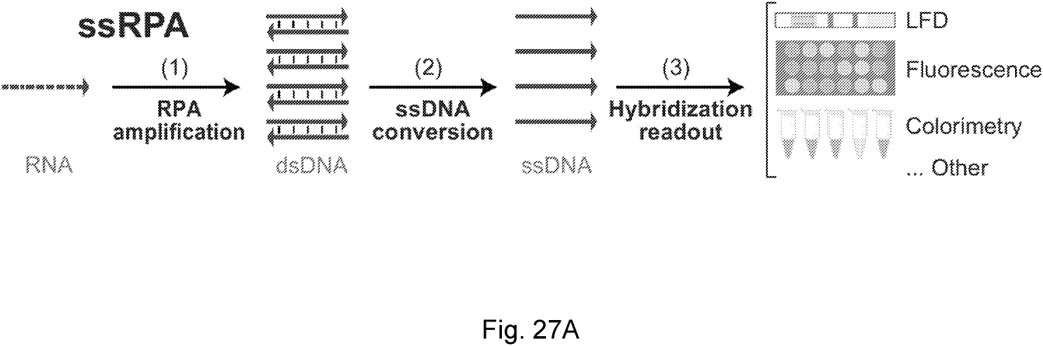

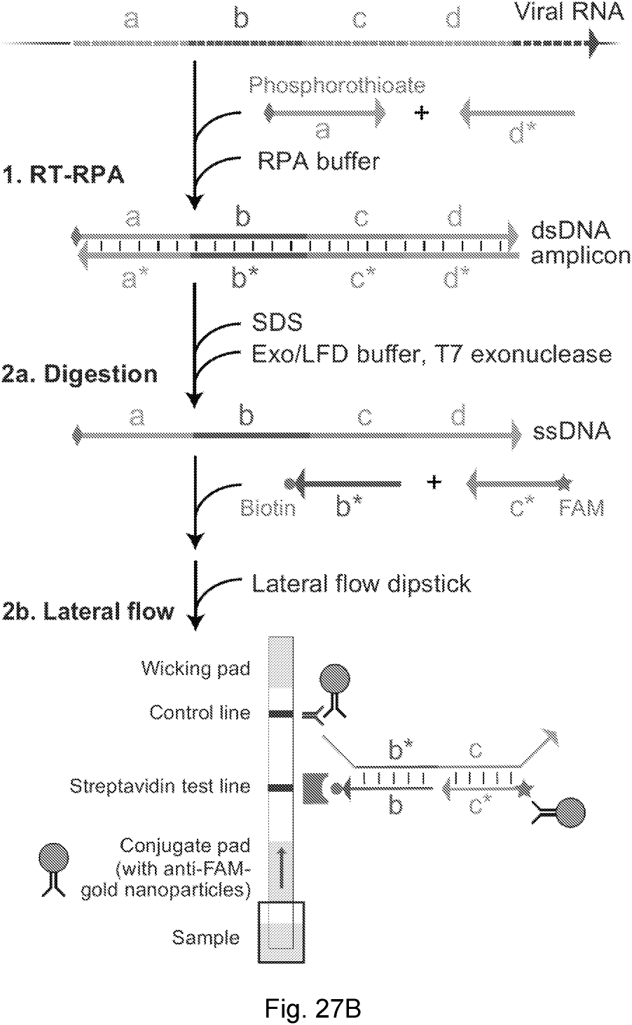

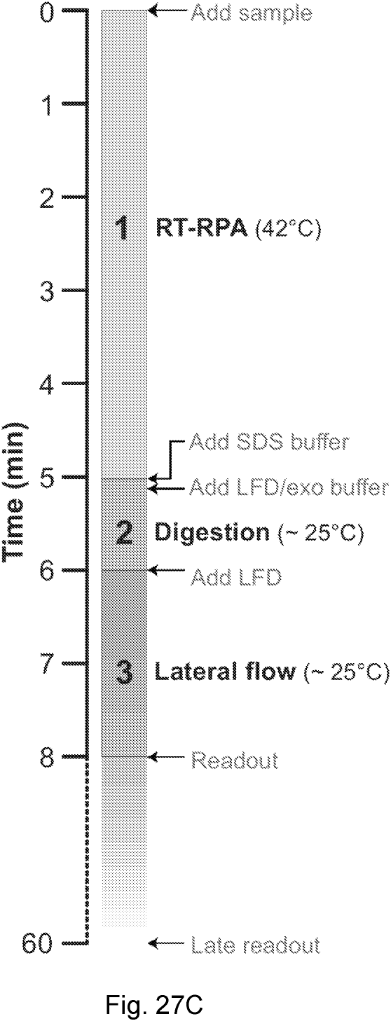

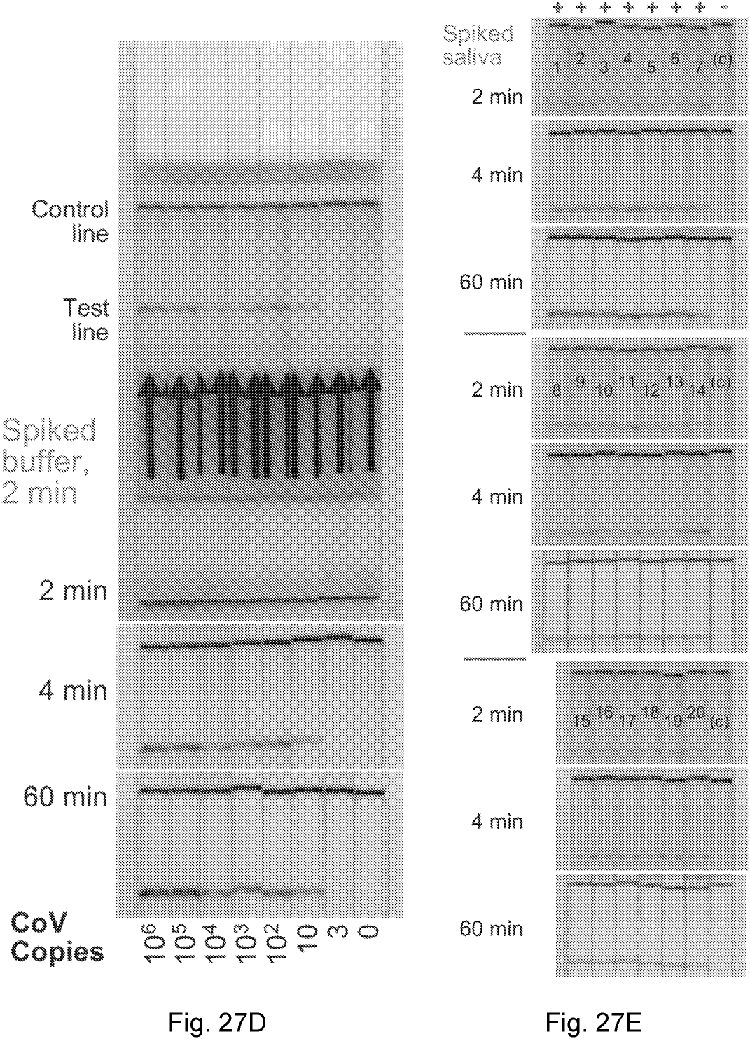

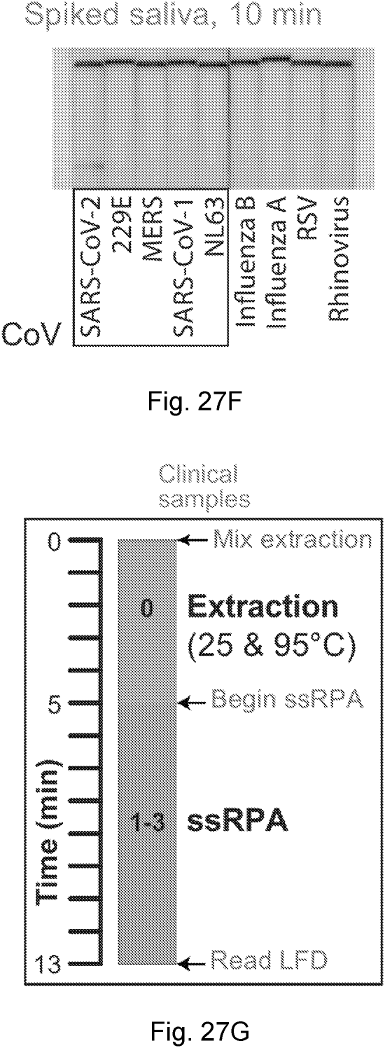

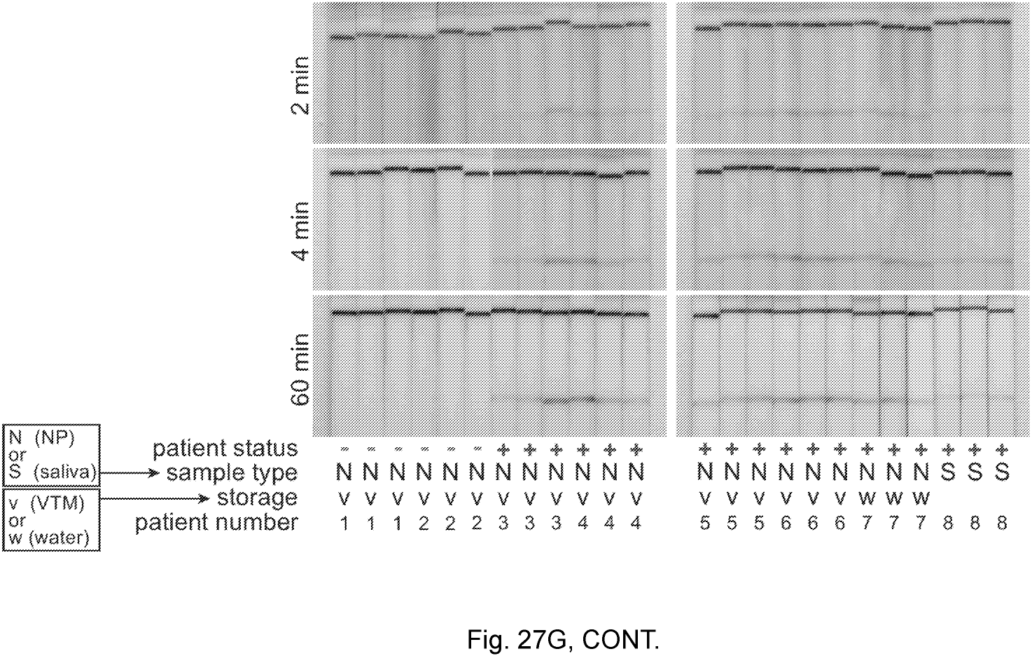

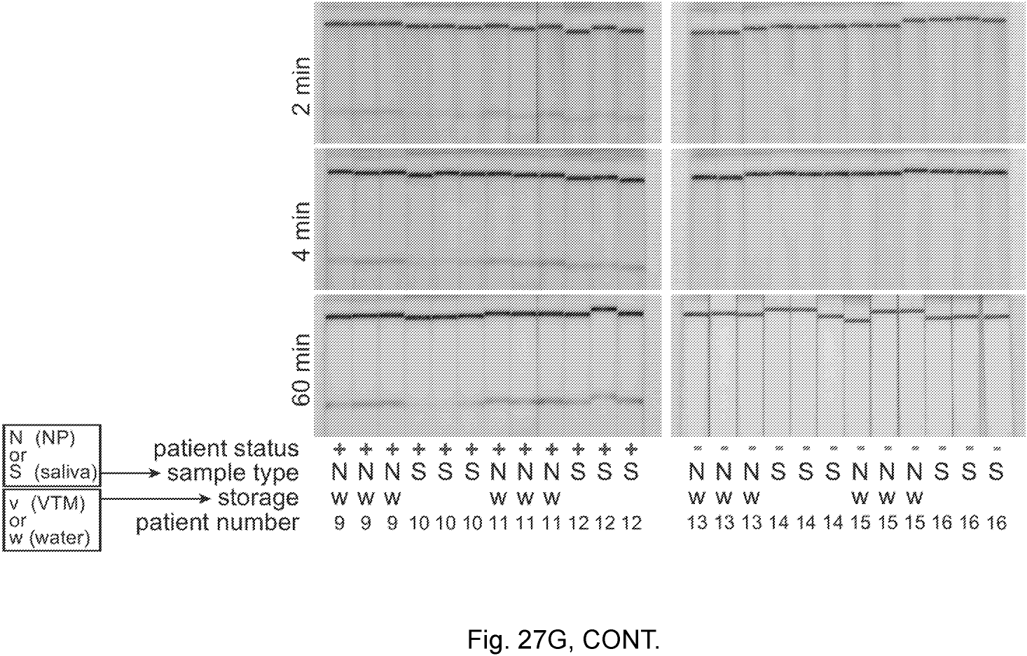

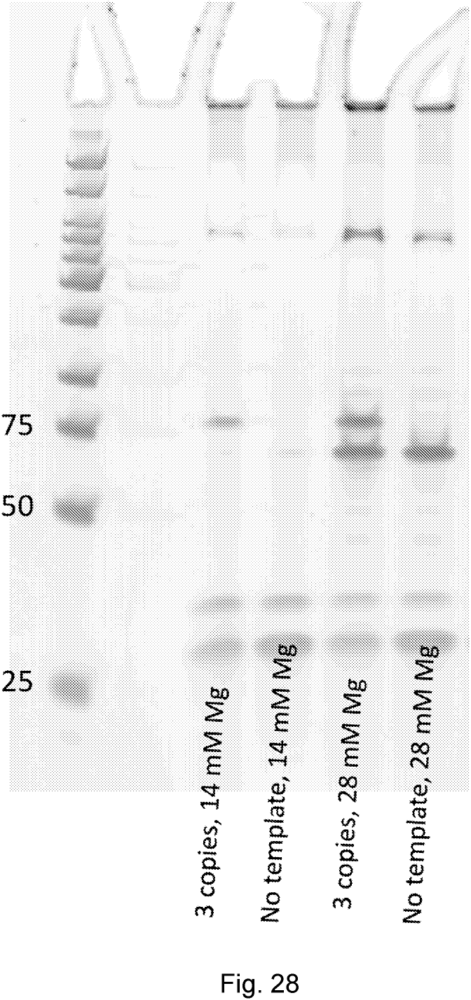

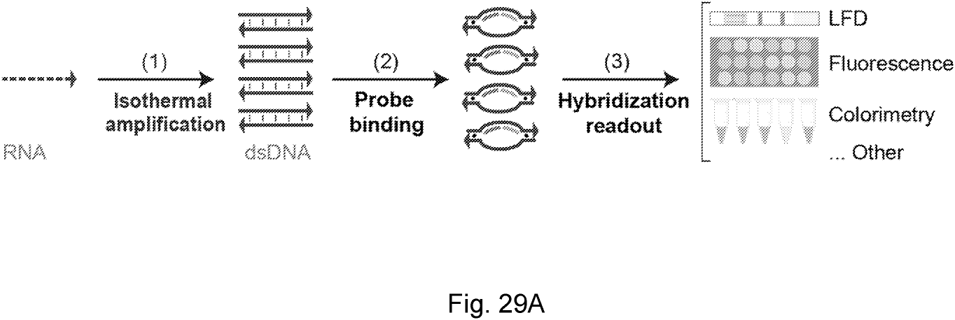

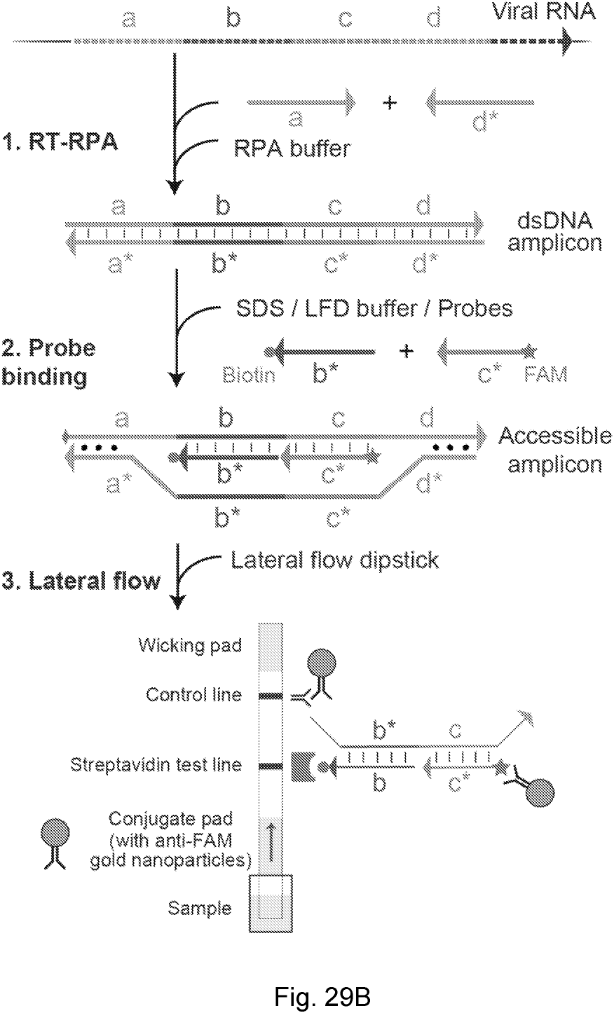

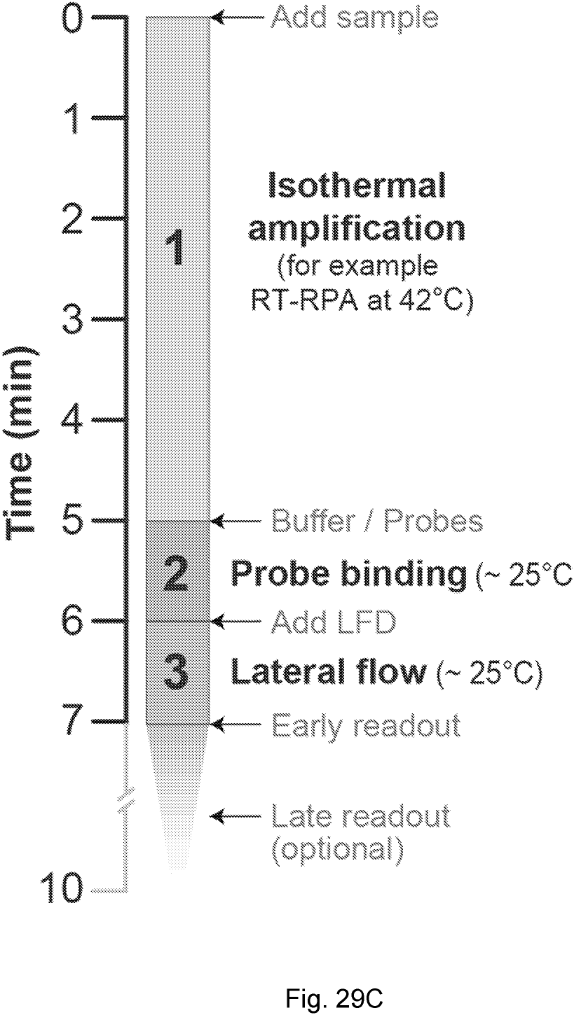

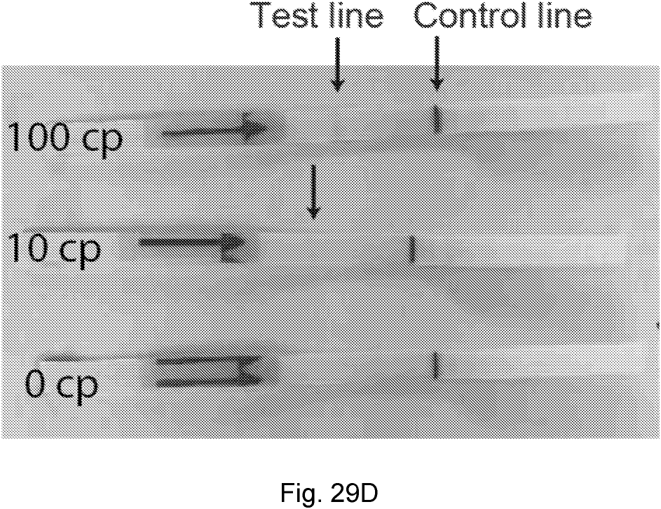

Effective and accessible mass testing can help to limit the spread of the SARS-CoV-2 pandemic. While serology testing reveals recent and past exposure, RNA testing allows early detection of active infection. Standard RT-qPCR achieves high analytical sensitivity (1-100 copies of viral RNA per input µl)¹, but takes hours and requires relatively complex equipment. Isothermal methods^{2,3}, such as Recombinase Polymerase Amplification (RPA)^{2,4} and Loop-mediated isothermal amplification (LAMP)³, can provide instrument-free detection of 10-1200 copies of RNA in 30-90 min⁵⁻⁸ (see review⁹). The RPA reaction can generate millions of copies of double-stranded DNA (dsDNA) amplicons within minutes, but its recombinase-driven priming process is prone to multi-base mismatching that necessitates an additional specificity check^{8,10}. Augmentation of RPA with conditionally extensible primers or cleavable inter-primer probes enhances specificity^{2,12,13,14}, but tends to reduce reaction speed. Alternatively, Cas12⁶ or Cas13^{5,15} nucleases applied to amplification products generate signal in a sequence specific manner, but incurs substantial increases in workflow complexity and reaction time. Described herein is the “single-strand RPA” (ssRPA) method, which serially applies (1) rapid amplification of dsDNA, (2) conversion to ssDNA, and (3) sequence-specific hybridization-based readout, arranged to maintain both optimal speed and accuracy (see e.g., FIG. 12A, FIG. 27A). For amplification, basic RT-RPA² was applied separate from specificity-enhancing components that can inhibit class-leading speed. For ssDNA conversion, exonuclease was used to digest all but chemically protected targets. Finally, hybridization based readout is demonstrated by LFDs.

### Results

The 5′ end of the SARS-CoV-2 spike protein sequence was selected as the main detection target. In the detailed protocol of FIG. 12B or FIG. 27B (see e.g., Protocol A or Protocol B) the sample was diluted in a basic RT-RPA reaction mixture, modifying the forward primer with a 5′ tail of 6 phosphorothioate-linked bases to confer exonuclease protection^{16,17}. The reaction was run for 5 min at 42° C. on a heating block, averaging a < 8 s doubling interval (few copies to > 10 nM, 50 µl within 5 min represents > 36 doublings). A sample of the product was then treated with sodium dodecyl sulfate (SDS) and diluted into an exonuclease and lateral flow (exo/LFD) buffer, where the unprotected strand in the dsDNA was rapidly (≤ 1 min) digested by a T7 exonuclease to yield the protected ssDNA target^{16,17}. A pair of 3′-biotin and 5′-FAM modified probes in the digestion buffer were available to target sequences in between - and therefore independent of - the amplification priming domains, providing specificity not achievable with RPA priming alone. The correct target ssDNA therefore acted as a bridge that co-localized both detection probes within an LFD, ultimately binding gold nanoparticles to the streptavidin line to produce visual readout as early as 1-2 minutes. Full reaction timelines for ssRPA and LFD detection are described in FIG. 12C and FIG. 27C. The protocol can be performed with a test tube, a heat block or water bath at 42° C., an LFD strip, and a micropipette (see e.g., FIG. 12D.).

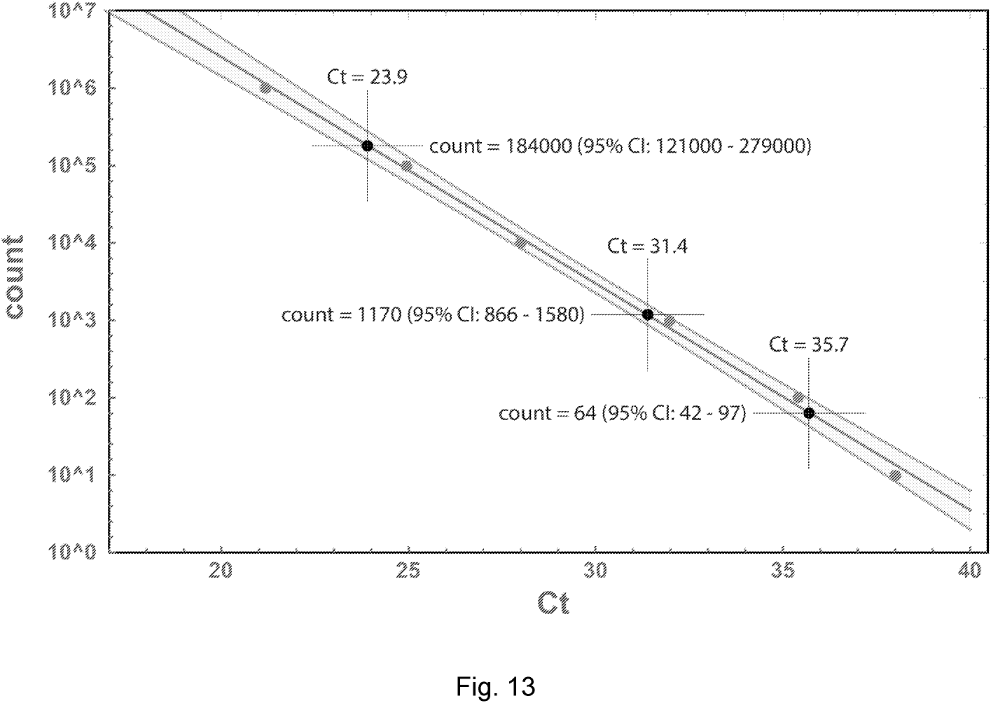

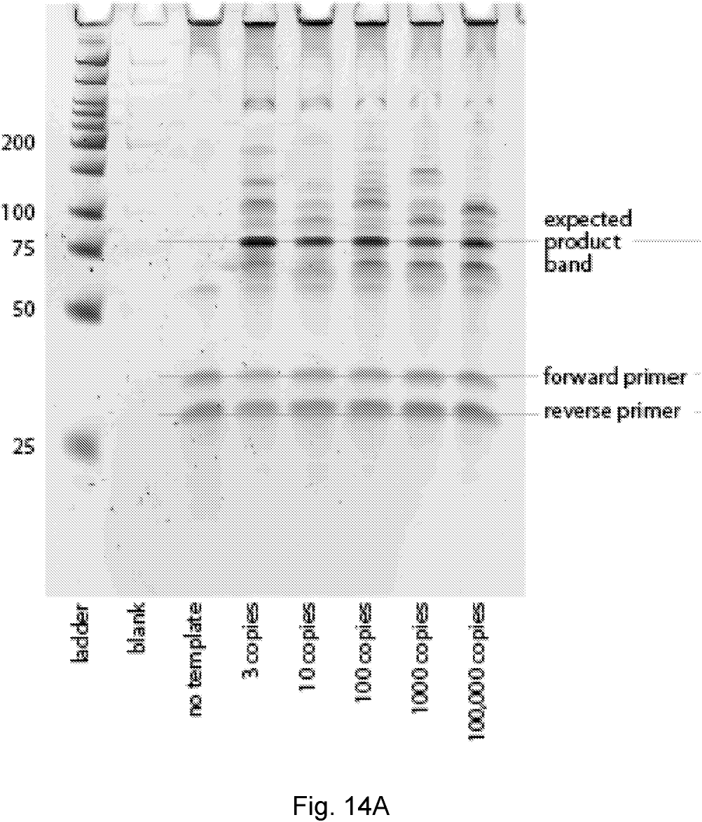

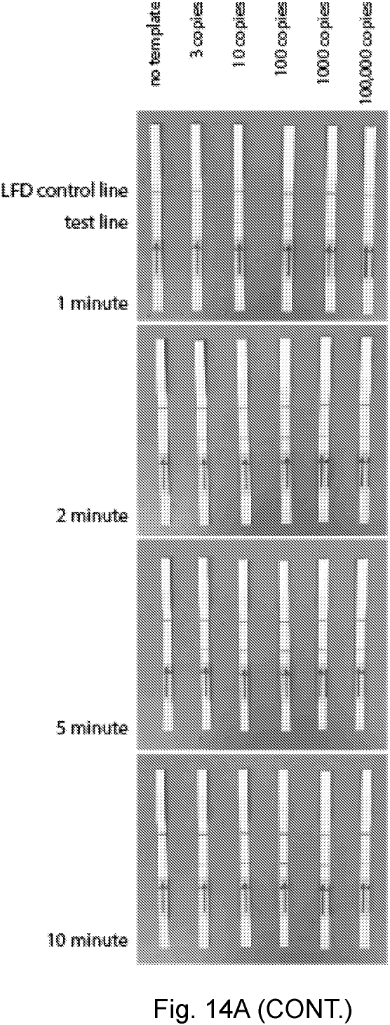

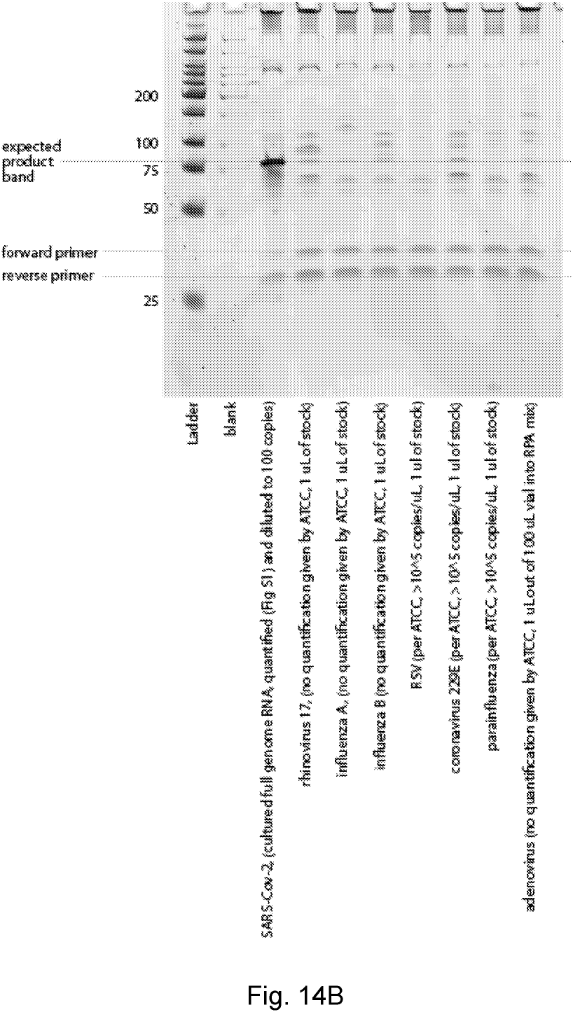

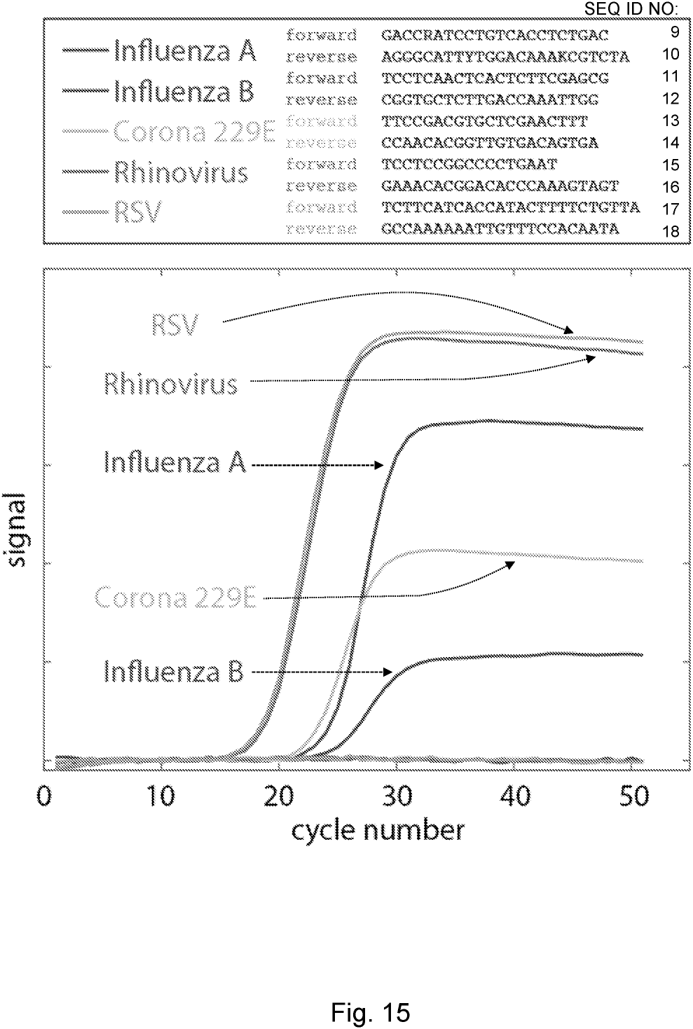

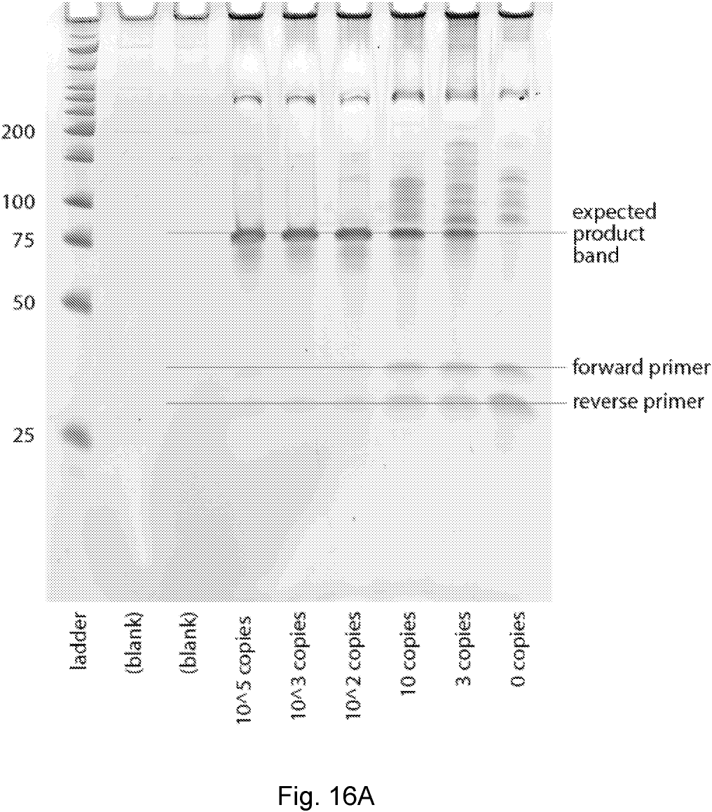

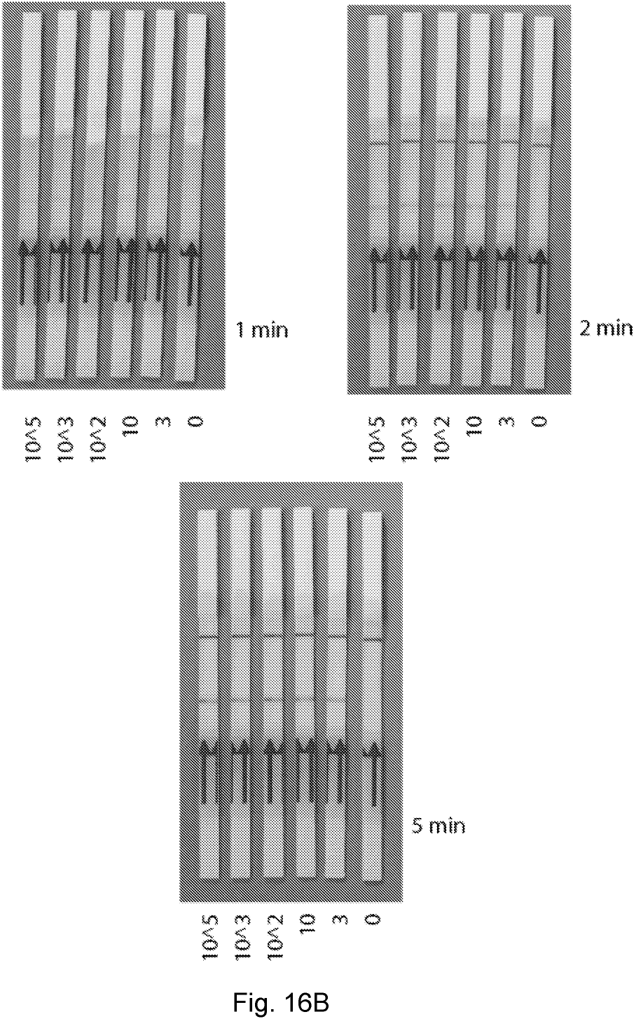

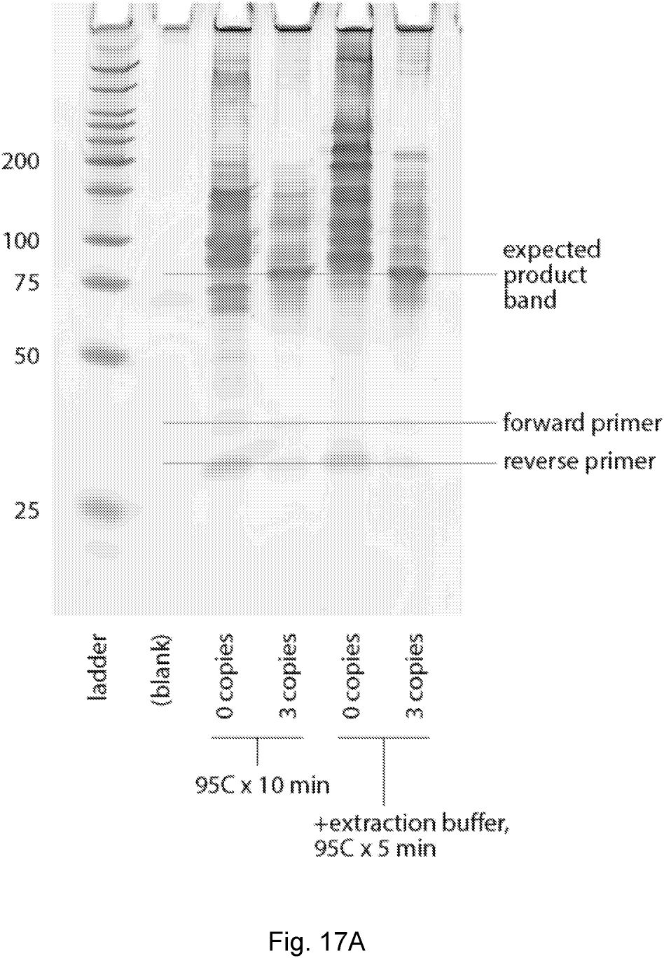

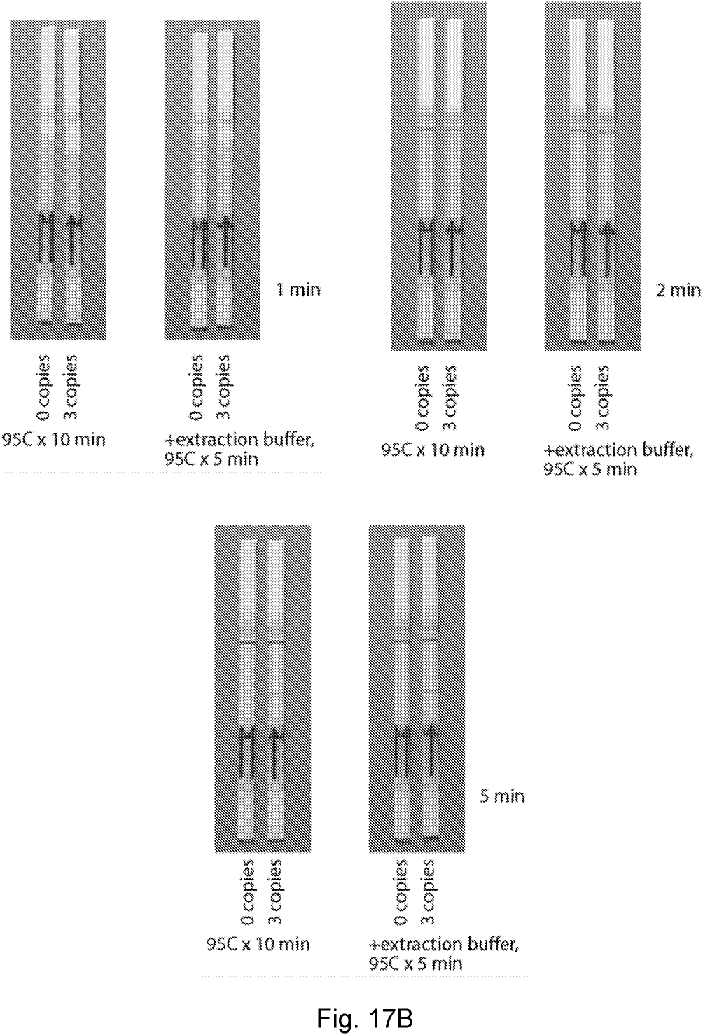

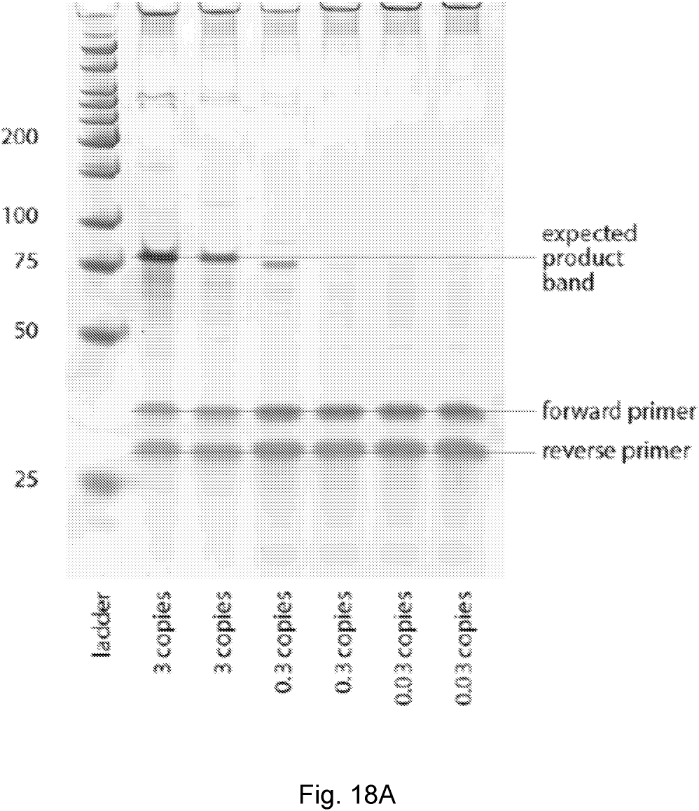

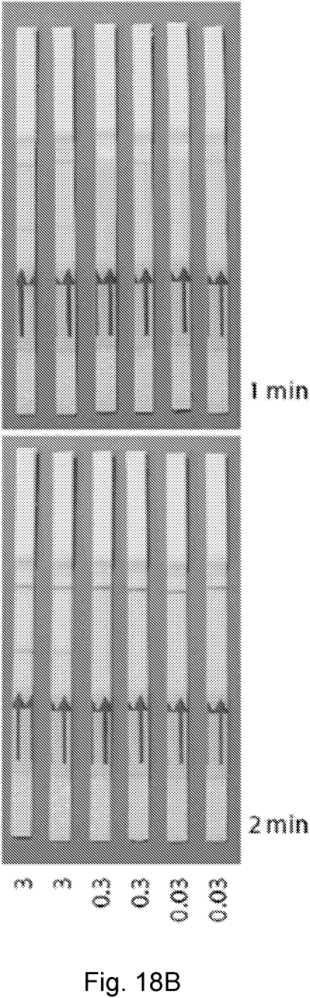

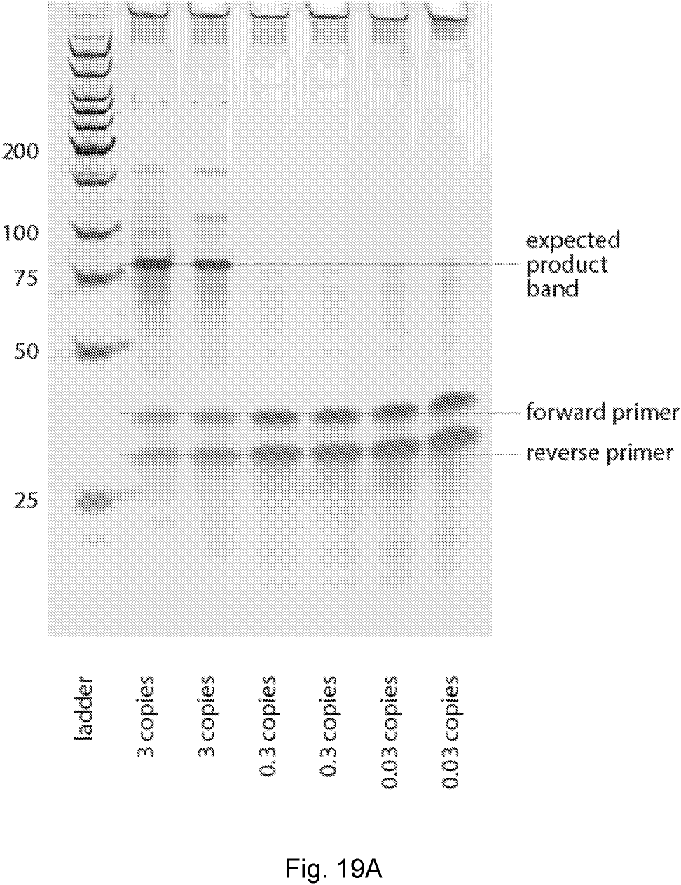

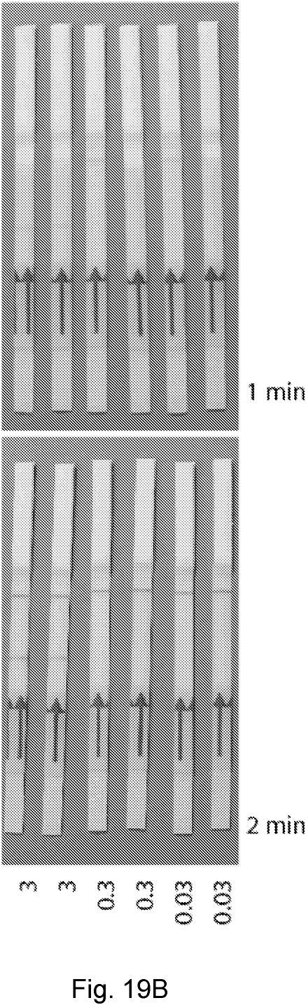

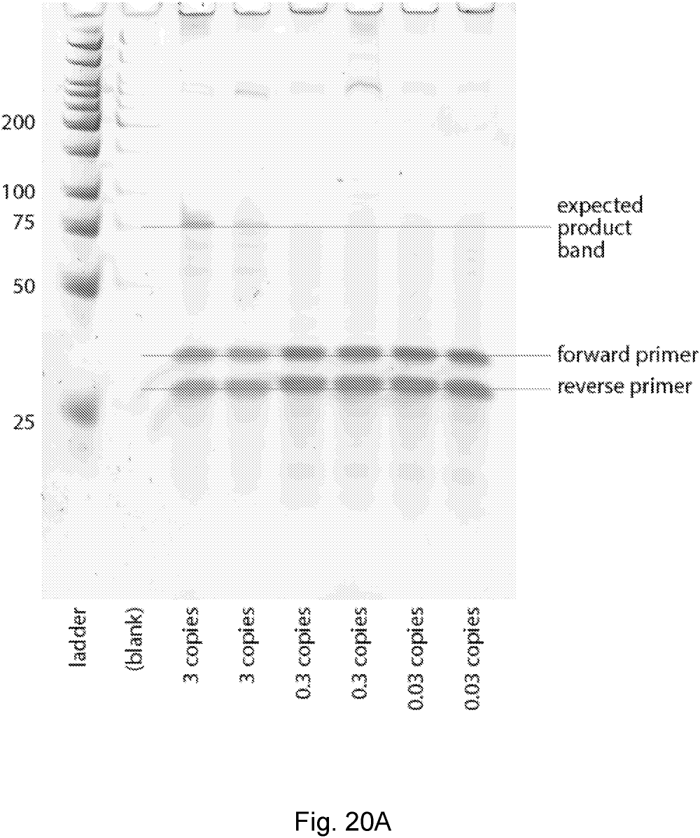

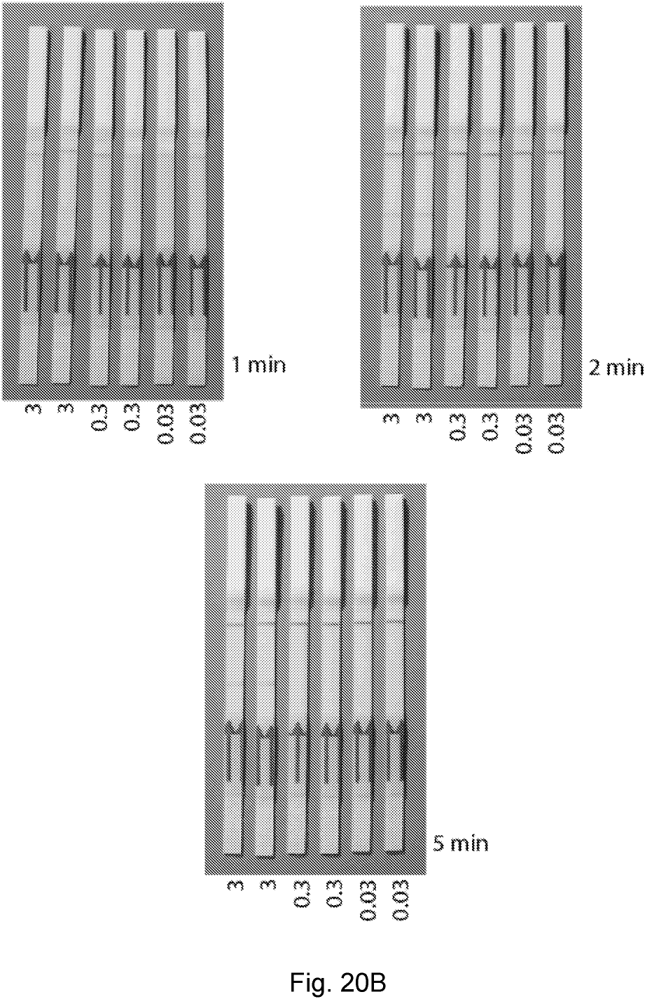

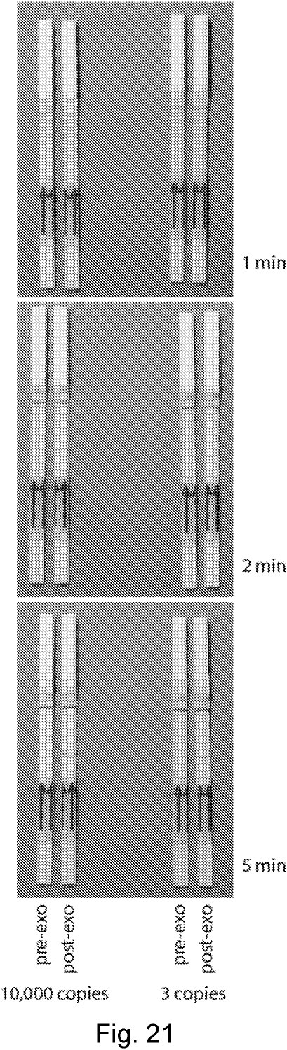

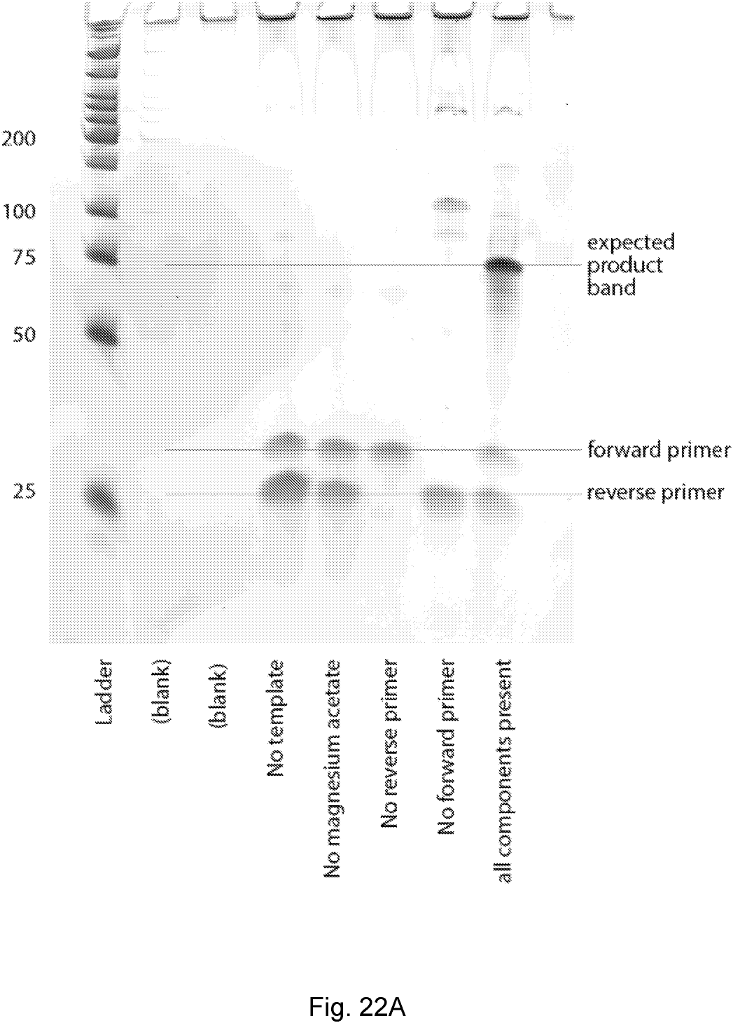

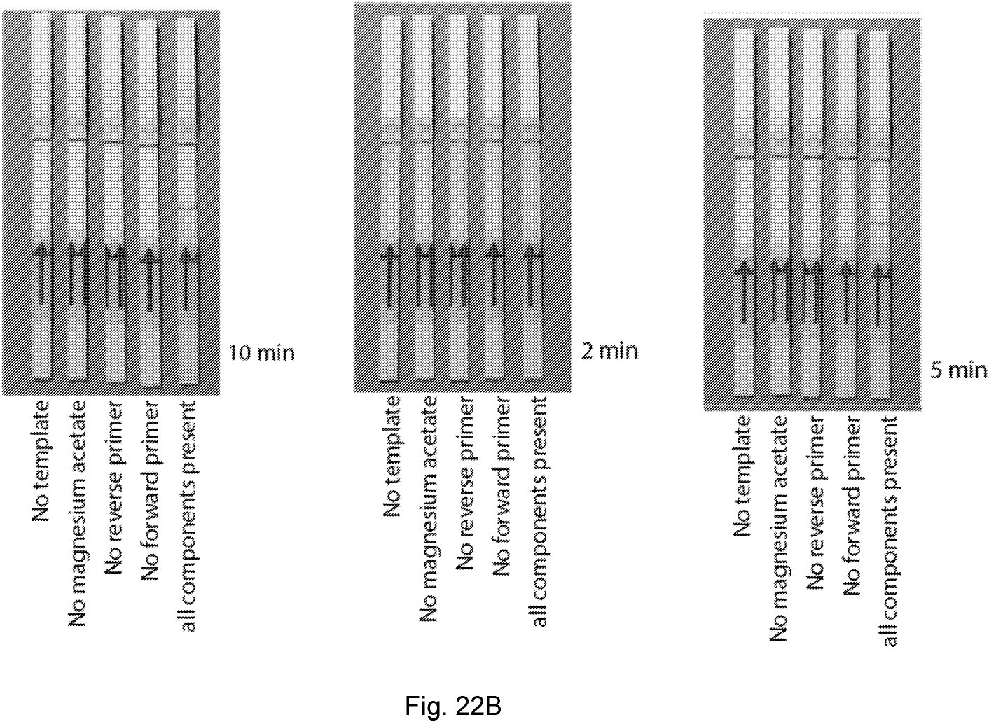

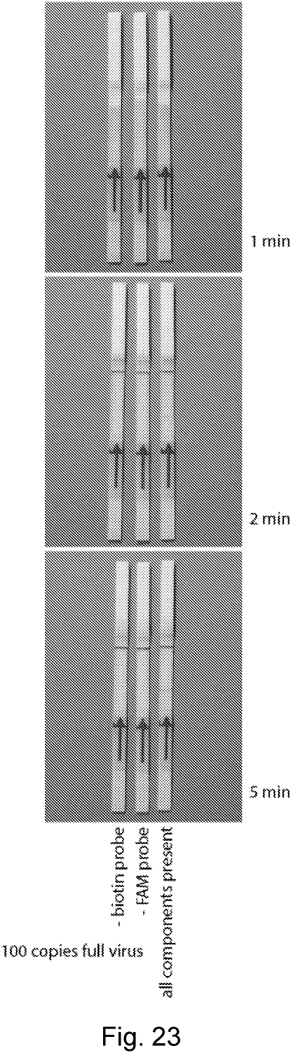

ssRPA was first tested on buffer-spiked samples. FIG. 27D (see also FIG. 12E, FIGS. 14A-14B) shows LFD detection of syntheticSARS-CoV-2 RNA serially diluted in DNase/RNase-free water, photographed at multiple intervals on the same strips. (See e.g., FIGS. 18A-18B, FIGS. 19A-19B, and FIGS. 20A-20B for other sample type dilutions, and FIG. 21, FIGS. 22A-22B, and FIG. 23 for demonstrations that exonuclease and other components are required.) Concentrations of input RNA were quantified by RT-qPCR and direct comparison to commercial standards (see e.g., FIG. 13). Results show detection sensitivity down to ~10 RTqPCR-detectable copies in a 50 µl assay volume, and a dynamic range of at least 5 orders of magnitude. True positive results were observed starting at 1-2 min, while absolutely no test lines formed for the no-template negative controls over > 60 min of LFD incubation. To demonstrate a Limit of Detection (LoD) under 10 (extracted) copies, human saliva was spiked with heat-inactivated, cultured virus and showed 20 of 20 positive tests (see e.g., FIG. 27E). To test for specificity, ssRPA was performed on DNase/RNase-free water spiked with viral RNA from 8 other respiratory viruses, including coronaviruses 229E, MERS, SARS-CoV-1, and NL63, and alternative diagnoses influenza B, influenza A, respiratory syncytial virus (RSV), and rhinovirus 17, each at >10⁵ copies per assay. There were no false positives after a 10 min LFD incubation (see e.g., FIG. 27F, FIG. 12F, FIGS. 14A-14B, and FIG. 15). Finally, the robustness of the assay was tested with client patient samples. A total of 16 positive and negative patient samples, taken as either nasopharyngeal (NP) swabs stored in viral transport media (VTM), NP swabs stored in water, or saliva, were processed by single-tube RNA extraction (1:1 mixture with extraction buffer, 95° C. × 5 min) and then used at 10% v/v in RT-RPA (see e.g., FIG. 27G, FIG. 12G, FIG. 16, and FIGS. 17A-17B). Results of this study demonstrated 100% sensitivity and 100% specificity across all sample types. Comparable sensitivity (e.g., 3-10 copies spiked in 5 µl saliva diluted to a 50 µl final reaction volume) and speed (e.g., 7 min reaction time post extraction) was achieved as in the spiked water. See e.g., FIG. 21 for demonstration of exonuclease requirement.

### Discussion

The ssRPA method combines the speed of RT-RPA² with the sequence specificity of ssDNA hybridization by serially applying RPA and exonuclease steps. As an alternative to single-strand conversion, post amplification-hybridization readout may also be achieved via high-temperature melting and re-hybridization to bind LFD probes. The ssRPA conceptual framework can be generalized to other isothermal readout methods with dsDNA output for achieving optimal sensitivity and speed. The present method can also be used to achieve single-nucleotide specificity e.g. by using toehold probe readout¹⁹ on LFDs with or without multiple test positions. ssRPA can also be implemented with a one-pot workflow or with the use of lyophilized reagents for ambient distribution and storage, which further facilitate mass testing.

### REFERENCES

1. Wölfel, R. et al. Virological assessment of hospitalized patients with COVID-2019. Nature (2020) doi:10.1038/s41586-020-2196-x.
2. Piepenburg, O., Williams, C. H., Stemple, D. L. & Armes, N. A. DNA Detection Using Recombination Proteins. PLOS Biol. 4, e204 (2006).
3. Notomi, T. Loop-mediated isothermal amplification of DNA. Nucleic Acids Res. 28, 63e-663 (2000).
4. Li, J., Macdonald, J. & von Stetten, F. Review: a comprehensive summary of a decade development of the recombinase polymerase amplification. The Analyst 144, 31-67 (2019).
5. Zhang, F., Abudayyeh, O. O. & Gootenberg, J. S. A protocol for detection of COVID-19 using CRISPR diagnostics. 8.
6. Broughton, J. P. et al. CRISPR-Cas12-based detection of SARS-CoV-2. Nat. Biotechnol. (2020) doi:10.1038/s41587-020-0513-4.
7. Rabe, B. A. & Cepko, C. SARS-CoV-2 Detection Using an Isothermal Amplification Reaction and a Rapid, Inexpensive Protocol for Sample Inactivation and Purification. doi:10.1101/2020.04.23.20076877.
8. Bhadra, S., Riedel, T. E., Lakhotia, S., Tran, N. D. & Ellington, A. D. High-surety isothermal amplification and detection of SARS-CoV-2, including with crude enzymes. doi:10.1101/2020.04.13.039941.
9. Esbin, M. N. et al. Overcoming the bottleneck to widespread testing: A rapid review of nucleic acid testing approaches for COVID-19 detection. RNA ma.076232.120 (2020) doi:10.1261/ma.076232.120.
10. TwistAmp DNA Amplification Kits: Assay Design Manual.
11. Mansfield, M. A. Design Considerations for Lateral Flow Test Strips. 32.
12. Powell, M. L. et al. New Fpg probe chemistry for direct detection of recombinase polymerase amplification on lateral flow strips. Anal. Biochem. 543, 108-115 (2018).
13. Xia, S. & Chen, X. Ultrasensitive and Whole-Course Encapsulated Field Detection of 2019-nCoV Gene Applying Exponential Amplification from RNA Combined with Chemical Probes. (2020) dol:10.26434/chemrxiv.12012789.v1.
14. Xia, X. et al. Rapid detection of infectious hypodermal and hematopoietic necrosis virus (IHHNV) by real-time, isothermal recombinase polymerase amplification assay. Arch. Virol. 160, 987-994 (2015).
15. Kellner, M. J., Koob, J. G., Gootenberg, J. S., Abudayyeh, O. O. & Zhang, F. SHERLOCK: nucleic acid detection with CRISPR nucleases. Nat. Protoc. 14, 2986-3012 (2019).
16. Han, D. et al. Single-stranded DNA and RNA origami. Science 358, eaao2648 (2017).
17. Sayers, J. R., Schmidt, W. & Eckstein, F. 5′-3′ Exonucleases in phosphorothioate-based oligonucleotide-directed mutagenesis. Nucleic Acids Res. 16, 791-802 (1988).
18. Wahed, A. A. E. et al. Recombinase Polymerase Amplification Assay for Rapid Diagnostics of Dengue Infection. PLOS ONE 10, e0129682 (2015).
19. Zhang, D. Y., Chen, S. X. & Yin, P. Optimizing the specificity of nucleic acid hybridization. Nat. Chem. 4, 208-214 (2012).

### Methods

Sample sources. Synthetic SARS-CoV-2 RNA (Twist Biosciences™, 102019) was used in sample qPCR quantification, and all synthetic primers and probes (IDT) were chemically synthesized with any specified modifications, ordered at 100 or 250 nmol scale, desalted or PAGE-purified, and used as is. Viral genomic RNA (isolated from infected cells) from coronaviruses 229E (ATCC, VR-740D), MERS (BEI, NR-50549), SARS-CoV-1 (BEI, NR-52346), and NL63 (BEI, NR-44105), as well as influenza A (ATCC, VR-1736D), influenza B (ATCC, VR-1535D), respiratory syncytial virus (ATCC, VR-1580DQ), and rhinovirus (ATCC, VR-1663D) were used for the specificity experiment in FIG. 12F and FIG. 27F. Heat-inactivated SARS-CoV-2 (BEI, NR-52286) was used in all SARS-CoV-2 spike-in experiments, including the saliva LoD assays. Pooled human saliva from ≥3 de-identified donors (Lee Biosolutions™, 991-05-P) was collected prior to November 2019 and used to prepare the contrived samples. All clinical samples were purchased from BioCollections Worldwide™, Inc., and heat-inactivated at 95° C. for 5 min before shipping.

Primer and probe design. Relatively clean RPA products are the result of multiple primer pairs designed in silico for the appropriate salt and temperature conditions (NUPACK.org) and tested empirically, with the goal of minimizing primer dimers and other unintended reactions that might slow the targeted amplification. RPA primer sequences and LFD probes for SARS-CoV-2 5′ spike were as follows. “*” denotes a phosphorothioate bond (for exonuclease protection), “/Phos/” denotes 5′ phosphate, “/56-FAM/” denotes 5′ FAM fluorophore (for nanoparticle capture), and “/3Bio/” denotes 3′ biotin (for test line capture).

SARS-CoV-2 5′ spike: Fwd primer: T*T*T*T*T*T* TGGGTTATCTTCAACCTAGGACTTTTCTAT (SEQ ID NO: 5), Rev primer: CCAACCTGAAGAAGAATCACCAGGAGTCAA (SEQ ID NO: 6, e.g., with or without 5′ Phos), FAM LFD probe: /56-FAM/ TTTTTTTTTTTTTTT AGGAGTCAA ATAACTTC (SEQ ID NO: 7), Biotin LFD probe: T*T*T*T*T*T*TATGTAAA GCAAGTAAAG TTTTTTTTTTTTTTT /3Bio/ (SEQ ID NO: 8).

The primer sequences shown in FIG. 15 are as follows: Influenza A forward GACCRATCCTGTCACCTCTGAC (SEQ ID NO: 9) and reverse AGGGCATTYTGGACAAAKCGTCTA (SEQ ID NO: 10); Influenza B forward TCCTCAACTCACTCTTCGAGCG (SEQ ID NO: 11) and reverse CGGTGCTCTTGACCAAATTGG (SEQ ID NO: 12); Corona 229E forward TTCCGACGTGCTCGAACTTT (SEQ ID NO: 13) and reverse CCAACACGGTTGTGACAGTGA (SEQ ID NO: 14); Rhinovirus forward TCCTCCGGCCCCTGAAT (SEQ ID NO: 15) and reverse GAAACACGGACACCCAAAGTAGT (SEQ ID NO: 16); RSV forward TCTTCATCACCATACTTTTCTGTTA (SEQ ID NO: 17) and reverse GCCAAAAAATTGTTTCCACAATA (SEQ ID NO: 18).

Sample preparation. SARS-CoV-2 samples were quantified in-house comparing BEI viral genome samples diluted in DNase/Rnase free water to fluorometrically-quantified Twist Biosciences™ qPCR RNA standards (detailed above). Ten-fold serial dilutions of the 10⁶ copies/µl standard were made down to 10 copies/µl in DNase/RNase-free water, using low binding tips and tubes to avoid sample loss. Genomic samples were also diluted to 0.1×, 0.001×, 0.0001×, 0.00001× and 0.000001× stock. Amplification by qPCR (Bio-Rad™, CFX connect™) was then performed with 4× TaqPath 1-Step RT-qPCR master mix™ (Life Science™, A15300) and the CDC N1 primer/probe pair (IDT, 10006713) at 50 µl total volume, including 1 µl sample volume, 100 nM primers, and 50 nM probes. A linear regression was performed on the Ct and expected dilution concentrations of the standard (R² = 0.999), and in turn used to convert sample Ct to absolute quantities of 184000, 1170, and 64 copies/µl, respectively with 95% confidence intervals spanning ~ 2:1-fold in counts. Simple and contrived samples were prepared by further diluting quantified samples as necessary, spiking into DNase/RNase-free water or pooled human saliva at ~ 3 or more copies per 5 µl, and used directly in RT-RPA reactions. The 64 copy/µl dilution was used to generate the 3 copy/sample experiments.

For the specificity experiments, genomic RNA from 7 respiratory viruses were spiked in DNase/RNase-free water at 10⁵ copies/µl, unless noted (in which case quantification was not supplied from source). As positive control, heat inactivated SARS-CoV-2 virus was used at 1000 copies/µl. All were diluted 1:50 (1 µl input into 50 µl total reaction volume) in the RT-RPA reaction mixture. Virus strains were further identified with qPCR. A reaction mixture composed of 5 µL of 4× TaqPath RT-PCR MM™, 1 µl of virus-specific primer mixtures (1 µM of each), 0.2 µl 100× EvaGreen™, and 12.8 µl of water was assembled. Mixtures were transferred into a PCR plate and run on a BioRad™ qPCR machine, following the CDC TaqPath™ RT protocol.

RT-RPA. A mixture of 2.5 µl each of 10 µM forward and reverse primers to the specified target, 29.5 µl of TwistAmp Basic RPA rehydration buffer (TwistDx™, TABAS03KIT), 7-11 µl of DNase/RNase-free water, and 1 µl of Protoscript II reverse transcriptase™ (NEB, M0368S) was vortexed briefly and added to the TwistAmp™ lyophilized reaction, pipetting several times to mix. 5 µl of 280 mM Magnesium Acetate and 1-5 µl of sample were added to the reaction tube lid. The 50 µl mixture was spun down, vortexed briefly, spun down again, and immediately incubated at 42° C. for 5 min in a standard PCR machine (Applied Biosystems™, 4484073) or heating block (Benchmark Scientific™, BSH300). In some embodiments, it was subsequently mixed thoroughly with a 10% Sodium Dodecyl Sulfate (SDS) at a ratio of 12 µl sample : 8 µl SDS to inactivate enzymes.

Amplicon digestion. During amplification, a digestion and LFD buffer mixture was prepared with 34.25 µl of LFD running buffer (Milenia™, MGHD 1), 5 µl of 100 nM biotin probes, 1.25 µl of 1 µM FAM probes, 5 µl of 10× NEBuffer 4™ (NEB, B7004S), and 2 µl of T7 exonuclease (NEB, M0263S). The mixture was vortexed briefly and added to a 2 mL tube (Eppendorf™). Once complete, 2.5 µl of the RT-RPA reaction was added to the 42.5 µl digestion mixture above and incubated for 1 min at room temperature.

Electrophoresis. All gels (8 × 8 cm) were denaturing PAGE at 15% polyacrylamide (Invitrogen™, EC6885BOX), run in 1× TBE buffer that was diluted from 10× TBE (Promega™, V4251) with filtered water, at 65° C., 200V, for 30 min. Gels were then removed from cassettes, stained in 1 × SybrGold™ (Life Technologies™) for 3 min, and imaged with a Typhoon™ scanner (General Electric™). Ladders are 25-766 nt DNA (NEB, #B7025).

Lateral flow assay. A standard HybriDetect™ LFD strip (Milenia Biotec™, MGHD 1) was inserted into the 2 mL Eppendorf tube above, arrows pointing up/away from the mixture, with care taken not to handle the strip roughly. It is covered with a membrane that protects the nitrocellulose, and supported by a semi-rigid backing card. The strip was incubated for 2 min or longer, as desired.

### Protocol A: ssRPA-LFD (Without SDS)

Materials needed: TwistAmp™ Basic Kit (thawed at ambient temperature); Forward primer 10 uM (IDT); Reverse primer 10 uM (IDT); RNA template; Protoscript II reverse transcriptase ™ (NEB, M0368S); DNase/RNase-free water; and lateral flow strips and buffer (Milenia™, MGHD 1)

Step 1: Set up and run RPA reaction (at PRE-AMPLIFICATION area). Have heating block set up before setting up reaction to ensure reaction times.

Step 1A. Prepare (per reaction) in the following order: 2.5 uL 10 uM forward primer; 2.5 uL 10 uM reverse primer; 7 uL DNase/RNase-free water; 29.5 uL Rehydration buffer (included in TwistDX™ kit); and 1 uL Protoscript II™ Reverse Transcriptase. Vortex and spin briefly.

Step 1B. Add above reaction to a TwistAmp Basic reaction (dried powder included in TwistDX kit). Pipette several times to mix (or vortex).

Step 1C. Add 5 uL of 280 mM Magnesium Acetate (included in TwistDX kit) and 5 uL of RNA template to tube lid (this way, RNA and MgOAc are kept separate in the tube lid prior to overall mixing). If you use less than 5 uL volume for the RNA template, then you can increase the volume of water accordingly, such that the total reaction volume is 50 uL. Close tube lid, spin down briefly, then vortex briefly to start reaction. Spin briefly before the next step.

Step ID. Immediately, incubate it at 42C for 5 minutes.

Step 2: Set up nanoparticles & exonuclease for LFD detection (at POST-AMPLIFICATION area)

Step 2A. While RPA is running, prepare (per reaction) the following in a separate “detection tube” (2 mL Eppendorf™ tube): 5 uL 100 nM biotin probes; 1.25 uL 1 uM FAM probes; 34.25 uL Milenia buffer; 5 uL NEB buffer 4™; and 2 uL T7 exonuclease. Vortex and spin briefly.

Step 2B. When RPA reaction is completed, take 2.5 uL of RPA sample and insert into detection tube. Vortex and spin briefly.

Step 2C. Wait 1 minute for exonuclease digestion.

Step 2D. Directly insert lateral flow strip into the detection tube. Wait 1-2 minutes and observe presence/absence of the test line.

### Protocol B: ssRPA-LFD (SDS)

Materials needed: TwistAmp Basic Kit (thawed at ambient temperature); Forward primer 10 uM (IDT); Reverse primer 10 uM (IDT); RNA template; Protoscript II reverse transcriptase (NEB, M0368S); DNase/RNase-free water; sodium dodecyl sulfate (SDS); and lateral flow strips and buffer (Milenia™, MGHD 1)

Quick RNA extraction protocol. Take 5 uL of patient sample (whether nasal in VTM, water, or saliva). Mix with 5 uL of Lucigen™ extraction buffer. Incubate at 95° C. for 5 minutes. Take out the tube and keep on ice. Use 5 uL (out of the total 10 uL per sample) for ssRPA.

### ssRPA Protocol

Step 1: Set up and run RPA (on PRE-AMPLIFICATION BENCH). Please have the heat block set up before setting up reaction to ensure reaction times.

Step 1.1: Prepare (per reaction) in the following order at room temperature: 5 uL DNase/RNase-free water; 29.5 uL Rehydration buffer (included in TwistDX™ kit); 2.5 uL 10 uM forward primer; 2.5 uL 10 uM reverse primer; and 0.5 uL Protoscript II™ Reverse Transcriptase. Vortex ~ 3 seconds and spin briefly (~ 3 seconds). If making a master mix, be sure to make ~(n+1)x master mix solution for n samples to ensure all samples get enough of the master mix without any pipetting error.

Step 1.2: Add above reaction to a TwistAmp Basic™ reaction (dried powder included in TwistDX™ kit). Then add 5 uL of RNA template to tube (or water into the negative control).

Step 1.3: Add 5 uL of 280 mM Magnesium Acetate (included in TwistDX™ kit) to tube lid (this way, MgOAc is kept separate in the tube lid prior to overall mixing). Close tube lid, spin down briefly (~ 3 seconds), then vortex ~ 3 seconds to start reaction. Spin briefly (~ 3 seconds) before the next step.

Step 1.4: Immediately, incubate it at 42° C. for 5 minutes.

### Step 2: Set Up LFD (On POST-AMPLIFICATION Bench)

Step 2.1. While RPA is running, prepare (per reaction) the following in a 2 mL low-bind tube: 1 uL 10 uM FAM probe; 1 uL 10 uM protected biotin probe; 64 uL running buffer; and 10 uL NEB buffer 4™. Vortex and spin briefly, then to each add: 4 uL T7 exonuclease. Vortex and spin briefly.

SDS step, 2.2: Add 12 uL of 10% SDS to lid of the tube. Once RPA reaction is complete, immediately vortex RPA samples for 3-5 seconds. Then add 8 uL of RPA sample to lid and pipette up and down 25-30 times quickly to mix RPA with SDS. Immediately, spin solution down, vortex for 5 seconds, spin for 5 seconds.

Step 2.3. Incubate at room temperature for 1 minute (for exonuclease digestion).

Step 2.4. Place the lateral flow strip into tube, incubate for 2 minutes (but can wait up to an hour).

Addition of a buffer additive can improve the accuracy of the LFD output. Non-limiting examples of buffer modifications include surfactant (e.g., SDS, LDS, alkyl sulfates, alkyl sulfonates or other detergents), bile salts, ionic salt, chaotropic agents (i.e., a compound which disrupts hydrogen bonding in aqueous solution), formamide, DNA duplex destabilizers, or reducing agents.

In some embodiments of any of the aspects, this protocol can comprise the following non-limiting variations: (1) SDS + RPA mixed into running solution (Protocol B as described herein); (2) RPA + exonuclease, mixed into SDS + running buffer; or (3) RPA + exonuclease + running buffer, SDS mixed last. In some embodiments of any of the aspects, an incubation step (e.g. 1 minute) can be added between any of the two steps, described above.

In some embodiments of any of the aspects, the LFD is pre-treated with SDS, e.g., dried onto the conjugate pad or nitrocellulose membrane. In some embodiments of any of the aspects, SDS is dried onto a piece of paper, nitrocellulose membrane, or other material, which is added into the solution just before or concurrently with the LFD. Optionally, the LFD is used to stir the SDS into the running solution. The SDS removes significant false positives that can show up when RPA is run on the LFD system.

### Example 3: Method for Rapid, Highly Sensitive Nucleic Acid Detection at Single-Base Resolution

### 1. Intended Use of This Technology

Rapid, accurate, highly sensitive nucleic acid detection is becoming increasingly important for disease diagnosis and disease prevention. During a pandemic, the diagnostic capability is a rate-limiting step to stop the spreading of the disease. A cost-effective, rapid, accurate, highly sensitive method that provides easy operation with no need for specialized equipment and expertise is highly desired. Described herein is a toehold switching based isothermal amplification LFD detection method (tsRPA) that fulfills this need.

### 2. How Does This Technology Work?

tsRPA technology (see e.g., FIGS. 25A-25C) utilizes rapid isothermal Recombinase Polymerase Amplification (RPA), nanoparticles, and Lateral Flow Detection (LFD) for ultrafast amplification and detection. The technology utilizes a Toehold Switching mechanism to achieve high accuracy.

Take viral RNA as an example. The target nucleic acid is first extracted or enzymatically released. The viral genome containing the target sequence x is then added into a reaction mix with reverse transcriptase, RPA reagents, Forward primer a labeled with nanoparticles, and reverse primer b. a binds to the complementary sequence a* in target and reverse transcription converts the RNA into RNA-cDNA hybrid. Recombinase then allows for primer b to bind to b* sequence on cDNA and to generate a full amplicon of a-x-b. RPA then exponentially amplifies the amplicon and generates a detectable signal in 5 minutes. The amplified product is added to an LFD pad where toehold switch allows for the nanoparticle-bound strand to bind to the probes on the LFD pad and allows for a band to show up on the device for signal verification.

Described herein are three non-limiting scenarios of tsRPA technology.

In the first scenario (see e.g., FIG. 25A), after RPA, the full amplicon is treated with Lambda exonuclease or T7 exonuclease to remove the a*-x-b strand. Since the a-x*-b* strand is bound to nanoparticles, it is protected from digestion. After a brief heat inactivation step, the sample is loaded on an LFD pad where the double-stranded probe with b overhang is printed. The b* on the target strand binds to the b overhang in the probe and displaces the a-x* probe strand due to longer base matching. The signal from the nanoparticles is then enriched on the LFD.

In the second scenario (see e.g., FIG. 25B), uracil containing reverse primer is used for RPA. After amplification, User enzyme is then added to fragment the reverse primer and expose the complementary sequence b* in the full amplicons. The digested product is then added to an LFD pad. The single-stranded b* binds to the b sequence in the probes and the complementary strand (a*-x) in the full amplicon is displaced by the probes due to longer base matching. The signal from the nanoparticles is then enriched on the LFD.

In the third scenario (see e.g., FIG. 25C), a stopper is placed on the nanoparticle strand between the sequence c and a. During the amplification, the stopper stops the extension of the DNA polymerase and generates a double-stranded full amplicon with a single-stranded overhang c. On the LFD pad, the c overhang binds to the c* sequence in the probes and the complementary strand (a*-x-b) of the full amplicons is displaced by the probes due to longer base matching. The signal from the nanoparticles is then enriched on the LFD.

### 3. How Does This Technology Fulfill an Unmet Need/Significant Improvement Over Existing Technology?

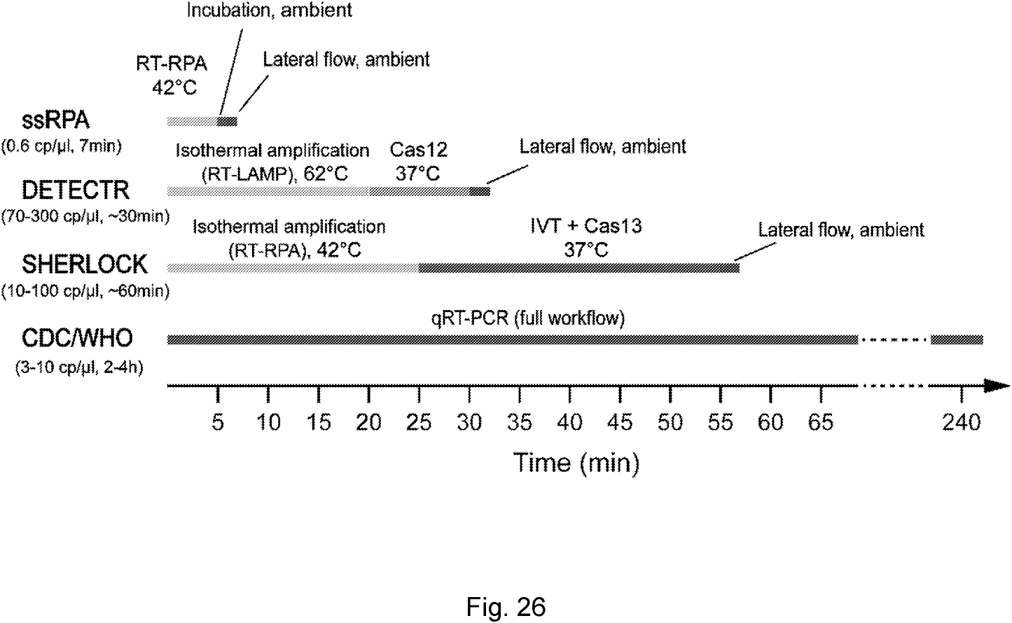

tsRPA technology provides a rapid detection time (see e.g., FIG. 26) with a total turnover time of ~10 minutes. The RT-RPA step is isothermal and needs only 5 minutes to obtain detectable products. Heat inactivation is only less than 1 minute, and the digestion time is only 1 minute. The final detection step needs 4 minutes. This scheme is much faster than the current FDA approved protocols or the state-of-the-art.

tsRPA technology is highly sensitive. The RPA reaction allows the detection of viral targets at a single copy resolution.

tsRPA technology is highly accurate. The toehold switching mechanism is mismatch sensitive. Probes can be designed that distinguish two different viral strains with only a few base difference.

tsRPA technology is very easy to operate without the need for special equipment and personnel. One only needs two heat blocks and pipettes to do the test. This makes the technology widely applicable in a variety of cases, such as where there is a shortage of equipment and expertise during a pandemic outbreak.

tsRPA technology is cheap. The cost of the test is much lower compared to assays that need specialized equipment and expertise.

### Example 4: Exemplary Isothermal Amplification Workflows

### RPA

The dsDNA amplicon generated by RPA can be rendered accessible to the sequence-specific probes via three alternative approaches (see e.g., FIG. 33).

(1) ssRPA: Action of a dsDNA specific monodirectional exonuclease (e.g., T exonuclease, lambda exonuclease etc.) specifically removes one of the strands of the amplicon. The ssDNA probes can then bind the target in a sequence specific manner. For this application, one of the strands is protected via phosphorothioate or other protective end or internal modification (e.g., bulk end groups such as proteins, antibodies, spacers, nonconventional nucleotide linking chemistries, crosslinks). The probe strands can also be optionally nuclease-protected by similar modifications.

(2) Strand invasion: Action of a recombinase, SSB, and/or helicase can mediate invasion of the dsDNA amplicon by the ssDNA probes via partial unwinding of the duplex structure. This process can be optionally aided by heat inactivation or use of buffer additives such as surfactants (e.g., SDS, LDS, alkyl sulfates, alkyl sulfonates or other detergents), bile salts, ionic salt, chaotropic agents, formamide, DNA duplex destabilizers, reducing agents, which can completely or selectively inactivate the components in the original amplification mixture in a concentration-dependent manner. Alternatively or additionally, adding or modulating the concentrations of the recombinase or single-strand-binding protein (SSB) or helicase after the amplification process can improve the probe invasion.

(3) dCas-mediated detection using CRISPR components: A nuclease-dead dCas protein (such as dCas9, dCas12, dCas13) can be utilized for sequence-specific binding of the gRNA probes to the dsDNA amplicon. gRNA probes can be modified directly to carry the modifications described below for readout (e.g., biotin, FAM, fluorophore, quencher, nanoparticle) and can be labeled through tails that will bind to oligos that carry these modifications.

Note: Nuclease-dead Cas proteins are obtained by mutations in their cleavage domains. For the example case of Cas9, nuclease-dead Cas9 (dCas9) can be obtained by rendering one or both of the RuvC and HNH nuclease domains inactive by point mutations (D10A and H840A in SpCas9). See e.g., Brezgin et al. International Journal of Molecular Sciences 20, no. 23 (2019): 6041; Hsu et al. Cell 157, 1262-1278 (2014); the contents of each of which are incorporated herein by reference in their entireties.

### Readout Methods for RPA

LFD detection: probes carry the functional groups that mediate binding to the lateral flow device (e.g., biotin and/or FAM/FITC end modifications that are compatible with common strip-format lateral flow devices where anti-FAM-nanoparticles are used for line formation on streptavidin test lines). The colocalization of the two probes immobilizes the nanoparticles on the test line giving a positive signal. In absence of the amplicon binding, the diffuse probes do not form the test line. Alternatively, the test line can be coated with a ssDNA oligo (x) which is complementary to one of the probes. In this case one of the probes carry the complementary sequence (x*), which is used to immobilize the nanoparticles indirectly, when it colocalizes with the nanoparticle binding probe.

Colorimetric readout: A color change in response to the presence of a DNA target can be induced by co-localizing plasmonic nanoparticles. In particular, the probes are modified by conjugating them to plasmonic nanoparticles. In the presence of the amplicon, and only in its presence, the probes hybridize to it and thus co-localize the nanoparticles, causing a color change which may be read by the naked eye or on instruments like the spectrophotometer.

Fluorescence detection: the toehold probe carries a fluorophore (e.g., FAM) on its 5′ (optionally also internal or 3′). A shorter protector strand, which carries a quencher (e.g., black hole quencher) on the 3′ (or alternatively at a position that will put the quencher into close proximity with the fluorophore on the toehold probe), is initially bound to the toehold probe via complementarity to the 5′ domain of the toehold probe. In presence of the amplicon binding, the protector strand is displaced, hence the quencher is no longer in the close vicinity of the fluorophore. Increased fluorescence is read out as the positive signal for amplicon. This fluorescence can be detected using a fluorimeter, a qPCR machine or plate reader equipped with a fluorescence detector. Alternatively, the detection assay can start with the probes in a fluorescent state, with one probe modified with a fluorophore and another with a quencher. The probes start out freely floating in solution and hence are excitable and produce a fluorescent signal. In the presence of a target amplicon the probes are co-localized, putting the fluorophore and quencher molecules in close proximity and thus resulting in loss of fluorescent signal, indicating the presence of a target. A third method for obtaining fluorescent readout involves a double strand specific exo or endonuclease (e.g., T7 exonuclease, lambda exonuclease, Endo IV) that detects and digests the probe after it binds to the target amplicon (alternatively a polymerase with internal exonuclease activity can accomplish this). The probe is modified with a quencher at one end and fluorophore at another. In the absence of a complementary target the probe is single stranded and hence randomly coiled, which keeps the fluorophore and the quencher molecules in close proximity, quenching fluorescent signal. When the probe hybridizes to the target, it is stretched out along a helical path, separating the fluorophore and quencher molecules at either end, which allows the fluorophore to emit photons in response to excitation light. See e.g., FIG. 33. FIGS. 38A-38C provides data to support the fluorescence application.

Other quencher probe designs such as molecular beacons with self-complementarity, or ZEN™ probes with internal quencher can be used as alternatives. Alternatively probes with fluorophore modifications that constitute a Förster resonance energy transfer (FRET) fluorophore pair can be used. In this case, their colocalization on the target yields the FRET signal.

Alternatively, for the case of Cas-mediated probe binding, the colorimetric or fluorescence detection can be achieved by use of split fusion proteins for the dCas such as split dCas9, split-GFP-dCas fusion, split-HRP (colorimetric) that assemble together when colocalized. These half-domains can alternatively be conjugated to the gRNA probes.

### LAMP

LAMP produces DNA amplicons of various lengths, in the form of concatemers. Each concatemer is a single strand of DNA. The concatemers have a strong secondary structure and fold up into double hairpins (see e.g., Amplicon 1 in FIG. 34) or hairpins (see e.g., Amplicons 2, 3 ... N in FIG. 34). The double hairpins have exposed single stranded regions in the center, to which probes may hybridize. The hairpins have a long double stranded stem region that is not typically available for probe hybridization. Described herein are five different ways (e.g., 2A through 2E in FIG. 34) in which probes may bind to various LAMP amplicons. The probes can be modified with other molecules/particles to permit readout, as described herein.

Direct probe binding: Some fraction of LAMP amplicons are intermediate double hairpin molecules whose target region (e.g., labeled as b* c* in FIG. 34) exists as single strands. Amplification is halted by adding a chemical to inactivate the LAMP reaction (e.g., typical chemicals include detergents like SDS, Triton-X or denaturing agents like formamide, sodium hydroxide etc.) and then probes (e.g., labeled b and c in FIG. 34) are introduced to hybridize to the targets. Alternatively, instead of halting amplification by adding a chemical reagent, the LAMP reaction can naturally exhaust itself by consuming all primers.

Single strand conversion by exonuclease digestion: Some fraction of LAMP amplicons exists as DNA hairpins with long double stranded stems containing the target region. The target is exposed by digesting one of the two arms of the stem using a DNA exonuclease, thus exposing the target. A double strand specific exonuclease can be used so that after digesting one of the arms of the stem, as the exonuclease reaches the single stranded loop region, it cannot proceed further.

Probe binding by recombinase and single stranded binding protein action: Recombinases are DNA enzymes that allow a single strand of DNA to invade (hybridize) into a homologous double stranded DNA region. This action is further assisted by single strand binding proteins that bind to the displaced single stranded DNA region and stabilize the complex. The LAMP reaction is inactivated (or it exhausts itself) and then recombinases, single strand binding proteins, fuel (ATPs) and probes are introduced. The probes find the target by sequence homology and are hybridized to them by recombinase action. Instead of using the recombinase enzyme, one can also use a helicase enzyme that locally denatures double stranded DNA by unwinding it, allowing probes to invade and hybridize. This process can be optionally aided by use of buffer additives such as surfactants (e.g., SDS, LDS, alkyl sulfates, alkyl sulfonates or other detergents), bile salts, ionic salt, chaotropic agents, formamide, DNA duplex destabilizers, reducing agents, which can completely or selectively inactivate the components in the original amplification mixture in a concentration-dependent manner.

Heat denaturation: Probes are added and then the LAMP reaction is heated to denature (i.e., melt) the LAMP amplicons and disrupt their secondary structure. The reaction is then cooled quickly in the presence of high probe concentration, which allows probes to bind to the exposed single stranded regions before the secondary structure of the amplicons is reestablished. The heating step can involve temperatures between 80° C. and 90° C. for a period of 1 minute to 15 minutes. The cooling step can involve temperatures between 60° C. and 10° C. for a period of less than 1 minute to as much as 60 minutes. Heat denaturation may be combined with any of the three above methods, either before, with or after the above techniques are applied.

dCas mediated probe binding. A nuclease-dead dCas protein (e.g., dCas9, dCas12, dCas13) can be utilized for sequence-specific binding of the gRNA (guide RNA) probes to the double stranded regions of the amplicon. gRNA probes can be modified directly to carry the modifications described below for readout.

### Readout Methods for LAMP

LFD readout. Each of two probes (e.g., labeled b* and c* in FIG. 34) are complementary to the target of interest. Described above are various ways in which these are hybridized to the target. The probes may be functionally modified so that they produce a signal on a lateral flow device if and only if both of them bind to the amplicon. One of the probes is modified with a biotin molecule or a DNA handle (e.g., labeled x in FIG. 34) such that it is captured by streptavidin molecules immobilized at the test line of a lateral flow device. The other probe is modified with a FAM molecule. A colored nanoparticle (e.g., gold nanoparticle or latex bead) coated with an anti-FAM antibody is introduced into the buffer containing the amplicons and the hybridized probes. The particles bind to the FAM molecule on the probe. The colocalization of the two probes immobilizes the nanoparticles on the test line giving a positive signal. In absence of the amplicon binding, the diffuse probes do not form the test line. Alternatively, the test line can be coated with a ssDNA oligo (x) which is complementary to one of the probes. In this case one of the probes carry the complementary sequence (x*), which is used to immobilize the nanoparticles indirectly, when it colocalizes with the nanoparticle binding probe.

Colorimetric readout: A color change in response to the presence of a DNA target can be induced by co-localizing plasmonic nanoparticles. In particular, the probes are modified by conjugating them to plasmonic nanoparticles. In the presence of the amplicon, and only in its presence, the probes hybridize to it and thus co-localize the nanoparticles, causing a color change which may be read by the naked eye or on instruments like the spectrophotometer.

Fluorescence readout: A fluorescent readout may be obtained by displacing a quencher molecule from the proximity of a fluorophore molecule. The probe can be modified with a fluorophore molecule, and a protector molecule, which carries a quencher, is hybridized to the probe. In the absence of the target amplicon, the fluorophore is quenched and does not emit photons in response to incoming photon excitations. In the presence of the target amplicon, the probe hybridizes to it and thus displaces the quencher bearing protector strand, allowing the fluorescent molecule to emit photons in response to excitation light. This fluorescence can be detected using a fluorimeter or a qPCR machine equipped with a fluorescence detector. Alternatively, the detection assay can start with the probes in a fluorescent state, with one probe modified with a fluorophore and another with a quencher. The probes start out freely floating in solution and hence are excitable and produce a fluorescent signal. In the presence of a target amplicon the probes are co-localized, putting the fluorophore and quencher molecules in close proximity and thus resulting in loss of fluorescent signal, indicating the presence of a target. A third method for obtaining fluorescent readout involves a double strand specific exonuclease that detects and digests the probe if and only if it is a part of a double stranded DNA molecule. The probe is modified with a quencher at one end and fluorophore at another. In the absence of a complementary target, the probe is single stranded and hence randomly coiled, which keeps the fluorophore and the quencher molecules in close proximity, quenching fluorescent signal. When the probe hybridizes to the target, it is stretched out along a helical path, separating the fluorophore and quencher molecules at either end, which allowing the fluorophore to emit photons in response to excitation light.

### HDA

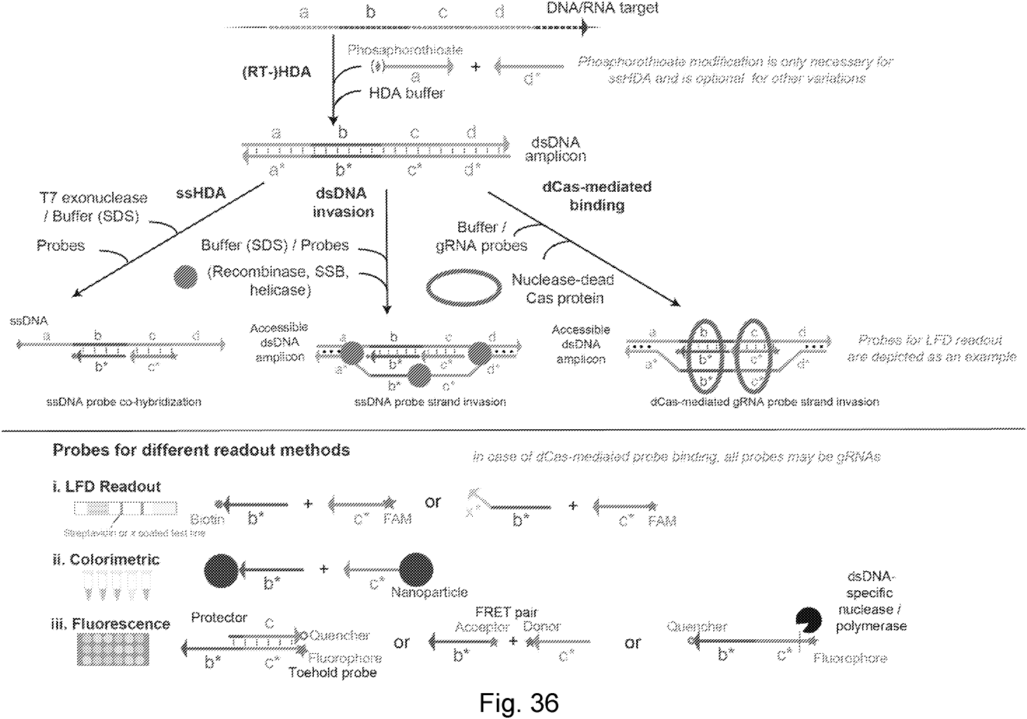

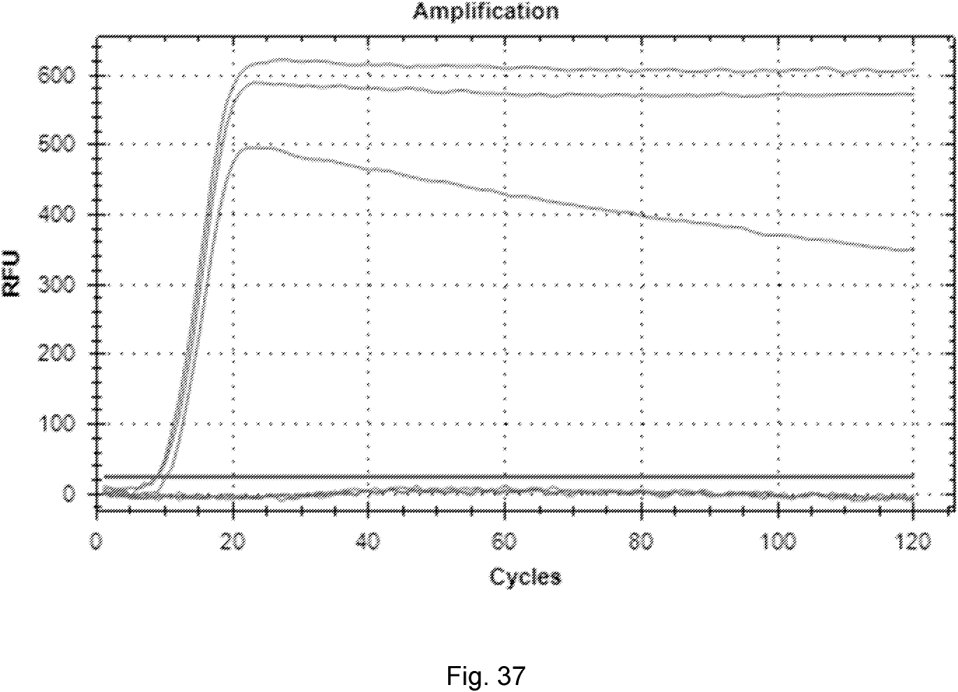

Helicase dependent amplification (HAD) utilizes DNA helicase to unwind the double stranded DNA. Single stranded binding proteins then stabilize the structure by binding to the unwinded single stranded regions, allowing for primer binding. With high displacement activity of the BST DNA polymerase, the method can achieve isothermal exponential amplification of the target region. FIG. 35 and FIG. 36 show the reaction mechanism for HDA and exemplary work flows. For HDA, crowding agents (e.g., PEG, PEG8000, dextran of different molecular weights, dextran sulfate, ficoll, glycerol) can be added to promote the reaction speed and/or improve the kinetics of target binding. FIG. 37 shows exemplary data of the effect of crowding agents on the reaction efficiency for HDA.

The dsDNA amplicon generated by HDA can be rendered accessible to the sequence-specific probes via three alternative approaches (see e.g., FIG. 36).

(1) ssHDA: Action of a dsDNA specific monodirectional exonuclease (e.g., T exonuclease, lambda exonuclease etc.) specifically removes one of the strands of the amplicon. The ssDNA probes can then bind the target in a sequence specific manner. For this application, one of the strands is protected via phosphorothioate or other protective end or internal modification (e.g., bulk end groups such as proteins, antibodies, spacers, nonconventional nucleotide linking chemistries, crosslinks). The probe strands can also be optionally nuclease-protected by similar modifications.

(2) Strand invasion: Action of a recombinase, SSB, and/or helicase can mediate invasion of the dsDNA amplicon by the ssDNA probes via partial unwinding of the duplex structure. This process can be optionally aided by heat inactivation or use of buffer additives such as surfactants (e.g., SDS, LDS, alkyl sulfates, alkyl sulfonates or other detergents), bile salts, ionic salt, chaotropic agents, formamide, DNA duplex destabilizers, reducing agents, which can completely or selectively inactivate the components in the original amplification mixture in a concentration-dependent manner. Alternatively or additionally adding or modulating the concentrations of the recombinase or single-strand-binding protein (SSB) or helicase after the amplification process can improve the probe invasion.

(3) dCas-mediated detection using CRISPR components: A nuclease-dead dCas protein (such as dCas9, dCas12, dCas13) can be utilized for sequence-specific binding of the gRNA probes to the dsDNA amplicon. gRNA probes can be modified directly to carry the modifications described below for readout (e.g., biotin, FAM, fluorophore, quencher, nanoparticle) and can be labeled through tails that will bind to oligos that carry these modifications.

### Readout Methods for HDA

LFD detection: probes carry the functional groups that mediate binding to the lateral flow device (e.g., biotin and/or FAM/FITC end modifications that are compatible with common strip-format lateral flow devices where anti-FAM-nanoparticles are used for line formation on streptavidin test lines). The colocalization of the two probes immobilizes the nanoparticles on the test line giving a positive signal. In absence of the amplicon binding, the diffuse probes do not form the test line. Alternatively, the test line can be coated with a ssDNA oligo (x) which is complementary to one of the probes. In this case one of the probes carry the complementary sequence (x*), which is used to immobilize the nanoparticles indirectly, when it colocalizes with the nanoparticle binding probe.

Colorimetric readout: A color change in response to the presence of a DNA target can be induced by co-localizing plasmonic nanoparticles. In particular, the probes are modified by conjugating them to plasmonic nanoparticles. In the presence of the amplicon, and only in its presence, the probes hybridize to it and thus co-localize the nanoparticles, causing a color change which may be read by the naked eye or on instruments like the spectrophotometer.

Fluorescence detection: the toehold probe carries a fluorophore (e.g., FAM) on its 5′ (optionally also internal or 3′). A shorter protector strand, which carries a quencher (e.g., black hole quencher) on the 3′ (or alternatively at a position that will put the quencher into close proximity with the fluorophore on the toehold probe), is initially bound to the toehold probe via complementarity to the 5′ domain of the toehold probe. In presence of the amplicon binding, the protector strand is displaced, hence the quencher is no longer in the close vicinity of the fluorophore. Increased fluorescence is read out as the positive signal for amplicon. This fluorescence can be detected using a fluorimeter, a qPCR machine or plate reader equipped with a fluorescence detector. Alternatively, the detection assay can start with the probes in a fluorescent state, with one probe modified with a fluorophore and another with a quencher. The probes start out freely floating in solution and hence are excitable and produce a fluorescent signal. In the presence of a target amplicon the probes are co-localized, putting the fluorophore and quencher molecules in close proximity and thus resulting in loss of fluorescent signal, indicating the presence of a target. A third method for obtaining fluorescent readout involves a double strand specific exo or endonuclease (e.g., T7 exonuclease, lambda exonuclease, Endo IV) that detects and digests the probe after it binds to the target amplicon (alternatively a polymerase with internal exonuclease activity can accomplish this). The probe is modified with a quencher at one end and fluorophore at another. In the absence of a complementary target the probe is single stranded and hence randomly coiled, which keeps the fluorophore and the quencher molecules in close proximity, quenching fluorescent signal. When the probe hybridizes to the target, it is stretched out along a helical path, separating the fluorophore and quencher molecules at either end, which allows the fluorophore to emit photons in response to excitation light.

Other quencher probe designs such as molecular beacons with self-complementarity, or ZEN™ probes with internal quencher can be used as alternatives. Alternatively probes with fluorophore modifications that constitute a Förster resonance energy transfer (FRET) fluorophore pair can be used. In this case, their colocalization on the target yields the FRET signal.

Alternatively, for the case of Cas-mediated probe binding, the colorimetric or fluorescence detection can be achieved by use of split fusion proteins for the dCas such as split dCas9, split-GFP-dCas fusion, split-HRP (colorimetric) that assemble together when colocalized. These half-domains can alternatively be conjugated to the gRNA probes.

### Isothermal Amplification Methods

Any isothermal amplification method as described herein can be used in combination with any of the detection methods described herein. Non-limiting examples of isothermal amplification methods include: Recombinase Polymerase Amplification (RPA), Loop Mediated Isothermal Amplification (LAMP), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3SR), Single primer isothermal amplification (SPIA), and cross-priming amplification (CPA).

In any of the amplification and/or detection methods as described herein, buffer additives such as surfactants (e.g., SDS, LDS, alkyl sulfates, alkyl sulfonates or other detergents), bile salts, ionic salt, chaotropic agents, formamide, DNA duplex destabilizers, reducing agents can be used: i) to pretreat the lateral flow devices, ii) be added together with the second step (e.g., exonuclease, or recombinase, or Cas binding) or iii) added as the last step after (e.g., exonuclease, or recombinase, or Cas binding) and before the readout.

Optionally heat inactivation or heat-denaturation can be performed in between the isothermal amplification and the second step or after the second step and before readout.

The probes for the detection via all methods can have the functional groups (e.g., fluorophore, quencher, biotin, nanoparticles etc.) as end modifications or internal modifications or indirectly via a tail domain (3′ or 5′) that hybridizes to another oligo carrying the functional group. In the case of Cas-mediated detection, the tails or the single-stranded loop domains of the guide RNA (gRNA) can be directly or indirectly (e.g., via hybridization) modified with functional groups.

For LFD detection with Cas-mediated probe binding, the detection can be alternatively achieved by only one gRNA probe. In this case, the target strand of the amplicon can be synthesized using a biotinylated primer. A second strand can that may be modified by a fluorophore or nanoparticle can be immobilized on the same complex by binding to single-stranded portion of the gRNA probe (such as loop or tails). The assembled complex can form the test line by immobilization of the nanoparticles to the test line in presence of the amplicon (for example via binding of the biotin group to the streptavidin coated test line.)

For the amplification and/or detection process for all variations, crowding agents (e.g., PEG, PEG8000, dextran of different molecular weights, dextran sulfate, ficoll, glycerol) can be added to promote the reaction speed and/or improve the kinetics of target binding. Optionally other blocker sequences, or common blocking agents (BSA, IgGs, tRNA, single stranded excess DNA or RNA, excess orthogonal or random primers, double-stranded excess DNA or similar) can be included for the reactions or for detection step.

### Example 5: Digest-Detect

Described herein is a scheme for sequence specific reporting of nucleic acid targets using catalytic probe digestion. An enzyme capable of double strand specific 5 prime to 3 prime exonuclease activity (e.g. Bst Full Length DNA strand displacing polymerase with intact 5 prime to 3 prime exonuclease activity) was introduced at a wide range of reaction temperatures (e.g. 20° C. to 70° C.). A double-labeled probe whose sequence is complementary to a ‘detection’ region (5 bases to 40 bases) of the target was also introduced (see e.g., FIGS. 39A-39B).

The two labels, one at each end of the probe, can be a fluorophore and quencher pair (other cases described below) in which case the probe is quenched when freely floating (unbound) in solution. In the presence of the target, the probe hybridizes to the detection region of the target because of sequence complementarity. This results in a slight increase in fluorescent signal, since the average distance between fluorophore and quencher is increased when the probe is in the double stranded form. Now, the double strand specific exonuclease enzyme that was introduced can use its 5 prime to 3 prime exonuclease activity to digest the probe and hence release the fluorophore and quencher into solution, greatly increasing the average distance between them, resulting in a greatly increased fluorescence. The progress of the reaction can be monitored using a fluorescence reader, like commonly found in real time PCR machines. Note that this fluorescence is sequence specific and only occurs when the probe recognizes the detection region of the target through complementary hybridization. Spurious targets will not bind the probe and hence the probe will remain undigested. Critically, each digestion event acts to ‘check’ the sequence of the target or amplicon it binds to, thus ensuring a very sequence specific output signal.

### Example 6: Digest-LAMP

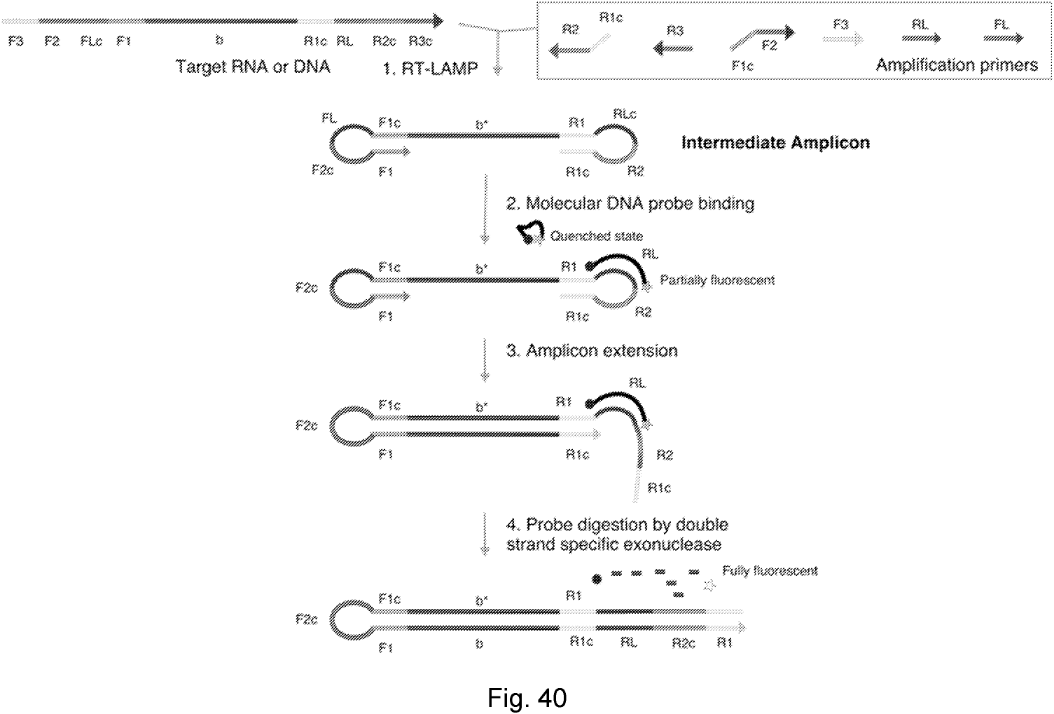

Digest-Detect was applied to LAMP isothermal amplification to produce Digest-LAMP, a sequence specific method for detecting nucleic acid targets by amplifying them and reporting their presence in a sequence specific manner. FIG. 40 illustrates the mechanism of Digest-LAMP.

In FIG. 40, the detection region lies in the loop region of the LAMP amplicon, but in general it may lie in any exposed single stranded region of the amplicon. The two labels, one at each end of the probe, can be a fluorophore and quencher pair (other cases described below) in which case the probe is quenched when freely floating (unbound) in solution. In the presence of the target, the detection region is amplified in the LAMP reaction by using amplification primers and strand displacing polymerase, creating many copies. The probe hybridizes to the detection region of these intermediate amplicons because of sequence complementarity. This results in a slight increase in fluorescent signal, since the average distance between fluorophore and quencher is increased when the probe is in the double stranded form. Now, the double strand specific thermostable enzyme that was introduced can use its 5 prime to 3 prime exonuclease activity to digest the probe and hence release the fluorophore and quencher into solution, greatly increasing the average distance between them, resulting in a greatly increased fluorescence. The progress of the reaction can be monitored using a fluorescence reader, like commonly found in real time PCR machines. Note that this fluorescence is sequence specific and only occurs when the probe recognizes the detection region of the target through complementary hybridization. Spurious amplifications (e.g., due to primer dimers) do not produce copies of the detection region and hence the probe remains undigested. Note that for fluorescent reporting the probe may also be a double quenched (e.g. Zen probes by IDT) by having an additional quencher as an internal modification to the probe. This can further reduce background fluorescence of the unbound probes. Alternatively, a FRET pair may be localized on the probe such that the fluorescence signal changes upon cleavage.

### Example 7: Alternate Reporting Mechanism

Lateral flow device (LFD): The probe is labeled with two different affinity chemicals, for example biotin and FAM. Biotin has strong affinity to streptavidin while FAM has strong affinity to Anti-FAM antibodies. Typically, nanoparticles coated with Anti-FAM antibodies are used to further enhance signal, in which case they are the effective label. The LFD contains two lines, which have affinity molecules immobilized on them. The first line is called the control line and has affinity to one of the labels (label 1, e.g., biotin), while the second line is called the test line and has affinity to the other label (label 2, e.g., Anti-FAM coated nanoparticle). When the probe is undigested, it is captured at the control line through affinity to label 1 (e.g., streptavidin captures biotin which is contained in undigested probe) and thus the nanoparticle is localized at the control line and the line become visible, indicating a negative. On the other hand, when the probe is digested the nanoparticles are not linked to label 1 and hence they are not immobilized at the control line. They diffuse further and are captured at the test line (which has affinity to label 2) and a test line becomes visible, indicating a positive.

Colorimetric readout with plasmon shift: The probe is double-labeled with plasmonic nanoparticles (e.g., gold nanoparticles) at either end. Co-localized plasmonic nanoparticles undergo a ‘peak shift’ in absorbance, causing a visible color change. Thus, as the probe is digested the plasmon nanoparticles decouple, causing a color change which can be detected with the naked eye or a spectrophotometer.

Multiplexed fluorescent readout: As noted earlier, the probe is sequence specific. Thus, the presence of multiple targets can be independently reported in the same tube by simply conjugating probes with spectrally non-overlapping fluorophores. Thus, a fluorescent channel could be reserved for a target. Real time PCR machines can support up to five spectrally non-overlapping channels, allowing detection of five distinct targets in one tube.

Alternative readout strategies: the results of Digest-LAMP may also be readout using alternative secondary strategies, including but not limited to: sequence-specific detection of remaining probe sequence using e.g. toehold-mediated strand displacement, probe-based electrochemical readouts, micro-array detection, or sequence-specific amplification schemes. Digest-LAMP results may also be read out using gel electrophoresis or sequencing.

### Example 8: Multiplexed Detection of SARS-COV-2 RNA and RNaseP Control Gene With Digest-LAMP

As few as 50 copies of SARS-CoV-2 can be detected using Digest-LAMP inside 30 minutes using a FAM-labeled double quenched digestion probe. In addition, a COVID positive patient was successfully identified from an anonymized saliva sample that was heat inactivated but not otherwise processed for RNA extraction. A LAMP primer set for amplifying a ubiquitous human control RNA, RNAseP, was also included in the reaction mix to rule out inhibition of amplification due to contaminants. The RNAseP amplicon was detected with a double quenched probe labeled with a HEX fluorophore, and was detected in an orthogonal wavelength channel using a standard real time PCR instrument.

### List of Enzymes Compatible With Digest-Detect And/or Digest-LAMP

Enzymes compatible with Digest-Detect and/or Digest-LAMP include but are not limited to: Bst Full Length, Taq DNA polymerase, T7 Exonuclease, Exonuclease VIII truncated, Lambda exonuclease and T5 Exonuclease.

### Protection of Primers and Amplicons From Digestion

Amplicons and primers may form some double stranded regions (spurious or otherwise) that may need to be protected from digestion to improve the performance of the assay. In some versions of the technique, primers can be used that are protected from enzymatic digestion by the use of DNA modifications like phosphorothioate nucleotides, inverted dT, 5 primer phosphorylation, non-canonical bases (isoC, isoG) etc.

### Probe Modifications to Increase Melting Temperature

Probes may be made out of DNA and/or RNA and/or contain modifications that increase their melting temperature. Some modifications include LNA bases (locked nucleic acids), MGBs (minor groove binder), SuperT (5-hydroxybutynyl-2′-deoxyuridine), 5-Me-pyridines, 2-amino-deoxyadenosine, Trimethoxystilbene, Pyrene etc.

### Probe Modifications to Increase Reported Signal

Probes may be modified with multiple reporting moieties (e.g. fluorophores, gold nanoparticles, latex nanobeads, biotin, streptavidin, FAM, etc.) to increase the reported signal. For instance, multiple fluorophores and/or gold nanoparticles and/or latex nanobeads may be attached per probe, increasing the net fluorescent and/or colorimetric and/or LFD signal obtained when the probe is digested. Alternately, multiple affinity moieties like biotin, streptavidin, FAM etc. may be attached to the probe to increase the affinity capture efficiency of the probe on LFDs. These multiple reporting moieties may be attached by means of modification chemistries (Trebler phosphoramidite, internal modifications, fluorescent nucleotides, aminopurine modification etc.).

### LAMP Reaction Conditions

Digest-LAMP may be carried out in any suitable vessel, including but not limited to test tubes (e.g. 200 uL, 1.5 mL, 2 mL), cuvettes, microfluidic chambers, or custom chambers (e.g. 3D printed containers). Multiple input sources (e.g. samples from different patients) may be pooled together within a single reaction chamber.

### Example 9: Alternative Assay Design

### Probe Sequence

In some embodiments of any of the aspects provided herein, the nucleic acid probe sequence is substantially similar to one of the primer sequences. In some embodiments, the nucleic acid probe sequence is complementary to a region of the target sequence within the primer regions.

### Probe Structure

In some embodiments, the nucleic acid probe comprises RNA, LNA, or other bases.

In some embodiments, the nucleic acid probe comprises a plurality of fluorophores (e.g., of the same or different type) for further signal enhancement. In some embodiments, the fluorophores are on the same sequence. In some embodiments, the fluorophores are on a mix of the same sequence but with different fluorophores/moieties.

In some embodiments, the nucleic acid probe comprises both lateral flow detectable moieties and fluorophores combined in the same probe strand. In some embodiments, the lateral flow detectable moieties and fluorophores are comprised by different probe strands (e.g., with the same sequence) mixed together in order to permit both fluorescence readout and LFA readout from the same reaction.

In some embodiments, a mix of similar probe sequences, e.g., differing by one or two bases from each other, are utilized within the same solution to detect single nucleotide polymorphisms (SNP’s) or different variants of a target nucleic acid (e.g., viral sequence).

In some embodiments, individual probes are immobilized to a surface to localize signal readout to a specific surface (e.g. glass slide, side of tube). In this way, all distinct targets use the same moiety for readout (e.g., the same fluorophore for each different probe sequence), as the particular spatial configuration of the signal indicates which targets were detected in solution.

In some embodiments, individual primers are immobilized to a surface to localize some or all amplification at the site of the surface.

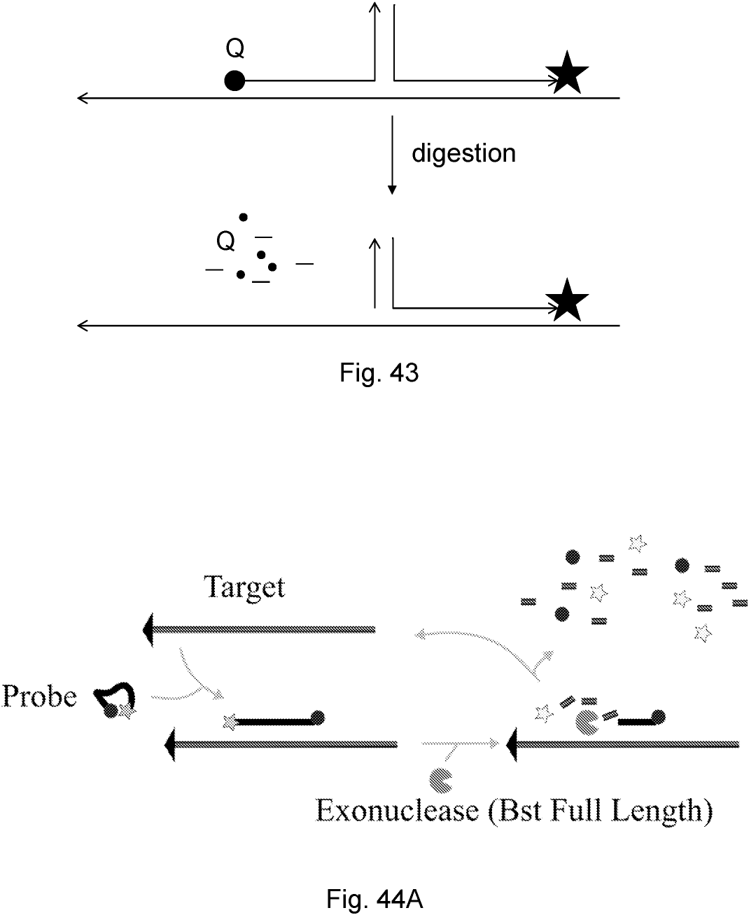

In some embodiments, the nucleic acid probe comprises at least two strands hybridized together such that the exonuclease digestion still displaces one moiety from being attached to another (see, e.g. FIG. 43).

### Probe Readout

In some embodiments, multiple probes each comprise different fluorophores or detectable moieties (e.g., multiplexing).

For applications such as disease diagnostics, where in most cases it is expected that only one of a possible set of diseases would test positive, a combinatorial readout can be utilized to achieve higher multiplexing with a limited number of spectrally detectable channels. For example, one disease can cause fluorescence in only the Cy5 channel, whereas another disease can only cause fluorescence in the FITC channel, and a third disease would cause fluorescence in both channels.

### Enzyme

In some embodiments, Bst full length enzyme is used as the only enzyme in the Digest-LAMP assay. In some embodiments, Bst full length enzyme is supplemented in addition to another enzyme (such as Bst Large Fragment, Bst 2.0, Bst 2.0 WarmStart, Bst 3.0).

### Example 10: Digest-LAMP Assays

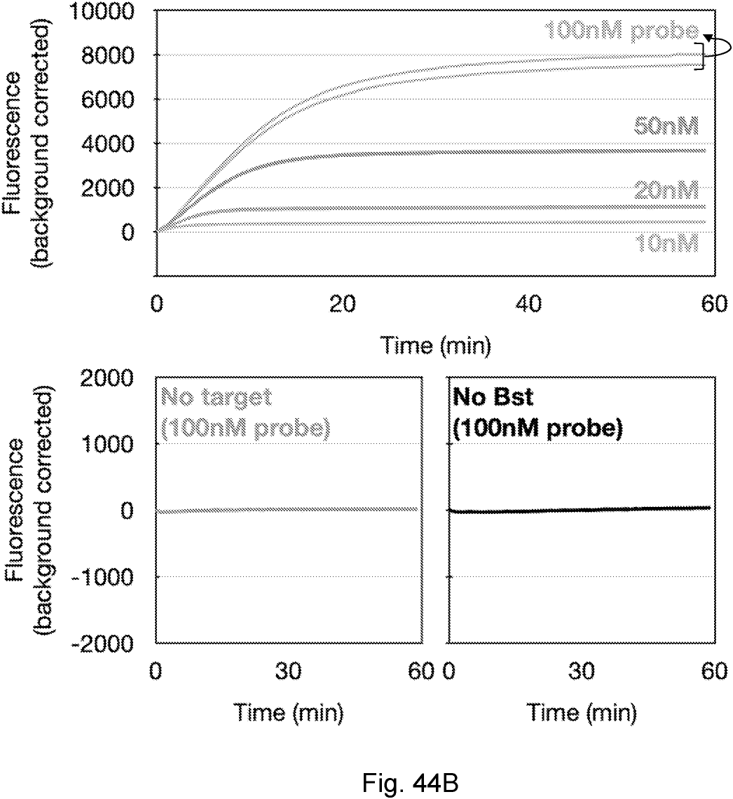

Described herein is specific detection of target and amplified signal production by catalytic turnover of digest probes (see e.g., FIGS. 44A-44B). Experimental setup: (see e.g., FIG. 44A) A target DNA amplicon (e.g., with sequence CGG TGG ACA AAT TGT CAC CTG TGC AAA GGA AAT TAA GGA GAG TGT TCA GAC ATT CTT TAA GCT TGT AAA TAA ATT TTT GGC TTT GTG TGC TGA CTC TAT CAT TAT TGG TGG AGC TAA ACT TAA AGC CTT GAA TTT AGG TGA AAC ATT TGT CAC GCA CTC AAA GGG ATT GTA CAG AAA GTG TGT TAA ATC CAG AGA AG, SEQ ID NO: 4, which corresponds to nt 1980-2176 of SEQ ID NO: 3 (SARS-CoV-2 ORFlab) was mixed with a Digest probe (e.g., with sequence /56-FAM/ CCA CCA ATA /ZEN/ ATG ATA GAG TCA GCA CAC A /3IABkFQ/, SEQ ID NO: 19, where /56-FAM/ is a FAM fluorescent molecule and /ZEN/ and /3IABkFQ/ are quencher molecules) at a temperature of 60° C. The concentration of the target was 10 nM while the concentration of the probe was varied as 10 nM, 20 nM, 50 nM and 100 nM respectively in four different tubes. Bst Full Length enzyme was included (except in No Bst conditions) at a concentration of 0.1 U/µL. The progress of probe digestion was monitored using a real time PCR machine that monitors fluorescence. Results: FIG. 44B shows an increase in fluorescent signal in proportion to increasing probe concentration, while the target concentration was fixed. This shows the catalytic turnover of probes by digestion due to the double-strand specific 5 prime to 3 prime exonuclease activity of the included Bst Full Length enzyme. In the absence of the enzyme (No Bst, bottom right of FIG. 44B) or absence of the target (No target, bottom left of FIG. 44C) there is no discernible signal increase even in the presence of 100 nM of probe, showing that probe digestion is target specific and driven by the Bst Full Length enzyme. Thus, the Digest probe technology can detect a target and produce high (amplified) signals far in excess of what can be achieved by mere stoichiometric probes (e.g. Taqman™ probe or Molecular Beacons).

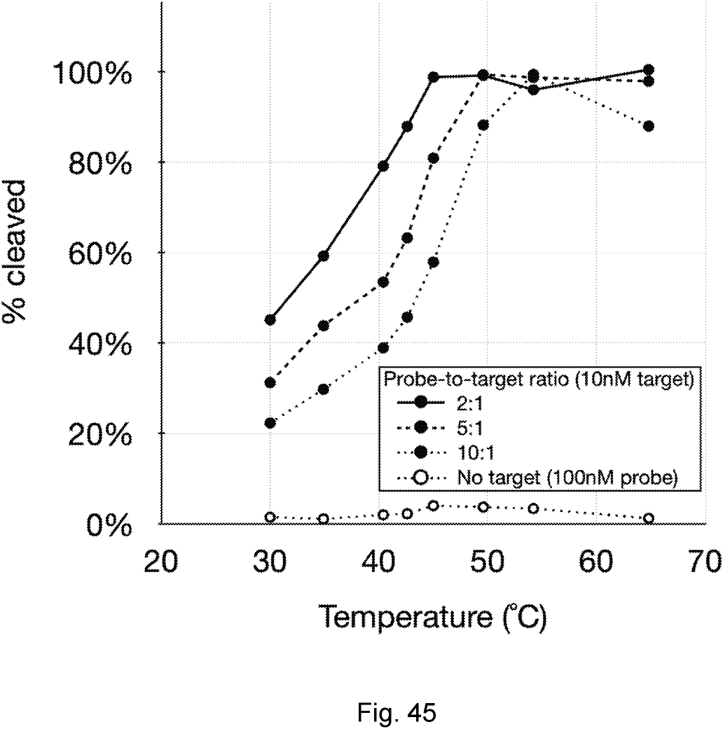

Digest probes exhibit robustness under a range of temperatures (see e.g., FIG. 45). Experimental setup: Like in FIGS. 44A-44B, for FIG. 45 a target DNA amplicon (10 nM) was mixed with Digest-probes at various concentrations: 20 nM (2:1), 50 nM (5:1), and 100 nM (10:1) in the presence of Bst Full Length enzyme (0.1 U/µL). The progress of probe digestion was monitored using a real time PCR machine for 30 minutes. At the end of 30 minutes, a non-specific DNA endonuclease was introduced to digest all the probes, and the end point fluorescence was recorded. The percentage of probes digested at the end of 30 minutes were calculated against this end point. Results: Probe digestion was observed over a wide range of temperatures, ranging from 30° C. to 65° C. Probe digestion was most efficient in the temperature range of 50° C. to 65° C., where up to a 5-fold excess of probes was 100% digested in 30 minutes (see e.g., FIG. 45). The digestion efficiency depends on various factors like probe sequence, probe length, buffer composition, salt concentration, target sequence, target length etc.

Digest-LAMP exhibits superior specificity compared to LAMP detection (see e.g., FIG. 46). Experiment setup: The specificity of Digest-LAMP was compared to conventional LAMP amplification where a dye that fluoresces only on binding to dsDNA (STYO-9) is used to generate signal. Digest probes (As1e.mid28.Cy5, E1.mid29.Cy5, and S-123.mid28.Cy5) and SYTO-9 were both included in the same LAMP reaction. The digest probes contain a Cy5 dye which fluoresces in the red channel while the SYTO-9 dye fluoresces in the blue channel. The fluorescence was simultaneously recorded in both these independent channels using a real time PCR machine. The experimental conditions mimic the conditions under which a diagnostic for the presence of SARS-CoV-2 virus operates. In particular, the target was synthetic SARS-CoV-2 RNA in a matrix of pooled human nasal fluid (labeled positive control, three repeats) or a matrix of pooled human nasal fluid with no SARS-CoV-2 RNA (labeled NTC, i.e. no template control, 93 repeats). LAMP primer sets As1e, S-123 and E1 were used for amplification. Results: On the left of FIG. 46, Digest-LAMP only produces signal in the presence of the target (SARS-CoV-2 RNA), while no signal above the detection threshold is produced in the absence of target. In contrast, on the right of FIG. 46, LAMP results in non-specific amplification in the absence of target, resulting in a false positive signal above detection threshold. Note that even in cases where there is strong a false positive signal in the SYTO-9 channel, no signal above detection threshold is produced in the Cy5 digest-probe channel, implying that the probe has the ability to distinguish spurious amplicons from genuine amplicons generated from the target.

Digest-LAMP allows for specific detection of infectious diseases (see e.g., FIG. 47).

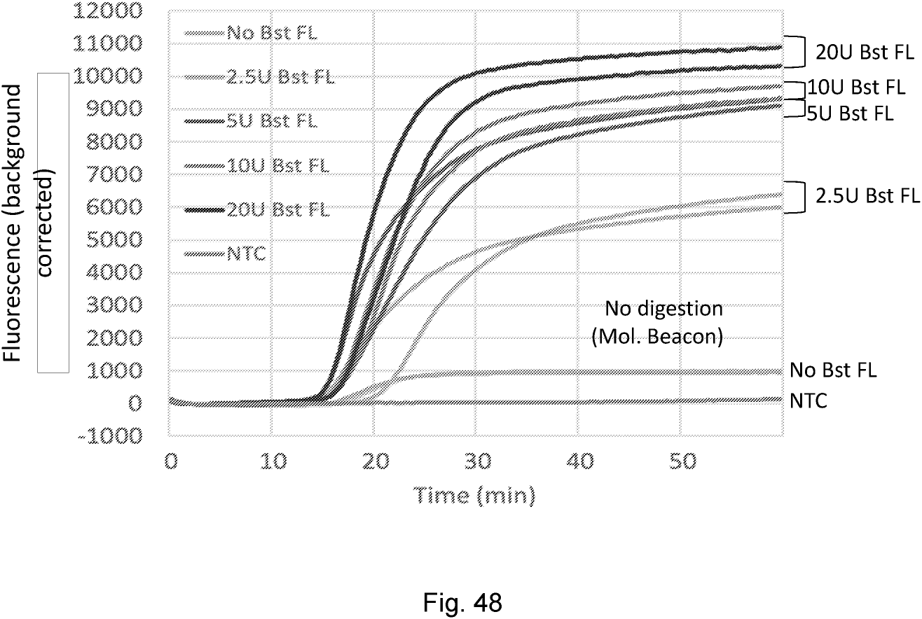

Digest-LAMP exhibits superior signal compared to molecular beacon technology (see e.g., FIG. 48). Experimental setup: A Digest-LAMP reaction for detecting SARS-CoV-2 RNA was set up in a total volume of 50 µL as follows: 200 copies of synthetic SARS-CoV-2 RNA fragments, NEB Warm Start LAMP master mix (25 µL), RNase Inhibitor Murine (0.5 U/µL), Guanidine Hydrochloride (40 mM), Bst Full Length (at the concentration as indicated in FIG. 48), As1e.FIP (1.6 µM), As1e.BIP (1.6 µM), As1e.F3 (0.2 µM), As1e.B3 (0.2 µM), As1e.LF (0.4 µM), As1e.LB (0.4 µM), and As1e.mid28 digest probe (0.2 µM). The reaction was incubated at a 65° C. in a real time PCR machine and fluorescence was recorded approximately every 30 seconds for an hour.

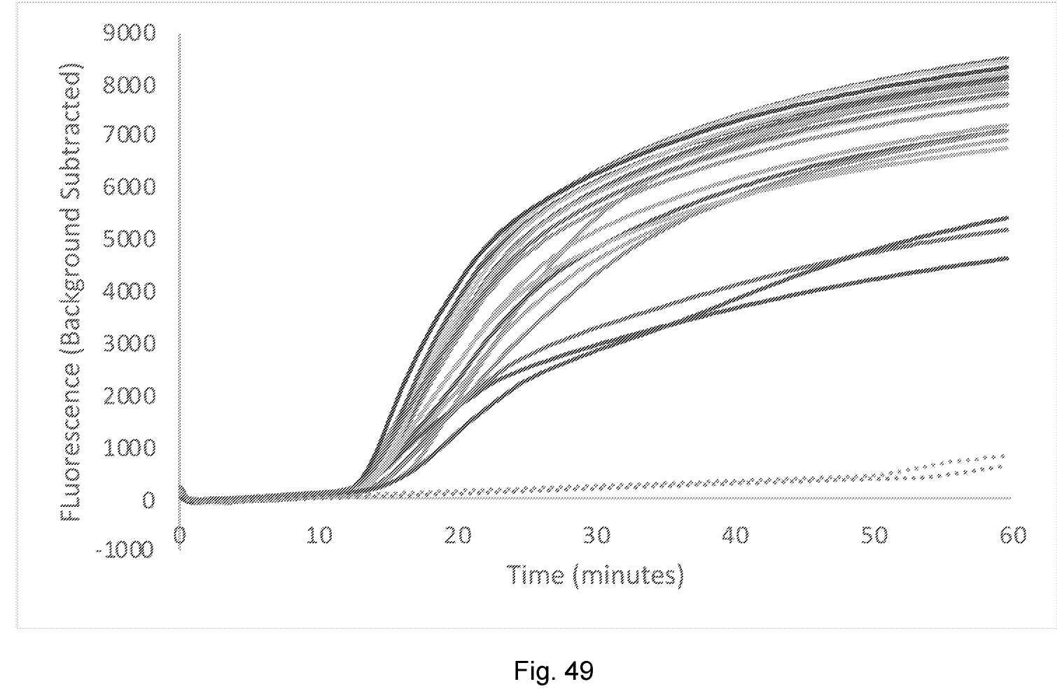

Digest-LAMP robustly detects SARAs-COV-2 (see e.g., FIG. 49). Experimental conditions: A Digest-LAMP reaction for detecting SARS-CoV-2 RNA was set up in a total volume of 50 µL as follows: 100 copies of synthetic SARS-CoV-2 RNA fragments, NEB Warm Start LAMP master mix (25 µL), RNase Inhibitor Murine (0.5 U/µL), Guanidine Hydrochloride (40 mM), Bst Full Length (0.2 U/µL), As1e.FIP (1.6 µM), As1e.BIP (1.6 µM), As1e.F3 (0.2 µM), As1e.B3 (0.2 µM), As1e.LF (0.4 µM), As1e.LB (0.4 µM), As1e.mid28 digest probe (0.2 µM), E1.FIP (1.6 µM), E1.BIP (1.6 µM), E1.F3 (0.2 µM), E1.B3 (0.2 µM), E1.LF (0.4 µM), E1.LB (0.4 µM), E1.mid29 digest probe (0.2 µM), S-123.FIP (1.6 µM), S-123.BIP (1.6 µM), S-123.F3 (0.2 µM), S-123.B3 (0.2 µM), S-123.LF (0.4 µM), S-123.LB (0.4 µM), and S-123.mid28 digest probe (0.2 µM). The reaction was incubated at a 65° C. in a real time PCR machine and fluorescence was recorded approximately every 30 seconds for an hour.

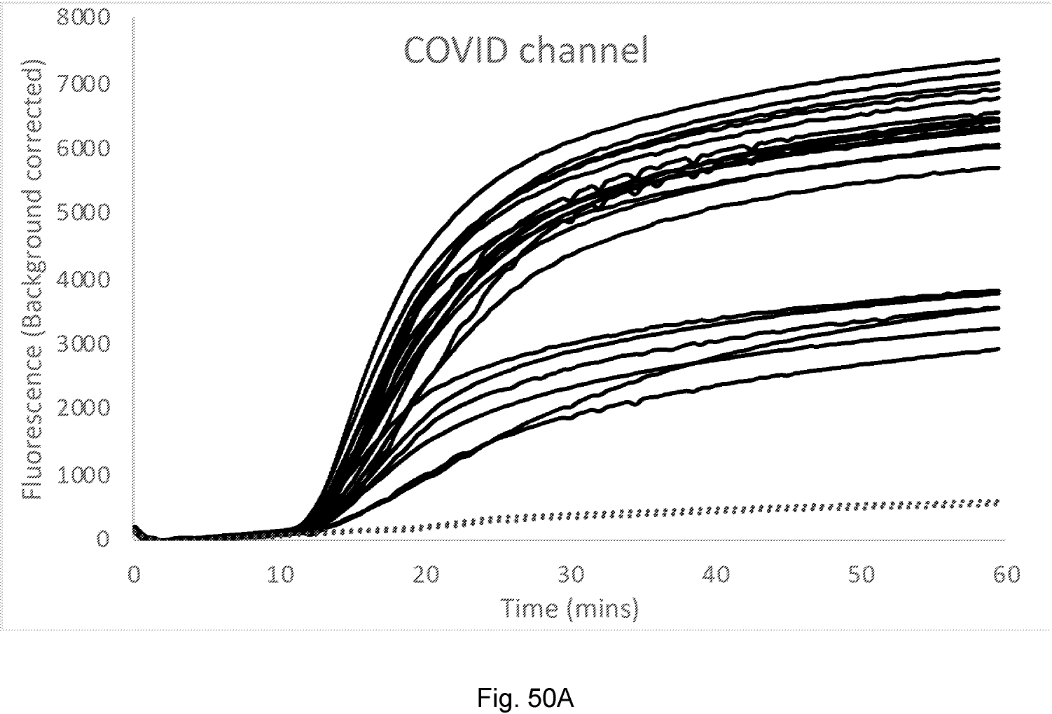

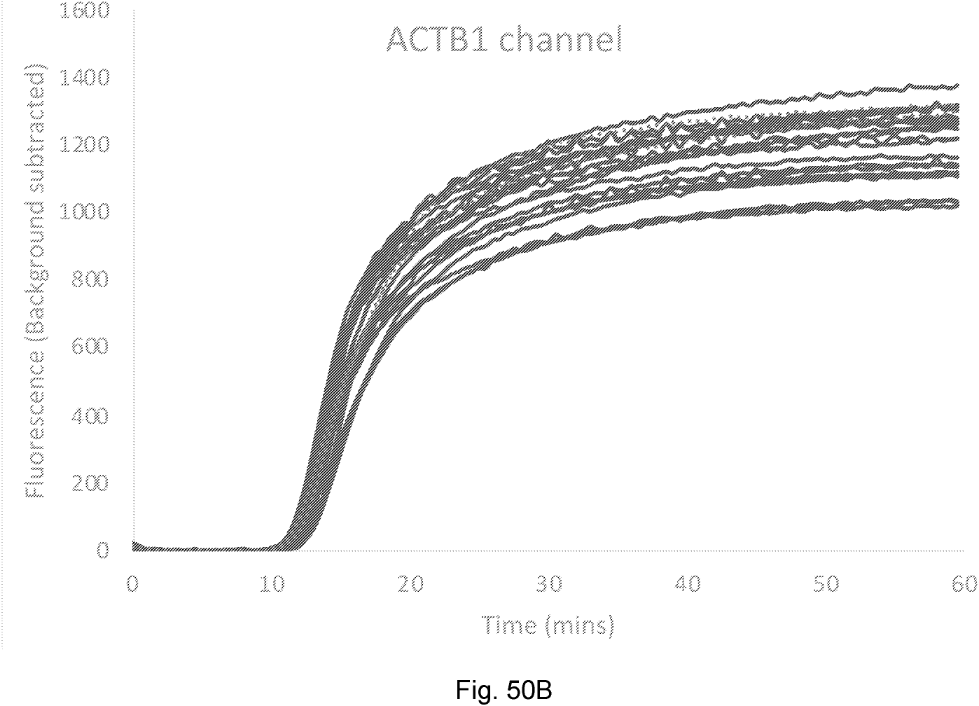

Multiplexed Digest-LAMP can detect SARS-CoV-2 RNA and a human specimen control (e.g., ACTB1) in the same tube (see e.g., FIGS. 50A-50B). Experimental conditions: A Digest-LAMP reaction for detecting SARS-CoV-2 RNA and human specimen control was setup in a total volume of 40 µL as follows: 100 copies of synthetic SARS-CoV-2 RNA fragments, 10 µL of clinical nasal elute, NEB Warm Start LAMP master mix (20 µL), RNase Inhibitor Murine (0.5 U/µL), Guanidine Hydrochloride (40 mM), Bst Full Length (0.2 U/µL), As1e.FIP (1.6 µM), As1e.BIP (1.6 µM), As1e.F3 (0.2 µM), As1e.B3 (0.2 µM), As1e.LF (0.4 µM), As1e.LB (0.4 µM), As1e.mid28 digest probe (0.2 µM), E1.FIP (1.6 µM), E1.BIP (1.6 µM), E1.F3 (0.2 µM), E1.B3 (0.2 µM), E1.LF (0.4 µM), E1.LB (0.4 µM), E1.mid29 digest probe (0.2 µM), S-123.FIP (1.6 µM), S-123.BIP (1.6 µM), S-123.F3 (0.2 µM), S-123.B3 (0.2 µM), S-123.LF (0.4 µM), S-123.LB (0.4 µM), and S-123.mid28 digest probe (0.2 µM). The reaction was incubated at a 65° C. in a real time PCR machine and fluorescence was recorded approximately every 30 seconds for an hour.

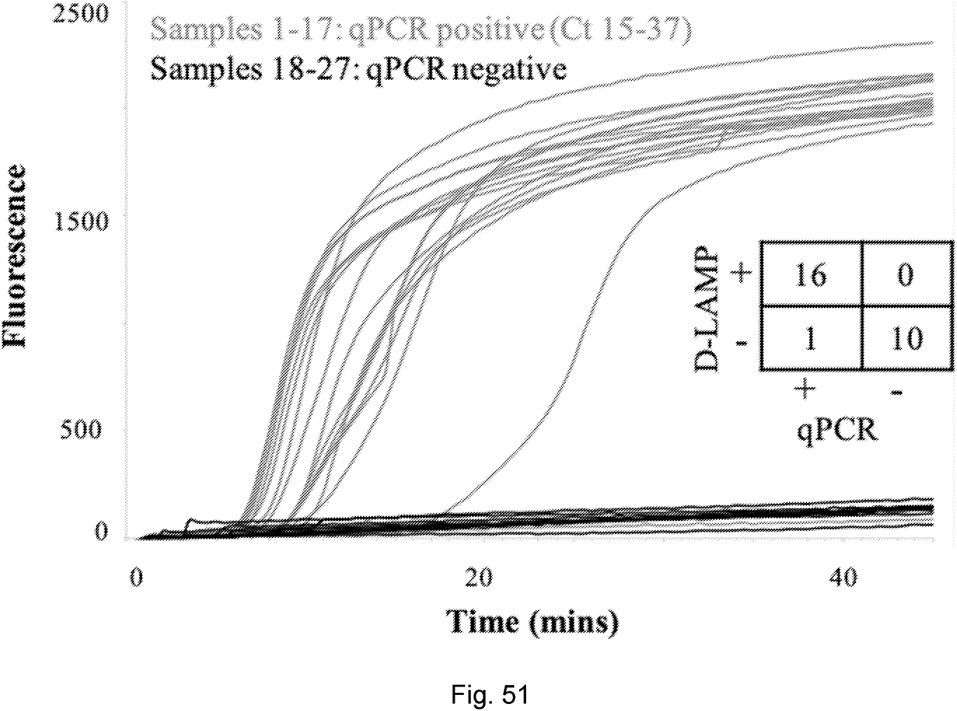

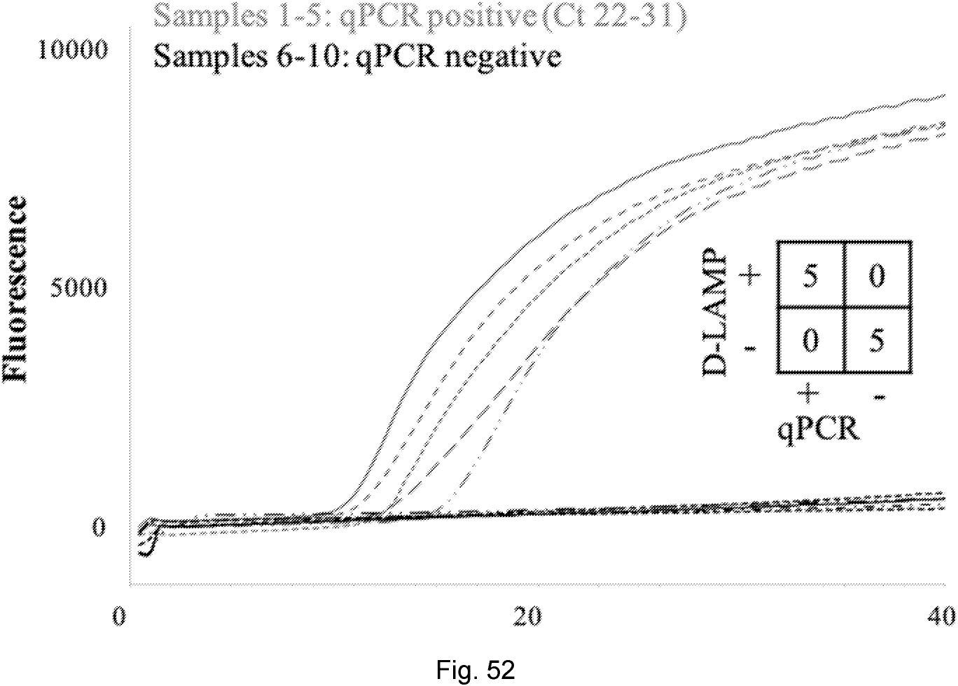

Digest-LAMP detects SARS-CoV-2 in nasal samples (see e.g., FIG. 51). Digest-LAMP detects SARS-CoV-2 in saliva samples (see e.g., FIG. 52).

The probe can be designed to bind to any fully or partially exposed single stranded region of the amplicon (see e.g., FIGS. 53A-53D). In FIG. 53A the probe binds to the single stranded stem region of the double hairpin while in FIG. 53B it binds to one of the single stranded loop regions, and in FIG. 53C it binds to a partially exposed single stranded region in the stem and displaces the double stranded amplicon regions. As shown in FIG. 53D, the fluorophore and quencher regions can each be located either at 5′ or 3′ ends on the probe, or they can be located internal to the probe. More than one quencher or fluorophore can be contained in one probe for obtaining an enhanced signal. As shown in FIG. 53E, the probe can comprise a plurality of strands hybridized together, for instance as shown in the top panel of FIG. 53E. The probe can also have an internal partial secondary structure like a hairpin. The probe can have a 3′ overhang when hybridized to the target.

Table 3: Primer sets used herein in various detection experiments. As1e is a primer set that targets the ORF1a gene, E1 targets the E gene, and S-123 targets the S gene respectively of SARS-CoV-2. ACTB1 is a primer set that targets the human ACTB1 gene, which serves as a human specimen control in diagnostic assays.

| SEQ ID NO: | Strand | Sequence | Length |
|---|---|---|---|
| 21 | As1e.F3 | CGGTGGACAAATTGTCAC | 18 |
| 22 | As1e.B3 | CTTCTCTGGATTTAACACACTT | 22 |
| 23 | As1e.FIP | TCAGCACACAAAGCCAAAAATTTATTTTTCTGTGCAAA GGAAATTAAGGAG | 51 |
| 24 | As1e.BIP | TATTGGTGGAGCTAAACTTAAAGCCTTTTCTGTACAATC CCTTTGAGTG | 49 |
| 25 | As1e.LF | TTACAAGCTTAAAGAATGTCTGAACACT | 28 |
| 26 | As1e.LB | TTGAATTTAGGTGAAACATTTGTCACG | 27 |
| 27 | E1.F3 | TGAGTACGAACTTATGTACTCAT | 23 |
| 28 | E1.B3 | TTCAGATTTTTAACACGAGAGT | 22 |
| 29 | E1.FIP | ACCACGAAAGCAAGAAAAAGAAGTTCGTTTCGGAAGA GACAG | 42 |
| 30 | E1.BIP | TTGCTAGTTACACTAGCCATCCTTAGGTTTTACAAGACT CACGT | 44 |
| 31 | E1.LF | CGCTATTAACTATTAACG | 18 |
| 32 | E1.LB | GCGCTTCGATTGTGTGCGT | 19 |
| 33 | S-123.F3 | TCTATTGCCATACCCACAA | 19 |
| 34 | S-123.B3 | GGTGTTTTGTAAATTTGTTTGAC | 23 |
| 35 | S-123.FIP | CATTCAGTTGAATCACCACAAATGTGTGTTACCACAGA AATTCTACC | 47 |
| 36 | S-123.BIP | GTTGCAATATGGCAGTTTTTGTACATTGGGTGTTTTTGT CTTGTT | 45 |
| 37 | S-123.LF | ACTGATGTCTTGGTCATAGACACT | 24 |
| 38 | S-123.LB | TAAACCGTGCTTTAACTGGAATAGC | 25 |
| 39 | ACTB1.F3 | AAGATGAGATTGGCATGGC | 19 |
| 40 | ACTB1.B3 | GCAAGGGACTTCCTGTAAC | 19 |
| 41 | ACTB1.FI P | CTCCAACCGACTGCTGTCTTTGGCTTGACTCAGGATTT | 38 |
| 42 | ACTB1.BI P | CCCAAAGTTCACAATGTGGCCGCATCTCATATTTGGAAT GAC | 42 |
| 43 | ACTB1.LF | ACCTTCACCGTTCCAGTT | 18 |
| 44 | ACTB1.LB | GGACTTTGATTGCACATTGTTG | 22 |
| 45 | DetRP.F3 | TTGATGAGCTGGAGCCA | 17 |
| 46 | DetRP.B3 | CACCCTCAATGCAGAGTC | 18 |
| 47 | DetRP.FIP | GTGTGACCCTGAAGACTCGGTTTTAGCCACTGACTCGG ATC | 41 |
| 48 | DetRP.BIP | CCTCCGTGATATGGCTCTTCGTTTTTTTCTTACATGGCTC TGGTC | 45 |
| 49 | DetRP.LF | ATGTGGATGGCTGAGTTGTT | 20 |
| 50 | DetRP.LB | CATGCTGAGTACTGGACCTC | 20 |

Table 4: Probes used herein in various detection experiments. As1e.mid28 is a probe that targets the ORF1a gene, E1.mid29 targets the E gene, and S-123.mid28 targets the S gene respectively of SARS-CoV-2. ACTB1.mid28 is a probe that targets the human ACTB gene, while DetRP.mid30 is a probe that targets human ribozyme RNAseP, both of which may serve as human specimen control in diagnostic assays.

| SEQ ID NO: | Strand | Sequence | Length |
|---|---|---|---|
| 51 | As1e.mid28.Cy5 | /5Cy5/TGTGTGCTG/TAO/ACTCTATCATTATTGGTGG /3IAbRQSp/ | 28 |
| 52 | E1.mid29.Cy5 | /5Cy5/TTGCTTTCG/TAO/TGGTATTCTTGCTAGTTAC A/3IAbRQSp/ | 29 |
| 53 | S-123.mid28.Cy5 | /5Cy5/ACTGAATGC/TAO/AGCAATCTTTTGTTGCAAT /3IAbRQSp/ | 28 |
| 54 | ACTB1.mid28.FA M | /56-FAM/CGGTTGGAG/ZEN/CGAGCATCCCCCAAAGTTC /3IABkFQ/ | 28 |
| 55 | DetRP.mid30.FAM | /56-FAM/AAGTAATTG/ZEN/AAAAGACACTCCTCCACTT AT/3IABkFQ/ | 30 |

### Example 11: Double Stranded Target/amplicon and Signal Amplification by Digestion

Double-stranded nucleic acid targets can be detected using nucleic acid probes as described herein (see e.g., FIGS. 54A-54C). Experimental setup: Unless specified otherwise, the double-stranded target detection reaction is composed of 20 nM of dsDNA target molecules (except for the no target control), 100 nM of digest probe, 1x Isothermal Amplification Buffer (NEB #B0537S), 2 mM MgSO₄, and 0.1 U/µL of Bst DNA Polymerase Full Length (NEB #M0328S) in a final reaction volume of 25 µL. The reaction was incubated at 65° C. for 1 hour and a fluorescence readout of digest probes was recorded at every 1 min using a real-time PCR system (Bio-Rad™ CFX96) from the beginning of the incubation. The sequences of the digest probe and the dsDNA target are given below.

SEQ ID NO: 19, Digest probe (28nt): (from 5′ to 3′) /6-FAM/ CCACCAATA /ZEN/ ATGATAGAGTCAGCACACA /IABkFQ/, where /6-FAM/ is a FAM fluorescent molecule and /ZEN/ and /IABkFQ/ are quencher molecules.

SEQ ID NO: 4, dsDNA target (197 bp): Target strand (5′ to 3′); the digest probe binding site is indicated by bolded, double-underlined text (e.g., nt 84-111 of SEQ ID NO: 4):

CGGTGGACAAATTGTCACCTGTGCAAAGGAAATTAAGGAGAGTGTTCAGACATTCTTTA
AGCTTGTAAATAAATTTTTGGCTTTGTGTGCTGACTCTATCATTATTGGTGGAGCTAAA
CTTAAAGCCTTGAATTTAGGTGAAACATTTGTCACGCACTCAAAGGGATTGTACAGAAAG
TGTGTTAAATCCAGAGAAG

SEQ ID NO: 20, dsDNA target (197 bp): Complementary strand (5′ to 3′):

CTTCTCTGGATTTAACACACTTTCTGTACAATCCCTTTGAGTGCGTGACAAATGTTTCACC
TAAATTCAAGGCTTTAAGTTTAGCTCCACCAATAATGATAGAGTCAGCACACAAAGCCA
AAAATTTATTTACAAGCTTAAAGAATGTCTGAACACTCTCCTTAATTTCCTTTGCACAGGT
GACAATTTGTCCACCG

Unless noted otherwise, in Example 11 and the associated figures (e.g., FIGS. 54-57) “dsDNA target” refers to this 197-bp dsDNA target sequence (i.e., SEQ ID NOs: 4,20). SEQ ID NOs: 4 and 20 correspond to nt 1980-2176 of SEQ ID NO: 3 (SARS-CoV-2 ORF1ab).

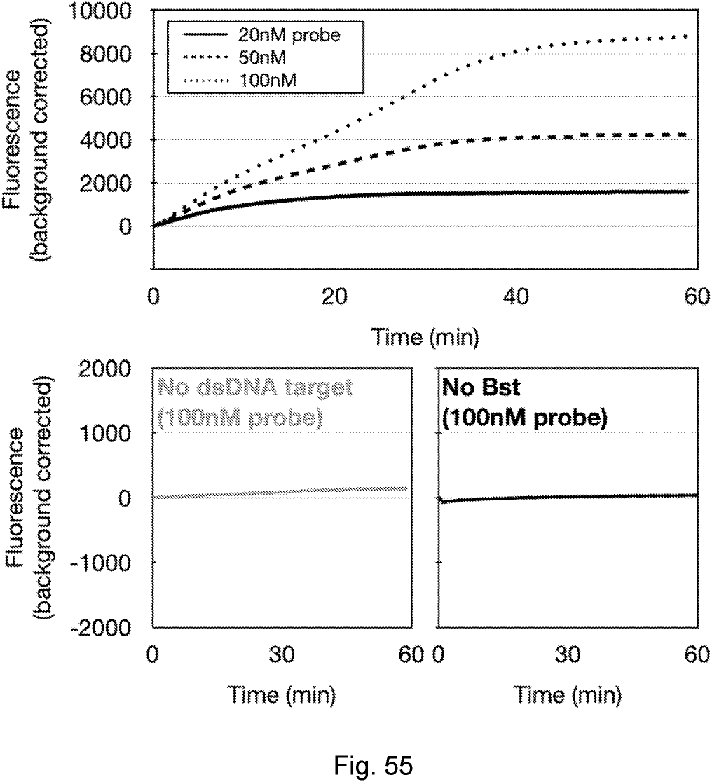

A range of probe concentrations can be used when detecting a double-stranded target (see e.g., FIG. 55). Experimental setup: The reaction conditions were the same as for FIG. 54 except that the concentration of the Bst Full Length polymerase was increased to 0.2 U/µL. The unprotected dsDNA target molecules were used for all reactions in FIG. 55. Results: The positive proportionality between the end-point fluorescence level and the probe concentration indicates the catalytic digestion turnover of probes (see e.g. top graph of FIG. 55). When no dsDNA target (see e.g., bottom left graph of FIG. 55) was added, the signal remained close to the background level even in the presence of 100 nM of probe, which indicates that the signal production is target-specific. The signal also remained close to zero when no Bst Full Length polymerase (see e.g., bottom right graph of FIG. 55) was added, which indicates the binding of probe is assisted by the partial-digestion of dsDNA target by the Bst Full Length polymerase. These results indicate that double-stranded molecules can be efficiently detected in a single incubation reaction without any pre-processing to expose the target strand for probe binding.

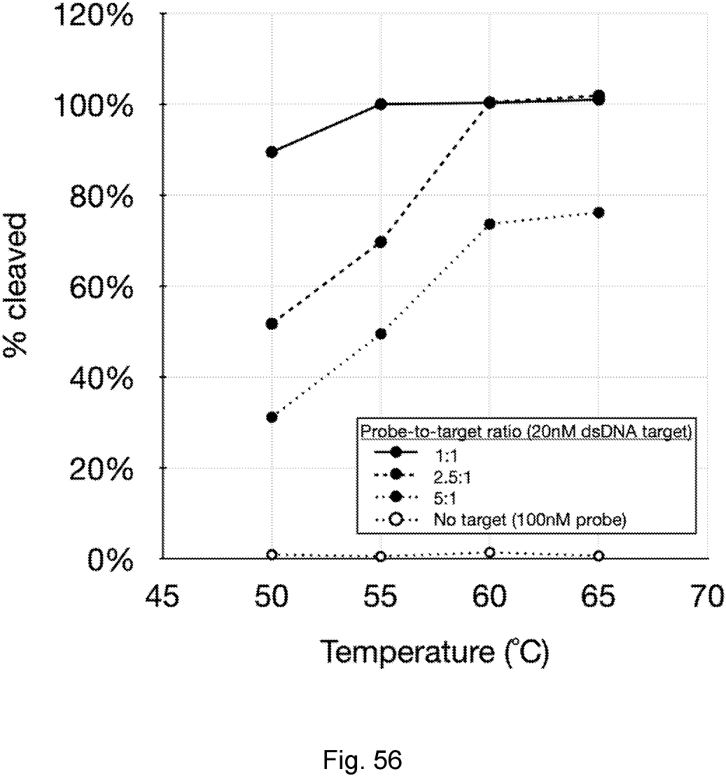

Detectible probe digestion was observed over a range of temperatures using dsDNA target (see e.g., FIG. 56). Experimental setup: The reaction conditions were the same as noted for FIGS. 54-55, but with 20 nM dsDNA target (unprotected) mixed with digest probes at three different concentrations: 20 nM (1:1 probe-to-target ratio), 50 nM (2.5:1) and 100 nM (5:1) in the presence of 0.2 U/µL Bst Full Length polymerase. The progress of probe digestion was monitored every 1 min using a real time PCR machine for 30 min at four different temperatures, 50° C., 55° C., 60° C., and 65° C. At the end of the 30 min incubation, 20 units of T5 exonuclease (NEB #M0663S) was added to completely digest all the unreacted probes in the reaction and the completely-digested (i.e., 100% cleavage efficiency) fluorescence was also recorded. The cleavage efficiency of probes was then calculated from the end-point fluorescence after the 30-min incubation divided by the fluorescence after the T5 treatment. Results: Detectible probe digestion was observed over a range of temperatures, from 50° C. to 65° C. (see e.g., FIG. 56). Probe digestion was most efficient in the temperature range of 60° C. to 65° C., where up to a 2.5-fold excess of probes was 100% digested in 30 minutes. The digestion efficiency depends on various factors like probe sequence, probe length, buffer composition, salt concentration, dsDNA target sequence, dsDNA target length, etc.

Additional detection methods for a double-stranded nucleic acid target includes combining digest probes with single-strand binding (SSB) proteins (see e.g., FIG. 57).

What is claimed herein is:

1. A method for detecting an amplicon from amplification of a target nucleic acid in a sample, the method comprising:
hybridizing a nucleic acid probe to an amplicon from amplification of a target nucleic acid, wherein the nucleic acid probe comprises a nucleotide sequence substantially complementary or identical to a nucleotide sequence of the target nucleic acid or a primer in used in the amplification of the target nucleic acid, wherein the nucleic acid probe comprises a reporter molecule capable of producing a detectable signal, and wherein the detectable signal from the reporter molecule is partially quenched when the nucleic acid probe is hybridized to the amplicon;
cleaving the hybridized nucleic acid probe with a double-strand specific exonuclease having 5′ to 3′ exonuclease activity; and
detecting the reporter molecule from the cleaved nucleic acid probe or detecting any remaining uncleaved nucleic acid probe.

2. The method of claim 1, wherein said hybridizing the nucleic acid probe or cleaving the hybridized nucleic acid probe is simultaneous with the amplification of the target nucleic acid.

3. The method of claim 1, wherein said hybridizing the nucleic acid probe or cleaving the hybridized nucleic acid probe is after the amplification of the target nucleic acid.

4. The method of claim 1, wherein the reporter molecule is selected from the group consisting of fluorescent molecules, radioisotopes, chromophores, enzymes, enzyme substrates, chemiluminescent moieties, bioluminescent moieties, echogenic substances, non-metallic isotopes, optical reporters, paramagnetic metal ions, and ferromagnetic metals.

5. The method of claim 1, wherein the nucleic acid probe further comprises a quencher molecule.

6. The method of claim 5, wherein the quencher molecule quenches the detectable signal from the reporter molecule when the nucleic acid probe is not hybridized to the amplicon.

7. The method of claim 5, wherein the quencher molecule quenches the detectable signal from the reporter molecule when the nucleic acid probe is hybridized to the amplicon.

8. The method of any one of claims 5, wherein the nucleic acid probe further comprises at least one additional quencher molecule.

9. The method of claim 1, wherein the nucleic acid probe comprises a plurality of reporter molecules.

10. The method of claim 9, wherein at least two reporter molecules in the plurality of reporter molecules are different.

11. The method of claim 1, wherein at least one primer used in the amplification comprises a nucleic acid modification capable of inhibiting the 5′->3′ exonuclease activity of the exonuclease.

12. The method of claim 1, wherein the nucleic acid probe comprises at least one nucleic acid modification.

13. The method of claim 1, wherein the nucleic acid probe comprises at least one nucleic acid modification capable of increasing a melting temperature (Tm) of the nucleic acid probe for hybridizing with a complementary strand relative to a nucleic acid probe lacking said modification.

14. The method of claim 1, wherein the nucleic acid probe comprises at least one nucleic acid modification capable of inhibiting extension by a polymerase.

15. The method of claim 1, wherein the exonuclease lacks polymerase activity.

16. The method of claim 1, wherein the exonuclease has polymerase activity.

17. The method of claim 1, wherein the exonuclease is selected from the group consisting of Bst Full Length, Taq DNA polymerase, T7 Exonuclease, Exonuclease VIII, Exonuclease VIII truncated, Lambda exonuclease, T5 Exonuclease, RecJf, and any combination thereof.

18. The method of claim 1, wherein said amplification is isothermal amplification.

19. The method of claim 1, wherein said amplification is selected from the group consisting of: Loop Mediated Isothermal Amplification (LAMP), Recombinase Polymerase Amplification (RPA), Helicase-dependent isothermal DNA amplification (HDA), Rolling Circle Amplification (RCA), Nucleic acid sequence-based amplification (NASBA), strand displacement amplification (SDA), nicking enzyme amplification reaction (NEAR), polymerase Spiral Reaction (PSR), Hybridization Chain Reaction (HCR), Primer Exchange Reaction (PER), Signal Amplification by Exchange Reaction (SABER), transcription-based amplification system (TAS), Self-sustained sequence replication reaction (3SR), Single primer isothermal amplification (SPIA), and cross-priming amplification (CPA).

20. The method of claim 1, wherein said amplification is Loop-mediated Isothermal Amplification (LAMP).

21. The method of claim 1, wherein the amplicon is single-stranded.

22. The method of claim 21, wherein the method further comprises a step of preparing the single-stranded amplicon from the target nucleic acid prior to hybridizing the nucleic acid probe with the amplicon.

23. The method of claim 1, wherein said detecting the reporter molecule comprises detecting a detectable signal produced by the reporter molecule.

24. The method of claim 1, wherein said detecting the reporter molecule comprises fluorescence detection, luminescence detection, chemiluminescence detection, colorimetric detection, or immunofluorescence detection.

25. The method of claim 1, wherein said detecting the reporter molecule comprises a lateral flow assay.

26. The method of claim 1, wherein the nucleic acid probe comprises a ligand for a ligand binding molecule.

27. The method of claim 1, wherein the nucleic acid probe comprises a lateral flow detectable moiety.

28. The method of claim 1, wherein said detecting the uncleaved nucleic acid probe comprises sequence-specific detection.

29. The method of claim 28, wherein said sequence-specific detection comprises toehold-mediated strand displacement, probe-based electrochemical readout, micro-array detection, sequence-specific amplification, hybridization with conjugated or unconjugated nucleic acid strand, colorimetric assays, gel electrophoresis, molecular beacons, fluorophore-quencher pairs, microarrays, sequencing or any combinations thereof.

30. The method of claim 1, wherein said detecting the uncleaved nucleic acid probe comprises lateral flow detection.

31. The method of claim 1, wherein the nucleic acid probe is immobilized on a surface.

32. The method of claim 1, wherein at least one primer used in the amplification is immobilized on a surface.

33. The method of claim 1, wherein the nucleic acid probe comprises a nucleotide sequence substantially complementary to a primer used in the amplification of the target nucleic acid.

34. The method of claim 1, wherein the nucleic acid probe comprises a nucleotide sequence substantially identical to a primer used in the amplification of the target nucleic acid.

35. The method of claim 1, wherein the nucleic acid probe comprises a nucleotide sequence substantially complementary to a nucleotide sequence at an internal position of the amplicon.

36. The method of claim 1, wherein the nucleic acid probe comprises a first nucleic acid strand and a second nucleic acid strand, wherein the first strand comprises a region that is substantially complementary to a region in the second strand.

37. The method of claim 36, wherein the first and second strands are linked to each other.

38. The method of claim 1, wherein the nucleic acid probe forms a hairpin structure when hybridized to the amplicon.

39. The method of claim 1, wherein the nucleic acid probe comprises a single-stranded region when hybridized to the amplicon.

40. The method of claim 1, wherein said detection is multiplexed detection of at least two target nucleic acids.

41. The method of claim 1, wherein the method is performed in a device comprising two or more chambers and means for irreversibly moving a fluid from a first chamber to a second chamber.

42. The method of claim 41, wherein the means for irreversibly moving the fluid from the first to the second chamber can be actuated by a built-in spring whose potential energy is released by a solenoid trigger.

43. The method of claim 42, wherein the device further comprises means for detecting the detectable signal from the reporter molecule.

44. A kit for detecting a target nucleic acid in a sample, the kit comprising
a) an exonuclease having 5′->3′ cleaving activity;
b) a primer set for amplifying a target nucleic acid; and
c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set.

45. The kit of claim 44, wherein said amplification is LAMP and the primer set comprises a forward outer primer (F3), a reverse outer primer (R3), a forward inner primer (FIP), and a reverse inner primer (RIP).

46. The kit of claim 45, wherein the primer set further comprises a forward loop primer (LF), and a reverse loop primer (LR).

47. The kit of claim 44, wherein the nucleic acid probe comprises further comprises a quencher molecule.

48. The kit of claim 47, wherein the quencher molecule quenches the detectable signal from the reporter molecule when the nucleic acid probe is not hybridized to a complementary nucleic acid strand.

49. The kit of claim 47, wherein the quencher molecule quenches the detectable signal from the reporter molecule when the nucleic acid probe is hybridized to a complementary nucleic acid strand.

50. The kit of claim 47, wherein the nucleic acid probe further comprises at least one additional quencher molecule.

51. The kit of claim 44, wherein the nucleic acid probe comprises a plurality of reporter molecules.

52. The kit of claim 51, wherein at least two reporter molecules in the plurality of reporter molecules are different.

53. The kit of claim 44, wherein the nucleic acid probe comprises at least one nucleic acid modification capable of increasing a melting temperature (Tm) of the nucleic acid probe for hybridizing with a complementary strand relative to a nucleic acid probe lacking said modification.

54. The kit of claim 44, wherein the nucleic acid probe comprises at least one nucleic acid modification capable of inhibiting extension by a polymerase.

55. The kit of claim 44, wherein the kit further comprises a reference nucleic acid.

56. The kit of claim 44, wherein the kit further comprises a lateral flow device for detecting the reporter molecule.

57. The kit of claim 44, wherein the kit further comprises means for detecting a detectable signal from the reporter molecule.

58. The kit of claim 44, further comprising reagents for preparing a double-stranded amplicon from the target nucleic acid.

59. The kit of claim 44, further comprising reagents for preparing a single-stranded amplicon from the target nucleic acid.

60. The kit of claim 44, wherein the kit further comprises a DNA polymerase having strand displacement activity.

61. The kit of claim 44, wherein the kit further comprises dNTPs.

62. The kit of claim 44, wherein the kit further comprises a buffer.

63. The kit of claim 44, wherein the kit further comprises a device comprising two or more chambers and means for irreversibly moving a fluid from a first chamber to a second chamber.

64. The kit of claim 44, wherein at least one component of the kit is disposed in a device comprising two or more chambers and means for irreversibly moving a fluid from a first chamber to a second chamber.

65. The kit of claim 63 wherein the means for irreversibly moving the fluid from the first to the second chamber can be actuated by a built-in spring whose potential energy is released by a solenoid trigger.

66. The kit of claim 63, wherein the device further comprises means for detecting the detectable signal from the reporter molecule.

67. The kit of claim 44, wherein the nucleic acid probe comprises a nucleotide sequence substantially complementary to a primer in the primer set.

68. The kit of claim 44, wherein the nucleic acid probe comprises a nucleotide sequence substantially identical to a primer in the primer set.

69. The kit of claim 44, wherein the nucleic acid probe comprises a nucleotide sequence substantially complementary to a nucleotide sequence at an internal position of an amplicon prepared using the primer set.

70. The kit of claim 44, wherein the nucleic acid probe comprises a first nucleic acid strand and a second nucleic acid strand, wherein the first strand comprises a region that is substantially complementary to a region in the second strand.

71. The kit of claim 70, wherein the first and second strand are linked to each other.

72. The kit of claim 44, wherein the nucleic acid probe forms a hairpin structure when hybridized to a complementary nucleic acid.

73. A composition comprising:
a) an exonuclease having 5′->3′ cleaving activity;
b) a primer set for amplifying a target nucleic acid; and
c) a nucleic acid probe comprising a reporter molecule, wherein the reporter molecule is capable of producing a detectable signal, and wherein the probe comprises a nucleotide sequence substantially complementary or identical to a nucleotide sequence of the target nucleic acid or a primer in the primer set.

74. The composition of claim 73, wherein said amplification is LAMP and the primer set comprises a forward outer primer (F3), a reverse outer primer (R3), a forward inner primer (FIP), and a reverse inner primer (RIP).

75. The composition of claim 74, wherein the primer set further comprises a forward loop primer (LF), and a reverse loop primer (LR).

76. The composition of claim 73, wherein the nucleic acid probe further comprises a quencher molecule.

77. The composition of claim 76, wherein the quencher molecule quenches the detectable signal from the reporter molecule when the nucleic acid probe is not hybridized to a complementary strand.

78. The composition of claim 76, wherein the quencher molecule quenches the detectable signal from the reporter molecule when the nucleic acid probe is hybridized to a complementary nucleic acid strand.

79. The composition of claim 76, wherein the nucleic acid probe further comprises at least one additional quencher molecule.

80. The composition of claim 73, wherein the nucleic acid probe comprises a plurality of reporter molecules.

81. The composition of claim 80, wherein at least two reporter molecules in the plurality of reporter molecules are different.

82. The composition of claim 73, wherein the nucleic acid probe comprises at least one nucleic acid modification capable of increasing a melting temperature (Tm) of the nucleic acid probe for hybridizing with a complementary strand relative to a nucleic acid probe lacking said modification.

83. The composition of claim 73, wherein the nucleic acid probe comprises at least one nucleic acid modification capable of inhibiting extension by a polymerase.

84. The composition of claim 73, wherein the composition further comprises a reference nucleic acid.

85. The composition of claim 73, wherein the composition further comprises the target nucleic acid.

86. The composition of claim 73, further comprising reagents for preparing a double-stranded amplicon from the target nucleic acid.

87. The composition of claim 73, further comprising reagents for preparing a single-stranded amplicon from the target nucleic acid.

88. The composition of claim 73, wherein the composition further comprises a DNA polymerase having strand displacement activity.

89. The composition of claim 73, wherein the composition further comprises dNTPs.

90. The composition of claim 73, wherein the composition further comprises a buffer.

91. The composition of claim 73, wherein the composition is in lyophilized form.

92. The composition of claim 73, wherein one or more components of the composition is disposed in a device comprising two or more chambers and means for irreversibly moving a fluid from a first chamber to a second chamber.

93. The composition of claim 92, wherein the means for irreversibly moving the fluid from the first to the second chamber can be actuated by a built-in spring whose potential energy is released by a solenoid trigger.

94. The composition of claim 92, wherein the device further comprises means for detecting the detectable signal from the reporter molecule.

95. The composition of claim 73, wherein the nucleic acid probe comprises a nucleotide sequence substantially complementary to a primer used in the amplification of the target nucleic acid.

96. The composition of claim 73, wherein the nucleic acid probe comprises a nucleotide sequence substantially identical to a primer used in the amplification of the target nucleic acid.

97. The composition of claim 73, wherein the nucleic acid probe comprises a nucleotide sequence substantially complementary to a nucleotide sequence at an internal position of the amplicon.

98. The composition of claim 73, wherein the nucleic acid probe comprises a first nucleic acid strand and a second nucleic acid strand, wherein the first strand comprises a region that is substantially complementary to a region in the second strand.

99. The composition of claim 98, wherein the first and second strand are linked to each other.

100. The composition of claim 73, wherein the nucleic acid probe forms a hairpin structure when hybridized to a complementary nucleic acid.

101. The composition of claim 73, further comprising a single-stranded amplicon produced from the target nucleic acid.

102. The composition of claim 73, further comprising a double-stranded amplicon produced from the target nucleic acid.

103. A kit for detecting a target nucleic acid in a sample, the kit comprising a nucleic acid probe and wherein the nucleic acid probe comprises a nucleotide sequence selected from the group consisting of SEQ ID NOs: 51-55.

104. The kit of claim 103, wherein the kit further comprises an exonuclease having 5′->3′ cleaving activity.

105. The kit of claim 103, wherein the kit further comprise a primer set for amplifying a target nucleic acid.

106. The kit of claim 105, wherein said amplification is LAMP and the primer set comprises a forward outer primer (F3), a reverse outer primer (R3), a forward inner primer (FIP), and a reverse inner primer (RIP).

107. The kit of claim 106, wherein the primer set further comprises a forward loop primer (LF), and a reverse loop primer (LR).

108. The kit of claim 103, wherein the kit further comprises a reference nucleic acid.

109. The kit of claim 103, wherein the kit further comprises a lateral flow device.

110. The kit of claim 103, wherein the kit further comprises means for detecting a detectable signal from the nucleic acid probe.

111. The kit of claim 103, further comprising reagents for preparing a double-stranded amplicon from the target nucleic acid.

112. The kit of claim 103, further comprising reagents for preparing a single-stranded amplicon from the target nucleic acid.

113. The kit of claim 103, wherein the kit further comprises a DNA polymerase having strand displacement activity.

114. The kit of claim 103, wherein the kit further comprises dNTPs.

115. The kit of claim 103, wherein the kit further comprises a buffer.

116. The kit of claim 103, wherein the kit further comprises a device comprising two or more chambers and means for irreversibly moving a fluid from a first chamber to a second chamber.

117. The kit of claim 103, wherein at least one component of the kit is disposed in a device comprising two or more chambers and means for irreversibly moving a fluid from a first chamber to a second chamber.

118. The kit of claim 116 wherein the means for irreversibly moving the fluid from the first to the second chamber can be actuated by a built-in spring whose potential energy is released by a solenoid trigger.

119. The kit of claim 116, wherein the device further comprises means for detecting the detectable signal from the nucleic acid probe.

120. The kit of claim 105, wherein a primer in the primer set comprise a nucleotide sequence substantially complementary to the nucleic acid probe.

121. The kit of claim 105, wherein a primer in the primer set comprise a nucleotide sequence substantially identical to the nucleic acid probe.

122. The kit of claim 103, wherein an internal position of an amplicon prepared using the primer set comprises a nucleotide sequence substantially complementary to the nucleic acid probe.
